(19) United States
(12) Patent Application Publication
(10) Pub. No.: US 2024/0174764 A1
(43) Pub. Date: May 30, 2024

(54) METHODS FOR TREATING GYNECOLOGIC CANCER USING COMBINATION THERAPY WITH ANTI-MUC16 x CD3 MULTISPECIFIC ANTIBODIES AND VEGF INHIBITORS

(71) Applicants: MEMORIAL SLOAN-KETTERING CANCER CENTER, New York, NY (US); THE GENERAL HOSPITAL CORPORATION, Boston, MA (US); MEMORIAL HOSPITAL FOR CANCER AND ALLIED DISEASES, New York, NY (US); SLOAN-KETTERING INSTITUTE FOR CANCER RESEARCH, New York, NY (US)

(72) Inventors: David Spriggs, Boston, MA (US); Oladapo Yeku, Boston, MA (US)

(21) Appl. No.: 18/550,914

(22) PCT Filed: Mar. 17, 2022
(86) PCT No.: PCT/US2022/020722

§ 371 Date: Sep. 15, 2023

Related U.S. Application Data
(60) Provisional application No. 63/162,822, filed on Mar. 18, 2021.

(51) Int. Cl. C07K 16/30; A61K 45/06; A61P 35/00; C07K 16/22; C07K 16/28

(52) U.S. Cl. CPC C07K 16/3092; A61K 45/06; A61P 35/00; C07K 16/22; C07K 16/2809; C07K 2317/21; C07K 2317/24; C07K 2317/31

(57) ABSTRACT

The present technology provides methods for treating gynecologic cancers using combination therapy with an anti-MUC16×CD3 multispecific (e.g., bispecific) immunoglobulin-related composition that specifically binds to the C-terminal 114 amino acid residues of mature MUC16 (e.g., MUC16^{C114}) and T cells, and a VEGF inhibitor. Kits for use in practicing the methods are also provided.

### CROSS-REFERENCE TO RELATED APPLICATIONS

This application is the national stage application of International Application No. PCT/US2022/020722, filed Mar. 17, 2022, which claims the benefit of and priority to U.S. Provisional Patent Application No. 63/162,822, filed Mar. 18, 2021, the entire contents of each of which are incorporated herein by reference.

### STATEMENT OF GOVERNMENT SUPPORT

This invention was made with government support under grant number CA190174 awarded by the National Institutes of Health (NIH). The government has certain rights in the invention.

### TECHNICAL FIELD

The present technology relates to methods for treating gynecologic cancers using an anti-MUC16×CD3 multispecific (e.g., bispecific) immunoglobulin-related composition that specifically binds to the C-terminal 114 amino acid residues of mature MUC16 (e.g., MUC16^{c114}) and T cells, and an VEGF inhibitor. Kits for use in practicing the methods are also provided.

### SEQUENCE LISTING

The instant application contains a Sequence Listing which has been submitted electronically in ASCII format and is hereby incorporated by reference in its entirety. Said ASCII copy, created on Apr. 19, 2022, is named 115872-2494_SL.txt and is 457,745 bytes in size.

### BACKGROUND

The following description of the background of the present technology is provided simply as an aid in understanding the present technology and is not admitted to describe or constitute prior art to the present technology.

Mucins are important biomolecules for cellular homeostasis and protection of epithelial surfaces. Changes in expression of mucins in cancers, such as ovarian cancer, are useful as a biomarker for diagnosis, prognosis and treatment (Singh A P, et al, Lancet Oncol 2008; 9(11): 1076-85). MUC16 is a mucin that is over expressed on most ovarian carcinoma cells. Since MUC16 antigen is otherwise expressed only at low levels in normal tissues of the uterus, endometrium, fallopian tubes, ovaries, and serosa of the abdominal and thoracic cavities, MUC16 is a potentially attractive target for immune-based therapies, including the targeting and treatment of gynecologic cancer.

MUC16 is a highly glycosylated mucin composed of a large extracellular domain region that is cleaved at a cleavage site and released (CA-125), a retained ectodomain region lying proximal to the cleavage site (MUC16^{ecto}), a transmembrane domain, and a cytoplasmic tail with potential phosphorylation sites (FIG. 1(a)). As used herein, MUC16^{c114} refers to the C-terminal 114 amino acid residues of mature MUC16 (e.g., MUC16^{c114}) and comprises MUC16^{ecto}, the transmembrane domain, and the cytoplasmic tail. The released extracellular domain (CA-125) contains 16-20 tandem repeats of 156 amino acids, each with many potential glycosylation sites (O'Brien T J, et al, Tumor Biol 22(6):348-66 (2001)). Because a significant portion of the extracellular domain of MUC16 is cleaved and secreted (i.e., CA-125), the utility of this portion of MUC16 as a target antigen on ovarian carcinomas is limited. Many reported MUC16 monoclonal antibodies bind to epitopes present on the large secreted CA-125 fraction of the glycoprotein, and not to the retained MUC16 ectodomain (Bellone S Am J Obstet Gynecol 200(1):75 el-10 (2009), Berek J S. Expert Opin Biol Ther. 4(7): 1159-65 (2004); O'Brien T J, et al, Int J Biol Markers 13(4): 188-95 (1998)).

Thus, there is an urgent need for therapeutic methods that effectively target MUC16(+) gynecologic cancers, such as ovarian cancers.

### SUMMARY OF THE PRESENT TECHNOLOGY

In one aspect, the present disclosure provides a method for treating gynecologic cancer in a subject in need thereof, comprising administering to the subject an effective amount of an anti-MUC16×CD3 multispecific (e.g., bispecific) antibody or antigen binding fragment thereof, and an effective amount of a VEGF inhibitor, wherein the anti-MUC16×CD3 multispecific (e.g., bispecific) antibody or antigen binding fragment comprises a first antigen-binding site that specifically binds to a MUC16 polypeptide comprising an MUC16 ectodomain sequence, wherein the MUC16 ectodomain sequence consists of SEQ ID NO: 95. In some embodiments, the MUC16 polypeptide has the amino acid sequence of SEQ ID NO: 3. The anti-MUC16×CD3 multispecific (e.g., bispecific) antigen binding fragment may be a Fab, a Fab′, a F(ab′)2, an Fv, or a single chain Fv (scFv).

Additionally or alternatively, in some embodiments of the methods disclosed herein, the first antigen-binding site comprises a heavy chain immunoglobulin variable domain (V_{H}) and a light chain immunoglobulin variable domain (V_{L}), wherein (a) the V_{H} comprises a V_{H}-CDR1 sequence of SEQ ID NO: 4, a V_{H}-CDR2 sequence of SEQ ID NO: 5, and a V_{H}-CDR3 sequence of SEQ ID NO: 6; and the V_{L} comprises a V_{L}-CDR1 sequence of SEQ ID NO: 7, a V_{L}-CDR2 sequence of SEQ ID NO: 8, and a V_{L}-CDR3 sequence of SEQ ID NO: 9; or (b) the V_{H} comprises a V_{H}-CDR1 sequence of SEQ ID NO: 10, a V_{H}-CDR2 sequence of SEQ ID NO: 11, and a V_{H}-CDR3 sequence of SEQ ID NO: 12; and the V_{L} comprises a V_{L}-CDR1 sequence of SEQ ID NO: 13, a V_{L}-CDR2 sequence of SEQ ID NO: 14, and a V_{L}-CDR3 sequence of SEQ ID NO: 15; or (c) the V_{H} comprises a V_{H}-CDR1 sequence of SEQ ID NO: 16, a V_{H}-CDR2 sequence of SEQ ID NO: 17, and a V_{H}-CDR3 sequence of SEQ ID NO: 18; and the V_{L} comprises a V_{L}-CDR1 sequence of SEQ ID NO: 19, a V_{L}-CDR2 sequence of SEQ ID NO: 20, and a V_{L}-CDR3 sequence of SEQ ID NO: 21; or (d) the V_{H} comprises a V_{H}-CDR1 sequence of SEQ ID NO: 22, a V_{H}-CDR2 sequence of SEQ ID NO: 23, and a V_{H}-CDR3 sequence of SEQ ID NO: 24; and the V_{L} comprises a V_{L}-CDR1 sequence of SEQ ID NO: 25, a V_{L}-CDR2 sequence of SEQ ID NO: 26, and a V_{L}-CDR3 sequence of SEQ ID NO: 27.

Additionally or alternatively, in certain embodiments of the methods disclosed herein, the first antigen-binding site comprises (a) a heavy chain immunoglobulin variable domain (V_{H}) comprising a V_{H}-CDR1 sequence, a V_{H}-CDR2 sequence, and a V_{H}-CDR3 sequence of SEQ ID NO: 28 or SEQ ID NO: 29 and a light chain immunoglobulin variable domain (V_{L}) comprising a V_{L}-CDR1 sequence, a V_{L}-CDR2 sequence, and a V_{L}-CDR3 sequence of SEQ ID NO: 30 or SEQ ID NO: 31; or (b) a heavy chain immunoglobulin variable domain (V_{H}) comprising a V_{H}-CDR1 sequence, a V_{H}-CDR2 sequence, and a V_{H}-CDR3 sequence of SEQ ID NO: 32 or SEQ ID NO: 33 and a light chain immunoglobulin variable domain (V_{L}) comprising a V_{L}-CDR1 sequence, a V_{L}-CDR2 sequence, and a V_{L}-CDR3 sequence of SEQ ID NO: 34 or SEQ ID NO: 35; or (c) a heavy chain immunoglobulin variable domain (V_{H}) comprising a V_{H}-CDR1 sequence, a V_{H}-CDR2 sequence, and a V_{H}-CDR3 sequence of SEQ ID NO: 36 and a light chain immunoglobulin variable domain (V_{L}) comprising a V_{L}-CDR1 sequence, a V_{L}-CDR2 sequence, and a V_{L}-CDR3 sequence of SEQ ID NO: 37; or (d) a heavy chain immunoglobulin variable domain (V_{H}) comprising a V_{H}-CDR1 sequence, a V_{H}-CDR2 sequence, and a V_{H}-CDR3 sequence of SEQ ID NO: 38 and a light chain immunoglobulin variable domain (V_{L}) comprising a V_{L}-CDR1 sequence, a V_{L}-CDR2 sequence, and a V_{L}-CDR3 sequence of SEQ ID NO: 39.

In any of the preceding embodiments of the methods disclosed herein, the first antigen-binding site comprises (a) a heavy chain immunoglobulin variable domain (V_{H}) comprising the amino acid sequence of SEQ ID NO: 28 or SEQ ID NO: 29 and a light chain immunoglobulin variable domain (V_{L}) comprising the amino acid sequence of SEQ ID NO: 30 or SEQ ID NO: 31; or (b) a heavy chain immunoglobulin variable domain (V_{H}) comprising the amino acid sequence of SEQ ID NO: 32 or SEQ ID NO: 33 and a light chain immunoglobulin variable domain (V_{L}) comprising the amino acid sequence of SEQ ID NO: 34 or SEQ ID NO: 35; or (c) a heavy chain immunoglobulin variable domain (V_{H}) comprising the amino acid sequence of SEQ ID NO: 36 and a light chain immunoglobulin variable domain (V_{L}) comprising the amino acid sequence of SEQ ID NO: 37; or (d) a heavy chain immunoglobulin variable domain (V_{H}) comprising the amino acid sequence of SEQ ID NO: 38 and a light chain immunoglobulin variable domain (V_{L}) comprising the amino acid sequence of SEQ ID NO: 39.

Additionally or alternatively, in some embodiments of the methods disclosed herein, the anti-MUC16×CD3 multispecific (e.g., bispecific) antibody or antigen binding fragment further comprises a Fc domain of an isotype selected from the group consisting of IgG1, IgG2, IgG3, IgG4, IgA1, IgA2, IgM, IgD, and IgE. In some embodiments of the methods disclosed herein, the anti-MUC16×CD3 multispecific (e.g., bispecific) antibody or antigen binding fragment is human, or humanized. Additionally or alternatively, in certain embodiments of the methods disclosed herein, the anti-MUC16×CD3 multispecific (e.g., bispecific) antibody or antigen binding fragment is a tandem scFv, a diabody (db), a single chain diabody (scDb), a dual-affinity retargeting (DART) antibody, a F(ab′)2, a dual variable domain (DVD) antibody, a knob-into-hole (KiH) antibody, a dock and lock (DNL) antibody, a chemically cross-linked antibody, a heteromultimeric antibody, a monoclonal antibody, a full-length antibody, or a heteroconjugate antibody.

In any and all embodiments of the methods disclosed herein, the anti-MUC16×CD3 multispecific (e.g., bispecific) antibody or antigen binding fragment comprises a second antigen-binding site that specifically binds to T cells. In certain embodiments, the second antigen-binding site comprises a heavy chain immunoglobulin variable domain (V_{H}) comprising the amino acid sequence of SEQ ID NO: 70 and a light chain immunoglobulin variable domain (V_{L}) comprising the amino acid sequence of SEQ ID NO: 71. Additionally or alternatively, in some embodiments, the second antigen-binding site comprises the amino acid sequence of SEQ ID NO: 72. In other embodiments, the anti-MUC16×CD3 multispecific (e.g., bispecific) antibody or antigen binding fragment comprises the amino acid sequence of any one of SEQ ID NOs: 73-92.

Additionally or alternatively, in some embodiments of the methods disclosed herein, the VEGF inhibitor is a small molecule inhibitor, a siRNA, an antisense oligonucleotide, a shRNA, a sgRNA, a ribozyme, or an antibody or antigen binding fragment thereof. Examples of VEGF inhibitors include, but are not limited to, bevacizumab, ranibizumab, vanucizumab, brolucizumab, hPV19, IBI305, VEGF Trap, linifanib, AEE-788, axitinib (AG-13736), AG-028262, Angiostatin, combretastatin A4, cediranib, sorafenib, Thalidomide, vatalanib, DC-101, SNS-032, sunitinib malate, semaxanib, CEP-7055, dovitinib, CP-547632, CP-564959, lenvatinib, pazopanib, GW-654652, tivozanib, benzoylstaurosporine, orantinib, tesevatinib, XL-999, foretinib, vandetanib, and ZK-304709. In some embodiments, the gynecologic cancer is ovarian cancer, fallopian tube cancer, uterine cancer, or endometrial cancer.

Additionally or alternatively, in some embodiments, the subject exhibits decreased tumor growth, reduced tumor proliferation, lower tumor burden, or increased survival after administration of the VEGF inhibitor and the anti-MUC16×CD3 multispecific (e.g., bispecific) antibody or antigen binding fragment. Additionally or alternatively, in some embodiments of the combination therapy methods disclosed herein, the time to response and/or duration of response is improved relative to that observed with VEGF inhibitor monotherapy or monotherapy with the anti-MUC16×CD3 multispecific (e.g., bispecific) antibody or antigen binding fragment.

In any and all embodiments of the methods disclosed herein, the VEGF inhibitor and the anti-MUC16×CD3 multispecific (e.g., bispecific) antibody or antigen binding fragment are administered separately, sequentially, or simultaneously. The anti-MUC16×CD3 multispecific (e.g., bispecific) antibody or antigen binding fragment and/or the VEGF inhibitor may be administered orally, intranasally, parenterally, intravenously, intramuscularly, intraperitoneally, intramuscularly, intraarterially, subcutaneously, intrathecally, intracapsularly, intraorbitally, intratumorally, intradermally, transtracheally, intracerebroventricularly, topically, or via an implanted reservoir.

### BRIEF DESCRIPTION OF THE DRAWINGS

FIGS. 1(a)-1(d) describe the screening and identification of human MUC16^{ecto} specific antibodies. FIG. 1(a): Schematic representation of MUC16 antigen and the c114 retained ectodomain (MUC16^{ecto}) which serves as the antibody target. FIG. 1(b): Phage display binding analysis by FACS. Only targets that bind to MUC16^{ecto} (blue) and not the mutant MUC16 (orange) were selected for sequencing and further development. Examples of negative control (top left) and non-specific binding (top right) are shown. FIG. 1(c): Purity of CD3F-conjugated MUC16^{ecto}-specific Bispecific T-cell engaging diabodies (BiTEDs) were validated by SDS-PAGE. Expected band size 50-55 KDa. FIG. 1(d): Kinetic analysis was performed to determine the dissociation constant of MUC16^{ecto}-BiTEDs to the highly conserved 55mer MUC16 ectodomain.

FIGS. 2(a)-2(c) show validation of MUC16^{ecto}-BITEDs binding to MUC16 ectodomain and evaluation of potential interference by soluble CA-125.

FIG. 2(a): BTM protein was immunoprecipitated (IP) with MUC16^{ecto}-BiTEDs (line 4). Control conditions; Protein G agarose beads alone (line 1), protein G beads with BTM protein (line 2) or with MUC16^{ecto}-BiTEDs (line 3) did not show the expected 55 Kda band representing MUC16^{ecto}-BiTEDs. Unbound MUC16^{ecto}-BiTEDs but not BTM protein were detected in flow through (lines 5-7). FIG. 2(b): ELISA showing binding of increasing concentrations of MUC16^{ecto}-BiTEDs in the presence of BTM. FIG. 2(c): ELISA showing binding of diluent and MUC16^{ecto}-BiTEDs in the presence of increasing concentrations of recombinant CA-125. Results from FIG. 2(b) and FIG. 2(c) are pooled from 3 independent experiments. Data are plotted as mean±SEM.

FIGS. 3(a)-3(f) demonstrate that MUC16^{ecto}-BITEDs display cytotoxicity against a panel of Muc16 positive ovarian cancer cell line in vitro.

FIG. 3(a): Evaluation of Cytotoxicity via 1:1 E:T coculture of SKOV3, OVCAR3 and activated T-cells with the indicated BiTEDs. FIG. 3(b): Coculture of MUC16^{ecto}-BITEDs and activated T-cells and SKOV3-MUC16^{ecto} at the indicated E:T ratios; FIG. 3(c): Coculture of MUC16^{ecto}-BITEDs and activated T-cells and OVCAR3 at the indicated E:T ratios, and FIG. 3(d): Coculture of MUC16^{ecto}-BITEDs and activated T-cells and SKOV3 tumor cells at the indicated E:T ratios. Data are plotted as mean±SEM. Data shown are pooled results from 3 independent experiments. At least 4 separate donors were used for each experiment. Statistics performed using unpaired two-sided T test. FIG. 3(e): Percentages of Granzyme B positive CD4 and CD8 T-cells cocultured with SKOV3-MUC16^{ecto} cells with or without MUC16^{ecto}-BITEDs. FIG. 3(f): In vitro cytokine analysis of supernatants obtained from SKOV3-MUC16^{ecto} cells alone, and coculture of SKOV3-MUC16^{ecto} cells with T-cells with or without MUC16^{ecto}-BITEDs. For FIG. 3(e) and FIG. 3(f), data shown are pooled results from 5 independent experiments with at least 4 independent donors. Data are plotted as mean±SEM. *p<0.05.

FIGS. 4(a)-4(c) demonstrate that MUC16^{ecto} specific BiTEDs delay ovarian cancer progression in vivo.

FIG. 4(a): Female NSG mice were inoculated i.p. with SKOV3-MUC16^{ecto}-GFP-LUC tumor cells and subsequently treated with T-cells alone, or T-cells and MUC16^{ecto} BiTEDs and were imaged for tumor burden over time. FIG. 4(b): Quantification of bioluminescence from FIG. 4(a). FIG. 4(c): Female NSG tumor bearing mice were treated with T-cells alone or T-cells and MUC16^{ecto}-BiTEDs. Serum cytokines were evaluated 7-days after treatment. Data shown are pooled results from 2 independent experiments with at least 3 independent donors. Data are plotted as mean±SEM. *p<0.01, **p<0.05.

FIGS. 5(a)-5(c) demonstrate that MUC16^{ecto}-BiTEDs improve overall survival in tumor-bearing mice as monotherapy and in combination with PD-1 immune checkpoint blockade.

FIG. 5(a): NSG tumor-bearing mice were inoculated i.p. with SKOV3-MUC16^{ecto} tumor cells and treated with either T-cells alone or T-cells and MUC16^{ecto}-BiTEDs. Data shown are pooled results from 3 independent experiments with at least 4 independent donors. Data are plotted as mean±SEM. **p<0.05. FIG. 5(b): Immunophenotyping of human T-cells from the spleens of female NSG SKOV3-MUC16^{ecto} tumor-bearing mice treated with BiTEDs who had either succumbed to disease (Non-Responders) or from those who lived longer (Responders). **p<0.05. FIG. 5(c): SKOV3-MUC16^{ecto} tumor-bearing mice were treated with T-cells and αPD-1 alone, T-cells and BiTEDs, or T-cells, αPD-1 and BiTEDs. Data shown are pooled results from 3 independent experiments with at least 4 independent donors. Data are plotted as mean±SEM. *p<0.05 T-cells+αPD-1 vs T-cells+BiTEDs, **p<0.05 T-cells+BiTEDs vs T-cells+BiTEDS+αPD-1.

FIGS. 6(a)-6(f) demonstrate that MUC16^{ecto}-BiTEDs in combination with VEGF inhibition significantly improves ascites and peritoneal tumor cells and overall survival.

FIG. 6(a): Female NSG mice were inoculated i.p with OVCAR3 tumor cells and treated with T-cells alone or T-cells and MUC16^{ecto}-BiTEDs. Data shown are pooled results from 2 independent experiments with at least 4 independent Leukopak donors. Data are plotted as mean±SEM. **p<0.05. FIG. 6(b): Expression level of VEGF in OVCAR3 cells by flow cytometry. FIG. 6(c): Evaluation of VEGF in supernatant collected from T-cells and OVCAR3. T-cells vs OVCAR3 (*p<0.05). FIG. 6(d): OVCAR3 tumor-bearing mice treated with T-cells+αVEGF, T-cells+BiTEDs or T-cells, +BiTEDs+αVEGF. Data are plotted as mean±SEM. T cells+αVEGF vs T cells+BiTEDs (*p<0.005). T cells+BiTEDs vs T cells+BiTEDs+αVEGF (**p<0.005). Statistical analysis for FIG. 6(a) and FIG. 6(d) were performed using a log-rank (Mantel-Cox) test. FIG. 6(e): Volume of ascites collected from OVCAR3 tumor-bearing mice treated with T-cells alone, T-cells+αPD-1, T-cells+αVEGF, T-cells+BiTEDs, T-cells+αPD-1+BiTEDs, or T-cells+αVEGF+BiTEDs. Data shown are pooled results from 2 independent experiments with at least 2 independent Leukopak donors. Data are plotted as mean±SEM. **p<0.05. FIG. 6(f): Number of peritoneal tumor cells corresponding to the same conditions as in FIG. 6(e). Data are plotted as mean±SEM. **p<0.05.

### DETAILED DESCRIPTION

It is to be appreciated that certain aspects, modes, embodiments, variations and features of the present technology are described below in various levels of detail in order to provide a substantial understanding of the present technology. It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

MUC16 protein is a heavily glycosylated member of the mucin family with normal Mullerian tissue expression and is overexpressed on High Grade Serous Epithelial Ovarian Cancer cells (HGSOC). MUC16 is post-translationally cleaved into a soluble antigenic fragment from the tandem repeat region (detected as CA-125) and a retained extracellular fragment-termed MUC16^{ecto} with independent pro-oncogenic properties (O'Brien T J et al., Tumour Biol. 2001; 22(6):348-66). The majority of antibody based anti-MUC16 clinical therapeutics target the shed portion of MUC16 (Bellone S et al., Am J Obstet Gynecol. 2009; 200(1):75 el-10) which may limit their specificity as targeted immunotherapy.

The present disclosure demonstrates that combination therapy with an anti-MUC16×CD3 multispecific (e.g., bispecific) immunoglobulin-related composition that specifically binds to the C-terminal 114 amino acid residues of mature MUC16 (e.g., MUC16^{c114}) and T cells, and a VEGF inhibitor results in the synergistic treatment of gynecologic cancer. Without wishing to be bound by theory, it is believed that the observed synergistic anti-tumor response may be attributable at least in part to a decrease in expression of exhaustion markers such as PD-1 on T-cells. See de Almeida P E et al., Cancer Immunol Res 8: 806-18 (2020). Another possible mechanism for improved survival in animals treated with the combination therapy methods of the present technology might be due to decreased ascites (which contribute to the immunosuppressive tumor microenvironment (see Yeku O O et al., Sci Rep 7: 10541 (2017)), and reduced T-cell dysfunction, leading to increased tumor debulking by T-cells. It is expected that as the peritoneal tumor burden decreases, the amount of ascites further decreases, leading to further improvement in T-cell function and treatment of gynecologic cancers.

### Definitions

The definitions of certain terms as used in this specification are provided below. Unless defined otherwise, all technical and scientific terms used herein generally have the same meaning as commonly understood by one of ordinary skill in the art to which this present technology belongs.

As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine. Amino acid analogs refer to agents that have the same basic chemical structure as a naturally occurring amino acid, i.e., an a carbon that is bound to a hydrogen, a carboxyl group, an amino group, and an R group, such as, homoserine, norleucine, methionine sulfoxide, methionine methyl sulfonium. Such analogs have modified R groups (such as norleucine) or modified peptide backbones, but retain the same basic chemical structure as a naturally occurring amino acid. In some embodiments, amino acids forming a polypeptide are in the D form. In some embodiments, the amino acids forming a polypeptide are in the L form. In some embodiments, a first plurality of amino acids forming a polypeptide are in the D form and a second plurality are in the L form.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter code.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. As used herein, “antibodies” (includes intact immunoglobulins) and “antigen binding fragments” specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴ M⁻¹ greater or at least 10⁵ M⁻¹ greater than a binding constant for other molecules in a biological sample). The term “antibody” also includes native antibodies, monoclonal antibodies, human antibodies, humanized antibodies, camelised antibodies, multispecific antibodies, bispecific antibodies, chimeric antibodies, Fab, Fab′, single chain V region fragments (scFv), single domain antibodies (e.g., nanobodies and single domain camelid antibodies), VNAR fragments, Bi-specific T-cell engager antibodies, minibodies, disulfide-linked Fvs (sdFv), and anti-idiotypic (anti-id) antibodies, intrabodies, fusion polypeptides, unconventional antibodies and antigen-binding fragments of any of the above. See also, Pierce Catalog and Handbook, 1994-1995 (Pierce Chemical Co., Rockford, Ill.); Kuby, J., Immunology, 3^{rd} Ed., W.H. Freeman & Co., New York, 1997.

More particularly, antibody refers to a polypeptide ligand comprising at least a light chain immunoglobulin variable region or heavy chain immunoglobulin variable region which specifically recognizes and binds an epitope of an antigen. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody. Typically, an immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. An antibody that binds a target protein (e.g., MUC16) will have a specific V_{H} region and the V_{L} region sequence, and thus specific CDR sequences. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs). “Immunoglobulin-related compositions” as used herein, refers to antibodies (including monoclonal antibodies, polyclonal antibodies, human antibodies, humanized antibodies, chimeric antibodies, recombinant antibodies, multi-specific antibodies, bispecific antibodies, etc.,) as well as antibody fragments. An antibody or antigen binding fragment thereof specifically binds to an antigen.

As used herein, the term “antibody-related polypeptide” means antigen-binding antibody fragments, including single-chain antibodies, that can comprise the variable region(s) alone, or in combination, with all or part of the following polypeptide elements: hinge region, CH₁, CH2, and CH3 domains of an antibody molecule. Also included in the technology are any combinations of variable region(s) and hinge region, CH₁, CH2, and CH3 domains. Antibody-related molecules useful in the present methods, e.g., but are not limited to, Fab, Fab′ and F(ab′)2, Fd, single-chain Fvs (scFv), single-chain antibodies, disulfide-linked Fvs (sdFv) and fragments comprising either a V_{L} or V_{H} domain. Examples include: (i) a Fab fragment, a monovalent fragment consisting of the V_{L}, V_{H}, CL and CH₁ domains; (ii) a F(ab′)2 fragment, a bivalent fragment comprising two Fab fragments linked by a disulfide bridge at the hinge region (A“F(ab′)₂” fragment can be split into two individual Fab′ fragments.); (iii) a Fd fragment consisting of the V_{H} and CH₁ domains; (iv) a Fv fragment consisting of the V_{L} and V_{H} domains of a single arm of an antibody, (v) a dAb fragment (Ward et al., Nature 341: 544-546, 1989), which consists of a V_{H} domain; and (vi) an isolated complementarity determining region (CDR). As such “antibody fragments” or “antigen binding fragments” can comprise a portion of a full length antibody, generally the antigen binding or variable region thereof. Examples of antibody fragments or antigen binding fragments include Fab, Fab′, F(ab′)₂, and Fv fragments; diabodies (dscFvs); linear antibodies; single-chain antibody molecules; and multi-specific antibodies formed from antibody fragments.

“Bispecific antibody” or “BsAb”, as used herein, refers to an antibody that can bind simultaneously to two targets that have a distinct structure, e.g., two different target antigens, two different epitopes on the same target antigen, or a hapten and a target antigen or epitope on a target antigen. A variety of different bispecific antibody structures are known in the art. In some embodiments, each antigen binding moiety in a bispecific antibody includes V_{H} and/or V_{L} regions; in some such embodiments, the V_{H} and/or V_{L} regions are those found in a particular monoclonal antibody. In some embodiments, the bispecific antibody contains two antigen binding moieties, each including V_{H} and/or V_{L} regions from different monoclonal antibodies. In some embodiments, the bispecific antibody comprises two antigen binding moieties, wherein one of the two antigen binding moieties includes an antibody fragment (e.g., Fab, F(ab′), F(ab′)₂, Fd, Fv, dAB, scFv, etc.) having a V_{H} region and/or a V_{L} region that contain CDRs from a first monoclonal antibody, and the other antigen binding moiety includes an antibody fragment (e.g., Fab, F(ab′), F(ab′)₂, Fd, Fv, dAB, scFv, etc.) having a V_{H} region and a V_{L} region that contain CDRs from a second monoclonal antibody. In some embodiments, the bispecific antibody contains two antigen binding moieties, wherein one of the two antigen binding moieties includes an immunoglobulin molecule having V_{H} and/or V_{L} regions that contain CDRs from a first monoclonal antibody, and the other antigen binding moiety includes an antibody fragment (e.g., Fab, F(ab′), F(ab′)₂, Fd, Fv, dAB, scFv, etc.) having V_{H} and/or V_{L} regions that contain CDRs from a second monoclonal antibody.

As used herein, an “antigen” refers to a molecule to which an antibody (or antigen binding fragment thereof) can selectively bind. The target antigen may be a protein, carbohydrate, nucleic acid, lipid, hapten, or other naturally occurring or synthetic compound. In some embodiments, the target antigen may be a polypeptide. An antigen may also be administered to an animal to generate an immune response in the animal.

The term “antigen binding fragment” refers to a fragment of the whole immunoglobulin structure which possesses a part of a polypeptide responsible for binding to antigen. Examples of the antigen binding fragment useful in the present technology include scFv, (scFv)₂, scFvFc, Fab, Fab′ and F(ab′)₂, but are not limited thereto. Any of the above-noted antibody fragments are obtained using conventional techniques known to those of skill in the art, and the fragments are screened for binding specificity and neutralization activity in the same manner as are intact antibodies.

As used herein, “binding affinity” means the strength of the total noncovalent interactions between a single binding site of a molecule (e.g., an antibody) and its binding partner (e.g., an antigen or antigenic peptide). The affinity of a molecule X for its partner Y can generally be represented by the dissociation constant (K_{D}). Affinity can be measured by standard methods known in the art, including those described herein. A low-affinity complex contains an antibody that generally tends to dissociate readily from the antigen, whereas a high-affinity complex contains an antibody that generally tends to remain bound to the antigen for a longer duration.

Without being bound to theory, affinity depends on the closeness of stereochemical fit between antibody combining sites and antigen determinants, on the size of the area of contact between them, and on the distribution of charged and hydrophobic groups. Affinity also includes the term “avidity,” which refers to the strength of the antigen-antibody bond after formation of reversible complexes (e.g., either monovalent or multivalent). Methods for calculating the affinity of an antibody for an antigen are known in the art, comprising use of binding experiments to calculate affinity. Antibody activity in functional assays (e.g., flow cytometry assay) is also reflective of antibody affinity. Antibodies and affinities can be phenotypically characterized and compared using functional assays (e.g., flow cytometry assay).

As used herein, the term “CDR grafting” means replacing at least one CDR of an “acceptor” antibody by a CDR “graft” from a “donor” antibody possessing a desirable antigen specificity. As used herein, the term “CDR-grafted antibody” means an antibody in which at least one CDR of an “acceptor” antibody is replaced by a CDR “graft” from a “donor” antibody possessing a desirable antigen specificity.

As used herein, the term “conjugated” refers to the association of two molecules by any method known to those in the art. Suitable types of associations include chemical bonds and physical bonds. Chemical bonds include, for example, covalent bonds and coordinate bonds. Physical bonds include, for instance, hydrogen bonds, dipolar interactions, van der Waal forces, electrostatic interactions, hydrophobic interactions and aromatic stacking.

As used herein, the term “consensus FR” means a framework (FR) antibody region in a consensus immunoglobulin sequence. The FR regions of an antibody do not contact the antigen.

As used herein, the term “constant region” or “constant domain” is interchangeable and has its meaning common in the art. The constant region is an antibody portion, e.g., a carboxyl terminal portion of a light and/or heavy chain which is not directly involved in binding of an antibody to antigen but which can exhibit various effector functions, such as interaction with the Fc receptor. The constant region of an immunoglobulin molecule generally has a more conserved amino acid sequence relative to an immunoglobulin variable domain.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, the term “diabodies” refers to small antibody fragments with two antigen-binding sites, which fragments comprise a heavy-chain variable domain (V_{H}) connected to a light-chain variable domain (V_{L}) in the same polypeptide chain (V_{H} V_{L}). By using a linker that is too short to allow pairing between the two domains on the same chain, the domains are forced to pair with the complementary domains of another chain and create two antigen binding sites. Diabodies are described more fully in, e.g., EP 404,097; WO 93/11161; and Hollinger et al., Proc Natl Acad Sci USA, 90: 6444-6448 (1993).

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, the term “effector cell” means an immune cell which is involved in the effector phase of an immune response, as opposed to the cognitive and activation phases of an immune response. Exemplary immune cells include a cell of a myeloid or lymphoid origin, e.g., lymphocytes (e.g., B cells and T cells including cytolytic T cells (CTLs)), killer cells, natural killer cells, macrophages, monocytes, eosinophils, neutrophils, polymorphonuclear cells, granulocytes, mast cells, and basophils. Effector cells express specific Fc receptors and carry out specific immune functions. An effector cell can induce antibody-dependent cell-mediated cytotoxicity (ADCC), e.g., a neutrophil capable of inducing ADCC. For example, monocytes, macrophages, neutrophils, eosinophils, and lymphocytes which express FcαR are involved in specific killing of target cells and presenting antigens to other components of the immune system, or binding to cells that present antigens.

As used herein, the term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and non-conformational epitopes are distinguished in that the binding to the former but not the latter is lost in the presence of denaturing solvents. In some embodiments, an “epitope” of the MUC16 protein is a region of the protein to which the anti-MUC16 antibodies of the present technology specifically bind (e.g., MUC16^{ecto}). In some embodiments, the epitope is a conformational epitope or a non-conformational epitope. To screen for anti-MUC16 antibodies which bind to an epitope, a routine cross-blocking assay such as that described in Antibodies, A Laboratory Manual, Cold Spring Harbor Laboratory, Ed Harlow and David Lane (1988), can be performed. This assay can be used to determine if an anti-MUC16 antibody binds the same site or epitope as an anti-MUC16 antibody of the present technology. Alternatively, or additionally, epitope mapping can be performed by methods known in the art. For example, the antibody sequence can be mutagenized such as by alanine scanning, to identify contact residues. In a different method, peptides corresponding to different regions of MUC16 protein can be used in competition assays with the test antibodies or with a test antibody and an antibody with a characterized or known epitope. An epitope can be, e.g., contiguous amino acids of a polypeptide (linear or contiguous epitope) or an epitope can, e.g., come together from two or more noncontiguous regions of a polypeptide or polypeptides (conformational, non-linear, discontinuous, or non-contiguous epitope).

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression can include splicing of the mRNA in a eukaryotic cell. The expression level of a gene can be determined by measuring the amount of mRNA or protein in a cell or tissue sample. In one aspect, the expression level of a gene from one sample can be directly compared to the expression level of that gene from a control or reference sample. In another aspect, the expression level of a gene from one sample can be directly compared to the expression level of that gene from the same sample following administration of the compositions disclosed herein. The term “expression” also refers to one or more of the following events: (1) production of an RNA template from a DNA sequence (e.g., by transcription) within a cell; (2) processing of an RNA transcript (e.g., by splicing, editing, 5′ cap formation, and/or 3′ end formation) within a cell; (3) translation of an RNA sequence into a polypeptide or protein within a cell; (4) post-translational modification of a polypeptide or protein within a cell; (5) presentation of a polypeptide or protein on the cell surface; and (6) secretion or presentation or release of a polypeptide or protein from a cell.

As used herein, the term “gene” means a segment of DNA that contains all the information for the regulated biosynthesis of an RNA product, including promoters, exons, introns, and other untranslated regions that control expression.

As used herein, “homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. A polynucleotide or polynucleotide region (or a polypeptide or polypeptide region) has a certain percentage (for example, at least 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art. In some embodiments, default parameters are used for alignment. One alignment program is BLAST, using default parameters. In particular, programs are BLASTN and BLASTP, using the following default parameters: Genetic code=standard; filter-none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Details of these programs can be found at the National Center for Biotechnology Information. Biologically equivalent polynucleotides are those having the specified percent homology and encoding a polypeptide having the same or similar biological activity. Two sequences are deemed “unrelated” or “non-homologous” if they share less than 40% identity, or less than 25% identity, with each other.

As used herein, “humanized” forms of non-human (e.g., murine) antibodies are chimeric antibodies which contain minimal sequence derived from non-human immunoglobulin. For the most part, humanized antibodies are human immunoglobulins in which hypervariable region residues of the recipient are replaced by hypervariable region residues from a non-human species (donor antibody) such as mouse, rat, rabbit or nonhuman primate having the desired specificity, affinity, and capacity. In some embodiments, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance such as binding affinity. Generally, the humanized antibody will comprise substantially all of at least one, and typically two, variable domains (e.g., Fab, Fab′, F(ab′)₂, or Fv), in which all or substantially all of the hypervariable loops correspond to those of a non-human immunoglobulin and all or substantially all of the FR regions are those of a human immunoglobulin consensus FR sequence although the FR regions may include one or more amino acid substitutions that improve binding affinity. The number of these amino acid substitutions in the FR are typically no more than 6 in the H chain, and in the L chain, no more than 3. The humanized antibody optionally may also comprise at least a portion of an immunoglobulin constant region (Fc), typically that of a human immunoglobulin. For further details, see Jones et al., Nature 321:522-525 (1986); Reichmann et al., Nature 332:323-329 (1988); and Presta, Curr. Op. Struct. Biol. 2:593-596 (1992). See e.g., Ahmed & Cheung, FEBS Letters 588(2):288-297 (2014). By way of example, a humanized version of a murine antibody to a given antigen has on both of its heavy and light chains (1) constant regions of a human antibody; (2) framework regions from the variable domains of a human antibody; and (3) CDRs from the murine antibody. When necessary, one or more residues in the human framework regions can be changed to residues at the corresponding positions in the murine antibody so as to preserve the binding affinity of the humanized antibody to the antigen. This change is sometimes called “back mutation.” Similarly, forward mutations may be made to revert back to murine sequence for a desired reason, e.g., stability or affinity to antigen.

As used herein, the term “hypervariable region” refers to the amino acid residues of an antibody which are responsible for antigen-binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35B (H1), 50-65 (H2) and 95-102 (H3) in the V_{H} (Kabat et al., Sequences of Proteins of Immunological Interest, 5th Ed. Public Health Service, National Institutes of Health, Bethesda, MD. (1991)) and/or those residues from a “hypervariable loop” (e.g., residues 26-32 (L1), 50-52 (L2) and 91-96 (L3) in the V_{L}, and 26-32 (H1), 52A-55 (H2) and 96-101 (H3) in the V_{H} (Chothia and Lesk J. Mol. Biol. 196:901-917 (1987)).

As used herein, the terms “identical” or percent “identity”, when used in the context of two or more nucleic acids or polypeptide sequences, refer to two or more sequences or subsequences that are the same or have a specified percentage of amino acid residues or nucleotides that are the same (i.e., about 60%, 65%, 70%, 75%, 80%, 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein)), when compared and aligned for maximum correspondence over a comparison window or designated region as measured using a BLAST or BLAST 2.0 sequence comparison algorithms with default parameters described below, or by manual alignment and visual inspection (e.g., NCBI web site). Such sequences are then said to be “substantially identical.” This term also refers to, or can be applied to, the complement of a test sequence. The term also includes sequences that have deletions and/or additions, as well as those that have substitutions. In some embodiments, identity exists over a region that is at least about 25 amino acids or nucleotides in length, or 50-100 amino acids or nucleotides in length.

As used herein, the terms “immunospecifically binds,” “immunospecifically recognizes,” “specifically binds,” and “specifically recognizes” are analogous terms in the context of antibodies and refer to antibodies and antigen-binding fragments thereof that bind to an antigen (e.g., epitope or immune complex) via the antigen-binding sites as understood by one skilled in the art, and does not exclude cross-reactivity of the antibody or antigen binding fragment with other antigens.

As used herein, the term “intact antibody” or “intact immunoglobulin” means an antibody that has at least two heavy (H) chain polypeptides and two light (L) chain polypeptides interconnected by disulfide bonds. Each heavy chain is comprised of a heavy chain variable region (abbreviated herein as HCVR or V_{H}) and a heavy chain constant region. The heavy chain constant region is comprised of three domains, CH₁, CH2 and CH3. Each light chain is comprised of a light chain variable region (abbreviated herein as LCVR or V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR₁, CDR₁, FR₂, CDR₂, FR₃, CDR₃, FR₄. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

As used herein, the term “ligand” refers to a molecule that binds to a receptor. In particular, the ligand binds a receptor on another cell, allowing for cell-to-cell recognition and/or interaction.

As used herein, the term “linker” refers to a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple V_{H} and V_{L} domains). In certain embodiments, the linker comprises amino acids having the sequence (GGGGS)ₙ(SEQ ID NO: 98), wherein n is at least 1, 2, 3, 4, 5, 6, 7, 8, 9, 10, 11, 12, 12, 14, or 15.

The term “monoclonal antibody” as used herein refers to an antibody obtained from a population of substantially homogeneous antibodies, i.e., the individual antibodies comprising the population are identical except for possible naturally occurring mutations that may be present in minor amounts. For example, a monoclonal antibody can be an antibody that is derived from a single clone, including any eukaryotic, prokaryotic, or phage clone, and not the method by which it is produced. A monoclonal antibody composition displays a single binding specificity and affinity for a particular epitope. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. Monoclonal antibodies can be prepared using a wide variety of techniques known in the art including, e.g., but not limited to, hybridoma, recombinant, and phage display technologies. For example, the monoclonal antibodies to be used in accordance with the present methods may be made by the hybridoma method first described by Kohler et al., Nature 256:495 (1975), or may be made by recombinant DNA methods (See, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example.

As used herein, the term “MUC16” or “MUC16 polypeptide” or “MUC16 peptide” refers to the MUC16 tethered mucin protein as described in Yin B W and Lloyd K O, 2001, J Biol Chem. 276(29):27371-5. GenBank™ accession number NP 078966.2 (SEQ ID NO: 1) provides an exemplary human MUC16 amino acid sequence.

Immature MUC16, NP 078966.2
(SEQ ID NO: 1)
MLKPSGLPGSSSPTRSLMTGSRSTKATPEMDSGLTGATLSPKTSTGAIVVTEHTLPFT
SPDKTLASPTSSVVGRTTQSLGVMSSALPESTSRGMTHSEQRTSPSLSPQVNGTPSR
NYPATSMVSGLSSPRTRTSSTEGNFTKEASTYTLTVETTSGPVTEKYTVPTETSTTEG
DSTETPWDTRYIPVKITSPMKTFADSTASKENAPVSMTPAETTVTDSHTPGRTNPSF
GTLYSSFLDLSPKGTPNSRGETSLELILSTTGYPFSSPEPGSAGHSRISTSAPLSSSASV
LDNKISETSIFSGQSLTSPLSPGVPEARASTMPNSAIPFSMTLSNAETSAERVRSTISSL
GTPSISTKQTAETILTFHAFAETMDIPSTHIAKTLASEWLGSPGTLGGTSTSALTTTSP
STTLVSEETNTHHSTSGKETEGTLNTSMTPLETSAPGEESEMTATLVPTLGFTTLDSK
IRSPSQVSSSHPTRELRTTGSTSGRQSSSTAAHGSSDILRATTSSTSKASSWTSESTAQ
QFSEPQHTQWVETSPSMKTERPPASTSVAAPITTSVPSVVSGFTTLKTSSTKGIWLEE
TSADTLIGESTAGPTTHQFAVPTGISMTGGSSTRGSQGTTHLLTRATASSETSADLTL
ATNGVPVSVSPAVSKTAAGSSPPGGTKPSYTMVSSVIPETSSLQSSAFREGTSLGLTP
LNTRHPFSSPEPDSAGHTKISTSIPLLSSASVLEDKVSATSTFSHHKATSSITTGTPEIS
TKTKPSSAVLSSMTLSNAATSPERVRNATSPLTHPSPSGEETAGSVLTLSTSAETTDS
PNIHPTGTLTSESSESPSTLSLPSVSGVKTTFSSSTPSTHLFTSGEETEETSNPSVSQPET
SVSRVRTTLASTSVPTPVFPTMDTWPTRSAQFSSSHLVSELRATSSTSVTNSTGSALP
KISHLTGTATMSQTNRDTENDSAAPQSTTWPETSPRFKTGLPSATTTVSTSATSLSAT
VMVSKFTSPATSSMEATSIREPSTTILTTETTNGPGSMAVASTNIPIGKGYITEGRLDT
SHLPIGTTASSETSMDFTMAKESVSMSVSPSQSMDAAGSSTPGRTSQFVDTFSDDVY
HLTSREITIPRDGTSSALTPQMTATHPPSPDPGSARSTWLGILSSSPSSPTPKVTMSST
FSTQRVTTSMIMDTVETSRWNMPNLPSTTSLTPSNIPTSGAIGKSTLVPLDTPSPATS
LEASEGGLPTLSTYPESTNTPSIHLGAHASSESPSTIKLTMASVVKPGSYTPLTFPSIET
HIHVSTARMAYSSGSSPEMTAPGETNTGSTWDPTTYITTTDPKDTSSAQVSTPHSVR
TLRTTENHPKTESATPAAYSGSPKISSSPNLTSPATKAWTITDTTEHSTQLHYTKLAE
KSSGFETQSAPGPVSVVIPTSPTIGSSTLELTSDVPGEPLVLAPSEQTTITLPMATWLS
TSLTEEMASTDLDISSPSSPMSTFAIFPPMSTPSHELSKSEADTSAIRNTDSTTLDQHL
GIRSLGRTGDLTTVPITPLTTTWTSVIEHSTQAQDTLSATMSPTHVTQSLKDQTSIPA
SASPSHLTEVYPELGTQGRSSSEATTFWKPSTDTLSREIETGPTNIQSTPPMDNTTTG
SSSSGVTLGIAHLPIGTSSPAETSTNMALERRSSTATVSMAGTMGLLVTSAPGRSISQ
SLGRVSSVLSESTTEGVTDSSKGSSPRLNTQGNTALSSSLEPSYAEGSQMSTSIPLTSS
PTTPDVEFIGGSTFWTKEVTTVMTSDISKSSARTESSSATLMSTALGSTENTGKEKLR
TASMDLPSPTPSMEVTPWISLTLSNAPNTTDSLDLSHGVHTSSAGTLATDRSLNTGV
TRASRLENGSDTSSKSLSMGNSTHTSMTYTEKSEVSSSIHPRPETSAPGAETTLTSTP
GNRAISLTLPFSSIPVEEVISTGITSGPDINSAPMTHSPITPPTIVWTSTGTIEQSTQPLH
AVSSEKVSVQTQSTPYVNSVAVSASPTHENSVSSGSSTSSPYSSASLESLDSTISRRN
AITSWLWDLTTSLPTTTWPSTSLSEALSSGHSGVSNPSSTTTEFPLFSAASTSAAKQR
NPETETHGPQNTAASTLNTDASSVTGLSETPVGASISSEVPLPMAITSRSDVSGLTSE
STANPSLGTASSAGTKLTRTISLPTSESLVSFRMNKDPWTVSIPLGSHPTTNTETSIPV
NSAGPPGLSTVASDVIDTPSDGAESIPTVSFSPSPDTEVTTISHFPEKTTHSFRTISSLT
HELTSRVTPIPGDWMSSAMSTKPTGASPSITLGERRTITSAAPTTSPIVLTASFTETST
VSLDNETTVKTSDILDARKTNELPSDSSSSSDLINTSIASSTMDVTKTASISPTSISGM
TASSSPSLFSSDRPQVPTSTTETNTATSPSVSSNTYSLDGGSNVGGTPSTLPPFTITHP
VETSSALLAWSRPVRTFSTMVSTDTASGENPTSSNSVVTSVPAPGTWTSVGSTTDLP
AMGFLKTSPAGEAHSLLASTIEPATAFTPHLSAAVVTGSSATSEASLLTTSESKAIHS
SPQTPTTPTSGANWETSATPESLLVVTETSDTTLTSKILVTDTILFSTVSTPPSKFPSTG
TLSGASFPTLLPDTPAIPLTATEPTSSLATSFDSTPLVTIASDSLGTVPETTLTMSETSN
GDALVLKTVSNPDRSIPGITIQGVTESPLHPSSTSPSKIVAPRNTTYEGSITVALSTLPA
GTTGSLVFSQSSENSETTALVDSSAGLERASVMPLTTGSQGMASSGGIRSGSTHSTG
TKTFSSLPLTMNPGEVTAMSEITTNRLTATQSTAPKGIPVKPTSAESGLLTPVSASSSP
SKAFASLTTAPPTWGIPQSTLTFEFSEVPSLDTKSASLPTPGQSLNTIPDSDASTASSS
LSKSPEKNPRARMMTSTKAISASSFQSTGFTETPEGSASPSMAGHEPRVPTSGTGDP
RYASESMSYPDPSKASSAMTSTSLASKLTTLFSTGQAARSGSSSSPISLSTEKETSFLS
PTASTSRKTSLFLGPSMARQPNILVHLQTSALTLSPTSTLNMSQEEPPELTSSQTIAEE
EGTTAETQTLTFTPSETPTSLLPVSSPTEPTARRKSSPETWASSISVPAKTSLVETTDG
TLVTTIKMSSQAAQGNSTWPAPAEETGSSPAGTSPGSPEMSTTLKIMSSKEPSISPEIR
STVRNSPWKTPETTVPMETTVEPVTLQSTALGSGSTSISHLPTGTTSPTKSPTENMLA
TERVSLSPSPPEAWTNLYSGTPGGTRQSLATMSSVSLESPTARSITGTGQQSSPELVS
KTTGMEFSMWHGSTGGTTGDTHVSLSTSSNILEDPVTSPNSVSSLTDKSKHKTETW
VSTTAIPSTVLNNKIMAAEQQTSRSVDEAYSSTSSWSDQTSGSDITLGASPDVTNTL
YITSTAQTTSLVSLPSGDQGITSLTNPSGGKTSSASSVTSPSIGLETLRANVSAVKSDI
APTAGHLSQTSSPAEVSILDVTTAPTPGISTTITTMGTNSISTTTPNPEVGMSTMDSTP
ATERRTTSTEHPSTWSSTAASDSWTVTDMTSNLKVARSPGTISTMHTTSFLASSTEL
DSMSTPHGRITVIGTSLVTPSSDASAVKTETSTSERTLSPSDTTASTPISTFSRVQRMSI
SVPDILSTSWTPSSTEAEDVPVSMVSTDHASTKTDPNTPLSTFLFDSLSTLDWDTGRS
LSSATATTSAPQGATTPQELTLETMISPATSQLPFSIGHITSAVTPAAMARSSGVTFSR
PDPTSKKAEQTSTQLPTTTSAHPGQVPRSAATTLDVIPHTAKTPDATFQRQGQTALT
TEARATSDSWNEKEKSTPSAPWITEMMNSVSEDTIKEVTSSSSVLRTLNTLDINLES
GTTSSPSWKSSPYERIAPSESTTDKEAIHPSTNTVETTGWVTSSEHASHSTIPAHSASS
KLTSPVVTTSTREQAIVSMSTTTWPESTRARTEPNSFLTIELRDVSPYMDTSSTTQTSI
ISSPGSTAITKGPRTEITSSKRISSSFLAQSMRSSDSPSEAITRLSNFPAMTESGGMILA
MQTSPPGATSLSAPTLDTSATASWTGTPLATTQRFTYSEKTTLFSKGPEDTSQPSPPS
VEETSSSSSLVPIHATTSPSNILLTSQGHSPSSTPPVTSVFLSETSGLGKTTDMSRISLE
PGTSLPPNLSSTAGEALSTYEASRDTKAIHHSADTAVTNMEATSSEYSPIPGHTKPSK
ATSPLVTSHIMGDITSSTSVFGSSETTEIETVSSVNQGLQERSTSQVASSATETSTVIT
HVSSGDATTHVTKTQATFSSGTSISSPHQFITSTNTFTDVSTNPSTSLIMTESSGVTITT
QTGPTGAATQGPYLLDTSTMPYLTETPLAVTPDFMQSEKTTLISKGPKDVSWTSPPS
VAETSYPSSLTPFLVTTIPPATSTLQGQHTSSPVSATSVLTSGLVKTTDMLNTSMEPV
TNSPQNLNNPSNEILATLAATTDIETIHPSINKAVTNMGTASSAHVLHSTLPVSSEPST
ATSPMVPASSMGDALASISIPGSETTDIEGEPTSSLTAGRKENSTLQEMNSTTESNIIL
SNVSVGAITEATKMEVPSFDATFIPTPAQSTKFPDIFSVASSRLSNSPPMTISTHMTTT
QTGSSGATSKIPLALDTSTLETSAGTPSVVTEGFAHSKITTAMNNDVKDVSQTNPPF
QDEASSPSSQAPVLVTTLPSSVAFTPQWHSTSSPVSMSSVLTSSLVKTAGKVDTSLE
TVTSSPQSMSNTLDDISVTSAATTDIETTHPSINTVVTNVGTTGSAFESHSTVSAYPE
PSKVTSPNVTTSTMEDTTISRSIPKSSKTTRTETETTSSLTPKLRETSISQEITSSTETST
VPYKELTGATTEVSRTDVTSSSSTSFPGPDQSTVSLDISTETNTRLSTSPIMTESAEITI
TTQTGPHGATSQDTFTMDPSNTTPQAGIHSAMTHGFSQLDVTTLMSRIPQDVSWTS
PPSVDKTSSPSSFLSSPAMTTPSLISSTLPEDKLSSPMTSLLTSGLVKITDILRTRLEPV
TSSLPNFSSTSDKILATSKDSKDTKEIFPSINTEETNVKANNSGHESHSPALADSETPK
ATTQMVITTTVGDPAPSTSMPVHGSSETTNIKREPTYFLTPRLRETSTSQESSFPTDTS
FLLSKVPTGTITEVSSTGVNSSSKISTPDHDKSTVPPDTFTGEIPRVFTSSIKTKSAEMT
ITTQASPPESASHSTLPLDTSTTLSQGGTHSTVTQGFPYSEVTTLMGMGPGNVSWMT
TPPVEETSSVSSLMSSPAMTSPSPVSSTSPQSIPSSPLPVTALPTSVLVTTTDVLGTTSP
ESVTSSPPNLSSITHERPATYKDTAHTEAAMHHSTNTAVTNVGTSGSGHKSQSSVLA
DSETSKATPLMSTTSTLGDTSVSTSTPNISQTNQIQTEPTASLSPRLRESSTSEKTSSTT
ETNTAFSYVPTGAITQASRTEISSSRTSISDLDRPTIAPDISTGMITRLFTSPIMTKSAE
MTVTTQTTTPGATSQGILPWDTSTTLFQGGTHSTVSQGFPHSEITTLRSRTPGDVSW
MTTPPVEETSSGFSLMSPSMTSPSPVSSTSPESIPSSPLPVTALLTSVLVTTTNVLGTTS
PEPVTSSPPNLSSPTQERLTTYKDTAHTEAMHASMHTNTAVANVGTSISGHESQSSV
PADSHTSKATSPMGITFAMGDTSVSTSTPAFFETRIQTESTSSLIPGLRDTRTSEEINT
VTETSTVLSEVPTTTTTEVSRTEVITSSRTTISGPDHSKMSPYISTETITRLSTFPFVTGS
TEMAITNQTGPIGTISQATLTLDTSSTASWEGTHSPVTQRFPHSEETTTMSRSTKGVS
WQSPPSVEETSSPSSPVPLPAITSHSSLYSAVSGSSPTSALPVTSLLTSGRRKTIDMLD
THSELVTSSLPSASSFSGEILTSEASTNTETIHFSENTAETNMGTTNSMHKLHSSVSIH
SQPSGHTPPKVTGSMMEDAIVSTSTPGSPETKNVDRDSTSPLTPELKEDSTALVMNS
TTESNTVFSSVSLDAATEVSRAEVTYYDPTFMPASAQSTKSPDISPEASSSHSNSPPL
TISTHKTIATQTGPSGVTSLGQLTLDTSTIATSAGTPSARTQDFVDSETTSVMNNDLN
DVLKTSPFSAEEANSLSSQAPLLVTTSPSPVTSTLQEHSTSSLVSVTSVPTPTLAKITD
MDTNLEPVTRSPQNLRNTLATSEATTDTHTMHPSINTAVANVGTTSSPNEFYFTVSP
DSDPYKATSAVVITSTSGDSIVSTSMPRSSAMKKIESETTFSLIFRLRETSTSQKIGSSS
DTSTVFDKAFTAATTEVSRTELTSSSRTSIQGTEKPTMSPDTSTRSVTMLSTFAGLTK
SEERTIATQTGPHRATSQGTLTWDTSITTSQAGTHSAMTHGFSQLDLSTLTSRVPEYI
SGTSPPSVEKTSSSSSLLSLPAITSPSPVPTTLPESRPSSPVHLTSLPTSGLVKTTDMLA
SVASLPPNLGSTSHKIPTTSEDIKDTEKMYPSTNIAVTNVGTTTSEKESYSSVPAYSEP
PKVTSPMVTSFNIRDTIVSTSMPGSSEITRIEMESTFSLAHGLKGTSTSQDPIVSTEKS
AVLHKLTTGATETSRTEVASSRRTSIPGPDHSTESPDISTEVIPSLPISLGITESSNMTII
TRTGPPLGSTSQGTFTLDTPTTSSRAGTHSMATQEFPHSEMTTVMNKDPEILSWTIPP
SIEKTSFSSSLMPSPAMTSPPVSSTLPKTIHTTPSPMTSLLTPSLVMTTDTLGTSPEPTT
SSPPNLSSTSHEILTTDEDTTAIEAMHPSTSTAATNVETTSSGHGSQSSVLADSEKTK
ATAPMDTTSTMGHTTVSTSMSVSSETTKIKRESTYSLTPGLRETSISQNASFSTDTSI
VLSEVPTGTTAEVSRTEVTSSGRTSIPGPSQSTVLPEISTRTMTRLFASPTMTESAEMT
IPTQTGPSGSTSQDTLTLDTSTTKSQAKTHSTLTQRFPHSEMTTLMSRGPGDMSWQS
SPSLENPSSLPSLLSLPATTSPPPISSTLPVTISSSPLPVTSLLTSSPVTTTDMLHTSPELV
TSSPPKLSHTSDERLTTGKDTTNTEAVHPSTNTAASNVEIPSSGHESPSSALADSETS
KATSPMFITSTQEDTTVAISTPHFLETSRIQKESISSLSPKLRETGSSVETSSAIETSAVL
SEVSIGATTEISRTEVTSSSRTSISGSAESTMLPEISTTRKIIKFPTSPILAESSEMTIKTQ
TSPPGSTSESTFTLDTSTTPSLVITHSTMTQRLPHSEITTLVSRGAGDVPRPSSLPVEET
SPPSSQLSLSAMISPSPVSSTLPASSHSSSASVTSLLTPGQVKTTEVLDASAEPETSSPP
SLSSTSVEILATSEVTTDTEKIHPFSNTAVTKVGTSSSGHESPSSVLPDSETTKATSAM
GTISIMGDTSVSTLTPALSNTRKIQSEPASSLTTRLRETSTSEETSLATEANTVLSKVS
TGATTEVSRTEAISFSRTSMSGPEQSTMSQDISIGTIPRISASSVLTESAKMTITTQTGP
SESTLESTLNLNTATTPSWVETHSIVIQGFPHPEMTTSMGRGPGGVSWPSPPFVKETS
PPSSPLSLPAVTSPHPVSTTFLAHIPPSPLPVTSLLTSGPATTTDILGTSTEPGTSSSSSL
STTSHERLTTYKDTAHTEAVHPSTNTGGTNVATTSSGYKSQSSVLADSSPMCTTST
MGDTSVLTSTPAFLETRRIQTELASSLTPGLRESSGSEGTSSGTKMSTVLSKVPTGAT
TEISKEDVTSIPGPAQSTISPDISTRTVSWFSTSPVMTESAEITMNTHTSPLGATTQGT
STLDTSSTTSLTMTHSTISQGFSHSQMSTLMRRGPEDVSWMSPPLLEKTRPSFSLMSS
PATTSPSPVSSTLPESISSSPLPVTSLLTSGLAKTTDMLHKSSEPVTNSPANLSSTSVEI
LATSEVTTDTEKTHPSSNRTVTDVGTSSSGHESTSFVLADSQTSKVTSPMVITSTME
DTSVSTSTPGFFETSRIQTEPTSSLTLGLRKTSSSEGTSLATEMSTVLSGVPTGATAEV
SRTEVTSSSRTSISGFAQLTVSPETSTETITRLPTSSIMTESAEMMIKTQTDPPGSTPES
THTVDISTTPNWVETHSTVTQRFSHSEMTTLVSRSPGDMLWPSQSSVEETSSASSLL
SLPATTSPSPVSSTLVEDFPSASLPVTSLLNPGLVITTDRMGISREPGTSSTSNLSSTSH
ERLTTLEDTVDTEDMQPSTHTAVTNVRTSISGHESQSSVLSDSETPKATSPMGTTYT
MGETSVSISTSDFFETSRIQIEPTSSLTSGLRETSSSERISSATEGSTVLSEVPSGATTEV
SRTEVISSRGTSMSGPDQFTISPDISTEAITRLSTSPIMTESAESAITIETGSPGATSEGT
LTLDTSTTTFWSGTHSTASPGFSHSEMTTLMSRTPGDVPWPSLPSVEEASSVSSSLSS
PAMTSTSFFSTLPESISSSPHPVTALLTLGPVKTTDMLRTSSEPETSSPPNLSSTSAEIL
ATSEVTKDREKIHPSSNTPVVNVGTVIYKHLSPSSVLADLVTTKPTSPMATTSTLGN
TSVSTSTPAFPETMMTQPTSSLTSGLREISTSQETSSATERSASLSGMPTGATTKVSR
TEALSLGRTSTPGPAQSTISPEISTETITRISTPLTTTGSAEMTITPKTGHSGASSQGTFT
LDTSSRASWPGTHSAATHRSPHSGMTTPMSRGPEDVSWPSRPSVEKTSPPSSLVSLS
AVTSPSPLYSTPSESSHSSPLRVTSLFTPVMMKTTDMLDTSLEPVTTSPPSMNITSDES
LATSKATMETEAIQLSENTAVTQMGTISARQEFYSSYPGLPEPSKVTSPVVTSSTIKD
IVSTTIPASSEITRIEMESTSTLTPTPRETSTSQEIHSATKPSTVPYKALTSATIEDSMTQ
VMSSSRGPSPDQSTMSQDISTEVITRLSTSPIKTESTEMTITTQTGSPGATSRGTLTLD
TSTTFMSGTHSTASQGFSHSQMTALMSRTPGDVPWLSHPSVEEASSASFSLSSPVMT
SSSPVSSTLPDSIHSSSLPVTSLLTSGLVKTTELLGTSSEPETSSPPNLSSTSAEILAITE
VTTDTEKLEMTNVVTSGYTHESPSSVLADSVTTKATSSMGITYPTGDTNVLTSTPAF
SDTSRIQTKSKLSLTPGLMETSISEETSSATEKSTVLSSVPTGATTEVSRTEAISSSRTS
IPGPAQSTMSSDTSMETITRISTPLTRKESTDMAITPKTGPSGATSQGTFTLDSSSTAS
WPGTHSATTQRFPQSVVTTPMSRGPEDVSWPSPLSVEKNSPPSSLVSSSSVTSPSPLY
STPSGSSHSSPVPVTSLFTSIMMKATDMLDASLEPETTSAPNMNITSDESLAASKATT
ETEAIHVFENTAASHVETTSATEELYSSSPGFSEPTKVISPVVTSSSIRDNMVSTTMPG
SSGITRIEIESMSSLTPGLRETRTSQDITSSTETSTVLYKMPSGATPEVSRTEVMPSSRT
SIPGPAQSTMSLDISDEVVTRLSTSPIMTESAEITITTQTGYSLATSQVTLPLGTSMTFL
SGTHSTMSQGLSHSEMTNLMSRGPESLSWTSPRFVETTRSSSSLTSLPLTTSLSPVSS
TLLDSSPSSPLPVTSLILPGLVKTTEVLDTSSEPKTSSSPNLSSTSVEIPATSEIMTDTEK
IHPSSNTAVAKVRTSSSVHESHSSVLADSETTITIPSMGITSAVDDTTVFTSNPAFSET
RRIPTEPTFSLTPGFRETSTSEETTSITETSAVLYGVPTSATTEVSMTEIMSSNRIHIPDS
DQSTMSPDIITEVITRLSSSSMMSESTQMTITTQKSSPGATAQSTLTLATTTAPLART
HSTVPPRFLHSEMTTLMSRSPENPSWKSSLFVEKTSSSSSLLSLPVTTSPSVSSTLPQSI
PSSSFSVTSLLTPGMVKTTDTSTEPGTSLSPNLSGTSVEILAASEVTTDTEKIHPSSSM
AVTNVGTTSSGHELYSSVSIHSEPSKATYPVGTPSSMAETSISTSMPANFETTGFEAE
PFSHLTSGFRKTNMSLDTSSVTPTNTPSSPGSTHLLQSSKTDFTSSAKTSSPDWPPAS
QYTEIPVDIITPFNASPSITESTGITSFPESRFTMSVTESTHHLSTDLLPSAETISTGTVM
PSLSEAMTSFATTGVPRAISGSGSPFSRTESGPGDATLSTIAESLPSSTPVPFSSSTFTT
TDSSTIPALHEITSSSATPYRVDTSLGTESSTTEGRLVMVSTLDTSSQPGRTSSSPILDT
RMTESVELGTVTSAYQVPSLSTRLTRTDGIMEHITKIPNEAAHRGTIRPVKGPQTSTS
PASPKGLHTGGTKRMETTTTALKTTTTALKTTSRATLTTSVYTPTLGTLTPLNASM
QMASTIPTEMMITTPYVFPDVPETTSSLATSLGAETSTALPRTTPSVFNRESETTASL
VSRSGAERSPVIQTLDVSSSEPDTTASWVIHPAETIPTVSKTTPNFFHSELDTVSSTAT
SHGADVSSAIPTNISPSELDALTPLVTISGTDTSTTFPTLTKSPHETETRTTWLTHPAE
TSSTIPRTIPNFSHHESDATPSIATSPGAETSSAIPIMTVSPGAEDLVTSQVTSSGTDRN
MTIPTLTLSPGEPKTIASLVTHPEAQTSSAIPTSTISPAVSRLVTSMVTSLAAKTSTTN
RALTNSPGEPATTVSLVTHPAQTSPTVPWTTSIFFHSKSDTTPSMTTSHGAESSSAVP
TPTVSTEVPGVVTPLVTSSRAVISTTIPILTLSPGEPETTPSMATSHGEEASSAIPTPTV
SPGVPGVVTSLVTSSRAVTSTTIPILTFSLGEPETTPSMATSHGTEAGSAVPTVLPEVP
GMVTSLVASSRAVTSTTLPTLTLSPGEPETTPSMATSHGAEASSTVPTVSPEVPGVV
TSLVTSSSGVNSTSIPTLILSPGELETTPSMATSHGAEASSAVPTPTVSPGVSGVVTPL
VTSSRAVTSTTIPILTLSSSEPETTPSMATSHGVEASSAVLTVSPEVPGMVTSLVTSSR
AVTSTTIPTLTISSDEPETTTSLVTHSEAKMISAIPTLAVSPTVQGLVTSLVTSSGSETS
AFSNITVASSQPETIDSWVAHPGTEASSVVPTLTVSTGEPFTNISLVTHPAESSSTLPR
TTSRFSHSELDTMPSTVTSPEAESSSAISTTISPGIPGVLTSLVTSSGRDISATFPTVPES
PHESEATASWVTHPAVTSTTVPRTTPNYSHSEPDTTPSIATSPGAEATSDFPTITVSPD
VPDMVTSQVTSSGTDTSITIPTLTLSSGEPETTTSFITYSETHTSSAIPTLPVSPGASKM
LTSLVISSGTDSTTTFPTLTETPYEPETTAIQLIHPAETNTMVPRTTPKFSHSKSDTTLP
VAITSPGPEASSAVSTTTISPDMSDLVTSLVPSSGTDTSTTFPTLSETPYEPETTATWL
THPAETSTTVSGTIPNFSHRGSDTAPSMVTSPGVDTRSGVPTTTIPPSIPGVVTSQVTS
SATDTSTAIPTLTPSPGEPETTASSATHPGTQTGFTVPIRTVPSSEPDTMASWVTHPPQ
TSTPVSRTTSSFSHSSPDATPVMATSPRTEASSAVLTTISPGAPEMVTSQITSSGAATS
TTVPTLTHSPGMPETTALLSTHPRTETSKTFPASTVFPQVSETTASLTIRPGAETSTAL
PTQTTSSLFTLLVTGTSRVDLSPTASPGVSAKTAPLSTHPGTETSTMIPTSTLSLGLLE
TTGLLATSSSAETSTSTLTLTVSPAVSGLSSASITTDKPQTVTSWNTETSPSVTSVGPP
EFSRTVTGTTMTLIPSEMPTPPKTSHGEGVSPTTILRTTMVEATNLATTGSSPTVAKT
TTTFNTLAGSLFTPLTTPGMSTLASESVTSRTSYNHRSWISTTSSYNRRYWTPATSTP
VTSTFSPGISTSSIPSSTAATVPFMVPFTLNFTITNLQYEEDMRHPGSRKFNATERELQ
GLLKPLFRNSSLEYLYSGCRLASLRPEKDSSATAVDAICTHRPDPEDLGLDRERLYW
ELSNLTNGIQELGPYTLDRNSLYVNGFTHRSSMPTTSTPGTSTVDVGTSGTPSSSPSP
TTAGPLLMPFTLNFTITNLQYEEDMRRTGSRKFNTMESVLQGLLKPLFKNTSVGPL
YSGCRLTLLRPEKDGAATGVDAICTHRLDPKSPGLNREQLYWELSKLTNDIEELGP
YTLDRNSLYVNGFTHQSSVSTTSTPGTSTVDLRTSGTPSSLSSPTIMAAGPLLVPFTL
NFTITNLQYGEDMGHPGSRKFNTTERVLQGLLGPIFKNTSVGPLYSGCRLTSLRSEK
DGAATGVDAICIHHLDPKSPGLNRERLYWELSQLTNGIKELGPYTLDRNSLYVNGF
THRTSVPTSSTPGTSTVDLGTSGTPFSLPSPATAGPLLVLFTLNFTITNLKYEEDMHR
PGSRKFNTTERVLQTLLGPMFKNTSVGLLYSGCRLTLLRSEKDGAATGVDAICTHR
LDPKSPGVDREQLYWELSQLTNGIKELGPYTLDRNSLYVNGFTHWIPVPTSSTPGTS
TVDLGSGTPSSLPSPTTAGPLLVPFTLNFTITNLKYEEDMHCPGSRKFNTTERVLQSL
LGPMFKNTSVGPLYSGCRLTLLRSEKDGAATGVDAICTHRLDPKSPGVDREQLYW
ELSQLTNGIKELGPYTLDRNSLYVNGFTHQTSAPNTSTPGTSTVDLGTSGTPSSLPSP
TSAGPLLVPFTLNFTITNLQYEEDMHHPGSRKENTTERVLQGLLGPMFKNTSVGLL
YSGCRLTLLRPEKNGAATGMDAICSHRLDPKSPGLNREQLYWELSQLTHGIKELGP
YTLDRNSLYVNGFTHRSSVAPTSTPGTSTVDLGTSGTPSSLPSPTTAVPLLVPFTLNF
TITNLQYGEDMRHPGSRKFNTTERVLQGLLGPLFKNSSVGPLYSGCRLISLRSEKDG
AATGVDAICTHHLNPQSPGLDREQLYWQLSQMTNGIKELGPYTLDRNSLYVNGFT
HRSSGLTTSTPWTSTVDLGTSGTPSPVPSPTTTGPLLVPFTLNFTITNLQYEENMGHP
GSRKFNITESVLQGLLKPLFKSTSVGPLYSGCRLTLLRPEKDGVATRVDAICTHRPD
PKIPGLDRQQLYWELSQLTHSITELGPYTLDRDSLYVNGFTQRSSVPTTSTPGTFTVQ
PETSETPSSLPGPTATGPVLLPFTLNFTITNLQYEEDMRRPGSRKFNTTERVLQGLLM
PLFKNTSVSSLYSGCRLTLLRPEKDGAATRVDAVCTHRPDPKSPGLDRERLYWKLS
QLTHGITELGPYTLDRHSLYVNGFTHQSSMTTTRTPDTSTMHLATSRTPASLSGPMT
ASPLLVLFTINFTITNLRYEENMHHPGSRKFNTTERVLQGLLRPVFKNTSVGPLYSG
CRLTLLRPKKDGAATKVDAICTYRPDPKSPGLDREQLYWELSQLTHSITELGPYTLD
RDSLYVNGFTQRSSVPTTSIPGTPTVDLGTSGTPVSKPGPSAASPLLVLFTLNFTITNL
RYEENMQHPGSRKFNTTERVLQGLLRSLFKSTSVGPLYSGCRLTLLRPEKDGTATG
VDAICTHHPDPKSPRLDREQLYWELSQLTHNITELGPYALDNDSLFVNGFTHRSSVS
TTSTPGTPTVYLGASKTPASIFGPSAASHLLILFTLNFTITNLRYEENMWPGSRKENT
TERVLQGLLRPLFKNTSVGPLYSGCRLTLLRPEKDGEATGVDAICTHRPDPTGPGLD
REQLYLELSQLTHSITELGPYTLDRDSLYVNGFTHRSSVPTTSTGVVSEEPFTLNFTI
NNLRYMADMGQPGSLKFNITDNVMQHLLSPLFQRSSLGARYTGCRVIALRSVKNG
AETRVDLLCTYLQPLSGPGLPIKQVFHELSQQTHGITRLGPYSLDKDSLYLNGYNEP
GPDEPPTTPKPATTFLPPLSEATTAMGYHLKTLTLNFTISNLQYSPDMGKGSATFNS
TEGVLQHLLRPLFQKSSMGPFYLGCQLISLRPEKDGAATGVDTTCTYHPDPVGPGL
DIQQLYWELSQLTHGVTQLGFYVLDRDSLFINGYAPQNLSIRGEYQINFHIVNWNLS
NPDPTSSEYITLLRDIQDKVTTLYKGSQLHDTFRFCLVTNLTMDSVLVTVKALFSSN
LDPSLVEQVFLDKTLNASFHWLGSTYQLVDIHVTEMESSVYQPTSSSSTQHFYLNFT
ITNLPYSQDKAQPGTTNYQRNKRNIEDALNQLFRNSSIKSYFSDCQVSTFRSVPNRH
HTGVDSLCNFSPLARRVDRVAIYEEFLRMTRNGTQLQNFTLDRSSVLVDGYSPNRN
EPLTGNSDLPFWAVILIGLAGLLGVITCLICGVLVTTRRRKKEGEYNVQQQCPGYYQ
SHLDLEDLQ

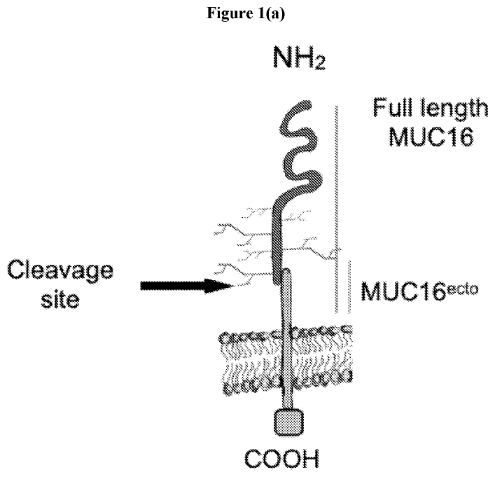

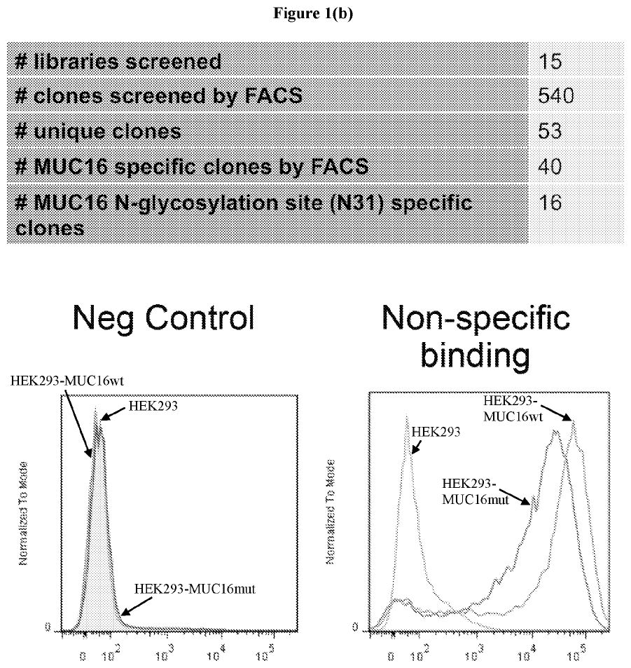

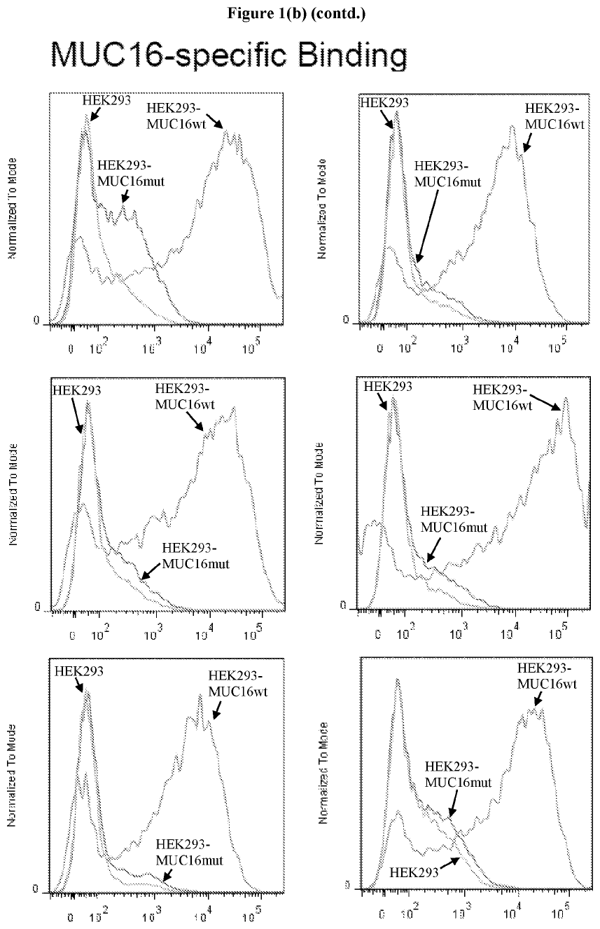

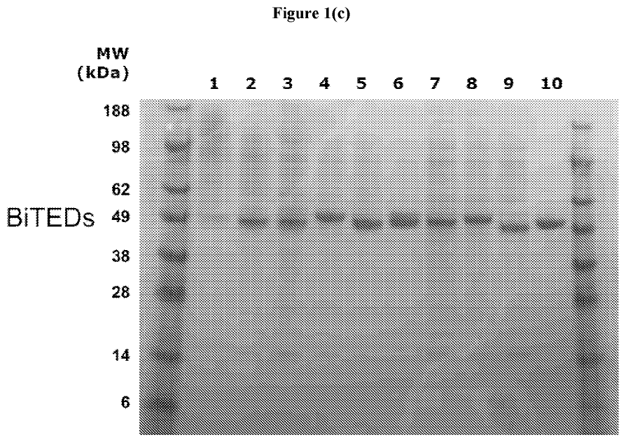

Native MUC16 comprises an intracellular domain, a transmembrane domain, an ectodomain proximal to the putative cleavage site, and a large, heavily glycosylated region of 12-20 repeats, each 156 amino acids long (FIG. 1(a)). “Immature” MUC16 refers to SEQ ID NO: 1, which comprises the MUC16 signal sequence (amino acid residues 1-60 of SEQ ID NO: 1). “Mature MUC16” refers to native MUC16 as expressed on the cell surface, i.e., where the signal sequence has been removed by cellular processing, for example, SEQ ID NO: 2, where the first 60 amino acid residues of SEQ ID NO: 1 have been removed (i.e., SEQ ID NO: 1 is the “immature” form of MUC16).

″Mature MUC16″
(SEQ ID NO: 2)
DKTLASPTSSVVGRTTQSLGVMSSALPESTSRGMTHSEQRTSPSLSPQVNGTPSRNYP
ATSMVSGLSSPRTRTSSTEGNFTKEASTYTLTVETTSGPVTEKYTVPTETSTTEGDSTE
TPWDTRYIPVKITSPMKTFADSTASKENAPVSMTPAETTVTDSHTPGRTNPSFGTLYS
SFLDLSPKGTPNSRGETSLELILSTTGYPFSSPEPGSAGHSRISTSAPLSSSASVLDNKIS
ETSIFSGQSLTSPLSPGVPEARASTMPNSAIPFSMTLSNAETSAERVRSTISSLGTPSIST
KQTAETILTFHAFAETMDIPSTHIAKTLASEWLGSPGTLGGTSTSALTTTSPSTTLVSEE
TNTHHSTSGKETEGTLNTSMTPLETSAPGEESEMTATLVPTLGFTTLDSKIRSPSQVSS
SHPTRELRTTGSTSGRQSSSTAAHGSSDILRATTSSTSKASSWTSESTAQQFSEPQHTQ
WVETSPSMKTERPPASTSVAAPITTSVPSVVSGFTTLKTSSTKGIWLEETSADTLIGES
TAGPTTHQFAVPTGISMTGGSSTRGSQGTTHLLTRATASSETSADLTLATNGVPVSVS
PAVSKTAAGSSPPGGTKPSYTMVSSVIPETSSLQSSAFREGTSLGLTPLNTRHPFSSPEP
DSAGHTKISTSIPLLSSASVLEDKVSATSTFSHHKATSSITTGTPEISTKTKPSSAVLSSM
TLSNAATSPERVRNATSPLTHPSPSGEETAGSVLTLSTSAETTDSPNIHPTGTLTSESSE
SPSTLSLPSVSGVKTTFSSSTPSTHLFTSGEETEETSNPSVSQPETSVSRVRTTLASTSVP
TPVFPTMDTWPTRSAQFSSSHLVSELRATSSTSVINSTGSALPKISHLTGTATMSQTN
RDTFNDSAAPQSTTWPETSPRFKTGLPSATTTVSTSATSLSATVMVSKFTSPATSSME
ATSIREPSTTILTTETTNGPGSMAVASTNIPIGKGYITEGRLDTSHLPIGTTASSETSMDF
TMAKESVSMSVSPSQSMDAAGSSTPGRTSQFVDTFSDDVYHLTSREITIPRDGTSSAL
TPQMTATHPPSPDPGSARSTWLGILSSSPSSPTPKVTMSSTFSTQRVTTSMIMDTVETS
RWNMPNLPSTTSLTPSNIPTSGAIGKSTLVPLDTPSPATSLEASEGGLPTLSTYPESTNT
PSIHLGAHASSESPSTIKLTMASVVKPGSYTPLTFPSIETHIHVSTARMAYSSGSSPEMT
APGETNTGSTWDPTTYITTTDPKDTSSAQVSTPHSVRTLRTTENHPKTESATPAAYSG
SPKISSSPNLTSPATKAWTITDTTEHSTQLHYTKLAEKSSGFETQSAPGPVSVVIPTSPT
IGSSTLELTSDVPGEPLVLAPSEQTTITLPMATWLSTSLTEEMASTDLDISSPSSPMSTF
AIFPPMSTPSHELSKSEADTSAIRNTDSTTLDQHLGIRSLGRTGDLTTVPITPLTTTWTS
VIEHSTQAQDTLSATMSPTHVTQSLKDQTSIPASASPSHLTEVYPELGTQGRSSSEATT
FWKPSTDTLSREIETGPTNIQSTPPMDNTTTGSSSSGVTLGIAHLPIGTSSPAETSTNMA
LERRSSTATVSMAGTMGLLVTSAPGRSISQSLGRVSSVLSESTTEGVTDSSKGSSPRL
NTQGNTALSSSLEPSYAEGSQMSTSIPLTSSPTTPDVEFIGGSTFWTKEVTTVMTSDIS
KSSARTESSSATLMSTALGSTENTGKEKLRTASMDLPSPTPSMEVTPWISLTLSNAPN
TTDSLDLSHGVHTSSAGTLATDRSLNTGVTRASRLENGSDTSSKSLSMGNSTHTSMT
YTEKSEVSSSIHPRPETSAPGAETTLTSTPGNRAISLTLPFSSIPVEEVISTGITSGPDINS
APMTHSPITPPTIVWTSTGTIEQSTQPLHAVSSEKVSVQTQSTPYVNSVAVSASPTHEN
SVSSGSSTSSPYSSASLESLDSTISRRNAITSWLWDLTTSLPTTTWPSTSLSEALSSGHS
GVSNPSSTTTEFPLFSAASTSAAKQRNPETETHGPQNTAASTLNTDASSVTGLSETPV
GASISSEVPLPMAITSRSDVSGLTSESTANPSLGTASSAGTKLTRTISLPTSESLVSFRM
NKDPWTVSIPLGSHPTTNTETSIPVNSAGPPGLSTVASDVIDTPSDGAESIPTVSFSPSP
DTEVTTISHFPEKTTHSFRTISSLTHELTSRVTPIPGDWMSSAMSTKPTGASPSITLGER
RTITSAAPTTSPIVLTASFTETSTVSLDNETTVKTSDILDARKTNELPSDSSSSSDLINTSI
ASSTMDVTKTASISPTSISGMTASSSPSLFSSDRPQVPTSTTETNTATSPSVSSNTYSLD
GGSNVGGTPSTLPPFTITHPVETSSALLAWSRPVRTFSTMVSTDTASGENPTSSNSVVT
SVPAPGTWTSVGSTTDLPAMGFLKTSPAGEAHSLLASTIEPATAFTPHLSAAVVTGSS
ATSEASLLTTSESKAIHSSPQTPTTPTSGANWETSATPESLLVVTETSDTTLTSKILVTD
TILFSTVSTPPSKFPSTGTLSGASFPTLLPDTPAIPLTATEPTSSLATSFDSTPLVTIASDS
LGTVPETTLTMSETSNGDALVLKTVSNPDRSIPGITIQGVTESPLHPSSTSPSKIVAPRN
TTYEGSITVALSTLPAGTTGSLVFSQSSENSETTALVDSSAGLERASVMPLTTGSQGM
ASSGGIRSGSTHSTGTKTFSSLPLTMNPGEVTAMSEITTNRLTATQSTAPKGIPVKPTS
AESGLLTPVSASSSPSKAFASLTTAPPTWGIPQSTLTFEFSEVPSLDTKSASLPTPGQSL
NTIPDSDASTASSSLSKSPEKNPRARMMTSTKAISASSFQSTGFTETPEGSASPSMAGH
EPRVPTSGTGDPRYASESMSYPDPSKASSAMTSTSLASKLTTLFSTGQAARSGSSSSPI
SLSTEKETSFLSPTASTSRKTSLFLGPSMARQPNILVHLQTSALTLSPTSTLNMSQEEPP
ELTSSQTIAEEEGTTAETQTLTFTPSETPTSLLPVSSPTEPTARRKSSPETWASSISVPAK
TSLVETTDGTLVTTIKMSSQAAQGNSTWPAPAEETGSSPAGTSPGSPEMSTTLKIMSS
KEPSISPEIRSTVRNSPWKTPETTVPMETTVEPVTLQSTALGSGSTSISHLPTGTTSPTK
SPTENMLATERVSLSPSPPEAWTNLYSGTPGGTRQSLATMSSVSLESPTARSITGTGQ
QSSPELVSKTTGMEFSMWHGSTGGTTGDTHVSLSTSSNILEDPVTSPNSVSSLTDKSK
HKTETWVSTTAIPSTVLNNKIMAAEQQTSRSVDEAYSSTSSWSDQTSGSDITLGASPD
VTNTLYITSTAQTTSLVSLPSGDQGITSLTNPSGGKTSSASSVTSPSIGLETLRANVSAV
KSDIAPTAGHLSQTSSPAEVSILDVTTAPTPGISTTITTMGTNSISTTTPNPEVGMSTMD
STPATERRTTSTEHPSTWSSTAASDSWTVTDMTSNLKVARSPGTISTMHTTSFLASST
ELDSMSTPHGRITVIGTSLVTPSSDASAVKTETSTSERTLSPSDTTASTPISTFSRVQRM
SISVPDILSTSWTPSSTEAEDVPVSMVSTDHASTKTDPNTPLSTFLFDSLSTLDWDTGR
SLSSATATTSAPQGATTPQELTLETMISPATSQLPFSIGHITSAVTPAAMARSSGVTFSR
PDPTSKKAEQTSTQLPTTTSAHPGQVPRSAATTLDVIPHTAKTPDATFQRQGQTALTT
EARATSDSWNEKEKSTPSAPWITEMMNSVSEDTIKEVTSSSSVLRTLNTLDINLESGT
TSSPSWKSSPYERIAPSESTTDKEAIHPSTNTVETTGWVTSSEHASHSTIPAHSASSKLT
SPVVTTSTREQAIVSMSTTTWPESTRARTEPNSFLTIELRDVSPYMDTSSTTQTSIISSP
GSTAITKGPRTEITSSKRISSSFLAQSMRSSDSPSEAITRLSNFPAMTESGGMILAMQTS
PPGATSLSAPTLDTSATASWTGTPLATTQRFTYSEKTTLFSKGPEDTSQPSPPSVEETS
SSSSLVPIHATTSPSNILLTSQGHSPSSTPPVTSVFLSETSGLGKTTDMSRISLEPGTSLPP
NLSSTAGEALSTYEASRDTKAIHHSADTAVTNMEATSSEYSPIPGHTKPSKATSPLVT
SHIMGDITSSTSVFGSSETTEIETVSSVNQGLQERSTSQVASSATETSTVITHVSSGDAT
THVTKTQATFSSGTSISSPHQFITSTNTFTDVSTNPSTSLIMTESSGVTITTQTGPTGAAT
QGPYLLDTSTMPYLTETPLAVTPDFMQSEKTTLISKGPKDVSWTSPPSVAETSYPSSL
TPFLVTTIPPATSTLQGQHTSSPVSATSVLTSGLVKTTDMLNTSMEPVTNSPQNLNNPS
NEILATLAATTDIETIHPSINKAVINMGTASSAHVLHSTLPVSSEPSTATSPMVPASSM
GDALASISIPGSETTDIEGEPTSSLTAGRKENSTLQEMNSTTESNIILSNVSVGAITEATK
MEVPSFDATFIPTPAQSTKFPDIFSVASSRLSNSPPMTISTHMTTTQTGSSGATSKIPLA
LDTSTLETSAGTPSVVTEGFAHSKITTAMNNDVKDVSQTNPPFQDEASSPSSQAPVLV
TTLPSSVAFTPQWHSTSSPVSMSSVLTSSLVKTAGKVDTSLETVTSSPQSMSNTLDDIS
VTSAATTDIETTHPSINTVVTNVGTTGSAFESHSTVSAYPEPSKVTSPNVTTSTMEDTT
ISRSIPKSSKTTRTETETTSSLTPKLRETSISQEITSSTETSTVPYKELTGATTEVSRTDVT
SSSSTSFPGPDQSTVSLDISTETNTRLSTSPIMTESAEITITTQTGPHGATSQDTFTMDPS
NTTPQAGIHSAMTHGFSQLDVTTLMSRIPQDVSWTSPPSVDKTSSPSSFLSSPAMTTPS
LISSTLPEDKLSSPMTSLLTSGLVKITDILRTRLEPVTSSLPNFSSTSDKILATSKDSKDT
KEIFPSINTEETNVKANNSGHESHSPALADSETPKATTQMVITTTVGDPAPSTSMPVH
GSSETTNIKREPTYFLTPRLRETSTSQESSFPTDTSFLLSKVPTGTITEVSSTGVNSSSKIS
TPDHDKSTVPPDTFTGEIPRVFTSSIKTKSAEMTITTQASPPESASHSTLPLDTSTTLSQ
GGTHSTVTQGFPYSEVTTLMGMGPGNVSWMTTPPVEETSSVSSLMSSPAMTSPSPVS
STSPQSIPSSPLPVTALPTSVLVTTTDVLGTTSPESVTSSPPNLSSITHERPATYKDTAHT
EAAMHHSTNTAVTNVGTSGSGHKSQSSVLADSETSKATPLMSTTSTLGDTSVSTSTP
NISQTNQIQTEPTASLSPRLRESSTSEKTSSTTETNTAFSYVPTGAITQASRTEISSSRTSI
SDLDRPTIAPDISTGMITRLFTSPIMTKSAEMTVTTQTTTPGATSQGILPWDTSTTLFQG
GTHSTVSQGFPHSEITTLRSRTPGDVSWMTTPPVEETSSGFSLMSPSMTSPSPVSSTSP
ESIPSSPLPVTALLTSVLVTTTNVLGTTSPEPVTSSPPNLSSPTQERLTTYKDTAHTEAM
HASMHTNTAVANVGTSISGHESQSSVPADSHTSKATSPMGITFAMGDTSVSTSTPAFF
ETRIQTESTSSLIPGLRDTRTSEEINTVTETSTVLSEVPTTTTTEVSRTEVITSSRTTISGP
DHSKMSPYISTETITRLSTFPFVTGSTEMAITNQTGPIGTISQATLTLDTSSTASWEGTH
SPVTQRFPHSEETTTMSRSTKGVSWQSPPSVEETSSPSSPVPLPAITSHSSLYSAVSGSS
PTSALPVTSLLTSGRRKTIDMLDTHSELVTSSLPSASSFSGEILTSEASTNTETIHFSENT
AETNMGTTNSMHKLHSSVSIHSQPSGHTPPKVTGSMMEDAIVSTSTPGSPETKNVDR
DSTSPLTPELKEDSTALVMNSTTESNTVFSSVSLDAATEVSRAEVTYYDPTFMPASAQ
STKSPDISPEASSSHSNSPPLTISTHKTIATQTGPSGVTSLGQLTLDTSTIATSAGTPSAR
TQDFVDSETTSVMNNDLNDVLKTSPFSAEEANSLSSQAPLLVTTSPSPVTSTLQEHST
SSLVSVTSVPTPTLAKITDMDTNLEPVTRSPQNLRNTLATSEATTDTHTMHPSINTAV
ANVGTTSSPNEFYFTVSPDSDPYKATSAVVITSTSGDSIVSTSMPRSSAMKKIESETTFS
LIFRLRETSTSQKIGSSSDTSTVFDKAFTAATTEVSRTELTSSSRTSIQGTEKPTMSPDTS
TRSVTMLSTFAGLTKSEERTIATQTGPHRATSQGTLTWDTSITTSQAGTHSAMTHGFS
QLDLSTLTSRVPEYISGTSPPSVEKTSSSSSLLSLPAITSPSPVPTTLPESRPSSPVHLTSL
PTSGLVKTTDMLASVASLPPNLGSTSHKIPTTSEDIKDTEKMYPSTNIAVTNVGTTTSE
KESYSSVPAYSEPPKVTSPMVTSFNIRDTIVSTSMPGSSEITRIEMESTFSLAHGLKGTS
TSQDPIVSTEKSAVLHKLTTGATETSRTEVASSRRTSIPGPDHSTESPDISTEVIPSLPISL
GITESSNMTIITRTGPPLGSTSQGTFTLDTPTTSSRAGTHSMATQEFPHSEMTTVMNKD
PEILSWTIPPSIEKTSFSSSLMPSPAMTSPPVSSTLPKTIHTTPSPMTSLLTPSLVMTTDTL
GTSPEPTTSSPPNLSSTSHEILTTDEDTTAIEAMHPSTSTAATNVETTSSGHGSQSSVLA
DSEKTKATAPMDTTSTMGHTTVSTSMSVSSETTKIKRESTYSLTPGLRETSISQNASFS
TDTSIVLSEVPTGTTAEVSRTEVTSSGRTSIPGPSQSTVLPEISTRTMTRLFASPTMTES
AEMTIPTQTGPSGSTSQDTLTLDTSTTKSQAKTHSTLTQRFPHSEMTTLMSRGPGDMS
WQSSPSLENPSSLPSLLSLPATTSPPPISSTLPVTISSSPLPVTSLLTSSPVTTTDMLHTSP
ELVTSSPPKLSHTSDERLTTGKDTTNTEAVHPSTNTAASNVEIPSSGHESPSSALADSE
TSKATSPMFITSTQEDTTVAISTPHFLETSRIQKESISSLSPKLRETGSSVETSSAIETSAV
LSEVSIGATTEISRTEVTSSSRTSISGSAESTMLPEISTTRKIIKFPTSPILAESSEMTIKTQ
TSPPGSTSESTFTLDTSTTPSLVITHSTMTQRLPHSEITTLVSRGAGDVPRPSSLPVEETS
PPSSQLSLSAMISPSPVSSTLPASSHSSSASVTSLLTPGQVKTTEVLDASAEPETSSPPSL
SSTSVEILATSEVTTDTEKIHPFSNTAVTKVGTSSSGHESPSSVLPDSETTKATSAMGTI
SIMGDTSVSTLTPALSNTRKIQSEPASSLTTRLRETSTSEETSLATEANTVLSKVSTGAT
TEVSRTEAISFSRTSMSGPEQSTMSQDISIGTIPRISASSVLTESAKMTITTQTGPSESTL
ESTLNLNTATTPSWVETHSIVIQGFPHPEMTTSMGRGPGGVSWPSPPFVKETSPPSSPL
SLPAVTSPHPVSTTFLAHIPPSPLPVTSLLTSGPATTTDILGTSTEPGTSSSSSLSTTSHER
LTTYKDTAHTEAVHPSTNTGGTNVATTSSGYKSQSSVLADSSPMCTTSTMGDTSVLT
STPAFLETRRIQTELASSLTPGLRESSGSEGTSSGTKMSTVLSKVPTGATTEISKEDVTS
IPGPAQSTISPDISTRTVSWFSTSPVMTESAEITMNTHTSPLGATTQGTSTLDTSSTTSL
TMTHSTISQGFSHSQMSTLMRRGPEDVSWMSPPLLEKTRPSFSLMSSPATTSPSPVSST
LPESISSSPLPVTSLLTSGLAKTTDMLHKSSEPVTNSPANLSSTSVEILATSEVTTDTEK
THPSSNRTVTDVGTSSSGHESTSFVLADSQTSKVTSPMVITSTMEDTSVSTSTPGFFET
SRIQTEPTSSLTLGLRKTSSSEGTSLATEMSTVLSGVPTGATAEVSRTEVTSSSRTSISG
FAQLTVSPETSTETITRLPTSSIMTESAEMMIKTQTDPPGSTPESTHTVDISTTPNWVET
HSTVTQRFSHSEMTTLVSRSPGDMLWPSQSSVEETSSASSLLSLPATTSPSPVSSTLVE
DFPSASLPVTSLLNPGLVITTDRMGISREPGTSSTSNLSSTSHERLTTLEDTVDTEDMQ
PSTHTAVTNVRTSISGHESQSSVLSDSETPKATSPMGTTYTMGETSVSISTSDFFETSRI
QIEPTSSLTSGLRETSSSERISSATEGSTVLSEVPSGATTEVSRTEVISSRGTSMSGPDQF
TISPDISTEAITRLSTSPIMTESAESAITIETGSPGATSEGTLTLDTSTTTFWSGTHSTASP
GFSHSEMTTLMSRTPGDVPWPSLPSVEEASSVSSSLSSPAMTSTSFFSTLPESISSSPHP
VTALLTLGPVKTTDMLRTSSEPETSSPPNLSSTSAEILATSEVTKDREKIHPSSNTPVVN
VGTVIYKHLSPSSVLADLVTTKPTSPMATTSTLGNTSVSTSTPAFPETMMTQPTSSLTS
GLREISTSQETSSATERSASLSGMPTGATTKVSRTEALSLGRTSTPGPAQSTISPEISTET
ITRISTPLTTTGSAEMTITPKTGHSGASSQGTFTLDTSSRASWPGTHSAATHRSPHSGM
TTPMSRGPEDVSWPSRPSVEKTSPPSSLVSLSAVTSPSPLYSTPSESSHSSPLRVTSLFT
PVMMKTTDMLDTSLEPVTTSPPSMNITSDESLATSKATMETEAIQLSENTAVTQMGTI
SARQEFYSSYPGLPEPSKVTSPVVTSSTIKDIVSTTIPASSEITRIEMESTSTLTPTPRETS
TSQEIHSATKPSTVPYKALTSATIEDSMTQVMSSSRGPSPDQSTMSQDISTEVITRLSTS
PIKTESTEMTITTQTGSPGATSRGTLTLDTSTTFMSGTHSTASQGFSHSQMTALMSRTP
GDVPWLSHPSVEEASSASFSLSSPVMTSSSPVSSTLPDSIHSSSLPVTSLLTSGLVKTTE
LLGTSSEPETSSPPNLSSTSAEILAITEVTTDTEKLEMTNVVTSGYTHESPSSVLADSVT
TKATSSMGITYPTGDTNVLTSTPAFSDTSRIQTKSKLSLTPGLMETSISEETSSATEKST
VLSSVPTGATTEVSRTEAISSSRTSIPGPAQSTMSSDTSMETITRISTPLTRKESTDMAIT
PKTGPSGATSQGTFTLDSSSTASWPGTHSATTQRFPQSVVTTPMSRGPEDVSWPSPLS
VEKNSPPSSLVSSSSVTSPSPLYSTPSGSSHSSPVPVTSLFTSIMMKATDMLDASLEPET
TSAPNMNITSDESLAASKATTETEAIHVFENTAASHVETTSATEELYSSSPGFSEPTKVI
SPVVTSSSIRDNMVSTTMPGSSGITRIEIESMSSLTPGLRETRTSQDITSSTETSTVLYK
MPSGATPEVSRTEVMPSSRTSIPGPAQSTMSLDISDEVVTRLSTSPIMTESAEITITTQT
GYSLATSQVTLPLGTSMTFLSGTHSTMSQGLSHSEMTNLMSRGPESLSWTSPRFVETT
RSSSSLTSLPLTTSLSPVSSTLLDSSPSSPLPVTSLILPGLVKTTEVLDTSSEPKTSSSPNL
SSTSVEIPATSEIMTDTEKIHPSSNTAVAKVRTSSSVHESHSSVLADSETTITIPSMGITS
AVDDTTVFTSNPAFSETRRIPTEPTFSLTPGFRETSTSEETTSITETSAVLYGVPTSATTE
VSMTEIMSSNRIHIPDSDQSTMSPDIITEVITRLSSSSMMSESTQMTITTQKSSPGATAQ
STLTLATTTAPLARTHSTVPPRFLHSEMTTLMSRSPENPSWKSSLFVEKTSSSSSLLSLP
VTTSPSVSSTLPQSIPSSSFSVTSLLTPGMVKTTDTSTEPGTSLSPNLSGTSVEILAASEV
TTDTEKIHPSSSMAVTNVGTTSSGHELYSSVSIHSEPSKATYPVGTPSSMAETSISTSM
PANFETTGFEAEPFSHLTSGFRKTNMSLDTSSVTPTNTPSSPGSTHLLQSSKTDFTSSA
KTSSPDWPPASQYTEIPVDIITPFNASPSITESTGITSFPESRFTMSVTESTHHLSTDLLPS
AETISTGTVMPSLSEAMTSFATTGVPRAISGSGSPFSRTESGPGDATLSTIAESLPSSTP
VPFSSSTFTTTDSSTIPALHEITSSSATPYRVDTSLGTESSTTEGRLVMVSTLDTSSQPG
RTSSSPILDTRMTESVELGTVTSAYQVPSLSTRLTRTDGIMEHITKIPNEAAHRGTIRPV
KGPQTSTSPASPKGLHTGGTKRMETTTTALKTTTTALKTTSRATLTTSVYTPTLGTLT
PLNASMQMASTIPTEMMITTPYVFPDVPETTSSLATSLGAETSTALPRTTPSVFNRESE
TTASLVSRSGAERSPVIQTLDVSSSEPDTTASWVIHPAETIPTVSKTTPNFFHSELDTVS
STATSHGADVSSAIPTNISPSELDALTPLVTISGTDTSTTFPTLTKSPHETETRTTWLTH
PAETSSTIPRTIPNFSHHESDATPSIATSPGAETSSAIPIMTVSPGAEDLVTSQVTSSGTD
RNMTIPTLTLSPGEPKTIASLVTHPEAQTSSAIPTSTISPAVSRLVTSMVTSLAAKTSTT
NRALTNSPGEPATTVSLVTHPAQTSPTVPWTTSIFFHSKSDTTPSMTTSHGAESSSAVP
TPTVSTEVPGVVTPLVTSSRAVISTTIPILTLSPGEPETTPSMATSHGEEASSAIPTPTVS
PGVPGVVTSLVTSSRAVTSTTIPILTFSLGEPETTPSMATSHGTEAGSAVPTVLPEVPG
MVTSLVASSRAVTSTTLPTLTLSPGEPETTPSMATSHGAEASSTVPTVSPEVPGVVTSL
VTSSSGVNSTSIPTLILSPGELETTPSMATSHGAEASSAVPTPTVSPGVSGVVTPLVTSS
RAVTSTTIPILTLSSSEPETTPSMATSHGVEASSAVLTVSPEVPGMVTSLVTSSRAVTST
TIPTLTISSDEPETTTSLVTHSEAKMISAIPTLAVSPTVQGLVTSLVTSSGSETSAFSNLT
VASSQPETIDSWVAHPGTEASSVVPTLTVSTGEPFTNISLVTHPAESSSTLPRTTSRFSH
SELDTMPSTVTSPEAESSSAISTTISPGIPGVLTSLVTSSGRDISATFPTVPESPHESEATA
SWVTHPAVTSTTVPRTTPNYSHSEPDTTPSIATSPGAEATSDFPTITVSPDVPDMVTSQ
VTSSGTDTSITIPTLTLSSGEPETTTSFITYSETHTSSAIPTLPVSPGASKMLTSLVISSGT
DSTTTFPTLTETPYEPETTAIQLIHPAETNTMVPRTTPKFSHSKSDTTLPVAITSPGPEAS
SAVSTTTISPDMSDLVTSLVPSSGTDTSTTFPTLSETPYEPETTATWLTHPAETSTTVSG
TIPNFSHRGSDTAPSMVTSPGVDTRSGVPTTTIPPSIPGVVTSQVTSSATDTSTAIPTLTP
SPGEPETTASSATHPGTQTGFTVPIRTVPSSEPDTMASWVTHPPQTSTPVSRTTSSFSHS
SPDATPVMATSPRTEASSAVLTTISPGAPEMVTSQITSSGAATSTTVPTLTHSPGMPET
TALLSTHPRTETSKTFPASTVFPQVSETTASLTIRPGAETSTALPTQTTSSLFTLLVTGT
SRVDLSPTASPGVSAKTAPLSTHPGTETSTMIPTSTLSLGLLETTGLLATSSSAETSTST
LTLTVSPAVSGLSSASITTDKPQTVTSWNTETSPSVTSVGPPEFSRTVTGTTMTLIPSE
MPTPPKTSHGEGVSPTTILRTTMVEATNLATTGSSPTVAKTTTTFNTLAGSLFTPLTTP
GMSTLASESVTSRTSYNHRSWISTTSSYNRRYWTPATSTPVTSTFSPGISTSSIPSSTAA
TVPFMVPFTLNFTITNLQYEEDMRHPGSRKFNATERELQGLLKPLFRNSSLEYLYSGC
RLASLRPEKDSSATAVDAICTHRPDPEDLGLDRERLYWELSNLTNGIQELGPYTLDRN
SLYVNGFTHRSSMPTTSTPGTSTVDVGTSGTPSSSPSPTTAGPLLMPFTLNFTITNLQY
EEDMRRTGSRKFNTMESVLQGLLKPLFKNTSVGPLYSGCRLTLLRPEKDGAATGVD
AICTHRLDPKSPGLNREQLYWELSKLINDIEELGPYTLDRNSLYVNGFTHQSSVSTTS
TPGTSTVDLRTSGTPSSLSSPTIMAAGPLLVPFTLNFTITNLQYGEDMGHPGSRKFNTT
ERVLQGLLGPIFKNTSVGPLYSGCRLTSLRSEKDGAATGVDAICIHHLDPKSPGLNRE
RLYWELSQLTNGIKELGPYTLDRNSLYVNGFTHRTSVPTSSTPGTSTVDLGTSGTPFS
LPSPATAGPLLVLFTLNFTITNLKYEEDMHRPGSRKFNTTERVLQTLLGPMFKNTSVG
LLYSGCRLTLLRSEKDGAATGVDAICTHRLDPKSPGVDREQLYWELSQLTNGIKELG
PYTLDRNSLYVNGFTHWIPVPTSSTPGTSTVDLGSGTPSSLPSPTTAGPLLVPFTLNFTI
TNLKYEEDMHCPGSRKFNTTERVLQSLLGPMFKNTSVGPLYSGCRLTLLRSEKDGAA
TGVDAICTHRLDPKSPGVDREQLYWELSQLTNGIKELGPYTLDRNSLYVNGFTHQTS
APNTSTPGTSTVDLGTSGTPSSLPSPTSAGPLLVPFTLNFTITNLQYEEDMHHPGSRKF
NTTERVLQGLLGPMFKNTSVGLLYSGCRLTLLRPEKNGAATGMDAICSHRLDPKSPG
LNREQLYWELSQLTHGIKELGPYTLDRNSLYVNGFTHRSSVAPTSTPGTSTVDLGTSG
TPSSLPSPTTAVPLLVPFTLNFTITNLQYGEDMRHPGSRKFNTTERVLQGLLGPLFKNS
SVGPLYSGCRLISLRSEKDGAATGVDAICTHHLNPQSPGLDREQLYWQLSQMTNGIK
ELGPYTLDRNSLYVNGFTHRSSGLTTSTPWTSTVDLGTSGTPSPVPSPTTTGPLLVPFT
LNFTITNLQYEENMGHPGSRKFNITESVLQGLLKPLFKSTSVGPLYSGCRLTLLRPEK
DGVATRVDAICTHRPDPKIPGLDRQQLYWELSQLTHSITELGPYTLDRDSLYVNGFT
QRSSVPTTSTPGTFTVQPETSETPSSLPGPTATGPVLLPFTLNFTITNLQYEEDMRRPGS
RKFNTTERVLQGLLMPLFKNTSVSSLYSGCRLTLLRPEKDGAATRVDAVCTHRPDPK
SPGLDRERLYWKLSQLTHGITELGPYTLDRHSLYVNGFTHQSSMTTTRTPDTSTMHL
ATSRTPASLSGPMTASPLLVLFTINFTITNLRYEENMHHPGSRKENTTERVLQGLLRPV
FKNTSVGPLYSGCRLTLLRPKKDGAATKVDAICTYRPDPKSPGLDREQLYWELSQLT
HSITELGPYTLDRDSLYVNGFTQRSSVPTTSIPGTPTVDLGTSGTPVSKPGPSAASPLL
VLFTLNFTITNLRYEENMQHPGSRKFNTTERVLQGLLRSLFKSTSVGPLYSGCRLTLL
RPEKDGTATGVDAICTHHPDPKSPRLDREQLYWELSQLTHNITELGPYALDNDSLFV
NGFTHRSSVSTTSTPGTPTVYLGASKTPASIFGPSAASHLLILFTLNFTITNLRYEENM
WPGSRKFNTTERVLQGLLRPLFKNTSVGPLYSGCRLTLLRPEKDGEATGVDAICTHR
PDPTGPGLDREQLYLELSQLTHSITELGPYTLDRDSLYVNGFTHRSSVPTTSTGVVSEE
PFTLNFTINNLRYMADMGQPGSLKFNITDNVMQHLLSPLFQRSSLGARYTGCRVIAL
RSVKNGAETRVDLLCTYLQPLSGPGLPIKQVFHELSQQTHGITRLGPYSLDKDSLYLN
GYNEPGPDEPPTTPKPATTFLPPLSEATTAMGYHLKTLTLNFTISNLQYSPDMGKGSA
TFNSTEGVLQHLLRPLFQKSSMGPFYLGCQLISLRPEKDGAATGVDTTCTYHPDPVGP
GLDIQQLYWELSQLTHGVTQLGFYVLDRDSLFINGYAPQNLSIRGEYQINFHIVNWN
LSNPDPTSSEYITLLRDIQDKVTTLYKGSQLHDTFRFCLVTNLTMDSVLVTVKALFSS
NLDPSLVEQVFLDKTLNASFHWLGSTYQLVDIHVTEMESSVYQPTSSSSTQHFYLNFT
ITNLPYSQDKAQPGTTNYQRNKRNIEDALNQLFRNSSIKSYFSDCQVSTFRSVPNRHH
TGVDSLCNFSPLARRVDRVAIYEEFLRMTRNGTQLQNFTLDRSSVLVDGYSPNRNEP
LTGNSDLPFWAVILIGLAGLLGVITCLICGVLVTTRRRKKEGEYNVQQQCPGYYQSH
LDLEDLQ

The polypeptide represented by the amino acid sequence of SEQ ID NO: 3 is referred to herein as MUC16^{c114} and consists of the C-terminal 114 amino acid residues of mature MUC16 (SEQ ID NO: 2 being the sequence of mature MUC16). MUC16^{c114} comprises a 58 amino acid ectodomain, a 25 amino acid transmembrane domain and a 31 amino acid cytoplasmic tail. MUC16^{c114} is capable of being N-glycosylated at the asparagine amino acid residues at positions 1, 24, and 30 of SEQ ID NO: 3 (also referred to as amino acid positions Asn1777, Asn1800, and Asn1806 according the original MUC16 publication Yin B W and Lloyd K O, 2001, J Biol Chem. 276(29):27371-5).

MUC16^{cl14}
(SEQ ID NO: 3)
NFSPLARRVDRVAIYEEFLRMTRNGTQLQNFTLDRSSVLVDGYSPNRNE
PLTGNSDLPFWAVILIGLAGLLGLITCLICGVLVTTRRRKKEGEYNVQQ
QCPGYYQSHLDLEDLQ

The 58 amino acid ectodomain sequence present in MUC16^{c114} is represented as SEQ ID NO: 95:

(SEQ ID NO: 95)
NFSPLARRVDRVAIYEEFLRMTRNGTQLQNFTLDRSSVLVDGYSPNRNE
PLIGNSDLP

As used herein, the term “nucleic acid” or “polynucleotide” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration. Pharmaceutically-acceptable carriers and their formulations are known to one skilled in the art and are described, for example, in Remington's Pharmaceutical Sciences (20^{th} edition, ed. A. Gennaro, 2000, Lippincott, Williams & Wilkins, Philadelphia, PA.).

As used herein, the term “polyclonal antibody” means a preparation of antibodies derived from at least two (2) different antibody-producing cell lines. The use of this term includes preparations of at least two (2) antibodies that contain antibodies that specifically bind to different epitopes or regions of an antigen.

As used herein, the terms “polypeptide,” “peptide” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art. Such modifications are well described in basic texts and in more detailed monographs, as well as in a voluminous research literature.

As used herein, the term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the material is derived from a cell so modified. Thus, for example, recombinant cells express genes that are not found within the native (non-recombinant) form of the cell or express native genes that are otherwise abnormally expressed, under expressed or not expressed at all.

As used herein, a “sample” or “biological sample” refers to a body fluid or a tissue sample isolated from a subject. In some cases, a biological sample may consist of or comprise whole blood, platelets, red blood cells, white blood cells, plasma, sera, urine, feces, epidermal sample, vaginal sample, skin sample, cheek swab, sperm, amniotic fluid, cultured cells, bone marrow sample, tumor biopsies, aspirate and/or chorionic villi, cultured cells, endothelial cells, synovial fluid, lymphatic fluid, ascites fluid, interstitial or extracellular fluid and the like. The term “sample” may also encompass the fluid in spaces between cells, including gingival crevicular fluid, bone marrow, cerebrospinal fluid (CSF), saliva, mucus, sputum, semen, sweat, urine, or any other bodily fluids. Samples can be obtained from a subject by any means including, but not limited to, venipuncture, excretion, ejaculation, massage, biopsy, needle aspirate, lavage, scraping, surgical incision, or intervention or other means known in the art. A blood sample can be whole blood or any fraction thereof, including blood cells (red blood cells, white blood cells or leukocytes, and platelets), serum and plasma.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, the terms “single-chain antibodies” or “single-chain Fv (scFv)” refer to an antibody fusion molecule of the two domains of the Fv fragment, V_{L} and V_{H}. Single-chain antibody molecules may comprise a polymer with a number of individual molecules, for example, dimer, trimer or other polymers. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single-chain Fv (scFv)). Bird et al. (1988) Science 242:423-426 and Huston et al. (1988) Proc Natl Acad Sci 85:5879-5883. Such single-chain antibodies can be prepared by recombinant techniques or enzymatic or chemical cleavage of intact antibodies.

The V_{H} and V_{L} domains are either joined directly or joined by a peptide-encoding linker (e.g., about 10, 15, 20, 25 amino acids), which connects the N-terminus of the V_{H} with the C-terminus of the V_{L}, or the C-terminus of the V_{H} with the N-terminus of the V_{L}. In some embodiments, the linker is usually rich in glycine for flexibility, as well as serine or threonine for solubility. The linker can link the heavy chain variable region and the light chain variable region of the extracellular antigen binding domain.

Despite removal of the constant regions and the introduction of a linker, scFv proteins retain the specificity of the original immunoglobulin. Single chain Fv polypeptide antibodies can be expressed from a nucleic acid comprising V_{H}- and V_{L}-encoding sequences as described by Huston, et al, Proc. Nat. Acad. Sci. USA, 85:5879-5883 (1988)). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al, Hybridoma (Larchmt) 27(6):455-51 (2008); Peter et al, J Cachexia Sarcopenia Muscle (2012); Shieh et al, J Imunol 183(4):2277-85 (2009); Giomarelli et al, Thromb Haemost 97(6):955-63 (2007); Fife et al, J Clin Invst 116(8):2252-61 (2006); Brocks et al, Immunotechnology 3(3): 173-84 (1997); Moosmayer et al, Ther Immunol 2(10):31-40 (1995). Agonistic scFvs having stimulatory activity have been described (see, e.g., Peter et al, J Biol Chem 25278(38):36740-7 (2003); Xie et al, Nat Biotech 15(8):768-71 (1997); Ledbetter et al, Crit Rev Immunol 17(5-6):427-55 (1997); Ho et al, Bio Chim Biophys Acta 1638(3):257-66 (2003)).

As used herein, “specifically binds” refers to a molecule (e.g., an antibody or antigen binding fragment thereof) which recognizes and binds another molecule (e.g., an antigen), but that does not substantially recognize and bind other molecules. The terms “specific binding,” “specifically binds to,” or is “specific for” a particular molecule (e.g., a polypeptide, or an epitope on a polypeptide), as used herein, can be exhibited, for example, by a molecule having a K_{D} for the molecule to which it binds to of about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, or 10⁻¹²M. The term “specifically binds” may also refer to binding where a molecule (e.g., an antibody or antigen binding fragment thereof) binds to a particular polypeptide (e.g., a MUC16 polypeptide), or an epitope on a particular polypeptide, without substantially binding to any other polypeptide, or polypeptide epitope.

As used herein, the terms “subject,” “individual,” or “patient” are used interchangeably and refer to an individual organism, a vertebrate, a mammal, or a human. In certain embodiments, the individual, patient or subject is a human.

As used herein, a “synergistic therapeutic effect” reflects a greater-than-additive therapeutic effect that is produced by a combination of at least two agents, and which exceeds that which would otherwise result from the individual administration of the agents. For example, lower doses of one or more agents may be used in treating a disease or disorder, resulting in increased therapeutic efficacy and decreased side-effects.

“Treating”, “treat”, or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission. In some embodiments, “inhibiting,” means reducing or slowing the growth of a tumor. In some embodiments, the inhibition of tumor growth may be, for example, by 5% or more, 10% or more, 20% or more, 30% or more, 40% or more, 50% or more, 60% or more, 70% or more, 80% or more, or 90% or more. In some embodiments, the inhibition may be complete.

It is also to be appreciated that the various modes of treatment of medical diseases and conditions as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

Amino acid sequence modification(s) of the anti-MUC16 antibodies described herein are contemplated. Such modifications can be introduced to improve the binding affinity and/or other biological properties of the antibody, for example, to render the encoded amino acid aglycosylated, or to destroy the antibody's ability to bind to C1q, Fc receptor, or to activate the complement system. Amino acid sequence variants of an anti-MUC16 antibody are prepared by introducing appropriate nucleotide changes into the antibody nucleic acid, by peptide synthesis, or by chemical modifications. Such modifications include, for example, deletions from, and/or insertions into and/or substitutions of, residues within the amino acid sequences of the antibody. Any combination of deletion, insertion, and substitution is made to obtain the antibody of interest, as long as the obtained antibody possesses the desired properties. The modification also includes the change of the pattern of glycosylation of the protein. The sites of greatest interest for substitutional mutagenesis include the hypervariable regions, but FR alterations are also contemplated.

Conservative amino acid substitutions are amino acid substitutions that change a given amino acid to a different amino acid with similar biochemical properties (e.g., charge, hydrophobicity and size). “Conservative substitutions” are shown in the Table below.

Amino Acid Substitutions

| Original | | Conservative |
|---|---|---|
| Residue | Exemplary Substitutions | Substitutions |
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; norleucine | leu |
| Leu (L) | norleucine; ile; val; met; ala; phe | ile |
| Lys (K) | arg; gln; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; norleucine | leu |

One type of substitutional variant involves substituting one or more hypervariable region residues of a parent antibody. A convenient way for generating such substitutional variants involves affinity maturation using phage display. Specifically, several hypervariable region sites (e.g., 6-7 sites) are mutated to generate all possible amino acid substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and the antigen. Such contact residues and neighboring residues are candidates for substitution according to the techniques elaborated herein. Once such variants are generated, the panel of variants is subjected to screening as described herein and antibodies with similar or superior properties in one or more relevant assays may be selected for further development.

### Anti-MUC16^{c114} Immunoglobin-related Compositions of the Present Technology

Provided herein are anti-MUC16 immunoglobulin-related compositions that immunospecifically bind to the C-terminal 114 amino acid residues of mature MUC16 (e.g., MUC16^{c114}) In some embodiments, the anti-MUC16 immunoglobulin-related composition is an anti-MUC16 construct that comprises an antibody moiety that immunospecifically binds to MUC16, such as the C-terminal 114 amino acid residues of mature MUC16 (e.g., MUC16^{c114}). Examples of such anti-MUC16 immunoglobulin-related compositions are described in WO2020/102555 and WO2020/227538, which are herein incorporated by reference in their entirety. In some embodiments, the anti-MUC16 immunoglobulin-related composition is an anti-MUC16 antibody (e.g., a full-length anti-MUC16 antibody) or an antigen binding fragment thereof. In some embodiments, the anti-MUC16 immunoglobulin-related composition binds to an MUC16-expressing cell (e.g., an MUC16-expressing cancer cell). In some embodiments, the anti-MUC16 immunoglobulin-related composition is a full-length antibody (e.g., full-length IgG) or an antigen-binding fragment thereof, which specifically binds to MUC16.

Anti-MUC16 immunoglobulin-related compositions of the present technology, such as anti-MUC16 antibodies or antigen-binding fragments thereof that immunospecifically bind to the C-terminal 114 amino acid residues of mature MUC16 (e.g., MUC16^{c114}), can include, e.g., monoclonal antibodies, polyclonal antibodies, recombinantly produced antibodies, monospecific antibodies, multispecific antibodies (including bispecific antibodies (BsAb)), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, single domain antibodies, monovalent antibodies, single chain antibodies or single-chain variable fragments (scFv), camelized antibodies, affibodies, and disulfide-linked Fvs (dsFv), Fc fusion proteins, immunoconjugates, or fragments thereof. Such antibodies and antigen-binding fragments can be made by methods known in the art. The anti-MUC16 immunoglobulin-related compositions of the present technology may be a tandem scFv, a diabody (db), a single chain diabody (scDb), a dual-affinity retargeting (DART) antibody, a F(ab′)₂, a dual variable domain (DVD) antibody, a knob-into-hole (KiH) antibody, a dock and lock (DNL) antibody, a chemically cross-linked antibody, a heteromultimeric antibody, a monoclonal antibody, a full-length antibody, or a heteroconjugate antibody.

The exemplary CDR sequences in Table 1 are predicted using the IgBLAST algorithm. See, for example, Ye J. et al., Nucleic Acids Research 41:W34-W40 (2013), the disclosure of which is incorporated herein by reference in its entirety. Those skilled in the art will recognize that many algorithms are known for prediction of CDR positions in antibody heavy chain and light chain variable regions, and immunoglobulin-related compositions comprising CDRs from antibodies described herein, but based on prediction algorithms other than IgBLAST, are within the scope of the present disclosure.

TABLE 1
Exemplary anti-MUC16 antibody CDR sequences.

| Antibody | | | |
|---|---|---|---|
| ID | HC-CDR1 | HC-CDR2 | HC-CDR3 |
| 4H11 | GFTFSSYA | ISSAGGYI | ARQGFGNYGDYYAMDY |
| | (SEQ ID NO: 4) | (SEQ ID NO: 5) | (SEQ ID NO: 6) |
| 18C6 | GFSLSTVGMG | IWWDDEDK | TRIGTAQATDALDY |
| | (SEQ ID NO: 10) | (SEQ ID NO: 11) | (SEQ ID NO: 12) |
| 8 | GGSFSGYY | INHSGST | ARQSYITDS |
| | (SEQ ID NO: 16) | (SEQ ID NO: 17) | (SEQ ID NO: 18) |
| 12 | GGSFSGYY | INHSGST | ARWSPFSYKQMYDY |
| | (SEQ ID NO: 22) | (SEQ ID NO: 23) | (SEQ ID NO: 24) |
| Antibody | | | |
| ID | LC-CDR1 | LC-CDR2 | LC-CDR3 |
| 4H11 | QSLLNSRTRKNQ | WAS | QQSYNLLT |
| | (SEQ ID NO: 7) | (SEQ ID NO: 8) | (SEQ ID NO: 9) |
| 18C6 | KSLLHSNGNTY | YMS | MQSLEYPLT |
| | (SEQ ID NO: 13) | (SEQ ID NO: 14) | (SEQ ID NO: 15) |
| 8 | QDVSKW | AAS | QQANSFPWT |
| | (SEQ ID NO: 19) | (SEQ ID NO: 20) | (SEQ ID NO: 21) |
| 12 | RGSIASAY | EDY | QSYDDNDHVI |
| | (SEQ ID NO: 25) | (SEQ ID NO: 26) | (SEQ ID NO: 27) |

In one aspect, the present disclosure provides an anti-MUC16 antibody or antigen binding fragment thereof comprising a heavy chain immunoglobulin variable domain (V_{H}) and a light chain immunoglobulin variable domain (V_{L}), wherein (a) the V_{H} comprises a V_{H}-CDR1 sequence of SEQ ID NO: 4, a V_{H}-CDR2 sequence of SEQ ID NO: 5, and a V_{H}-CDR3 sequence of SEQ ID NO: 6; and/or; (b) the V_{L} comprises a V_{L}-CDR1 sequence of SEQ ID NO: 7, a V_{L}-CDR2 sequence of SEQ ID NO: 8, and a V_{L}-CDR3 sequence of SEQ ID NO: 9.

In one aspect, the present disclosure provides an anti-MUC16 antibody or antigen binding fragment thereof comprising a heavy chain immunoglobulin variable domain (V_{H}) and a light chain immunoglobulin variable domain (V_{L}), wherein (a) the V_{H} comprises a V_{H}-CDR1 sequence of SEQ ID NO: 10, a V_{H}-CDR2 sequence of SEQ ID NO: 11, and a V_{H}-CDR3 sequence of SEQ ID NO: 12; and/or; (b) the V_{L} comprises a V_{L}-CDR1 sequence of SEQ ID NO: 13, a V_{L}-CDR2 sequence of SEQ ID NO: 14, and a V_{L}-CDR3 sequence of SEQ ID NO: 15.

In another aspect, the present disclosure provides an anti-MUC16 antibody or antigen binding fragment thereof comprising a heavy chain immunoglobulin variable domain (V_{H}) and a light chain immunoglobulin variable domain (V_{L}), wherein (a) the V_{H} comprises a V_{H}-CDR1 sequence of SEQ ID NO: 16, a V_{H}-CDR2 sequence of SEQ ID NO: 17, and a V_{H}-CDR3 sequence of SEQ ID NO: 18; and/or; (b) the V_{L} comprises a V_{L}-CDR1 sequence of SEQ ID NO: 19, a V_{L}-CDR2 sequence of SEQ ID NO: 20, and a V_{L}-CDR3 sequence of SEQ ID NO: 21.

In yet another aspect, the present disclosure provides an anti-MUC16 antibody or antigen binding fragment thereof comprising a heavy chain immunoglobulin variable domain (V_{H}) and a light chain immunoglobulin variable domain (V_{L}), wherein (a) the V_{H} comprises a V_{H}-CDR1 sequence of SEQ ID NO: 22, a V_{H}-CDR2 sequence of SEQ ID NO: 23, and a V_{H}-CDR3 sequence of SEQ ID NO: 24; and/or; (b) the V_{L} comprises a V_{L}-CDR1 sequence of SEQ ID NO: 25, a V_{L}-CDR2 sequence of SEQ ID NO: 26, and a V_{L}-CDR3 sequence of SEQ ID NO: 27.

In one aspect, the present disclosure provides an anti-MUC16 antibody or antigen binding fragment thereof comprising (a) a heavy chain immunoglobulin variable domain (V_{H}) comprising a V_{H}-CDR1 sequence, a V_{H}-CDR2 sequence, and a V_{H}-CDR3 sequence of SEQ ID NO: 28 or SEQ ID NO: 29 and a light chain immunoglobulin variable domain (V_{L}) comprising a V_{L}-CDR1 sequence, a V_{L}-CDR2 sequence, and a V_{L}-CDR3 sequence of SEQ ID NO: 30 or SEQ ID NO: 31; or (b) a heavy chain immunoglobulin variable domain (V_{H}) comprising a V_{H}-CDR1 sequence, a V_{H}-CDR2 sequence, and a V_{H}-CDR3 sequence of SEQ ID NO: 32 or SEQ ID NO: 33 and a light chain immunoglobulin variable domain (V_{L}) comprising a V_{L}-CDR1 sequence, a V_{L}-CDR2 sequence, and a V_{L}-CDR3 sequence of SEQ ID NO: 34 or SEQ ID NO: 35; or (c) a heavy chain immunoglobulin variable domain (V_{H}) comprising a V_{H}-CDR1 sequence, a V_{H}-CDR2 sequence, and a V_{H}-CDR3 sequence of SEQ ID NO: 36 and a light chain immunoglobulin variable domain (V_{L}) comprising a V_{L}-CDR1 sequence, a V_{L}-CDR2 sequence, and a V_{L}-CDR3 sequence of SEQ ID NO: 37; or (d) a heavy chain immunoglobulin variable domain (V_{H}) comprising a V_{H}-CDR1 sequence, a V_{H}-CDR2 sequence, and a V_{H}-CDR3 sequence of SEQ ID NO: 38 and a light chain immunoglobulin variable domain (V_{L}) comprising a V_{L}-CDR1 sequence, a V_{L}-CDR2 sequence, and a V_{L}-CDR3 sequence of SEQ ID NO: 39.

In one aspect, the present disclosure provides an anti-MUC16 antibody or antigen binding fragment thereof comprising a heavy chain immunoglobulin variable domain (V_{H}) and a light chain immunoglobulin variable domain (V_{L}), wherein: (a) the V_{H} comprises an amino acid sequence selected from any one of SEQ ID NOs: 28-29, 32-33, 36 or 38; and/or (b) the V_{L} comprises an amino acid sequence selected from any one of SEQ ID NOs: 30-31, 34-35, 37 or 39. The V_{H} and/or V_{L} domains of the anti-MUC16 antibody or antigen binding fragment may comprise a leader sequence. Examples of leader sequences include, but are not limited to METDTLLLWVLLLWVPGSTG (SEQ ID NO: 93) and MGWSCIILFLVATATGKL (SEQ ID NO: 94).

In some embodiments, the anti-MUC16 antibody or antigen binding fragment thereof comprising (a) a heavy chain immunoglobulin variable domain (V_{H}) comprising the amino acid sequence of SEQ ID NO: 28 or SEQ ID NO: 29 and a light chain immunoglobulin variable domain (V_{L}) comprising the amino acid sequence of SEQ ID NO: 30 or SEQ ID NO: 31; or (b) a heavy chain immunoglobulin variable domain (V_{H}) comprising the amino acid sequence of SEQ ID NO: 32 or SEQ ID NO: 33 and a light chain immunoglobulin variable domain (V_{L}) comprising the amino acid sequence of SEQ ID NO: 34 or SEQ ID NO: 35; or (c) a heavy chain immunoglobulin variable domain (V_{H}) comprising the amino acid sequence of SEQ ID NO: 36 and a light chain immunoglobulin variable domain (V_{L}) comprising the amino acid sequence of SEQ ID NO: 37; or (d) a heavy chain immunoglobulin variable domain (V_{H}) comprising the amino acid sequence of SEQ ID NO: 38 and a light chain immunoglobulin variable domain (V_{L}) comprising the amino acid sequence of SEQ ID NO: 39.

In certain embodiments, the immunoglobulin-related composition includes one or more of the following characteristics: (a) a light chain immunoglobulin variable domain sequence that is at least 80%, at least 85%, at least 90%, at least 95%, or at least 99% identical to the light chain immunoglobulin variable domain sequence of any one of SEQ ID NOs: 30-31, 34-35, 37 or 39; and/or (b) a heavy chain immunoglobulin variable domain sequence that is at least 80%, at least 85%, at least 90%, at least 95%, or at least 99% identical to the heavy chain immunoglobulin variable domain sequence of any one of SEQ ID NOs: 28-29, 32-33, 36 or 38. See Table 2. In another aspect, one or more amino acid residues in the immunoglobulin-related compositions provided herein are substituted with another amino acid. The substitution may be a “conservative substitution” as defined herein.

TABLE 2
Exemplary anti-MUC16 antibody V_{H} and V_{L} domain sequences.

| Clone | | |
|---|---|---|
| ID | Description | Sequence |
| 4H11 | VH | EVKLQESGGGFVKPGGSLRVSCAASGFTFSSYAMSWVRLAPEMRL |
| | Domain | EWVATISSAGGYIFYSDSVQGRFTISRDNAKNSLHLQMGSLRSGDT |
| | (H1) | AMYYCARQGFGNYGDYYAMDYWGQGTTVTVSS (SEQ ID NO: 28) |
| 4H11 | VH | EVQLVESGGGLVKPGGSLRVSCAASGFTFSSYAMSWVRLAPGKGL |
| | Domain | EWVATISSAGGYIFYSDSVQGRFTISRDNAKNSLYLQMNSLRAEDT |
| | (H2) | AMYYCARQGFGNYGDYYAMDYWGQGTLVTVSS (SEQ ID NO: 29) |
| 4H11 | VL | DIELTQSPSSLAVSAGERVTMNCKSSQSLLNSRTRKNQLAWYQQKP |
| | Domain | GQSPELLIYWASTRQSGVPDRFSGSGSGTDFTLTISSVQAEDVAVYY |
| | (L1) | CQQSYNLLTFGPGTKLEIKR (SEQ ID NO: 30) |
| 4H11 | VL | DIVLTQSPDSLAVSLGERVTMNCKSSQSLLNSRTRKNQLAWYQQK |
| | Domain | PGQSPELLIYWASTRQSGVPDRFSGSGSGTDFTLTISSVQAEDVAVY |
| | (L2) | YCQQSYNLLTFGQGTKLEIKR (SEQ ID NO: 31) |
| 18C6 | VH | QVTLKESGPGILQPTQTLTLTCTFSGFSLSTVGMGVGWSRQPSGKG |
| | Domain | LEWLAHIWWDDEDKYYNPALKSRLTITKDTSKNQVFLKITNVDTA |
| | (H1) | DTATYYCTRIGTAQATDALDYWGQGTLVTVSS (SEQ ID NO: 32) |
| 18C6 | VH | QVTLKESGPTLVKPTQTLTLTCTFSGFSLSTVGMGVGWSRQPSGKG |
| | Domain | LEWLAHIWWDDEDKYYNPALKSRLTITKDTSKNQVVLTITNVDPV |
| | (H2) | DTATYYCTRIGTAQATDALDYWGQGTLVTVSS |
| | | (SEQ ID NO: 33) |
| 18C6 | VL | DIVMTQSAPSVPVTPGESVSISCRSSKSLLHSNGNTYLYWFLQKPGQ |
| | Domain | SPQRLIYYMSNLASGVPDRFSGRGSGTDFTLKISRVEAEDVGVYYC |
| | (L1) | MQSLEYPLTFGGGTKLEIKR (SEQ ID NO: 34) |
| 18C6 | VL | DIVMTQSALSLPVTPGEPVSISCRSSKSLLHSNGNTYLYWFLQKPGQ |
| | Domain | SPQRLIYYMSNLASGVPDRFSGSGSGTDFTLKISRVEAEDVGVYYC |
| | (L2) | MQSLEYPLTFGGGTKLEIKR (SEQ ID NO: 35) |
| 8 | VH domain | QVQLQQWGAGLLKPSETLSLTCAVYGGSFSGYYWSWIRQPPGKGL |
| | | EWIGEINHSGSTNYNPSLKSRVTISVDTSKNQFSLKLSSVTAADTAV |
| | | YYCARQSYITDSWGQGTLVTVSS (SEQ ID NO: 36) |
| 8 | VL | DIQLTQSPSAVSASVGDRVTITCRASQDVSKWLAWYQQKPGKAPR |
| | domain | LLISAASGLQSWVPSRFSGSGSGTEFTLSISSLQPEDFATYYCQQANS |
| | | FPWTFGQGTKVEIKR (SEQ ID NO: 37) |
| 12 | VH domain | QVQLQQWGAGLLKPSETLSLTCAVYGGSFSGYYWSWIRQPPGKGL |
| | | EWIGEINHSGSTNYNPSLKSRIIMSVDTSKRQFSLKLRSATAADTAV |
| | | YYCARWSPFSYKQMYDYWGQGTLVTVSS (SEQ ID NO: 38) |
| 12 | VL | NFMLTQPHSVSESPGKTVTISCTRSRGSIASAYVQWYQQRPGSAPIT |
| | domain | VIYEDYERPSEIPDRFSGSIDSSSNSASLTISGLKTEDEADYYCQSYD |
| | | DNDHVIFGGGTKVTVLG (SEQ ID NO: 39) |

V_{H} and V_{L} CDR sequences are underlined.

In any of the above embodiments, the antibody further comprises a Fc domain of any isotype, e.g., but are not limited to, IgG (including IgG1, IgG2, IgG3, and IgG4), IgA (including IgA1 and IgA2), IgD, IgE, or IgM, and IgY. Non-limiting examples of constant region sequences include:

Human IgD constant region, Uniprot: P01880
(SEQ ID NO: 40)
APTKAPDVFPIISGCRHPKDNSPVVLACLITGYHPTSVTVTWYMGTQSQPQRT
FPEIQRRDSYYMTSSQLSTPLQQWRQGEYKCVVQHTASKSKKEIFRWPESPKAQASS
VPTAQPQAEGSLAKATTAPATTRNTGRGGEEKKKEKEKEEQEERETKTPECPSHTQP
LGVYLLTPAVQDLWLRDKATFTCFVVGSDLKDAHLTWEVAGKVPTGGVEEGLLER
HSNGSQSQHSRLTLPRSLWNAGTSVTCTLNHPSLPPQRLMALREPAAQAPVKLSLNL
LASSDPPEAASWLLCEVSGFSPPNILLMWLEDQREVNTSGFAPARPPPQPGSTTFWA
WSVLRVPAPPSPQPATYTCVVSHEDSRTLLNASRSLEVSYVTDHGPMK
Human IgG1 constant region, Uniprot: P01857
(SEQ ID NO: 41)
ASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKKVEPKSCDKTHTCPP
CPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK
Human IgG2 constant region, Uniprot: P01859
(SEQ ID NO: 42)
ASTKGPSVFPLAPCSRSTSESTAALGCLVKDYFPEPVTVSWNSGALTSGVHTF
PAVLQSSGLYSLSSVVTVPSSNFGTQTYTCNVDHKPSNTKVDKTVERKCCVECPPCP
APPVAGPSVFLFPPKPKDTLMISRTPEVTCVVVDVSHEDPEVQFNWYVDGVEVHNA
KTKPREEQFNSTFRVVSVLTVVHQDWLNGKEYKCKVSNKGLPAPIEKTISKTKGQPR
EPQVYTLPPSREEMTKNQVSLTCLVKGFYPSDISVEWESNGQPENNYKTTPPMLDSD
GSFFLYSKLTVDKSRWQQGNVFSCSVMHEALHNHYTQKSLSLSPGK
Human IgG3 constant region, Uniprot: P01860
(SEQ ID NO: 43)
ASTKGPSVFPLAPCSRSTSGGTAALGCLVKDYFPEPVTVSWNSGALTSGVHTF
PAVLQSSGLYSLSSVVTVPSSSLGTQTYTCNVNHKPSNTKVDKRVELKTPLGDTTHT
CPRCPEPKSCDTPPPCPRCPEPKSCDTPPPCPRCPEPKSCDTPPPCPRCPAPELLGGPSV
FLFPPKPKDTLMISRTPEVTCVVVDVSHEDPEVQFKWYVDGVEVHNAKTKPREEQY
NSTFRVVSVLTVLHQDWLNGKEYKCKVSNKALPAPIEKTISKTKGQPREPQVYTLPP
SREEMTKNQVSLTCLVKGFYPSDIAVEWESSGQPENNYNTTPPMLDSDGSFFLYSKL
TVDKSRWQQGNIFSCSVMHEALHNRFTQKSLSLSPGK
Human IgM constant region, Uniprot: P01871
(SEQ ID NO: 44)
GSASAPTLFPLVSCENSPSDTSSVAVGCLAQDFLPDSITLSWKYKNNSDISSTR
GFPSVLRGGKYAATSQVLLPSKDVMQGTDEHVVCKVQHPNGNKEKNVPLPVIAELP
PKVSVFVPPRDGFFGNPRKSKLICQATGFSPRQIQVSWLREGKQVGSGVTTDQVQAE
AKESGPTTYKVTSTLTIKESDWLGQSMFTCRVDHRGLTFQQNASSMCVPDQDTAIRV
FAIPPSFASIFLTKSTKLTCLVTDLTTYDSVTISWTRQNGEAVKTHTNISESHPNATFSA
VGEASICEDDWNSGERFTCTVTHTDLPSPLKQTISRPKGVALHRPDVYLLPPAREQLN
LRESATITCLVTGFSPADVFVQWMQRGQPLSPEKYVTSAPMPEPQAPGRYFAHSILTV
SEEEWNTGETYTCVAHEALPNRVTERTVDKSTGKPTLYNVSLVMSDTAGTCY
Human IgG4 constant region, Uniprot: P01861
(SEQ ID NO: 45)
ASTKGPSVFPLAPCSRSTSESTAALGCLVKDYFPEPVTVSWNSGALTSGVHTF
PAVLQSSGLYSLSSVVTVPSSSLGTKTYTCNVDHKPSNTKVDKRVESKYGPPCPSCPA
PEFLGGPSVFLFPPKPKDTLMISRTPEVTCVVVDVSQEDPEVQFNWYVDGVEVHNAK
TKPREEQFNSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTTPPVLDSDG
SFFLYSRLTVDKSRWQEGNVFSCSVMHEALHNHYTQKSLSLSLGK
Human IgA1 constant region, Uniprot: P01876
(SEQ ID NO: 46)
ASPTSPKVFPLSLCSTQPDGNVVIACLVQGFFPQEPLSVTWSESGQGVTARNFP
PSQDASGDLYTTSSQLTLPATQCLAGKSVTCHVKHYTNPSQDVTVPCPVPSTPPTPSP
STPPTPSPSCCHPRLSLHRPALEDLLLGSEANLTCTLTGLRDASGVTFTWTPSSGKSAV
QGPPERDLCGCYSVSSVLPGCAEPWNHGKTFTCTAAYPESKTPLTATLSKSGNTFRPE
VHLLPPPSEELALNELVTLTCLARGFSPKDVLVRWLQGSQELPREKYLTWASRQEPS
QGTTTFAVTSILRVAAEDWKKGDTFSCMVGHEALPLAFTQKTIDRLAGKPTHVNVS
VVMAEVDGTCY
Human IgA2 constant region, Uniprot: P01877
(SEQ ID NO: 47)
ASPTSPKVFPLSLDSTPQDGNVVVACLVQGFFPQEPLSVTWSESGQNVTARNF
PPSQDASGDLYTTSSQLTLPATQCPDGKSVTCHVKHYTNPSQDVTVPCPVPPPPPCCH
PRLSLHRPALEDLLLGSEANLTCTLTGLRDASGATFTWTPSSGKSAVQGPPERDLCGC
YSVSSVLPGCAQPWNHGETFTCTAAHPELKTPLTANITKSGNTFRPEVHLLPPPSEEL
ALNELVTLTCLARGFSPKDVLVRWLQGSQELPREKYLTWASRQEPSQGTTTFAVTSI
LRVAAEDWKKGDTFSCMVGHEALPLAFTQKTIDRMAGKPTHVNVSVVMAEVDGTC
Y
Human Ig kappa constant region, Uniprot: P01834
(SEQ ID NO: 48)
TVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
Human Ig lamda constant region, Uniprot: P01834
(SEQ ID NO: 49)
PKANPTVTLFPPSSEELQANKATLVCLISDFYPGAVTVAWKADGSPVKAGV
ETTKPSKQSNNKYAASSYLSLTPEQWKSHRSYSCQVTHEGSTVEKTVAPTECS

In some embodiments, the immunoglobulin-related compositions of the present technology comprise a heavy chain constant region that is at least 80%, at least 85%, at least 90%, at least 95%, at least 99%, or is 100% identical to SEQ ID NOS: 40-47. Additionally or alternatively, in some embodiments, the immunoglobulin-related compositions of the present technology comprise a light chain constant region that is at least 80%, at least 85%, at least 90%, at least 95%, at least 99%, or is 100% identical to SEQ ID NO: 48 or SEQ ID NO: 49.

In some embodiments, the immunoglobulin-related compositions of the present technology bind to the extracellular domain of a MUC16 polypeptide. In certain embodiments, the epitope is a conformational epitope or non-conformational epitope. In some embodiments, the MUC16 polypeptide has the amino acid sequence of SEQ ID NO: 3.

In one aspect, the present disclosure provides an immunoglobulin-related composition comprising an amino acid sequence that is at least 80%, at least 85%, at least 90%, at least 95%, at least 99%, or 100% identical to an amino acid sequence selected from any one of SEQ ID NOS: 50-69.

| SEQ | | |
|---|---|---|
| ID | | |
| NO | Description | Sequence |
| 50 | Clone 8 | DIQLTQSPSAVSASVGDRVTITCRASQDVSKWLAWYQQKPGKAPRLLISA |
| | scFv (LH) | ASGLQSWVPSRFSGSGSGTEFTLSISSLQPEDFATYYCQQANSFPWTFG |
| | | QGTKVEIKRSRGGGGSGGGGSGGGGSLEMAQVQLQQWGAGLLKPSE |
| | | TLSLTCAVYGGSFSGYYWSWIRQPPGKGLEWIGEINHSGSTNYNPSLKSR |
| | | VTISVDTSKNQFSLKLSSVTAADTAVYYCARQSYITDSWGQGTLVTVSS |
| 51 | Clone 8 | QVQLQQWGAGLLKPSETLSLTCAVYGGSFSGYYWSWIRQPPGKGLEWI |
| | scFv(HL) | GEINHSGSTNYNPSLKSRVTISVDTSKNQFSLKLSSVTAADTAVYYCARQS |
| | | YITDSWGQGTLVTVSSSRGGGGSGGGGSGGGGSLEMADIQLTQSPSA |
| | | VSASVGDRVTITCRASQDVSKWLAWYQQKPGKAPRLLISAASGLQSWVP |
| | | SRFSGSGSGTEFTLSISSLQPEDFATYYCQQANSFPWTFGQGTKVEIKR |
| 52 | Clone 12 | NFMLTQPHSVSESPGKTVTISCTRSRGSIASAYVQWYQQRPGSAPITVIYE |
| | scFv (LH) | DYERPSEIPDRFSGSIDSSSNSASLTISGLKTEDEADYYCQSYDDNDHVIF |
| | | GGGTKVTVLGSRGGGGSGGGGSGGGGSLEMAQVQLQQWGAGLLKP |
| | | SETLSLTCAVYGGSFSGYYWSWIRQPPGKGLEWIGEINHSGSTNYNPSLK |
| | | SRIIMSVDTSKRQFSLKLRSATAADTAVYYCARWSPFSYKQMYDYWGQG |
| | | TLVTVSS |
| 53 | Clone 12 | QVQLQQWGAGLLKPSETLSLTCAVYGGSFSGYYWSWIRQPPGKGLEWI |
| | scFv (HL) | GEINHSGSTNYNPSLKSRIIMSVDTSKRQFSLKLRSATAADTAVYYCARWS |
| | | PFSYKQMYDYWGQGTLVTVSSSRGGGGSGGGGSGGGGSLEMANFML |
| | | TQPHSVSESPGKTVTISCTRSRGSIASAYVQWYQQRPGSAPITVIYEDYER |
| | | PSEIPDRFSGSIDSSSNSASLTISGLKTEDEADYYCQSYDDNDHVIFGGGT |
| | | KVTVLG |
| 54 | 4H11 L1H1- | DIELTQSPSSLAVSAGERVTMNCKSSQSLLNSRTRKNQLAWYQQKPGQS |
| | scFv | PELLIYWASTRQSGVPDRFSGSGSGTDFTLTISSVQAEDVAVYYCQQSYN |
| | | LLTFGPGTKLEIKRGGGGSGGGGSGGGGSEVKLQESGGGFVKPGGSL |
| | | RVSCAASGFTFSSYAMSWVRLAPEMRLEWVATISSAGGYIFYSDSVQGRF |
| | | TISRDNAKNSLHLQMGSLRSGDTAMYYCARQGFGNYGDYYAMDYWGQ |
| | | GTTVTVSS |
| 55 | 4H11 L2H1- | DIVLTQSPDSLAVSLGERVTMNCKSSQSLLNSRTRKNQLAWYQQKPGQS |
| | scFv | PELLIYWASTRQSGVPDRFSGSGSGTDFTLTISSVQAEDVAVYYCQQSYN |
| | | LLTFGQGTKLEIKRGGGGSGGGGSGGGGSEVKLQESGGGFVKPGGSL |
| | | RVSCAASGFTESSYAMSWVRLAPEMRLEWVATISSAGGYIFYSDSVQGRF |
| | | TISRDNAKNSLHLQMGSLRSGDTAMYYCARQGFGNYGDYYAMDYWGQ |
| | | GTTVTVSS |
| 56 | 4H11 L1H2- | DIELTQSPSSLAVSAGERVTMNCKSSQSLLNSRTRKNQLAWYQQKPGQS |
| | scFv | PELLIYWASTRQSGVPDRFSGSGSGTDFTLTISSVQAEDVAVYYCQQSYN |
| | | LLTFGPGTKLEIKRGGGGSGGGGSGGGGSEVQLVESGGGLVKPGGSL |
| | | RVSCAASGFTFSSYAMSWVRLAPGKGLEWVATISSAGGYIFYSDSVQGRF |
| | | TISRDNAKNSLYLQMNSLRAEDTAMYYCARQGFGNYGDYYAMDYWGQ |
| | | GTLVTVSS |
| 57 | 4H11 L2H2- | DIVLTQSPDSLAVSLGERVTMNCKSSQSLLNSRTRKNQLAWYQQKPGQS |
| | scFv | PELLIYWASTRQSGVPDRFSGSGSGTDFTLTISSVQAEDVAVYYCQQSYN |
| | | LLTFGQGTKLEIKRGGGGSGGGGSGGGGSEVQLVESGGGLVKPGGSL |
| | | RVSCAASGFTESSYAMSWVRLAPGKGLEWVATISSAGGYIFYSDSVQGRF |
| | | TISRDNAKNSLYLQMNSLRAEDTAMYYCARQGFGNYGDYYAMDYWGQ |
| | | GTLVTVSS |
| 58 | 18C6 | DIVMTQSAPSVPVTPGESVSISCRSSKSLLHSNGNTYLYWFLQKPGQSPQ |
| | L1H1-scFv | RLIYYMSNLASGVPDRFSGRGSGTDFTLKISRVEAEDVGVYYCMQSLEY |
| | | PLTFGGGTKLEIKRGGGGSGGGGSGGGGSQVTLKESGPGILQPTQTLT |
| | | LTCTFSGFSLSTVGMGVGWSRQPSGKGLEWLAHIWWDDEDKYYNPAL |
| | | KSRLTITKDTSKNQVFLKITNVDTADTATYYCTRIGTAQATDALDYWGQ |
| | | GTLVTVSS |
| 59 | 18C6 | DIVMTQSALSLPVTPGEPVSISCRSSKSLLHSNGNTYLYWFLQKPGQSPQ |
| | L2H1-scFv | RLIYYMSNLASGVPDRFSGSGSGTDFTLKISRVEAEDVGVYYCMQSLEYP |
| | | LTFGGGTKLEIKRGGGGSGGGGSGGGGSQVTLKESGPGILQPTQTLTL |
| | | TCTFSGFSLSTVGMGVGWSRQPSGKGLEWLAHIWWDDEDKYYNPALK |
| | | SRLTITKDTSKNQVFLKITNVDTADTATYYCTRIGTAQATDALDYWGQG |
| | | TLVTVSS |
| 60 | 18C6 | DIVMTQSAPSVPVTPGESVSISCRSSKSLLHSNGNTYLYWFLQKPGQSPQ |
| | L1H2-scFv | RLIYYMSNLASGVPDRFSGRGSGTDFTLKISRVEAEDVGVYYCMQSLEY |
| | | PLTFGGGTKLEIKRGGGGSGGGGSGGGGSQVTLKESGPTLVKPTQTL |
| | | TLTCTFSGFSLSTVGMGVGWSRQPSGKGLEWLAHIWWDDEDKYYNPA |
| | | LKSRLTITKDTSKNQVVLTITNVDPVDTATYYCTRIGTAQATDALDYWGQ |
| | | GTLVTVSS |
| 61 | 18C6 | DIVMTQSALSLPVTPGEPVSISCRSSKSLLHSNGNTYLYWFLQKPGQSPQ |
| | L2H2-scFv | RLIYYMSNLASGVPDRFSGSGSGTDFTLKISRVEAEDVGVYYCMQSLEYP |
| | | LTFGGGTKLEIKRGGGGSGGGGSGGGGSQVTLKESGPTLVKPTQTLTL |
| | | TCTFSGFSLSTVGMGVGWSRQPSGKGLEWLAHIWWDDEDKYYNPALK |
| | | SRLTITKDTSKNQVVLTITNVDPVDTATYYCTRIGTAQATDALDYWGQGT |
| | | LVTVSS |
| 62 | 4H11 H1L1- | EVKLQESGGGFVKPGGSLRVSCAASGFTFSSYAMSWVRLAPEMRLEWV |
| | scFv | ATISSAGGYIFYSDSVQGRFTISRDNAKNSLHLQMGSLRSGDTAMYYCAR |
| | | QGFGNYGDYYAMDYWGQGTTVTVSSGGGGSGGGGSGGGGSDIELTQ |
| | | SPSSLAVSAGERVTMNCKSSQSLLNSRTRKNQLAWYQQKPGQSPELLIY |
| | | WASTRQSGVPDRFSGSGSGTDFTLTISSVQAEDVAVYYCQQSYNLLTFG |
| | | PGTKLEIKR |
| 63 | 4H11 H1L2- | EVKLQESGGGFVKPGGSLRVSCAASGFTESSYAMSWVRLAPEMRLEWV |
| | scFv | ATISSAGGYIFYSDSVQGRFTISRDNAKNSLHLQMGSLRSGDTAMYYCAR |
| | | QGFGNYGDYYAMDYWGQGTTVTVSSGGGGSGGGGSGGGGSDIVLTQ |
| | | SPDSLAVSLGERVTMNCKSSQSLLNSRTRKNQLAWYQQKPGQSPELLIY |
| | | WASTRQSGVPDRFSGSGSGTDFTLTISSVQAEDVAVYYCQQSYNLLTFG |
| | | QGTKLEIKR |
| 64 | 4H11 H2L1- | EVQLVESGGGLVKPGGSLRVSCAASGFTFSSYAMSWVRLAPGKGLEWV |
| | scFv | ATISSAGGYIFYSDSVQGRFTISRDNAKNSLYLQMNSLRAEDTAMYYCAR |
| | | QGFGNYGDYYAMDYWGQGTLVTVSSGGGGSGGGGSGGGGSDIELTQ |
| | | SPSSLAVSAGERVTMNCKSSQSLLNSRTRKNQLAWYQQKPGQSPELLIY |
| | | WASTRQSGVPDRFSGSGSGTDFTLTISSVQAEDVAVYYCQQSYNLLTFG |
| | | PGTKLEIKR |
| 65 | 4H11 H2L2- | EVQLVESGGGLVKPGGSLRVSCAASGFTFSSYAMSWVRLAPGKGLEWV |
| | scFv | ATISSAGGYIFYSDSVQGRFTISRDNAKNSLYLQMNSLRAEDTAMYYCAR |
| | | QGFGNYGDYYAMDYWGQGTLVTVSSGGGGSGGGGSGGGGSDIVLTQ |
| | | SPDSLAVSLGERVTMNCKSSQSLLNSRTRKNQLAWYQQKPGQSPELLIY |
| | | WASTRQSGVPDRFSGSGSGTDFTLTISSVQAEDVAVYYCQQSYNLLTFG |
| | | QGTKLEIKR |
| 66 | 18C6 | QVTLKESGPGILQPTQTLTLTCTFSGFSLSTVGMGVGWSRQPSGKGLE |
| | H1L1- scFv | WLAHIWWDDEDKYYNPALKSRLTITKDTSKNQVFLKITNVDTADTATYY |
| | | CTRIGTAQATDALDYWGQGTLVTVSSGGGGSGGGGSGGGGSDIVMTQ |
| | | SAPSVPVTPGESVSISCRSSKSLLHSNGNTYLYWFLQKPGQSPQRLIYYMS |
| | | NLASGVPDRFSGRGSGTDFTLKISRVEAEDVGVYYCMQSLEYPLTFGGG |
| | | TKLEIKR |
| 67 | 18C6 | QVTLKESGPGILQPTQTLTLTCTFSGFSLSTVGMGVGWSRQPSGKGLE |
| | H1L2- scFv | WLAHIWWDDEDKYYNPALKSRLTITKDTSKNQVFLKITNVDTADTATYY |
| | | CTRIGTAQATDALDYWGQGTLVTVSSGGGGSGGGGSGGGGSDIVMTQ |
| | | SALSLPVTPGEPVSISCRSSKSLLHSNGNTYLYWFLQKPGQSPQRLIYYMS |
| | | NLASGVPDRFSGSGSGTDFTLKISRVEAEDVGVYYCMQSLEYPLTFGGG |
| | | TKLEIKR |
| 68 | 18C6 | QVTLKESGPTLVKPTQTLTLTCTFSGFSLSTVGMGVGWSRQPSGKGLE |
| | H2L1- scFv | WLAHIWWDDEDKYYNPALKSRLTITKDTSKNQVVLTITNVDPVDTATYY |
| | | CTRIGTAQATDALDYWGQGTLVTVSSGGGGSGGGGSGGGGSDIVMTQ |
| | | SAPSVPVTPGESVSISCRSSKSLLHSNGNTYLYWFLQKPGQSPQRLIYYMS |
| | | NLASGVPDRFSGRGSGTDFTLKISRVEAEDVGVYYCMQSLEYPLTFGGG |
| | | TKLEIKR |
| 69 | 18C6 | QVTLKESGPTLVKPTQTLTLTCTFSGFSLSTVGMGVGWSRQPSGKGLE |
| | H2L2- scFv | WLAHIWWDDEDKYYNPALKSRLTITKDTSKNQVVLTITNVDPVDTATYY |
| | | CTRIGTAQATDALDYWGQGTLVTVSSGGGGSGGGGSGGGGSDIVMTQ |
| | | SALSLPVTPGEPVSISCRSSKSLLHSNGNTYLYWFLQKPGQSPQRLIYYMS |
| | | NLASGVPDRFSGSGSGTDFTLKISRVEAEDVGVYYCMQSLEYPLTFGGG |
| | | TKLEIKR |

V_{H} and V_{L} domains are italicized, CDR sequences are underlined, linker sequences are indicated in regular font.

Additionally or alternatively, in some embodiments, the anti-MUC16 immunoglobulin-related compositions of the present technology are multispecific and bind to the extracellular domain of a CD3 polypeptide. In certain embodiments, the anti-MUC16 immunoglobulin-related compositions of the present technology include an anti-CD3 antibody moiety comprising a V_{H} domain comprising the sequence DVQLVQSGAEVKKPGASVKVSCKASGYTFTRYTMHWVRQAPGQGLEWIGYINPSR GYTNYADSVKGRFTITTDKSTSTAYMELSSLRSEDTATYYCARYYDDHYCLDYWGQ GTTVTVSS (SEQ ID NO: 70) and a V_{L} domain comprising the sequence DIVLTQSPATLSLSPGERATLSCRASQSVSYMNWYQQKPGKAPKRWIYDTSKVASGV PARFSGSGSGTDYSLTINSLEAEDAATYYCQQWSSNPLTFGGGTKVEIK (SEQ ID NO: 71). Additionally or alternatively, in certain embodiments, the anti-MUC16 immunoglobulin-related compositions of the present technology include an anti-CD3 antibody moiety comprising the amino acid sequence of

(SEQ ID NO: 72)
DVQLVQSGAEVKKPGASVKVSCKASGYTFTRYTMHWVRQAPGQGLEWIG
YINPSRGYTNYADSVKGRFTITTDKSTSTAYMELSSLRSEDTATYYCAR
YYDDHYCLDYWGQGTTVTVSSGEGTSTGSGGSGGSGGADDIVLTQSPAT
LSLSPGERATLSCRASQSVSYMNWYQQKPGKAPKRWIYDTSKVASGVPA
RFSGSGSGTDYSLTINSLEAEDAATYYCQQWSSNPLTFGGGTKVEIK.

Additionally or alternatively, in some embodiments, the multispecific anti-MUC16×CD3 immunoglobulin-related compositions of the present technology comprise an amino acid sequence selected from any one of SEQ ID NOs: 73-92.

| | | SEQ |
|---|---|---|
| | | ID |
| Description | Sequence | NO. |
| 4H11 | DIELTQSPSSLAVSAGERVTMNCKSSQSLLNSRTRKNQLAWYQQKPGQSP | 73 |
| L1H1- | ELLIYWASTRQSGVPDRFSGSGSGTDFTLTISSVQAEDVAVYYCQQSYNLL | |
| BsAB | TFGPGTKLEIKRGGGGSGGGGSGGGGSEVKLQESGGGFVKPGGSLRVS | |
| | CAASGFTFSSYAMSWVRLAPEMRLEWVATISSAGGYIFYSDSVQGRFTISR | |
| | DNAKNSLHLQMGSLRSGDTAMYYCARQGFGNYGDYYAMDYWGQGTTV | |
| | TVSSTSGGGGSDVQLVQSGAEVKKPGASVKVSCKASGYTFTRYTMHWVR | |
| | QAPGQGLEWIGYINPSRGYTNYADSVKGRFTITTDKSTSTAYMELSSLRSE | |
| | DTATYYCARYYDDHYCLDYWGQGTTVTVSSGEGTSTGSGGSGGSGGAD | |
| | DIVLTQSPATLSLSPGERATLSCRASQSVSYMNWYQQKPGKAPKRWIYDTS | |
| | KVASGVPARFSGSGSGTDYSLTINSLEAEDAATYYCQQWSSNPLTFGGGT | |
| | KVEIK [symbol] | |
| 4H11 | DIVLTQSPDSLAVSLGERVTMNCKSSQSLLNSRTRKNQLAWYQQKPGQSP | 74 |
| L2H1- | ELLIYWASTRQSGVPDRFSGSGSGTDFTLTISSVQAEDVAVYYCQQSYNLL | |
| BsAB | TFGQGTKLEIKRGGGGSGGGGSGGGGSEVKLQESGGGFVKPGGSLRVS | |
| | CAASGFTFSSYAMSWVRLAPEMRLEWVATISSAGGYIFYSDSVQGRFTISR | |
| | DNAKNSLHLQMGSLRSGDTAMYYCARQGFGNYGDYYAMDYWGQGTTV | |
| | TVSSTSGGGGSDVQLVQSGAEVKKPGASVKVSCKASGYTFTRYTMHWVR | |
| | QAPGQGLEWIGYINPSRGYTNYADSVKGRFTITTDKSTSTAYMELSSLRSE | |
| | DTATYYCARYYDDHYCLDYWGQGTTVTVSSGEGTSTGSGGSGGSGGAD | |
| | DIVLTQSPATLSLSPGERATLSCRASQSVSYMNWYQQKPGKAPKRWIYDTS | |
| | KVASGVPARFSGSGSGTDYSLTINSLEAEDAATYYCQQWSSNPLTFGGGT | |
| | KVEIK [symbol] | |
| 4H11 | DIELTQSPSSLAVSAGERVTMNCKSSQSLLNSRTRKNQLAWYQQKPGQSP | 75 |
| L1H2- | ELLIYWASTRQSGVPDRFSGSGSGTDFTLTISSVQAEDVAVYYCQQSYNLL | |
| BsAB | TFGPGTKLEIKRGGGGSGGGGSGGGGSEVQLVESGGGLVKPGGSLRVS | |
| | CAASGFTFSSYAMSWVRLAPGKGLEWVATISSAGGYIFYSDSVQGRFTISR | |
| | DNAKNSLYLQMNSLRAEDTAMYYCARQGFGNYGDYYAMDYWGQGTLVT | |
| | VSSTSGGGGSDVQLVQSGAEVKKPGASVKVSCKASGYTFTRYTMHWVRQ | |
| | APGQGLEWIGYINPSRGYTNYADSVKGRFTITTDKSTSTAYMELSSLRSED | |
| | TATYYCARYYDDHYCLDYWGQGTTVTVSSGEGTSTGSGGSGGSGGADD | |
| | IVLTQSPATLSLSPGERATLSCRASQSVSYMNWYQQKPGKAPKRWIYDTSK | |
| | VASGVPARFSGSGSGTDYSLTINSLEAEDAATYYCQQWSSNPLTFGGGTK | |
| | VEIK [symbol] | |
| 4H11 | DIVLTQSPDSLAVSLGERVTMNCKSSQSLLNSRTRKNQLAWYQQKPGQSP | 76 |
| L2H2- | ELLIYWASTRQSGVPDRFSGSGSGTDFTLTISSVQAEDVAVYYCQQSYNLL | |
| BsAB | TFGQGTKLEIKRGGGGSGGGGSGGGGSEVQLVESGGGLVKPGGSLRVS | |
| | CAASGFTFSSYAMSWVRLAPGKGLEWVATISSAGGYIFYSDSVQGRFTISR | |
| | DNAKNSLYLQMNSLRAEDTAMYYCARQGFGNYGDYYAMDYWGQGTLVT | |
| | VSSTSGGGGSDVQLVQSGAEVKKPGASVKVSCKASGYTFTRYTMHWVRQ | |
| | APGQGLEWIGYINPSRGYTNYADSVKGRFTITTDKSTSTAYMELSSLRSED | |
| | TATYYCARYYDDHYCLDYWGQGTTVTVSSGEGTSTGSGGSGGSGGADD | |
| | IVLTQSPATLSLSPGERATLSCRASQSVSYMNWYQQKPGKAPKRWIYDTSK | |
| | VASGVPARFSGSGSGTDYSLTINSLEAEDAATYYCQQWSSNPLTFGGGTK | |
| | VEIK [symbol] | |
| 18C6 | DIVMTQSAPSVPVTPGESVSISCRSSKSLLHSNGNTYLYWFLQKPGQSPQR | 77 |
| L1H1- | LIYYMSNLASGVPDRFSGRGSGTDFTLKISRVEAEDVGVYYCMQSLEYPLT | |
| BsAB | FGGGTKLEIKRGGGGSGGGGSGGGGSQVTLKESGPGILQPTQTLTLTCT | |
| | FSGFSLSTVGMGVGWSRQPSGKGLEWLAHIWWDDEDKYYNPALKSRLTI | |
| | TKDTSKNQVFLKITNVDTADTATYYCTRIGTAQATDALDYWGQGTLVTVS | |
| | STSGGGGSDVQLVQSGAEVKKPGASVKVSCKASGYTFTRYTMHWVRQA | |
| | PGQGLEWIGYINPSRGYTNYADSVKGRFTITTDKSTSTAYMELSSLRSEDT | |
| | ATYYCARYYDDHYCLDYWGQGTTVTVSSGEGTSTGSGGSGGSGGADDI | |
| | VLTQSPATLSLSPGERATLSCRASQSVSYMNWYQQKPGKAPKRWIYDTSK | |
| | VASGVPARFSGSGSGTDYSLTINSLEAEDAATYYCQQWSSNPLTFGGGTK | |
| | VEIK [symbol] | |
| 18C6 | DIVMTQSALSLPVTPGEPVSISCRSSKSLLHSNGNTYLYWFLQKPGQSPQR | 78 |
| L2H1- | LIYYMSNLASGVPDRFSGSGSGTDFTLKISRVEAEDVGVYYCMQSLEYPLT | |
| BsAB | FGGGTKLEIKRGGGGSGGGGSGGGGSQVTLKESGPGILQPTQTLTLTCT | |
| | FSGFSLSTVGMGVGWSRQPSGKGLEWLAHIWWDDEDKYYNPALKSRLTI | |
| | TKDTSKNQVFLKITNVDTADTATYYCTRIGTAQATDALDYWGQGTLVTVS | |
| | STSGGGGSDVQLVQSGAEVKKPGASVKVSCKASGYTFTRYTMHWVRQA | |
| | PGQGLEWIGYINPSRGYTNYADSVKGRFTITTDKSTSTAYMELSSLRSEDT | |
| | ATYYCARYYDDHYCLDYWGQGTTVTVSSGEGTSTGSGGSGGSGGADDI | |
| | VLTQSPATLSLSPGERATLSCRASQSVSYMNWYQQKPGKAPKRWIYDTSK | |
| | VASGVPARFSGSGSGTDYSLTINSLEAEDAATYYCQQWSSNPLTFGGGTK | |
| | VEIK [symbol] | |
| 18C6 | DIVMTQSAPSVPVTPGESVSISCRSSKSLLHSNGNTYLYWFLQKPGQSPQR | 79 |
| L1H2- | LIYYMSNLASGVPDRFSGRGSGTDFTLKISRVEAEDVGVYYCMQSLEYPLT | |
| BsAB | FGGGTKLEIKRGGGGSGGGGSGGGGSQVTLKESGPTLVKPTQTLTLTCT | |
| | FSGFSLSTVGMGVGWSRQPSGKGLEWLAHIWWDDEDKYYNPALKSRLTI | |
| | TKDTSKNQVVLTITNVDPVDTATYYCTRIGTAQATDALDYWGQGTLVTVS | |
| | STSGGGGSDVQLVQSGAEVKKPGASVKVSCKASGYTFTRYTMHWVRQA | |
| | PGQGLEWIGYINPSRGYTNYADSVKGRFTITTDKSTSTAYMELSSLRSEDT | |
| | ATYYCARYYDDHYCLDYWGQGTTVTVSSGEGTSTGSGGSGGSGGADDI | |
| | VLTQSPATLSLSPGERATLSCRASQSVSYMNWYQQKPGKAPKRWIYDTSK | |
| | VASGVPARFSGSGSGTDYSLTINSLEAEDAATYYCQQWSSNPLTFGGGTK | |
| | VEIK [symbol] | |
| 18C6 | DIVMTQSALSLPVTPGEPVSISCRSSKSLLHSNGNTYLYWFLQKPGQSPQR | 80 |
| L2H2- | LIYYMSNLASGVPDRFSGSGSGTDFTLKISRVEAEDVGVYYCMQSLEYPLT | |
| BsAB | FGGGTKLEIKRGGGGSGGGGSGGGGSQVTLKESGPTLVKPTQTLTLTCT | |
| | FSGFSLSTVGMGVGWSRQPSGKGLEWLAHIWWDDEDKYYNPALKSRLTI | |
| | TKDTSKNQVVLTITNVDPVDTATYYCTRIGTAQATDALDYWGQGTLVTVS | |
| | STSGGGGSDVQLVQSGAEVKKPGASVKVSCKASGYTFTRYTMHWVRQA | |
| | PGQGLEWIGYINPSRGYTNYADSVKGRFTITTDKSTSTAYMELSSLRSEDT | |
| | ATYYCARYYDDHYCLDYWGQGTTVTVSSGEGTSTGSGGSGGSGGADDI | |
| | VLTQSPATLSLSPGERATLSCRASQSVSYMNWYQQKPGKAPKRWIYDTSK | |
| | VASGVPARFSGSGSGTDYSLTINSLEAEDAATYYCQQWSSNPLTFGGGTK | |
| | VEIK [symbol] | |
| 4H11 | EVKLQESGGGFVKPGGSLRVSCAASGFTFSSYAMSWVRLAPEMRLEWVA | 81 |
| H1L1- | TISSAGGYIFYSDSVQGRFTISRDNAKNSLHLQMGSLRSGDTAMYYCARQ | |
| BsAb | GFGNYGDYYAMDYWGQGTTVTVSSGGGGSGGGGSGGGGSDIELTQSPS | |
| | SLAVSAGERVTMNCKSSQSLLNSRTRKNQLAWYQQKPGQSPELLIYWAST | |
| | RQSGVPDRFSGSGSGTDFTLTISSVQAEDVAVYYCQQSYNLLTFGPGTKL | |
| | EIKRTSGGGGSDVQLVQSGAEVKKPGASVKVSCKASGYTFTRYTMHWVR | |
| | QAPGQGLEWIGYINPSRGYTNYADSVKGRFTITTDKSTSTAYMELSSLRSE | |
| | DTATYYCARYYDDHYCLDYWGQGTTVTVSSGEGTSTGSGGSGGSGGAD | |
| | DIVLTQSPATLSLSPGERATLSCRASQSVSYMNWYQQKPGKAPKRWIYDTS | |
| | KVASGVPARFSGSGSGTDYSLTINSLEAEDAATYYCQQWSSNPLTFGGGT | |
| | KVEIK [symbol] | |
| 4H11 | EVKLQESGGGFVKPGGSLRVSCAASGFTFSSYAMSWVRLAPEMRLEWVA | 82 |
| H1L2- | TISSAGGYIFYSDSVQGRFTISRDNAKNSLHLQMGSLRSGDTAMYYCARQ | |
| BsAb | GFGNYGDYYAMDYWGQGTTVTVSSGGGGSGGGGSGGGGSDIVLTQSP | |
| | DSLAVSLGERVTMNCKSSQSLLNSRTRKNQLAWYQQKPGQSPELLIYWAS | |
| | TRQSGVPDRFSGSGSGTDFTLTISSVQAEDVAVYYCQQSYNLLTFGQGTK | |
| | LEIKRTSGGGGSDVQLVQSGAEVKKPGASVKVSCKASGYTFTRYTMHWV | |
| | RQAPGQGLEWIGYINPSRGYTNYADSVKGRFTITTDKSTSTAYMELSSLRS | |
| | EDTATYYCARYYDDHYCLDYWGQGTTVTVSSGEGTSTGSGGSGGSGGA | |
| | DDIVLTQSPATLSLSPGERATLSCRASQSVSYMNWYQQKPGKAPKRWIYD | |
| | TSKVASGVPARFSGSGSGTDYSLTINSLEAEDAATYYCQQWSSNPLTFGGG | |
| | TKVEIK [symbol] | |
| 4H11 | EVQLVESGGGLVKPGGSLRVSCAASGFTFSSYAMSWVRLAPGKGLEWVA | 83 |
| H2L1- | TISSAGGYIFYSDSVQGRFTISRDNAKNSLYLQMNSLRAEDTAMYYCARQG | |
| BsAb | FGNYGDYYAMDYWGQGTLVTVSSGGGGSGGGGSGGGGSDIELTQSPSS | |
| | LAVSAGERVTMNCKSSQSLLNSRTRKNQLAWYQQKPGQSPELLIYWASTR | |
| | QSGVPDRFSGSGSGTDFTLTISSVQAEDVAVYYCQQSYNLLTFGPGTKLEI | |
| | KRTSGGGGSDVQLVQSGAEVKKPGASVKVSCKASGYTFTRYTMHWVRQ | |
| | APGQGLEWIGYINPSRGYTNYADSVKGRFTITTDKSTSTAYMELSSLRSED | |
| | TATYYCARYYDDHYCLDYWGQGTTVTVSSGEGTSTGSGGSGGSGGADD | |
| | IVLTQSPATLSLSPGERATLSCRASQSVSYMNWYQQKPGKAPKRWIYDTSK | |
| | VASGVPARFSGSGSGTDYSLTINSLEAEDAATYYCQQWSSNPLTFGGGTK | |
| | VEIK [symbol] | |
| 4H11 | EVQLVESGGGLVKPGGSLRVSCAASGFTFSSYAMSWVRLAPGKGLEWVA | 84 |
| H2L2- | TISSAGGYIFYSDSVQGRFTISRDNAKNSLYLQMNSLRAEDTAMYYCARQG | |
| BsAb | FGNYGDYYAMDYWGQGTLVTVSSGGGGSGGGGSGGGGSDIVLTQSPDS | |
| | LAVSLGERVTMNCKSSQSLLNSRTRKNQLAWYQQKPGQSPELLIYWASTR | |
| | QSGVPDRFSGSGSGTDFTLTISSVQAEDVAVYYCQQSYNLLTFGQGTKLEI | |
| | KRTSGGGGSDVQLVQSGAEVKKPGASVKVSCKASGYTFTRYTMHWVRQ | |
| | APGQGLEWIGYINPSRGYTNYADSVKGRFTITTDKSTSTAYMELSSLRSED | |
| | TATYYCARYYDDHYCLDYWGQGTTVTVSSGEGTSTGSGGSGGSGGADD | |
| | IVLTQSPATLSLSPGERATLSCRASQSVSYMNWYQQKPGKAPKRWIYDTSK | |
| | VASGVPARFSGSGSGTDYSLTINSLEAEDAATYYCQQWSSNPLTFGGGTK | |
| | VEIK [symbol] | |
| 18C6 | QVTLKESGPGILQPTQTLTLTCTFSGFSLSTVGMGVGWSRQPSGKGLEW | 85 |
| H1L1- | LAHIWWDDEDKYYNPALKSRLTITKDTSKNQVFLKITNVDTADTATYYCT | |
| BsAb | RIGTAQATDALDYWGQGTLVTVSSGGGGSGGGGSGGGGSDIVMTQSAP | |
| | SVPVTPGESVSISCRSSKSLLHSNGNTYLYWFLQKPGQSPQRLIYYMSNLAS | |
| | GVPDRFSGRGSGTDFTLKISRVEAEDVGVYYCMQSLEYPLTFGGGTKLEI | |
| | KRTSGGGGSDVQLVQSGAEVKKPGASVKVSCKASGYTFTRYTMHWVRQ | |
| | APGQGLEWIGYINPSRGYTNYADSVKGRFTITTDKSTSTAYMELSSLRSED | |
| | TATYYCARYYDDHYCLDYWGQGTTVTVSSGEGTSTGSGGSGGSGGADD | |
| | IVLTQSPATLSLSPGERATLSCRASQSVSYMNWYQQKPGKAPKRWIYDTSK | |
| | VASGVPARFSGSGSGTDYSLTINSLEAEDAATYYCQQWSSNPLTFGGGTK | |
| | VEIK [symbol] | |
| 18C6 | QVTLKESGPGILQPTQTLTLTCTFSGFSLSTVGMGVGWSRQPSGKGLEW | 86 |
| H1L2- | LAHIWWDDEDKYYNPALKSRLTITKDTSKNQVFLKITNVDTADTATYYCT | |
| BsAb | RIGTAQATDALDYWGQGTLVTVSSGGGGSGGGGSGGGGSDIVMTQSAL | |
| | SLPVTPGEPVSISCRSSKSLLHSNGNTYLYWFLQKPGQSPQRLIYYMSNLAS | |
| | GVPDRFSGSGSGTDFTLKISRVEAEDVGVYYCMQSLEYPLTFGGGTKLEI | |
| | KRTSGGGGSDVQLVQSGAEVKKPGASVKVSCKASGYTFTRYTMHWVRQ | |
| | APGQGLEWIGYINPSRGYTNYADSVKGRFTITTDKSTSTAYMELSSLRSED | |
| | TATYYCARYYDDHYCLDYWGQGTTVTVSSGEGTSTGSGGSGGSGGADD | |
| | IVLTQSPATLSLSPGERATLSCRASQSVSYMNWYQQKPGKAPKRWIYDTSK | |
| | VASGVPARFSGSGSGTDYSLTINSLEAEDAATYYCQQWSSNPLTFGGGTK | |
| | VEIK [symbol] | |
| 18C6 | QVTLKESGPTLVKPTQTLTLTCTFSGFSLSTVGMGVGWSRQPSGKGLEW | 87 |
| H2L1- | LAHIWWDDEDKYYNPALKSRLTITKDTSKNQVVLTITNVDPVDTATYYCT | |
| BsAb | RIGTAQATDALDYWGQGTLVTVSSGGGGSGGGGSGGGGSDIVMTQSAP | |
| | SVPVTPGESVSISCRSSKSLLHSNGNTYLYWFLQKPGQSPQRLIYYMSNLAS | |
| | GVPDRFSGRGSGTDFTLKISRVEAEDVGVYYCMQSLEYPLTFGGGTKLEI | |
| | KRTSGGGGSDVQLVQSGAEVKKPGASVKVSCKASGYTFTRYTMHWVRQ | |
| | APGQGLEWIGYINPSRGYTNYADSVKGRFTITTDKSTSTAYMELSSLRSED | |
| | TATYYCARYYDDHYCLDYWGQGTTVTVSSGEGTSTGSGGSGGSGGADD | |
| | IVLTQSPATLSLSPGERATLSCRASQSVSYMNWYQQKPGKAPKRWIYDTSK | |
| | VASGVPARFSGSGSGTDYSLTINSLEAEDAATYYCQQWSSNPLTFGGGTK | |
| | VEIK [symbol] | |
| 18C6 | QVTLKESGPTLVKPTQTLTLTCTFSGFSLSTVGMGVGWSRQPSGKGLEW | 88 |
| H2L2- | LAHIWWDDEDKYYNPALKSRLTITKDTSKNQVVLTITNVDPVDTATYYCT | |
| BsAb | RIGTAQATDALDYWGQGTLVTVSSGGGGSGGGGSGGGGSDIVMTQSAL | |
| | SLPVTPGEPVSISCRSSKSLLHSNGNTYLYWFLQKPGQSPQRLIYYMSNLAS | |
| | GVPDRFSGSGSGTDFTLKISRVEAEDVGVYYCMQSLEYPLTFGGGTKLEI | |
| | KRTSGGGGSDVQLVQSGAEVKKPGASVKVSCKASGYTFTRYTMHWVRQ | |
| | APGQGLEWIGYINPSRGYTNYADSVKGRFTITTDKSTSTAYMELSSLRSED | |
| | TATYYCARYYDDHYCLDYWGQGTTVTVSSGEGTSTGSGGSGGSGGADD | |
| | IVLTQSPATLSLSPGERATLSCRASQSVSYMNWYQQKPGKAPKRWIYDTSK | |
| | VASGVPARFSGSGSGTDYSLTINSLEAEDAATYYCQQWSSNPLTFGGGTK | |
| | VEIK [symbol] | |
| Clone 8 | DIQLTQSPSAVSASVGDRVTITCRASQDVSKWLAWYQQKPGKAPRLLISAA | 89 |
| (LH)/anti- | SGLQSWVPSRFSGSGSGTEFTLSISSLQPEDFATYYCQQANSFPWTFGQG | |
| CD3 | TKVEIKRSRGGGGSGGGGSGGGGSLEMAQVQLQQWGAGLLKPSETLS | |
| bispecific | LTCAVYGGSFSGYYWSWIRQPPGKGLEWIGEINHSGSTNYNPSLKSRVTIS | |
| antibody | VDTSKNQFSLKLSSVTAADTAVYYCARQSYITDSWGQGTLVTVSSTSGGG | |
| | GSDVQLVQSGAEVKKPGASVKVSCKASGYTFTRYTMHWVRQAPGQGLE | |
| | WIGYINPSRGYTNYADSVKGRFTITTDKSTSTAYMELSSLRSEDTATYYCAR | |
| | YYDDHYCLDYWGQGTTVTVSSGEGTSTGSGGSGGSGGADDIVLTQSPA | |
| | TLSLSPGERATLSCRASQSVSYMNWYQQKPGKAPKRWIYDTSKVASGVPA | |
| | RFSGSGSGTDYSLTINSLEAEDAATYYCQQWSSNPLTFGGGTKVEIK [symbol] | |
| | [symbol] | |
| Clone 8 | QVQLQQWGAGLLKPSETLSLTCAVYGGSFSGYYWSWIRQPPGKGLEWIG | 90 |
| (HL)/anti- | EINHSGSTNYNPSLKSRVTISVDTSKNQFSLKLSSVTAADTAVYYCARQSYIT | |
| CD3 | DSWGQGTLVTVSSSRGGGGSGGGGSGGGGSLEMADIQLTQSPSAVSAS | |
| bispecific | VGDRVTITCRASQDVSKWLAWYQQKPGKAPRLLISAASGLQSWVPSRFSG | |
| antibody | SGSGTEFTLSISSLQPEDFATYYCQQANSFPWTFGQGTKVEIKRTSGGGG | |
| | SDVQLVQSGAEVKKPGASVKVSCKASGYTFTRYTMHWVRQAPGQGLEWI | |
| | GYINPSRGYTNYADSVKGRFTITTDKSTSTAYMELSSLRSEDTATYYCARYY | |
| | DDHYCLDYWGQGTTVTVSSGEGTSTGSGGSGGSGGADDIVLTQSPATLS | |
| | LSPGERATLSCRASQSVSYMNWYQQKPGKAPKRWIYDTSKVASGVPARFS | |
| | GSGSGTDYSLTINSLEAEDAATYYCQQWSSNPLTFGGGTKVEIK [symbol] | |
| | [symbol] | |
| Clone 12 | QVQLQQWGAGLLKPSETLSLTCAVYGGSFSGYYWSWIRQPPGKGLEWIG | 91 |
| (HL)/anti- | EINHSGSTNYNPSLKSRIIMSVDTSKRQFSLKLRSATAADTAVYYCARWSPF | |
| CD3 | SYKQMYDYWGQGTLVTVSSSRGGGGSGGGGSGGGGSLEMANFMLTQP | |
| bispecific | HSVSESPGKTVTISCTRSRGSIASAYVQWYQQRPGSAPITVIYEDYERPSEIP | |
| antibody | DRFSGSIDSSSNSASLTISGLKTEDEADYYCQSYDDNDHVIFGGGTKVTVL | |
| | GTSGGGGSDVQLVQSGAEVKKPGASVKVSCKASGYTFTRYTMHWVRQA | |
| | PGQGLEWIGYINPSRGYTNYADSVKGRFTITTDKSTSTAYMELSSLRSEDT | |
| | ATYYCARYYDDHYCLDYWGQGTTVTVSSGEGTSTGSGGSGGSGGADDI | |
| | VLTQSPATLSLSPGERATLSCRASQSVSYMNWYQQKPGKAPKRWIYDTSK | |
| | VASGVPARFSGSGSGTDYSLTINSLEAEDAATYYCQQWSSNPLTFGGGTK | |
| | VEIK [symbol] | |
| Clone 12 | NFMLTQPHSVSESPGKTVTISCTRSRGSIASAYVQWYQQRPGSAPITVIYED | 92 |
| (LH)/anti- | YERPSEIPDRFSGSIDSSSNSASLTISGLKTEDEADYYCQSYDDNDHVIFGG | |
| CD3 | GTKVTVLGSRGGGGSGGGGSGGGGSLEMAQVQLQQWGAGLLKPSETL | |
| bispecific | SLTCAVYGGSFSGYYWSWIRQPPGKGLEWIGEINHSGSTNYNPSLKSRIIM | |
| antibody | SVDTSKRQFSLKLRSATAADTAVYYCARWSPFSYKQMYDYWGQGTLVTVS | |
| | STSGGGGSDVQLVQSGAEVKKPGASVKVSCKASGYTFTRYTMHWVRQA | |
| | PGQGLEWIGYINPSRGYTNYADSVKGRFTITTDKSTSTAYMELSSLRSEDT | |
| | ATYYCARYYDDHYCLDYWGQGTTVTVSSGEGTSTGSGGSGGSGGADDI | |
| | VLTQSPATLSLSPGERATLSCRASQSVSYMNWYQQKPGKAPKRWIYDTSK | |
| | VASGVPARFSGSGSGTDYSLTINSLEAEDAATYYCQQWSSNPLTFGGGTK | |
| | VEIK [symbol] | |

V_{H} and V_{L} domains are italicized, V_{H} and V_{L} CDR sequences of MUC16 antigen binding fragment are underlined, linker sequences are indicated in regular font, and Histidine tag is indicated in dash underline

The immunoglobulin-related compositions of the present technology can further be recombinantly fused to a heterologous polypeptide at the N- or C-terminus or chemically conjugated (including covalently and non-covalently conjugations) to polypeptides or other compositions. For example, the immunoglobulin-related compositions of the present technology can be recombinantly fused or conjugated to molecules useful as labels in detection assays and effector molecules such as heterologous polypeptides, drugs, or toxins. See, e.g., WO 92/08495; WO 91/14438; WO 89/12624; U.S. Pat. No. 5,314,995; and EP 0 396 387.

In any of the above embodiments of the immunoglobulin-related compositions of the present technology, the antibody or antigen binding fragment may be optionally conjugated to an agent selected from the group consisting of isotopes, dyes, chromagens, contrast agents, imaging agents, cytotoxic agents, drugs, toxins, cytokines, enzymes, enzyme inhibitors, hormones, hormone antagonists, growth factors, radionuclides, metals, liposomes, nanoparticles, RNA, DNA or any combination thereof. For a chemical bond or physical bond, a functional group on the immunoglobulin-related composition typically associates with a functional group on the agent. Alternatively, a functional group on the agent associates with a functional group on the immunoglobulin-related composition.

In certain embodiments, the imaging agent is a detectable label, such as, a chromogenic, enzymatic, radioisotopic, isotopic, fluorescent, toxic, chemiluminescent, nuclear magnetic resonance contrast agent or other label.

Non-limiting examples of suitable chromogenic labels include diaminobenzidine and 4-hydroxyazo-benzene-2-carboxylic acid.

Non-limiting examples of suitable enzyme labels include malate dehydrogenase, staphylococcal nuclease, delta-5-steroid isomerase, yeast-alcohol dehydrogenase, alpha-glycerol phosphate dehydrogenase, triose phosphate isomerase, peroxidase, alkaline phosphatase, asparaginase, glucose oxidase, beta-galactosidase, ribonuclease, urease, catalase, glucose-6-phosphate dehydrogenase, glucoamylase, and acetylcholine esterase.

Suitable radioisotopes are well known to those skilled in the art and include beta-emitters, gamma-emitters, positron-emitters, and x-ray emitters. Non-limiting examples of suitable radioisotopic labels include ³H, ¹⁸F, ¹¹¹In, ¹²⁵I, ¹³¹I, ³²P, ³³P, ³⁵S, ¹¹C, ¹⁴C, ⁵¹Cr, ⁵⁷To, ⁵⁸Co, ⁵⁹Fe, ⁷⁵Se, ¹⁵²Eu, ⁹⁰Y ⁶⁷Cu, ²¹⁷Ci, ²¹¹At, ²¹²Pb, ⁴⁷Sc, ²²³Ra, ²²³Ra, ⁸⁹Zr, ¹⁷⁷Lu, and ¹⁰⁹Pd. In certain embodiments, ¹¹¹In is a preferred isotope for in vivo imaging as it avoids the problem of dehalogenation of ¹²⁵I or ¹³¹I-labeled anti-MUC16 immunoglobulin-related compositions or antigen-binding fragments thereof in the liver. In addition, ¹¹¹In has a more favorable gamma emission energy for imaging (Perkins et al, Eur. J. Nucl. Med. 70:296-301 (1985); Carasquillo et ah, J. Nucl. Med. 25:281-287 (1987)). For example, ¹¹¹In coupled to monoclonal antibodies with 1-(P-isothiocyanatobenzyl)-DPTA has shown little uptake in non-tumorous tissues, particularly the liver, and therefore enhances specificity of tumor localization (Esteban et al., J. Nucl. Med. 28:861-870 (1987)).

Non-limiting examples of suitable non-radioactive isotopic labels include 157Gd, ⁵⁵Mn, ¹⁶²Dy, ⁵²Tr, and ⁵⁶Fe.

Non-limiting examples of suitable fluorescent labels include a ¹⁵²Eu label, a fluorescein label, an isothiocyanate label, a rhodamine label, a phycoerythrin label, a phycocyanin label, an allophycocyanin label, a Green Fluorescent Protein (GFP) label, an o-phthaldehyde label, and a fluorescamine label.

Non-limiting examples of chemiluminescent labels include a luminol label, an isoluminol label, an aromatic acridinium ester label, an imidazole label, an acridinium salt label, an oxalate ester label, a luciferin label, a luciferase label, and an aequorin label.

Non-limiting examples of nuclear magnetic resonance contrasting agents include heavy metal nuclei such as Gd, Mn, and iron.

Techniques known to one of ordinary skill in the art for conjugating the above-described labels to said anti-MUC16 antibodies or antigen-binding fragments thereof, bispecific antibodies, antibody heavy chains, antibody light chains, and fusion proteins are described in, for example, Kennedy et at., Clin. CMm. Acta 70: 1-31 (1976), and Schurs et al, Clin. CMm. Acta 81: 1-40 (1977). Coupling techniques mentioned in the latter are the glutaraldehyde method, the periodate method, the dimaleimide method, the m-maleimidobenzoyl-N-hydroxy-succinimide ester method, all of which methods are incorporated by reference herein.

Nonlimiting examples of cytotoxic agents include a cytostatic or cytocidal agent, a radioactive metal ion, e.g., alpha-emitters, and toxins, e.g., pseudomonas exotoxin, abrin, cholera toxin, ricin A, and diphtheria toxin.

In certain embodiments, the agent is a diagnostic agent. A diagnostic agent is an agent useful in diagnosing or detecting a disease by locating the cells containing the antigen. Useful diagnostic agents include, but are not limited to, radioisotopes, dyes (such as with the biotin-streptavidin complex), contrast agents, fluorescent compounds or molecules and enhancing agents (e.g., paramagnetic ions) for magnetic resonance imaging (MRI). U.S. Pat. No. 6,331,175 describes MRI technique and the preparation of antibodies conjugated to a MRI enhancing agent and is incorporated in its entirety by reference. Preferably, the diagnostic agents are selected from the group consisting of radioisotopes, enhancing agents for use in magnetic resonance imaging, and fluorescent compounds. In order to load an anti-MUC16 immunoglobulin-related composition with radioactive metals or paramagnetic ions, it may be necessary to react it with a reagent having a long tail to which are attached a multiplicity of chelating groups for binding the ions. Such a tail can be a polymer such as a polylysine, polysaccharide, or other derivatized or derivatizable chain having pendant groups to which can be bound chelating groups such as, for example, ethylenediaminetetraacetic acid (EDTA), diethylenetriaminepentaacetic acid (DTPA), porphyrins, polyamines, crown ethers, bis-thiosemicarbazones, polyoximes, and like groups known to be useful for this purpose. Chelates are coupled to the antibodies using standard chemistries. The chelate is normally linked to the antibody by a group which enables formation of a bond to the molecule with minimal loss of immunoreactivity and minimal aggregation and/or internal cross-linking other, more unusual, methods and reagents for conjugating chelates to antibodies are disclosed in U.S. Pat. No. 4,824,659 to Hawthorne, entitled “Antibody Conjugates,” issued Apr. 25, 1989, the disclosure of which is incorporated herein in its entirety by reference. Particularly useful metal-chelate combinations include 2-benzyl-DTPA and its monomethyl and cyclohexyl analogs, used with diagnostic isotopes for radio-imaging. The same chelates, when complexed with non-radioactive metals, such as manganese, iron and gadolinium are useful for MRI, when used along with an anti-MUC16 immunoglobulin-related composition provided herein.

Macrocyclic chelates such as NOTA, DOTA, and TETA are of use with a variety of metals and radiometals, most particularly with radionuclides of gallium, yttrium and copper, respectively. Such metal-chelate complexes can be made very stable by tailoring the ring size to the metal of interest. Other ring-type chelates such as macrocyclic polyethers, which are of interest for stably binding nuclides, such as ²²³Ra for RAIT are encompassed herein.

The functional groups on the agent and immunoglobulin-related composition can associate directly. For example, a functional group (e.g., a sulfhydryl group) on an agent can associate with a functional group (e.g., sulfhydryl group) on an immunoglobulin-related composition to form a disulfide. Alternatively, the functional groups can associate through a cross-linking agent (i.e., linker). Some examples of cross-linking agents are described below. The cross-linker can be attached to either the agent or the immunoglobulin-related composition. The number of agents or immunoglobulin-related compositions in a conjugate is also limited by the number of functional groups present on the other. For example, the maximum number of agents associated with a conjugate depends on the number of functional groups present on the immunoglobulin-related composition. Alternatively, the maximum number of immunoglobulin-related compositions associated with an agent depends on the number of functional groups present on the agent.

In yet another embodiment, the conjugate comprises one immunoglobulin-related composition associated to one agent. In one embodiment, a conjugate comprises at least one agent chemically bonded (e.g., conjugated) to at least one immunoglobulin-related composition. The agent can be chemically bonded to an immunoglobulin-related composition by any method known to those in the art. For example, a functional group on the agent may be directly attached to a functional group on the immunoglobulin-related composition. Some examples of suitable functional groups include, for example, amino, carboxyl, sulfhydryl, maleimide, isocyanate, isothiocyanate and hydroxyl.

The agent may also be chemically bonded to the immunoglobulin-related composition by means of cross-linking agents, such as dialdehydes, carbodiimides, dimaleimides, and the like. Cross-linking agents can, for example, be obtained from Pierce Biotechnology, Inc., Rockford, Ill. The Pierce Biotechnology, Inc. web-site can provide assistance. Additional cross-linking agents include the platinum cross-linking agents described in U.S. Pat. Nos. 5,580,990; 5,985,566; and 6,133,038 of Kreatech Biotechnology, B.V., Amsterdam, The Netherlands.

Alternatively, the functional group on the agent and immunoglobulin-related composition can be the same. Homobifunctional cross-linkers are typically used to cross-link identical functional groups. Examples of homobifunctional cross-linkers include EGS (i.e., ethylene glycol bis[succinimidylsuccinate]), DSS (i.e., disuccinimidyl suberate), DMA (i.e., dimethyl adipimidate·2HCl), DTSSP (i.e., 3,3′-dithiobis[sulfosuccinimidylpropionate])), DPDPB (i.e., 1,4-di-[3′-(2′-pyridyldithio)-propionamido]butane), and BMH (i.e., bis-maleimidohexane). Such homobifunctional cross-linkers are also available from Pierce Biotechnology, Inc.

In other instances, it may be beneficial to cleave the agent from the immunoglobulin-related composition. The web-site of Pierce Biotechnology, Inc. described above can also provide assistance to one skilled in the art in choosing suitable cross-linkers which can be cleaved by, for example, enzymes in the cell. Thus the agent can be separated from the immunoglobulin-related composition. Examples of cleavable linkers include SMPT (i.e., 4-succinimidyloxycarbonyl-methyl-a-[2-pyridyldithio]toluene), Sulfo-LC-SPDP (i.e., sulfosuccinimidyl 6-(3-[2-pyridyldithio]-propionamido)hexanoate), LC-SPDP (i.e., succinimidyl 6-(3-[2-pyridyldithio]-propionamido)hexanoate), Sulfo-LC-SPDP (i.e., sulfosuccinimidyl 6-(3-[2-pyridyldithio]-propionamido)hexanoate), SPDP (i.e., N-succinimidyl 3-[2-pyridyldithio]-propionamidohexanoate), and AEDP (i.e., 3-[(2-aminoethyl)dithio]propionic acid HCl).

In another embodiment, a conjugate comprises at least one agent physically bonded with at least one immunoglobulin-related composition. Any method known to those in the art can be employed to physically bond the agents with the immunoglobulin-related compositions. For example, the immunoglobulin-related compositions and agents can be mixed together by any method known to those in the art. The order of mixing is not important. For instance, agents can be physically mixed with immunoglobulin-related compositions by any method known to those in the art. For example, the immunoglobulin-related compositions and agents can be placed in a container and agitated, by for example, shaking the container, to mix the immunoglobulin-related compositions and agents.

The immunoglobulin-related compositions can be modified by any method known to those in the art. For instance, the immunoglobulin-related composition may be modified by means of cross-linking agents or functional groups, as described above.

In some embodiments, the anti-MUC16 immunoglobulin-related compositions of the present technology bind to the C-terminal 114 amino acid residues of mature MUC16 (e.g., MUC16^{c114}) with an affinity that is at least about 10 times (including for example at least about any of 10, 10², 10³, 10⁴, 10⁵, 10⁶, or 10⁷ times) its binding affinity for non-target. In some embodiments, the non-target is an antigen that is not MUC16. Binding affinity can be determined by methods known in the art, such as ELISA, fluorescence activated cell sorting (FACS) analysis, or radioimmunoprecipitation assay (RIA). K_{d} can be determined by methods known in the art, such as surface plasmon resonance (SPR) assay utilizing, for example, Biacore instruments, or kinetic exclusion assay (KinExA) utilizing, for example, Sapidyne instruments.

In some embodiments, the anti-MUC16 immunoglobulin-related composition cross-reacts with MUC16 polypeptide from a species other than human. In some embodiments, the anti-MUC16 immunoglobulin-related composition is completely specific for human MUC16 and does not exhibit species or other types of non-human cross-reactivity. In some embodiments, the anti-MUC16 immunoglobulin-related composition specifically recognizes MUC16 expressed on the cell surface of a cancer cell (such as solid tumor). In some embodiments, the anti-MUC16 immunoglobulin-related composition specifically recognizes MUC16 expressed on the cell surface of one or more of ovarian cancer cells, breast cancer cells, prostate cancer cells, colon cancer cells, lung cancer cells, brain cancer cells, pancreatic cancer cells, kidney cancer cells, fallopian tube cancer cells, uterine (e.g., endometrial) cancer cells, primary peritoneum cancer cells or cancer cells of any other tissue that expresses MUC16. In some embodiments, the anti-MUC16 immunoglobulin-related composition specifically recognizes MUC16 expressed on the cell surface of a cancer cell line, e.g. ovarian cancer cell lines, such as OVCAR3, OVCA-432, OVCA-433 and CAOV3.

In some embodiments, the anti-MUC16 immunoglobulin-related composition cross-reacts with at least one allelic variant of the MUC16 protein, or fragments thereof. In some embodiments, the allelic variant has up to about 30, such as about any of 1, 2, 3, 4, 5, 6, 7, 8, 9, 10, 15, 20, 25, or 30, amino acid substitutions, such as a conservative amino acid substitution, when compared to the naturally occurring MUC16, or fragments thereof. In some embodiments, the anti-MUC16 immunoglobulin-related composition does not cross-react with any allelic variant of the MUC16 protein, or fragments thereof.

In some embodiments, the anti-MUC16 immunoglobulin-related composition cross-reacts with at least one interspecies variant of the MUC16 protein. In some embodiments, for example, the MUC16 protein, or fragments thereof is human MUC16 and the interspecies variant of the MUC16 protein, or fragments thereof, is a mouse or rat variant thereof. In some embodiments, the anti-MUC16 immunoglobulin-related composition does not cross-react with any interspecies variant of the MUC16 protein.

Binding affinity can be indicated by K_{d}, K_{off}, Kₒₙ, or K_{d}. The term “K_{off}”, as used herein, is intended to refer to the off-rate constant for dissociation of an immunoglobulin-related composition from the immunoglobulin-related composition/antigen complex, as determined from a kinetic selection set up. The term “Kₒₙ”, as used herein, is intended to refer to the on-rate constant for association of an immunoglobulin-related composition to the antigen to form the immunoglobulin-related composition/antigen complex. The term equilibrium dissociation constant “K_{d}”, as used herein, refers to the dissociation constant of a particular immunoglobulin-related composition-antigen interaction, and describes the concentration of antigen required to occupy one half of all of the antibody-binding domains present in a solution of immunoglobulin-related composition molecules at equilibrium, and is equal to K_{off}/Kₒₙ. The measurement of K_{d} presupposes that all binding agents are in solution. In the case where the immunoglobulin-related composition is tethered to a cell wall, e.g., in a yeast expression system, the corresponding equilibrium rate constant is expressed as EC50, which gives a good approximation of K_{d}. The affinity constant, K_{d}, is the inverse of the dissociation constant, K_{d}.

The dissociation constant (K_{d}) is used as an indicator showing affinity of antibody moieties to antigens. For example, easy analysis is possible by the Scatchard method using immunoglobulin-related compositions marked with a variety of marker agents, as well as by using Biacore (made by Amersham Biosciences), analysis of biomolecular interactions by surface plasmon resonance, according to the user's manual and attached kit. The K_{d} value that can be derived using these methods is expressed in units of M (Mols). An immunoglobulin-related composition that specifically binds to a target may have a K_{d} of, for example, ≤10⁻⁷ M, ≤10⁻⁸ M, ≤10⁻⁹ M, ≤10⁻¹⁰ M, ≤10⁻¹¹ M, ≤10⁻¹² M, or ≤10⁻¹³ M.

Binding specificity of the immunoglobulin-related composition can be determined experimentally by methods known in the art. Such methods comprise, but are not limited to, Western blots, ELISA-, RIA-, ECL-, IRMA-, EIA-, BIAcore-tests and peptide scans. In some embodiments, the binding affinity of the anti-MUC16 immunoglobulin-related composition is measured by testing the binding affinity of the anti-MUC16 immunoglobulin-related composition to cells expressing MUC16 on the surface (e.g., HepG2 cells).

In some embodiments, the anti-MUC16 immunoglobulin-related composition specifically binds to a target MUC16 (e.g., MUC16^{c114}) with a K_{d} of about 10⁻⁷ M to about 10⁻¹³ M (such as about 10⁻⁷ M to about 10⁻¹³ M, about 10⁹ M to about 10⁻¹³ M, or about 10⁻¹⁰ M to about 10⁻¹² M). Thus in some embodiments, the K_{d} of the binding between the anti-MUC16 immunoglobulin-related composition and target MUC16 (e.g., MUC16^{c114}), is about 10⁻⁷ M to about 10⁻¹³ M, about 1×10⁻⁷ M to about 5×10⁻¹³ M, about 10⁻⁷ M to about 10⁻¹² M, about 10⁻⁷ M to about 10⁻¹¹ M, about 10⁻⁷ M to about 10⁻¹⁰ M, about 10⁻⁷ M to about 10⁻⁹ M, about 10⁻⁸ M to about 10⁻¹³ M, about 1×10⁻⁸ M to about 5×10⁻¹³ M, about 10⁻⁸ M to about 10⁻¹² M, about 10⁻⁸M to about 10⁻¹¹ M, about 10⁻⁸M to about 10⁻¹⁰ M, about 10⁻⁸M to about 10⁻⁹ M, about 5×10⁻⁹ M to about 1×10⁻¹³ M, about 5×10⁻⁹ M to about 1×10⁻¹² M, about 5×10⁻⁹ M to about 1×10⁻¹¹ M, about 5×10⁻⁹ M to about 1×10¹⁰M, about 10⁻⁹M to about 10⁻¹³ M, about 10⁻⁹ M to about 10⁻¹² M, about 10⁻⁹M to about 10⁻¹¹M, about 10⁻⁹M to about 10⁻¹⁰ M, about 5×10⁻¹⁰ M to about 1×10⁻¹³ M, about 5×10⁻¹⁰ M to about 1×10⁻¹² M, about 5×10⁻¹⁰ M to about 1×10⁻¹¹ M, about 10⁻¹⁰ M to about 10⁻¹³ M, about 1×⁻¹⁰ M to about 5×10⁻¹³ M, about 1×10⁻¹⁰ M to about 1×10⁻¹² M, about 1×10⁻¹⁰ M to about 5×10⁻¹²M, about 1×10¹⁰ M to about 1×10⁻¹¹ M, about 10⁻¹¹ M to about 10⁻¹³ M, about 1×10⁻¹¹ M to about 5×10⁻¹³ M, about 10⁻¹¹ M to about 10⁻¹² M, or about 10⁻¹² M to about 10⁻¹³ M.

Functional Activities of anti-Muc6 immunoglobulin-related compositions. In certain embodiments, an anti-MUC16 immunoglobulin-related composition described herein inhibits Matrigel invasion in vitro of cells recombinantly expressing a MUC16 polypeptide (e.g., MUC16 c114). In certain embodiments, the cells recombinantly expressing glycosylated MUC16 c114 are SKOV3 cells. In certain embodiments, the MUC16 polypeptide is glycosylated.

In certain embodiments, the MUC16 polypeptide is N-glycosylated at amino acid residues Asn1, Asn24, and Asn30 of SEQ ID NO: 3 (also referred to as Asn1777, Asn1800, and Asn1806, respectively, in Yin and Lloyd (2001) J Biol Chem 276: 27371-27375). In certain embodiments, the glycosylation comprises N-linked chitobiose. In certain embodiments, the glycosylation consists of an N-linked chitobiose. In certain embodiments, Matrigel invasion is inhibited by at least 1.25, 1.5, 1.75, 2, 3, 4, 5, 6, 7, 8, 9, or 10-fold as compared to Matrigel invasion in vitro of the cells wherein the cells are treated with a control antibody (e.g., an antibody that does not target MUC16). In certain embodiments, Matrigel invasion is inhibited by about 1.25, 1.5, 1.75, 2, 3, 4, 5, 6, 7, 8, 9, or 10-fold as compared to Matrigel invasion in vitro of the cells wherein the cells are treated with a control antibody (e.g., an antibody that does not target MUC16).

Assays to determine the anti-MUC16 immunoglobulin-related composition-mediated inhibition of Matrigel invasion are known to a person skilled in the art. For example, BD BioCoat™ Matrigel™ Invasion Inserts or Chambers (catalog #354480 in 24 well plate) and Control Inserts (catalog #354578 in 24 well plate) can be purchased from BD Biosciences, MA. Matrigel Invasion assay can be performed as per manufacturer's protocol. Briefly, the Matrigel chambers in 24 well plates (stored at −20° C.) and control inserts (stored at 4° C.) are allowed to come to room temperature. Both inserts are rehydrated with 0.5 mL of serum free medium in the insert as well as in the outside well of the 24 well plate, for 2 hours at 37° C. 5% CO₂ humidified incubator. Cultured SKOV3 cells are trypsinized and washed with culture medium. A million cells are separated into another centrifuge tube and washed 3 times with serum free medium. These cells are later adjusted to give 5,000 cells in 0.5 mL serum free medium. The medium in the rehydrated inserts are removed and the insert was transferred into a new 24 well plate containing 0.75 mL of 10% Fetal Bovine Serum (FBS) containing culture medium in the well which serves as a chemo attractant. Immediately, 0.5 mL of the cells (5,000 cells) in serum free medium is added to the insert. Proper care is taken to see that there is no air bubble is trapped in the insert and the outside well. The 24 well plate is incubated at 37° C. 5% CO₂ humidified incubator for 48 hrs. After incubation, the non-invading cells are removed from the upper surface of the membrane by “scrubbing” by inserting a cotton tipped swab into Matrigel or control insert and gently applied pressure while moving the tip of the swab over the membrane surface. The scrubbing is repeated with a second swab moistened with medium. Then the inserts are stained in a new 24 well plate containing 0.5 mL of 0.5% crystal violet stain in distilled water for 30 minutes. Following staining the inserts are rinsed in 3 beakers of distilled water to remove excess stain. The inserts are air dried for in a new 24 well plate. The invaded cells are hand counted under an inverted microscope at 200× magnification. Several fields of triplicate membranes were counted and recorded in the figure.

In certain embodiments, an anti-MUC16 immunoglobulin-related composition described herein is capable of inhibiting or reducing metastasis, inhibiting tumor growth or inducing tumor regression in mouse model studies. For example, tumor cell lines can be introduced into athymic nude mice, and the athymic mice can be administered an anti-MUC16 immunoglobulin-related composition described herein one or more times, and tumor progression of the injected tumor cells can be monitored over a period of weeks and/or months. In some cases, administration of an anti-MUC16 immunoglobulin-related composition to the athymic nude mice can occur prior to introduction of the tumor cell lines. In a certain embodiment, SKOV3 cells expressing MUC16 c114 are utilized for the mouse xenograft models described herein.

In some embodiments, an anti-MUC16 immunoglobulin-related composition described herein inhibits tumor growth or induce tumor regression in a mouse model by at least about 5%, 10%, 15%, 20%, 25%, 30%, 35%, 40%, 45%, 50%, 55%, 60%, 65%, 70%, 75%, 80%, 85%, 90%, 95%, 98%, 99%, or 100% as assessed by methods described herein or known to one of skill in the art, as compared to mock-treated mice. In some embodiments, an anti-MUC16 immunoglobulin-related composition described herein inhibits tumor growth or induce tumor regression in a mouse model by at least about 25% or 35%, optionally to about 75%, as assessed by methods described herein or known to one of skill in the art, as compared to mock-treated mice. In some embodiments, an anti-MUC16 immunoglobulin-related composition described herein inhibits tumor growth or induce tumor regression in a mouse model by at least about 1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold as assessed by methods described herein or known to one of skill in the art, as compared to mock-treated mice. Mock-treated mice can, for example, be treated with phosphate buffered saline or a control (e.g., anti-IgG antibody).

Determining tumor growth inhibition or tumor regression can be assessed, for example, by monitoring tumor size over a period of time, such as by physical measurement of palpable tumors, or other visual detection methods. For example, tumor cell lines can be generated to recombinantly express a visualization agent, such as green fluorescent protein (GFP) or luciferase, then in vivo visualization of GFP can be carried out by microscopy, and in vivo visualization of luciferase can be carried out by administering luciferase substrate to the xenograft mice and detecting luminescent due to the luciferase enzyme processing the luciferase substrate. The degree or level of detection of GFP or luciferase correlates to the size of the tumor in the xenograft mice.

In certain embodiments, an anti-MUC16 immunoglobulin-related composition described herein can increase survival of animals in tumor xenograft models as compared to mock-treated mice. In some embodiments, an anti-MUC16 immunoglobulin-related composition described herein increases survival of mice in tumor xenograft models by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art, as compared to mock-treated mice. In some embodiments, an anti-MUC16 immunoglobulin-related composition described herein increases survival of mice in tumor xenograft models by at least about 25% or 35%, optionally to about 75%, as assessed by methods described herein or known to one of skill in the art, as compared to mock-treated mice in tumor xenograft models. In some embodiments, an anti-MUC16 immunoglobulin-related composition described herein increases survival of mice in tumor xenograft models by at least about 1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold as assessed by methods described herein or known to one of skill in the art, as compared to mock-treated mice in tumor xenograft models. Survival can, for example, be determined by plotting a survival curve of number of surviving mice against time (e.g., days or weeks) after tumor cell line injection. Mock-treated mice can, for example, be treated with phosphate buffered saline or a control (e.g., anti-IgG antibody).

In certain embodiments, an anti-MUC16 immunoglobulin-related composition described herein is internalized into a cell expressing a MUC16 polypeptide upon contacting the cell with the anti-MUC16 immunoglobulin-related composition. “Internalized” or “internalization,” when in reference to a molecule that is internalized by a cell, refers to passage of the molecule that is in contact with the extracellular surface of a cell membrane across the cell membrane to the intracellular surface of the cell membrane and/or into the cell cytoplasm. In certain embodiments, the cells recombinantly expressing glycosylated MUC16 c114 are SKOV3 cells. In certain embodiments, the glycosylated form of MUC16 c114 is N-glycosylated, e.g., at Asn1, Asn24, and Asn30 of SEQ ID NO: 3 (also referred to as Asn1777, Asn1800, and Asn1806, respectively, in Yin and Lloyd (2001) J Biol Chem 276: 27371-27375). In certain embodiments, the glycosylation comprises N-linked chitobiose. In certain embodiments, the glycosylation consists of an N-linked chitobiose.

Assays to determine internalization of an anti-MUC16 immunoglobulin-related composition described herein to a cell, such as, for example, using radiolabeled antibodies, are known to a person skilled in the art. For example, internalization of ⁸⁹Zr-labeled antibody can be investigated on SKOV3 cells expressing MUC16 c114. Briefly, approximately 1×10⁵ cells are seeded in a 12-well plate and incubated overnight at 37° C. 5% CO₂ incubator. A volume of radiolabeled protein is added to each well and the plates are incubated at 37° C. and 4° C. for 1, 5, 12, and 24 hours. Following each incubation period, the medium is collected and the cells are rinsed with 1 mL of phosphate buffered saline (PBS). Surface-bound activity is collected by washing the cells in 1 mL of 100 mM acetic acid with 100 mM glycine (1:1, pH 3.5) at 4° C. The adherent cells are then lysed with 1 mL of 1 M NaOH. Each wash is collected and counted for activity. The ratio of activity of the final wash to the total activity of all the washes is used to determine the % internalized. In certain embodiments, the assay is performed at 37° C. In certain embodiments, the anti-MUC16 immunoglobulin-related composition is internalized in at least 1, 2, 3, 5, 6, 7, 8, 9, or 10 percent of cells incubated with the anti-MUC16 immunoglobulin-related composition. In certain embodiments, the anti-MUC16 immunoglobulin-related composition is internalized in about 1, 2, 3, 5, 6, 7, 8, 9, or 10 percent of cells incubated with the anti-MUC16 immunoglobulin-related composition. In certain embodiments, the anti-MUC16 immunoglobulin-related composition is internalized within 1, 2, 3, 4, 8, 12, 16, 20, or 24 hours of contacting the cells with the anti-MUC16 immunoglobulin-related composition.

Nucleic Acids. Nucleic acid molecules encoding the anti-MUC16 immunoglobulin-related compositions are also contemplated. In some embodiments, the nucleic acid (or a set of nucleic acids) encoding the anti-MUC16 immunoglobulin-related composition described herein may further comprises a nucleic acid sequence encoding a peptide tag (such as protein purification tag, e.g., His-tag, HA tag).

Also contemplated here are isolated host cells comprising an anti-MUC16 immunoglobulin-related composition, an isolated nucleic acid encoding the polypeptide components of the anti-MUC16 immunoglobulin-related composition, or a vector comprising a nucleic acid encoding the polypeptide components of the anti-MUC16 immunoglobulin-related composition described herein.

The present application also includes variants to these nucleic acid sequences. For example, the variants include nucleotide sequences that hybridize to the nucleic acid sequences encoding the anti-MUC16 immunoglobulin-related compositions (such as anti-MUC16 antibodies, e.g., full-length anti-MUC16 antibodies, antigen-binding fragments thereof, or anti-MUC16 antibody moieties of the present application under at least moderately stringent hybridization conditions).

The present technology also provides vectors in which a nucleic acid of the present technology is inserted.

In brief summary, the expression of an anti-MUC16 immunoglobulin-related composition by a natural or synthetic nucleic acid encoding the anti-MUC16 immunoglobulin-related composition can be achieved by inserting the nucleic acid into an appropriate expression vector, such that the nucleic acid is operably linked to 5′ and 3′ regulatory elements, including for example a promoter (e.g., a lymphocyte-specific promoter) and a 3′ untranslated region (UTR). The vectors can be suitable for replication and integration in eukaryotic host cells. Typical cloning and expression vectors contain transcription and translation terminators, initiation sequences, and promoters useful for regulation of the expression of the desired nucleic acid sequence.

The nucleic acids of the present disclosure may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In some embodiments, the present technology provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Green and Sambrook (2013, Molecular Cloning: A Laboratory Manual, Cold Spring Harbor Laboratory, New York), and in other virology and molecular biology manuals. Viruses which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers (see, e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In some embodiments, lentivirus vectors are used. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the present technology should not be limited to the use of constitutive promoters. Inducible promoters are also contemplated as part of the present technology. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In some embodiments, the expression of the anti-MUC16 immunoglobulin-related composition is inducible. In some embodiments, a nucleic acid sequence encoding the anti-MUC16 immunoglobulin-related composition is operably linked to an inducible promoter, including any inducible promoter described herein.

Inducible Promoters

The use of an inducible promoter provides a molecular switch capable of turning on expression of the polynucleotide sequence which it is operatively linked when such expression is desired, or turning off the expression when expression is not desired. Exemplary inducible promoter systems for use in eukaryotic cells include, but are not limited to, hormone-regulated elements (e.g., see Mader, S. and White, J. H. Proc. Natl. Acad. Sci. USA 90:5603-5607 (1993)), synthetic ligand-regulated elements (see, e.g., Spencer, D. M. et al 1993) Science 262: 1019-1024) and ionizing radiation-regulated elements (e.g., see Manome, Y. et al., Biochemistry 32: 10607-10613 (1993); Datta, R. et al., Proc. Natl. Acad. Sci. USA 89: 1014-10153 (1992)). Further exemplary inducible promoter systems for use in in vitro or in vivo mammalian systems are reviewed in Gingrich et al., Annual Rev. Neurosci 21:377-405 (1998). In some embodiments, the inducible promoter system for use to express the anti-MUC16 immunoglobulin-related composition is the Tet system. In some embodiments, the inducible promoter system for use to express the anti-MUC16 immunoglobulin-related composition is the lac repressor system from E. coli.

An exemplary inducible promoter system for use in the present technology is the Tet system. Such systems are based on the Tet system described by Gossen et al., (1993). In an exemplary embodiment, a polynucleotide of interest is under the control of a promoter that comprises one or more Tet operator (TetO) sites. In the inactive state, Tet repressor (TetR) will bind to the TetO sites and repress transcription from the promoter. In the active state, e.g., in the presence of an inducing agent such as tetracycline (Tc), anhydrotetracycline, doxycycline (Dox), or an active analog thereof, the inducing agent causes release of TetR from TetO, thereby allowing transcription to take place. Doxycycline is a member of the tetracycline family of antibiotics having the chemical name of 1-dimethylamino-2,4a,5,7,12-pentahydroxy-11-methyl-4,6-dioxo-1,4a,11,11a,12,12a-hexahydrotetracene-3-carboxamide.

In one embodiment, a TetR is codon-optimized for expression in mammalian cells, e.g., murine or human cells. Most amino acids are encoded by more than one codon due to the degeneracy of the genetic code, allowing for substantial variations in the nucleotide sequence of a given nucleic acid without any alteration in the amino acid sequence encoded by the nucleic acid. However, many organisms display differences in codon usage, also known as “codon bias” (i.e., bias for use of a particular codon(s) for a given amino acid). Codon bias often correlates with the presence of a predominant species of tRNA for a particular codon, which in turn increases efficiency of mRNA translation. Accordingly, a coding sequence derived from a particular organism (e.g., a prokaryote) may be tailored for improved expression in a different organism (e.g., a eukaryote) through codon optimization.

Other specific variations of the Tet system include the following “Tet-Off” and “Tet-On” systems. In the Tet-Off system, transcription is inactive in the presence of Tc or Dox. In that system, a tetracycline-controlled transactivator protein (tTA), which is composed of TetR fused to the strong transactivating domain of VP16 from Herpes simplex virus, regulates expression of a target nucleic acid that is under transcriptional control of a tetracycline-responsive promoter element (TRE). The TRE is made up of TetO sequence concatamers fused to a promoter (commonly the minimal promoter sequence derived from the human cytomegalovirus (hCMV) immediate-early promoter). In the absence of Tc or Dox, tTA binds to the TRE and activates transcription of the target gene. In the presence of Tc or Dox, tTA cannot bind to the TRE, and expression from the target gene remains inactive.

Conversely, in the Tet-On system, transcription is active in the presence of Tc or Dox. The Tet-On system is based on a reverse tetracycline-controlled transactivator, rtTA. Like tTA, rtTA is a fusion protein comprised of the TetR repressor and the VP16 transactivation domain. However, a four amino acid change in the TetR DNA binding moiety alters rtTA's binding characteristics such that it can only recognize the tetO sequences in the TRE of the target transgene in the presence of Dox. Thus, in the Tet-On system, transcription of the TRE-regulated target gene is stimulated by rtTA only in the presence of Dox.

Another inducible promoter system is the lac repressor system from E. coli (See Brown et al., Cell 49:603-612 (1987)). The lac repressor system functions by regulating transcription of a polynucleotide of interest operably linked to a promoter comprising the lac operator (lacO). The lac repressor (lacR) binds to LacO, thus preventing transcription of the polynucleotide of interest. Expression of the polynucleotide of interest is induced by a suitable inducing agent, e.g., isopropyl-β-D-thiogalactopyranoside (IPTG).

In order to assess the expression of a polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, β-galactosidase, chloramphenicol acetyl transferase, secreted alkaline phosphatase, or the green fluorescent protein gene (e.g., Ui-Tel et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some embodiments, there is provided nucleic acid encoding any anti-MUC16 immunoglobulin-related compositions described herein. In some embodiments, the nucleic acid comprises one or more nucleic acid sequences encoding the heavy and light chains of the anti-MUC16 immunoglobulin-related compositions described herein. In some embodiments, each of the one or more nucleic acid sequences are contained in separate vectors. In some embodiments, at least some of the nucleic acid sequences are contained in the same vector. In some embodiments, all of the nucleic acid sequences are contained in the same vector. Vectors may be selected, for example, from the group consisting of mammalian expression vectors and viral vectors (such as those derived from retroviruses, adenoviruses, adeno-associated viruses, herpes viruses, and lentiviruses).

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Green and Sambrook (2013, Molecular Cloning: A Laboratory Manual, Cold Spring Harbor Laboratory, New York). In some embodiments, the introduction of a polynucleotide into a host cell is carried out by calcium phosphate transfection.

Biological methods for introducing a polynucleotide of interest into a host cell include the use of DNA and RNA vectors. Viral vectors, and especially retroviral vectors, have become the most widely used method of inserting genes into mammalian, e.g., human cells. Other viral vectors can be derived from lentivirus, poxviruses, herpes simplex virus 1, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Regardless of the method used to introduce exogenous nucleic acids into a host cell or otherwise expose a cell to the inhibitor of the present technology, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the present technology.

Preparation of anti-MUC16 immunoglobulin-related compositions and anti-MUC16 antibody moieties. In some embodiments, the anti-MUC16 immunoglobulin-related composition is a monoclonal antibody or derived from a monoclonal antibody. In some embodiments, the anti-MUC16 immunoglobulin-related composition comprises V_{H} and V_{L} domains, or variants thereof, from the monoclonal antibody. In some embodiments, the anti-MUC16 immunoglobulin-related composition further comprises CH₁ and CL domains, or variants thereof, from the monoclonal antibody. Monoclonal antibodies can be prepared, e.g., using known methods in the art, including hybridoma methods, phage display methods, or using recombinant DNA methods. Additionally, exemplary phage display methods are described herein.

In a hybridoma method, a hamster, mouse, or other appropriate host animal is typically immunized with an immunizing agent to elicit lymphocytes that produce or are capable of producing antibodies that will specifically bind to the immunizing agent. Alternatively, the lymphocytes can be immunized in vitro. The immunizing agent can include a polypeptide or a fusion protein of the protein of interest. Generally, peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. Immortalized cell lines are usually transformed mammalian cells, particularly myeloma cells of rodent, bovine, and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which prevents the growth of HGPRT-deficient cells.

In some embodiments, the immortalized cell lines fuse efficiently, support stable high-level expression of antibody by the selected antibody-producing cells, and are sensitive to a medium such as HAT medium. In some embodiments, the immortalized cell lines are murine myeloma lines, which can be obtained, for instance, from the Salk Institute Cell Distribution Center, San Diego, California and the American Type Culture Collection, Manassas, Virginia. Human myeloma and mouse-human heteromyeloma cell lines also have been described for the production of human monoclonal antibodies.

The culture medium in which the hybridoma cells are cultured can then be assayed for the presence of monoclonal antibodies directed against the polypeptide. The binding specificity of monoclonal antibodies produced by the hybridoma cells can be determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones can be sub cloned by limiting dilution procedures and grown by standard methods. Goding, supra. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the sub clones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

In some embodiments, according to any of the anti-MUC16 immunoglobulin-related compositions described herein, the anti-MUC16 immunoglobulin-related composition comprises sequences from a clone selected from an antibody library (such as a phage library presenting scFv or Fab fragments). The clone may be identified by screening combinatorial libraries for antibody fragments with the desired activity or activities. For example, a variety of methods are known in the art for generating phage display libraries and screening such libraries for antibodies possessing the desired binding characteristics. Such methods are reviewed, e.g., in Hoogenboom et al., Methods in Molecular Biology 178:1-37 (O'Brien et al., ed., Human Press, Totowa, N.J., 2001) and further described, e.g., in McCafferty et al., Nature 348:552-554; Clackson et al., Nature 352: 624-628 (1991); Marks et al., J. Mol. Biol. 222: 581-597 (1992); Marks and Bradbury, Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of V_{H} and V_{L} genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as scFv fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

The anti-MUC16 immunoglobulin-related compositions can be prepared using phage display to screen libraries for anti-MUC16 antibody moieties specific to the target MUC16 (e.g., MUC16^{c114}). The library can be a human scFv phage display library having a diversity of at least 1×10⁹ (such as at least about any of 1×10⁹, 2.5×10⁹, 5×10⁹, 7.5×10⁹, 1×10¹⁰, 2.5×10¹⁰, 5×10¹⁰, 7.5×10¹⁰, or 1×10¹¹) unique human antibody fragments. In some embodiments, the library is a naïve human library constructed from DNA extracted from human PMBCs and spleens from healthy donors, encompassing all human heavy and light chain subfamilies. In some embodiments, the library is a naïve human library constructed from DNA extracted from PBMCs isolated from patients with various diseases, such as patients with autoimmune diseases, cancer patients, and patients with infectious diseases. In some embodiments, the library is a semi-synthetic human library, wherein heavy chain CDR3 is completely randomized, with all amino acids (with the exception of cysteine) equally likely to be present at any given position (see, e.g., Hoet, R. M. et al., Nat. Biotechnol. 23(3):344-348, 2005). In some embodiments, the heavy chain CDR3 of the semi-synthetic human library has a length from about 5 to about 24 (such as about any of 5, 6, 7, 8, 9, 10, 11, 12, 13, 14, 15, 16, 17, 18, 19, 20, 21, 22, 23, or 24) amino acids. In some embodiments, the library is a fully-synthetic phage display library. In some embodiments, the library is a non-human phage display library.

Phage clones that bind to the target MUC16 (e.g., MUC16^{c114}) with high affinity can be selected by iterative binding of phage to the target MUC16, which is bound to a solid support (such as, for example, beads for solution panning or mammalian cells for cell panning), followed by removal of non-bound phage and by elution of specifically bound phage. The bound phage clones are then eluted and used to infect an appropriate host cell, such as E. coli XL1-Blue, for expression and purification. In an example of cell panning, HEK293 cells over-expressing MUC16 on cell surface are mixed with the phage library, after which the cells are collected and the bound clones are eluted and used to infect an appropriate host cell for expression and purification. The panning can be performed for multiple (such as about any of 2, 3, 4, 5, 6 or more) rounds with solution panning, cell panning, or a combination of both, to enrich for phage clones binding specifically to the target MUC16. Enriched phage clones can be tested for specific binding to the target MUC16 by any methods known in the art, including for example ELISA and FACS.

Monoclonal antibodies can also be made by recombinant DNA methods, such as those described in U.S. Pat. No. 4,816,567. DNA encoding the monoclonal antibodies of the present technology can be readily isolated and sequenced using conventional procedures (e.g., by using oligonucleotide probes that are capable of binding specifically to genes encoding the heavy and light chains of murine antibodies). Hybridoma cells as described above or MUC16-specific phage clones of the present technology can serve as a source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy- and light-chain constant domains and/or framework regions in place of the homologous non-human sequences (U.S. Pat. No. 4,816,567; Morrison et al., supra) or by covalently joining to the immunoglobulin coding sequence all or part of the coding sequence for a non-immunoglobulin polypeptide. Such a non-immunoglobulin polypeptide can be substituted for the constant domains of an immunoglobulin-related composition of the present technology, or can be substituted for the variable domains of one antigen-combining site of an immunoglobulin-related composition of the present technology to create a chimeric bivalent immunoglobulin-related composition.

The antibodies can be monovalent antibodies. Methods for preparing monovalent antibodies are known in the art. For example, one method involves recombinant expression of immunoglobulin light chain and modified heavy chain. The heavy chain is truncated generally at any point in the Fc region so as to prevent heavy-chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly Fab fragments, can be accomplished using any method known in the art.

Antibody variable domains with the desired binding specificities (antibody-antigen combining sites) can be fused to immunoglobulin constant-domain sequences. The fusion preferably is with an immunoglobulin heavy-chain constant domain, comprising at least part of the hinge, CH2, and CH3 regions. In some embodiments, the first heavy-chain constant region (CH1) containing the site necessary for light-chain binding is present in at least one of the fusions. DNAs encoding the immunoglobulin heavy-chain fusions and, if desired, the immunoglobulin light chain, are inserted into separate expression vectors, and are co-transfected into a suitable host organism.

Fc Region Variants. In some embodiments, one or more amino acid modifications may be introduced into the Fc region of an anti-MUC16 immunoglobulin-related composition provided herein, thereby generating an Fc region variant. In some embodiments, the Fc region variant has enhanced ADCC effector function, often related to binding to Fc receptors (FcRs). In some embodiments, the Fc region variant has decreased ADCC effector function. There are many examples of changes or mutations to Fc sequences that can alter effector function. For example, WO 00/42072 and Shields et al., J Biol. Chem. 9(2): 6591-6604 (2001) describe antibody variants with improved or diminished binding to FcRs. The contents of those publications are specifically incorporated herein by reference.

Antibody-Dependent Cell-Mediated Cytotoxicity (ADCC) is a mechanism of action of therapeutic antibodies against tumor cells. ADCC is a cell-mediated immune defense whereby an effector cell of the immune system actively lyses a target cell (e.g., a cancer cell), whose membrane-surface antigens have been bound by specific antibodies (e.g., an anti-MUC16 antibody). The typical ADCC involves activation of NK cells by antibodies. An NK cell expresses CD16 which is an Fc receptor. This receptor recognizes, and binds to, the Fc portion of an antibody bound to the surface of a target cell. The most common Fc receptor on the surface of an NK cell is called CD16 or FcγRIII. Binding of the Fc receptor to the Fc region of an antibody results in NK cell activation, release of cytolytic granules and consequent target cell apoptosis. The contribution of ADCC to tumor cell killing can be measured with a specific test that uses NK-92 cells that have been transfected with a high-affinity FcR. Results are compared to wild-type NK-92 cells that do not express the FcR.

In some embodiments, the present technology contemplates an anti-MUC16 immunoglobulin-related composition variant comprising an Fc region that possesses some but not all effector functions, which makes it a desirable candidate for applications in which the half-life of the anti-MUC16 immunoglobulin-related composition in vivo is important yet certain effector functions (such as CDC and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the immunoglobulin-related composition lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al., Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assay methods may be employed (see, for example, ACTI™ non-radioactive cytotoxicity assay for flow cytometry (CellTechnology, Inc. Mountain View, Calif; and CytoTox 96™ non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al., Proc. Nat'l Acad. Sci. USA 95:652-656 (1998). C1q binding assays may also be carried out to confirm that the immunoglobulin-related composition is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain immunoglobulin-related composition variants with improved or diminished binding to FcRs are described. (See, e.g., U.S. Pat. No. 6,737,056; WO 2004/056312, and Shields et al., J. Biol. Chem. 9(2): 6591-6604 (2001).)

In some embodiments, there is provided an anti-MUC16 immunoglobulin-related composition variant comprising a variant Fc region comprising one or more amino acid substitutions which improve ADCC. In some embodiments, the variant Fc region comprises one or more amino acid substitutions which improve ADCC, wherein the substitutions are at positions 298, 333, and/or 334 of the variant Fc region (EU numbering of residues). In some embodiments, the anti-MUC16 immunoglobulin-related composition variant comprises the following amino acid substitution in its variant Fc region: S298A, E333A, and K334A.

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al., J. Immunol. 164: 4178-4184 (2000).

In some embodiments, there is provided an anti-MUC16 immunoglobulin-related composition variant comprising a variant Fc region comprising one or more amino acid substitutions which increase half-life and/or improve binding to the neonatal Fc receptor (FcRn). Antibodies with increased half-lives and improved binding to FcRn are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40 (1988); U.S. Pat. Nos. 5,648,260; 5,624,821; and WO 94/29351 concerning other examples of Fc region variants.

Anti-MUC16 immunoglobulin-related compositions (such as full-length anti-MUC16 antibodies) comprising any of the Fc variants described herein, or combinations thereof, are contemplated.

Glycosylation Variants. In some embodiments, an anti-MUC16 immunoglobulin-related composition provided herein is altered to increase or decrease the extent to which the anti-MUC16 immunoglobulin-related composition is glycosylated. Addition or deletion of glycosylation sites to an anti-MUC16 immunoglobulin-related composition may be conveniently accomplished by altering the amino acid sequence of the anti-MUC16 immunoglobulin-related composition or polypeptide portion thereof such that one or more glycosylation sites is created or removed.

Where the anti-MUC16 immunoglobulin-related composition comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al., TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an anti-MUC16 immunoglobulin-related composition of the present technology may be made in order to create anti-MUC16 immunoglobulin-related composition variants with certain improved properties.

The N-glycans attached to the CH2 domain of Fc is heterogeneous. Antibodies or Fc fusion proteins generated in CHO cells are fucosylated by fucosyltransferase activity. See Shoji-Hosaka et al., J. Biochem. 140:777-83 (2006). Normally, a small percentage of naturally occurring afucosylated IgGs may be detected in human serum. N-glycosylation of the Fc is important for binding to FcγR; and afucosylation of the N-glycan increases Fc's binding capacity to FcγRIIIa. Increased FcγRIIIa binding can enhance ADCC, which can be advantageous in certain immunoglobulin-related composition therapeutic applications in which cytotoxicity is desirable.

In some embodiments, an enhanced effector function can be detrimental when Fc-mediated cytotoxicity is undesirable. In some embodiments, the Fc fragment or CH2 domain is not glycosylated. In some embodiments, the N-glycosylation site in the CH2 domain is mutated to prevent from glycosylation.

In some embodiments, anti-MUC16 immunoglobulin-related composition variants are provided comprising an Fc region wherein a carbohydrate structure attached to the Fc region has reduced fucose or lacks fucose, which may improve ADCC function. Specifically, anti-MUC16 immunoglobulin-related compositions are contemplated herein that have reduced fucose relative to the amount of fucose on the same anti-MUC16 immunoglobulin-related composition produced in a wild-type CHO cell. That is, they are characterized by having a lower amount of fucose than they would otherwise have if produced by native CHO cells (e.g., a CHO cell that produce a native glycosylation pattern, such as, a CHO cell containing a native FUT8 gene). In some embodiments, the anti-MUC16 immunoglobulin-related composition is one wherein less than about 50%, 40%, 30%, 20%, 10%, or 5% of the N-linked glycans thereon comprise fucose. For example, the amount of fucose in such an anti-MUC16 immunoglobulin-related composition may be from 1% to 80%, from 1% to 65%, from 5% to 65% or from 20% to 40%. In some embodiments, the anti-MUC16 immunoglobulin-related composition is one wherein none of the N-linked glycans thereon comprise fucose, i.e., wherein the anti-MUC16 immunoglobulin-related composition is completely without fucose, or has no fucose or is afucosylated. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” immunoglobulin-related composition variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al., J. Mol. Biol. 336:1239-1249 (2004); Yamane-Ohnuki et al., Biotech. Bioeng. 87: 614 (2004). Examples of cell lines capable of producing defucosylated antibodies include Lec13 CHO cells deficient in protein fucosylation (Ripka et al., Arch. Biochem. Biophys. 249:533-545 (1986); US Pat Appl No US 2003/0157108 A1, Presta, L; and WO 2004/056312 A1, Adams et al., especially at Example 11), and knockout cell lines, such as α-1,6-fucosyltransferase gene, FUT8, knockout CHO cells (see, e.g., Yamane-Ohnuki et al., Biotech. Bioeng. 87: 614 (2004); Kanda, Y. et al., Biotechnol. Bioeng., 94(4):680-688 (2006); and WO2003/085107).

Anti-MUC16 immunoglobulin-related composition variants are further provided with bisected oligosaccharides, e.g., in which a biantennary oligosaccharide attached to the Fc region of the anti-MUC16 immunoglobulin-related composition is bisected by GlcNAc. Such anti-MUC16 immunoglobulin-related composition variants may have reduced fucosylation and/or improved ADCC function. Examples of such immunoglobulin-related composition variants are described, e.g., in WO 2003/011878 (Jean-Mairet et al.); U.S. Pat. No. 6,602,684 (Umana et al.); US 2005/0123546 (Umana et al.), and Ferrara et al., Biotechnology and Bioengineering, 93(5): 851-861 (2006). Anti-MUC16 immunoglobulin-related composition variants with at least one galactose residue in the oligosaccharide attached to the Fc region are also provided. Such anti-MUC16 immunoglobulin-related composition variants may have improved CDC function. Such immunoglobulin-related composition variants are described, e.g., in WO 1997/30087 (Patel et al.); WO 1998/58964 (Raju, S.); and WO 1999/22764 (Raju, S.).

In some embodiments, the anti-MUC16 immunoglobulin-related composition variants comprising an Fc region are capable of binding to an FcγRIII. In some embodiments, the anti-MUC16 immunoglobulin-related composition variants comprising an Fc region have ADCC activity in the presence of human effector cells (e.g., T cell) or have increased ADCC activity in the presence of human effector cells compared to the otherwise same anti-MUC16 immunoglobulin-related composition comprising a human wild-type IgG1Fc region.

Cysteine Engineered Variants. In some embodiments, it may be desirable to create cysteine engineered anti-MUC16 immunoglobulin-related compositions in which one or more amino acid residues are substituted with cysteine residues. In some embodiments, the substituted residues occur at accessible sites of the anti-MUC16 immunoglobulin-related composition. By substituting those residues with cysteine, reactive thiol groups are thereby positioned at accessible sites of the anti-MUC16 immunoglobulin-related composition and may be used to conjugate the anti-MUC16 immunoglobulin-related composition to other moieties, such as drug moieties or linker-drug moieties, to create an anti-MUC16 immunoconjugate, as described further herein. Cysteine engineered anti-MUC16 immunoglobulin-related compositions (such as anti-MUC16 antibodies or antigen binding fragments thereof) may be generated as described, e.g., in U.S. Pat. No. 7,521,541.

Derivatives. In some embodiments, an anti-MUC16 immunoglobulin-related composition provided herein may be further modified to contain additional non-proteinaceous moieties that are known in the art and readily available. The moieties suitable for derivatization of the anti-MUC16 immunoglobulin-related composition include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the anti-MUC16 immunoglobulin-related composition may vary, and if more than one polymer are attached, they can be the same or different molecules. In general, the number and/or type of polymers used for derivatization can be determined based on considerations including, but not limited to, the particular properties or functions of the anti-MUC16 immunoglobulin-related composition to be improved, whether the anti-MUC16 immunoglobulin-related composition derivative will be used in a therapy under defined conditions, etc.

In some embodiments, conjugates of an anti-MUC16 immunoglobulin-related composition and nonproteinaceous moiety that may be selectively heated by exposure to radiation are provided. In some embodiments, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the anti-MUC16 immunoglobulin-related composition-nonproteinaceous moiety are killed.

### VEGF Inhibitors

A central pathway in the network regulating the growth and differentiation of the vascular system and its components, both during embryonic development and normal growth, and in a wide number of pathological anomalies and diseases, is mediated by Vascular Endothelial Growth Factor (“VEGF”) and the cellular receptors of VEGF (“VEGFRs”). (See G. Breier et al., Trends in Cell Biology, 6:454-456 (1996)).

VEGF is a dimeric, disulfide-linked 46-kDa glycoprotein related to Platelet-Derived Growth Factor (“PDGF”). It is produced by normal cell lines and tumor cell lines; is an endothelial cell-selective mitogen; shows angiogenic activity in in vivo test systems (e.g., rabbit cornea); is chemotactic for endothelial cells and monocytes; and induces plasminogen activators in endothelial cells, which are involved in the proteolytic degradation of the extracellular matrix during the formation of capillaries. A number of isoforms of VEGF are known, which while they show comparable biological activity, differ in the type of cells that secrete them and in their heparin-binding capacity. In addition, there are other members of the VEGF family, such as Placenta Growth Factor (“PGF”) and VEGF-C. The cellular receptors of VEGFs (VEGFRs) are transmembranous receptor tyrosine kinases, which are characterized by an extracellular domain with seven immunoglobulin-like domains and an intracellular tyrosine kinase domain. Various types of VEGF receptor have been characterized, including VEGFR-1 (also known as fit-1), VEGFR-2 (also known as KDR), and VEGFR-3.

A large number of human tumors, especially gliomas and carcinomas, express high levels of VEGF and VEGFRs. This has led to the hypothesis that VEGF released by tumor cells stimulates the growth of blood capillaries and the proliferation of tumor endothelium in a paracrine manner and, through the improved blood supply, accelerates tumor growth. Increased VEGF expression could explain the occurrence of cerebral edema in patients with glioma. VEGFs contribute to vascular hyperpermeability and the formation of edema. Indeed, vascular hyperpermeability and edema that is associated with the expression or administration of many other growth factors appears to be mediated via VEGF production.

Inflammatory cytokines stimulate VEGF production. Hypoxia results in a marked upregulation of VEGF in numerous tissues. Thus, situations involving infarct, occlusion, ischemia, anemia, or circulatory impairment typically invoke VEGF/VPF-mediated responses. Vascular hyperpermeability, associated edema, altered transendothelial exchange and macromolecular extravasation, which is often accompanied by diapedesis, can result in excessive matrix deposition, aberrant stromal proliferation, fibrosis, etc. Hence, VEGF-mediated hyperpermeability can significantly contribute to disorders with these etiologic features. Accordingly, these regulators of angiogenesis have become an important therapeutic target. See Hicklin and Ellis, J. Clin Oncology, 23:1011-1027 (2005).

A great many VEGF inhibitors have been described in the literature that may be used in the various embodiments of the present disclosure. The VEGF inhibitor may be a small molecule inhibitor, an inhibitory nucleic acid (e.g., siRNA, antisense oligonucleotide, shRNA, sgRNA, ribozyme), or an antibody or antigen binding fragment thereof.

In addition to those described in further detail below, VEGF inhibitors that may be used in this regard are described in the following patent documents: US 2003/0105091, US2006/0241115, U.S. Pat. Nos. 5,521,184, 5,770,599, 5,990,141, 6,235,764, 6,258,812, 6,515,004, 6,630,500, 6,713,485, 5,792,825 and 6,025,688, WO2005/070891, WO 01/32651, WO 02/68406, WO 02/66470, WO 02/55501, WO 04/05279, WO 04/07481, WO 04/07458, WO 04/09784, WO 02/59110, WO 99/450029, WO 00/59509, WO 99/61422, WO 00/12089, WO 00/02871, and WO 01/37820, all of which are incorporated herein in their entirety particularly in parts pertinent to VEGF inhibitors useful in the present technology herein described.

Exemplary VEGF inhibitors include, but are not limited to, Linifanib (ABT-869, Abbott), AEE-788 (Novartis) (also called AE-788 and NVP-AEE-788), axitinib (AG-13736, (Pfizer) (also called AG-013736), AG-028262 (Pfizer), Angiostatin (EntreMed) (also called CAS Registry Number 86090-08-6, K1-4, and rhuAngiostatin, among others), Avastin™ (Genentech) (also called bevacizumab, R-435, rhuMAB-VEGF, and CAS Registry Number 216974-75-3), ranibizumab (Lucentis, Genentech), Vanucizumab, Brolucizumab, hPV19, IBI305, AVE-8062 (Ajinomoto Co. and Sanofi-aventis) (also called AC-7700 and combretastatin A4 analog, among others), Cediranib (AZD-2171, AstraZeneca), Nexavar® (Bayer AG and Onyx) (also called CAS Registry Number 284461-73-0, BAY-43-9006, raf kinase inhibitor, sorafenib, sorafenib analogs, and IDDBCP150446), BMS-387032 (Sunesis and Bristol-Myers Squibb) (also called SNS-032 and CAS Registry Number 345627-80-7), CEP-7055 (Cephalon and Sanofi-aventis) (also called CEP-11981 and SSR-106462), Dovitinib (CHIR-258, Chiron) (also called CAS Registry Number 405169-16-6, GFKI, and GFKI-258), CP-547632 (OSI Pharmaceuticals and Pfizer) (also called CAS Registry Number 252003-65-9), CP-564959, Lenvatinib (E-7080, Eisai Co.) (also called CAS Registry Number 417716-92-8 and ER-203492-00), pazopanib (GW786034, GlaxoSmithKline), GW-654652 (GlaxoSmithKline) and closely related indazolylpyrimidine Kdr inhibitors, IMC-1C11 (ImClone) (also called DC-101 and c-p1C11), Tivozanib (KRN-951, Kirin Brewery Co.) and other closely related quinoline-urea VEGF inhibitors, PKC-412 (Novartis) (also called CAS Registry Number 120685-11-2, benzoylstaurosporine, CGP-41251, midostaurin, and STI-412), PTK-787 (Novartis and Schering) (also called CAS Registry Numbers 212141-54-3 and 212142-18-2, PTK/ZK, PTK-787/ZK-222584, ZK-22584, VEGF-TKI, VEGF-RKI, PTK-787A, DE-00268, CGP-79787, CGP-79787D, vatalanib, ZK-222584) and closely related anilinophthalazine derivative VEGF inhibitors, SU11248 (Sugen and Pfizer) (also called SU-11248, SU-011248, SU-11248J, Sutent®, and sunitinib malate), SU-5416 (Sugen and Pfizer/Pharmacia) (also called CAS Registry Number 194413-58-6, semaxanib, 204005-46-9), Orantinib (SU-6668, Sugen and Taiho) (also called CAS Registry Number 252916-29-3, SU-006668, and TSU-68, among others) and closely related VEGF inhibitors as described in WO-09948868, WO-09961422, and WO-00038519 (which are hereby incorporated by reference in their entireties), VEGF Trap (Regeneron and Sanofi-aventis) (also called AVE-0005 and Systemic VEGF Trap, among others) and closely related VEGF inhibitors as described in WO-2004110490, which is hereby incorporated by reference in its entirety, Thalidomide (Celgene) (also called CAS Registry Number 50-35-1, Synovir, Thalidomide Pharmion, and Thalomid), Tesevatinib (XL-647, Exelixis) (also called EXEL-7647), XL-999 (Exelixis) (also called EXEL-0999), Foretinib (XL-880, Exelixis) (also called EXEL-2880), Vandetanib (ZD-6474, AstraZeneca) (also called CAS Registry Number 443913-73-3, Zactima, and AZD-6474) and closely related anilinoquinazoline VEGF inhibitors, and ZK-304709 (Schering) (also called ZK-CDK, MTGI) and other closely related compounds including the indirubin derivative VEGF inhibitors described in WO-00234717, WO-02074742, WO-02100401, WO-00244148, WO-02096888, WO-03029223, WO-02092079, and WO-02094814 which are hereby incorporated by reference in their entireties.

Other VEGF inhibitors useful in the methods of the present technology include: (a) a compound described in US2003/0125339 or U.S. Pat. No. 6,995,162 which is herein incorporated by reference in its entirety, particularly in parts disclosing VEGF inhibitors (e.g., 4TBPPAPC); (b) a substituted alkylamine derivative described in US2003/0125339 or US2003/0225106 or U.S. Pat. No. 6,995,162 or U.S. Pat. No. 6,878,714 each of which is herein incorporated by reference in its entirety, particularly in parts disclosing VEGF inhibitors (e.g., AMG 706); and (c) VEGF inhibitors as described in US2006/0241115, including those of Formula IV therein.

Formulations Including the Anti-MUC16^{c114}×CD3 Multispecific Immunoglobulin-Related Compositions and/or the VEGF Inhibitors of the Present Technology

The pharmaceutical compositions of the present technology can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, tablets, capsules, syrup, suppositories, injections, emulsions, elixirs, suspensions or solutions. Formulations may optionally contain solvents, diluents, and other liquid vehicles, dispersion or suspension aids, surface active agents, pH modifiers, isotonic agents, thickening or emulsifying agents, stabilizers and preservatives, solid binders, lubricants and the like, as suited to the particular dosage form desired. In certain embodiments, the compositions disclosed herein are formulated for administration to a mammal, such as a human.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, cyclodextrins, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. Compositions formulated for parenteral administration may be injected by bolus injection or by timed push, or may be administered by continuous infusion.

In order to prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents such as phosphates or carbonates.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. They may optionally contain opacifying agents and can also be of a composition that they release the active ingredient(s) only, or in a certain part of the intestinal tract, optionally, in a delayed manner. Examples of embedding compositions that can be used include polymeric substances and waxes.

The active compounds can also be in micro-encapsulated form with one or more excipients as noted above. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or in a certain part of the intestinal tract, optionally, in a delayed manner. Examples of embedding compositions that can be used include polymeric substances and waxes.

### Modes of Administration and Effective Dosages

Any method known to those in the art for contacting a cell, organ or tissue with an anti-MUC16^{c114}×CD3 multispecific (e.g., bispecific) immunoglobulin-related composition and/or a VEGF inhibitor may be employed. Suitable methods include in vitro, ex vivo, or in vivo methods. In vivo methods typically include the administration of an anti-MUC16^{c114}×CD3 multispecific (e.g., bispecific) immunoglobulin-related composition and/or a VEGF inhibitor, such as those described herein, to a mammal, suitably a human. When used in vivo for therapy, the anti-MUC16^{c114}×CD3 multispecific (e.g., bispecific) immunoglobulin-related compositions and/or VEGF inhibitors are administered to the subject in effective amounts (i.e., amounts that have desired therapeutic effect). The dose and dosage regimen will depend upon the degree of the disease symptoms in the subject, the characteristics of the particular anti-MUC16^{c114}×CD3 multispecific (e.g., bispecific) immunoglobulin-related composition and/or VEGF inhibitor, e.g., its therapeutic index, the subject, and the subject's history.

The effective amount may be determined during pre-clinical trials and clinical trials by methods familiar to physicians and clinicians. An effective amount of an anti-MUC16^{c114}×CD3 multispecific (e.g., bispecific) immunoglobulin-related composition and/or a VEGF inhibitor useful in the methods may be administered to a mammal in need thereof by any of a number of well-known methods for administering pharmaceutical compounds. The anti-MUC16^{c114}×CD3 multispecific (e.g., bispecific) immunoglobulin-related composition and/or VEGF inhibitor may be administered systemically or locally.

The anti-MUC16^{c114}×CD3 multispecific (e.g., bispecific) immunoglobulin-related composition and/or VEGF inhibitor can be incorporated into pharmaceutical compositions for administration, singly or in combination, to a subject for the treatment or prevention of a disorder described herein. Such compositions typically include the active agent and a pharmaceutically acceptable carrier. As used herein, the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided in a kit containing all necessary equipment (e.g., vials of drug, vials of diluent, syringes and needles) for a treatment course (e.g., 7 days of treatment).

In some embodiments, the anti-MUC16^{c114}×CD3 multispecific (e.g., bispecific) immunoglobulin-related composition and/or VEGF inhibitor described herein is administered by a parenteral route or a topical route.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

The anti-MUC16^{c114}×CD3 multispecific (e.g., bispecific) immunoglobulin-related compositions and/or VEGF inhibitors described herein can include a carrier, which can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, isotonic agents are included, for example, sugars, polyalcohols such as mannitol, sorbitol, or sodium chloride in the composition. Prolonged absorption of the injectable compositions can be brought about by including in the composition an agent which delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, compositions including the anti-MUC16^{c114}×CD3 multispecific (e.g., bispecific) immunoglobulin-related compositions and/or VEGF inhibitors of the present technology can be delivered in the form of an aerosol spray from a pressurized container or dispenser which contains a suitable propellant, e.g., a gas such as carbon dioxide, or a nebulizer. Such methods include those described in U.S. Pat. No. 6,468,798.

Systemic administration of an anti-MUC16^{c114}×CD3 multispecific (e.g., bispecific) immunoglobulin-related composition and/or a VEGF inhibitor of the present technology as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

An anti-MUC16^{c114}×CD3 multispecific (e.g., bispecific) immunoglobulin-related composition and/or a VEGF inhibitor of the present technology can be formulated in a carrier system. The carrier can be a colloidal system. The colloidal system can be a liposome, a phospholipid bilayer vehicle. In one embodiment, the therapeutic anti-MUC16^{c114}×CD3 multispecific (e.g., bispecific) immunoglobulin-related composition and/or VEGF inhibitor is encapsulated in a liposome while maintaining structural integrity. As one skilled in the art would appreciate, there are a variety of methods to prepare liposomes. (See Lichtenberg et al., Methods Biochem. Anal., 33:337-462 (1988); Anselem et al., Liposome Technology, CRC Press (1993)). Liposomal formulations can delay clearance and increase cellular uptake (See Reddy, Ann. Pharmacother., 34(7-8):915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the anti-MUC16^{c114}×CD3 multispecific (e.g., bispecific) immunoglobulin-related composition and/or VEGF inhibitor can be embedded in the polymer matrix, while maintaining protein integrity. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy et al.), U.S. Pat. Nos. 5,674,534 and 5,716,644 (both to Zale et al.), PCT publication WO 96/40073 (Zale et al.), and PCT publication WO 00/38651 (Shah et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the anti-MUC16^{c114}×CD3 multispecific (e.g., bispecific) immunoglobulin-related compositions and/or VEGF inhibitors are prepared with carriers that will protect the anti-MUC16^{c114}×CD3 multispecific (e.g., bispecific) immunoglobulin-related compositions and/or VEGF inhibitors against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The anti-MUC16^{c114}×CD3 multispecific (e.g., bispecific) immunoglobulin-related compositions and/or VEGF inhibitors can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4(3):201-9 (1994); and Gregoriadis, “Engineering Liposomes for Drug Delivery: Progress and Problems,” Trends Biotechnol., 13(12):527-37 (1995). Mizguchi et al., Cancer Lett., 100:63-69 (1996), describes the use of fusogenic liposomes to deliver a protein to cells both in vivo and in vitro.

Dosage, toxicity and therapeutic efficacy of the anti-MUC16^{c114}×CD3 multispecific (e.g., bispecific) immunoglobulin-related compositions and/or VEGF inhibitors can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. In some embodiments, the anti-MUC16^{c114}×CD3 multispecific (e.g., bispecific) immunoglobulin-related compositions and/or VEGF inhibitors exhibit high therapeutic indices. While the anti-MUC16^{c114}×CD3 multispecific (e.g., bispecific) immunoglobulin-related compositions and/or VEGF inhibitors that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies within a range of circulating concentrations that include the ED50 with little or no toxicity. The dosage may vary within this range depending upon the dosage form employed and the route of administration utilized. For any anti-MUC16^{c114}×CD3 multispecific (e.g., bispecific) immunoglobulin-related composition and/or VEGF inhibitor, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Typically, an effective amount of the anti-MUC16^{c114}×CD3 multispecific (e.g., bispecific) immunoglobulin-related composition and/or VEGF inhibitor, sufficient for achieving a therapeutic or prophylactic effect, range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example, dosages can be 1 mg/kg body weight or 10 mg/kg body weight every day, every two days or every three days or within the range of 1-10 mg/kg every week, every two weeks or every three weeks. In one embodiment, a single dosage of an anti-MUC16^{c114}×CD3 multispecific (e.g., bispecific) immunoglobulin-related composition and/or VEGF inhibitor ranges from 0.001-10,000 micrograms per kg body weight. In one embodiment, the anti-MUC16^{c114}×CD3 multispecific (e.g., bispecific) immunoglobulin-related composition and/or VEGF inhibitor concentrations is in a carrier range from 0.2 to 2000 micrograms per delivered milliliter. An exemplary treatment regime entails administration once per day or once a week. In therapeutic applications, a relatively high dosage at relatively short intervals is sometimes required until progression of the disease is reduced or terminated, and until the subject shows partial or complete amelioration of symptoms of disease. Thereafter, the patient can be administered a prophylactic regime.

In some embodiments, a therapeutically effective amount of an anti-MUC16^{c114}×CD3 multispecific (e.g., bispecific) immunoglobulin-related composition and/or VEGF inhibitor may be defined as a concentration of an anti-MUC16^{c114}×CD3 multispecific (e.g., bispecific) immunoglobulin-related composition and/or VEGF inhibitor at the target tissue of 10⁻¹² to 10⁻⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue. In some embodiments, the doses are administered by single daily or weekly administration, but may also include continuous administration (e.g., parenteral infusion or transdermal application). In some embodiments, the dosage of anti-MUC16^{c114}×CD3 multispecific (e.g., bispecific) immunoglobulin-related composition and/or VEGF inhibitor of the present technology is provided at a “low,” “mid,” or “high” dose level. In one embodiment, the low dose is provided from about 0.0001 to about 0.5 mg/kg/h, suitably from about 0.001 to about 0.1 mg/kg/h. In one embodiment, the mid-dose is provided from about 0.01 to about 1.0 mg/kg/h, suitably from about 0.01 to about 0.5 mg/kg/h. In one embodiment, the high dose is provided from about 0.5 to about 10 mg/kg/h, suitably from about 0.5 to about 2 mg/kg/h.

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance present methods can be any mammal, including, for example, farm animals, such as sheep, pigs, cows, and horses; pet animals, such as dogs and cats; laboratory animals, such as rats, mice and rabbits. In some embodiments, the mammal is a human.

### Methods of Treatment of the Present Technology

In one aspect, the present disclosure provides a method for treating gynecologic cancer in a subject in need thereof, comprising administering to the subject an effective amount of at least one anti-MUC16^{c114}×CD3 multispecific (e.g., bispecific) immunoglobulin-related composition and an effective amount of at least one VEGF inhibitor. Examples of gynecologic cancers include, but are not limited to ovarian cancer, fallopian tube cancer, uterine cancer, or endometrial cancer. In certain embodiments, the subject is human.

Additionally or alternatively, in some embodiments, the subject exhibits decreased tumor growth, reduced tumor proliferation, lower tumor burden, or increased survival after administration of the at least one anti-MUC16^{c114}×CD3 multispecific (e.g., bispecific) immunoglobulin-related composition and the at least one VEGF inhibitor. Additionally or alternatively, in some embodiments of the combination therapy methods disclosed herein, the time to response and/or duration of response is improved relative to that observed with anti-MUC16^{c114}×CD3 multispecific (e.g., bispecific) immunoglobulin-related composition monotherapy or VEGF inhibitor monotherapy.

In any and all embodiments of the methods disclosed herein, the at least one anti-MUC16^{c114}×CD3 multispecific (e.g., bispecific) immunoglobulin-related composition and the at least one VEGF inhibitor are administered separately, sequentially, or simultaneously. The anti-MUC16^{c114}×CD3 multispecific (e.g., bispecific) immunoglobulin-related composition and/or the VEGF inhibitor may be administered orally, intranasally, parenterally, intravenously, intramuscularly, intraperitoneally, intramuscularly, intraarterially, subcutaneously, intrathecally, intracapsularly, intraorbitally, intratumorally, intradermally, transtracheally, intracerebroventricularly, topically, or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Formulations including any anti-MUC16^{c114}×CD3 multispecific (e.g., bispecific) immunoglobulin-related composition and/or VEGF inhibitor disclosed herein may be designed to be short-acting, fast-releasing, or long-acting. In other embodiments, compounds can be administered in a local rather than systemic means, such as administration (e.g., by injection) at a tumor site.

Additionally or alternatively, in some embodiments of the methods disclosed herein, the at least one anti-MUC16^{c114}×CD3 multispecific (e.g., bispecific) immunoglobulin-related composition can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), simultaneously with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a VEGF inhibitor to a subject suffering from gynecologic cancer.

In some embodiments, the anti-MUC16^{c114}×CD3 multispecific (e.g., bispecific) immunoglobulin-related composition and VEGF inhibitor are administered to a subject, for example, a mammal, such as a human, in a sequence and within a time interval such that the therapeutic agent that is administered first acts together with the therapeutic agent that is administered second to provide greater benefit than if each therapeutic agent were administered alone. For example, the anti-MUC16^{c114}×CD3 multispecific (e.g., bispecific) immunoglobulin-related composition and VEGF inhibitor can be administered at the same time or sequentially in any order at different points in time; however, if not administered at the same time, the anti-MUC16^{c114}×CD3 multispecific (e.g., bispecific) immunoglobulin-related composition and VEGF inhibitor are administered sufficiently close in time so as to provide the desired therapeutic or prophylactic effect of the combination of the two therapeutic agents. In one embodiment, the anti-MUC16^{c114}×CD3 multispecific (e.g., bispecific) immunoglobulin-related composition and VEGF inhibitor exert their effects at times which overlap. In some embodiments, the anti-MUC16^{c114}×CD3 multispecific (e.g., bispecific) immunoglobulin-related composition and VEGF inhibitor each are administered as separate dosage forms, in any appropriate form and by any suitable route. In other embodiments, the anti-MUC16^{c114}×CD3 multispecific (e.g., bispecific) immunoglobulin-related composition and VEGF inhibitor are administered simultaneously in a single dosage form.

It will be appreciated that the frequency with which any of these therapeutic agents can be administered can be once or more than once over a period of about 2 days, about 3 days, about 4 days, about 5 days, about 6 days, about 7 days, about 8 days, about 9 days, about 10 days, about 11 days, about 12 days, about 13 days, about 14 days, about 20 days, about 28 days, about a week, about 2 weeks, about 3 weeks, about 4 weeks, about a month, about every 2 months, about every 3 months, about every 4 months, about every 5 months, about every 6 months, about every 7 months, about every 8 months, about every 9 months, about every 10 months, about every 11 months, about every year, about every 2 years, about every 3 years, about every 4 years, or about every 5 years.

For example, an anti-MUC16^{c114}×CD3 multispecific (e.g., bispecific) immunoglobulin-related composition or VEGF inhibitor may be administered daily, weekly, biweekly, or monthly for a particular period of time. An anti-MUC16^{c114}×CD3 multispecific (e.g., bispecific) immunoglobulin-related composition or VEGF inhibitor may be dosed daily over a 14 day time period, or twice daily over a seven day time period. An anti-MUC16^{c114}×CD3 multispecific (e.g., bispecific) immunoglobulin-related composition or VEGF inhibitor may be administered daily for 7 days.

Alternatively, an anti-MUC16^{c114}×CD3 multispecific (e.g., bispecific) immunoglobulin-related composition or VEGF inhibitor may be administered daily, weekly, biweekly, or monthly for a particular period of time followed by a particular period of non-treatment. In some embodiments, the anti-MUC16^{c114}×CD3 multispecific (e.g., bispecific) immunoglobulin-related composition or VEGF inhibitor can be administered daily for 14 days followed by seven days of non-treatment, and repeated for two more cycles of daily administration for 14 days followed by seven days of non-treatment. In some embodiments, the anti-MUC16^{c114}×CD3 multispecific (e.g., bispecific) immunoglobulin-related composition or VEGF inhibitor can be administered twice daily for seven days followed by 14 days of non-treatment, which may be repeated for one or two more cycles of twice daily administration for seven days followed by 14 days of non-treatment.

In some embodiments, the anti-MUC16^{c114}×CD3 multispecific (e.g., bispecific) immunoglobulin-related composition or VEGF inhibitor is administered daily over a period of 14 days. In another embodiment, the anti-MUC16^{c114}×CD3 multispecific (e.g., bispecific) immunoglobulin-related composition or VEGF inhibitor is administered daily over a period of 12 days, or 11 days, or 10 days, or nine days, or eight days. In another embodiment, the anti-MUC16^{c114}×CD3 multispecific (e.g., bispecific) immunoglobulin-related composition or VEGF inhibitor is administered daily over a period of seven days. In another embodiment, the anti-MUC16^{c114}×CD3 multispecific (e.g., bispecific) immunoglobulin-related composition or VEGF inhibitor is administered daily over a period of six days, or five days, or four days, or three days.

In some embodiments, individual doses of the anti-MUC16^{c114}×CD3 multispecific (e.g., bispecific) immunoglobulin-related composition and the VEGF inhibitor are administered within a time interval such that the two therapeutic agents can work together (e.g., within 1 hour, 2 hours, 4 hours, 6 hours, 12 hours, 24 hours, 48 hours, 72 hours, 96 hours, 5 days, 6 days, 1 week, or 2 weeks). In some embodiments, the treatment period during which the therapeutic agents are administered is then followed by a non-treatment period of a particular time duration, during which the therapeutic agents are not administered to the subject. This non-treatment period can then be followed by a series of subsequent treatment and non-treatment periods of the same or different frequencies for the same or different lengths of time. In some embodiments, the treatment and non-treatment periods are alternated. It will be understood that the period of treatment in cycling therapy may continue until the subject has achieved a complete response or a partial response, at which point the treatment may be stopped. Alternatively, the period of treatment in cycling therapy may continue until the subject has achieved a complete response or a partial response, at which point the period of treatment may continue for a particular number of cycles. In some embodiments, the length of the period of treatment may be a particular number of cycles, regardless of subject response. In some other embodiments, the length of the period of treatment may continue until the subject relapses.

In some embodiments, the anti-MUC16^{c114}×CD3 multispecific (e.g., bispecific) immunoglobulin-related composition and the VEGF inhibitor are cyclically administered to a subject. Cycling therapy involves the administration of a first agent (e.g., a first prophylactic or therapeutic agent) for a period of time, followed by the administration of a second agent and/or third agent (e.g., a second and/or third prophylactic or therapeutic agent) for a period of time and repeating this sequential administration. Cycling therapy can reduce the development of resistance to one or more of the therapies, avoid or reduce the side effects of one of the therapies, and/or improve the efficacy of the treatment.

In some embodiments, the anti-MUC16^{c114}×CD3 multispecific (e.g., bispecific) immunoglobulin-related composition is administered for a particular length of time prior to administration of the VEGF inhibitor. For example, in a 21-day cycle, the anti-MUC16^{c114}×CD3 multispecific (e.g., bispecific) immunoglobulin-related composition may be administered on days 1 to 5, days 1 to 7, days 1 to 10, or days 1 to 14, and the VEGF inhibitor may be administered on days 6 to 21, days 8 to 21, days 11 to 21, or days 15 to 21. In other embodiments, the anti-MUC16^{c114}×CD3 multispecific (e.g., bispecific) immunoglobulin-related composition is administered for a particular length of time prior to administration of the VEGF inhibitor. For example, in a 21-day cycle, the anti-MUC16^{c114}×CD3 multispecific (e.g., bispecific) immunoglobulin-related composition may be administered on days 1 to 5, days 1 to 7, days 1 to 10, or days 1 to 14, and the VEGF inhibitor may be administered on days 6 to 21, days 8 to 21, days 11 to 21, or days 15 to 21.

In one embodiment, the administration is on a 21-day dose schedule in which a once daily dose of anti-MUC16^{c114}×CD3 multispecific (e.g., bispecific) immunoglobulin-related composition is administered beginning on day eight for seven days, followed by seven days of non-treatment, in combination with twice-daily administration of the VEGF inhibitor for seven days followed by 14 days of non-treatment (e.g., the anti-MUC16^{c114}×CD3 multispecific (e.g., bispecific) immunoglobulin-related composition is administered on days 8-14 and the VEGF inhibitor is administered on days 1-7 of the 21-day schedule).

In another embodiment, the administration is on a 21-day dose schedule in which a once daily dose of VEGF inhibitor is administered beginning on day eight for seven days, followed by seven days of non-treatment, in combination with twice-daily administration of the anti-MUC16^{c114}×CD3 multispecific (e.g., bispecific) immunoglobulin-related composition for seven days followed by 14 days of non-treatment (e.g., the VEGF inhibitor is administered on days 8-14 and the anti-MUC16^{c114}×CD3 multispecific (e.g., bispecific) immunoglobulin-related composition is administered on days 1-7 of the 21-day schedule).

In some embodiments, the anti-MUC16^{c114}×CD3 multispecific (e.g., bispecific) immunoglobulin-related composition and VEGF inhibitor each are administered at a dose and schedule typically used for that agent during monotherapy. In other embodiments, when the anti-MUC16^{c114}×CD3 multispecific (e.g., bispecific) immunoglobulin-related composition and VEGF inhibitor are administered concomitantly, one or both of the agents can advantageously be administered at a lower dose than typically administered when the agent is used during monotherapy, such that the dose falls below the threshold that an adverse side effect is elicited.

The therapeutically effective amounts or suitable dosages of the anti-MUC16^{c114}×CD3 multispecific (e.g., bispecific) immunoglobulin-related composition and the VEGF inhibitor in combination depends upon a number of factors, including the nature of the severity of the condition to be treated, the particular inhibitor, the route of administration and the age, weight, general health, and response of the individual subject. In certain embodiments, the suitable dose level is one that achieves a therapeutic response as measured by tumor regression or other standard measures of disease progression, progression free survival, or overall survival. In other embodiments, the suitable dose level is one that achieves this therapeutic response and also minimizes any side effects associated with the administration of the therapeutic agent.

Suitable daily dosages of anti-MUC16^{c114}×CD3 multispecific (e.g., bispecific) immunoglobulin-related composition can generally range, in single or divided or multiple doses, from about 10% to about 120% of the maximum tolerated dose as a single agent. In certain embodiments, the suitable dosages of anti-MUC16^{c114}×CD3 multispecific (e.g., bispecific) immunoglobulin-related composition are from about 20% to about 100% of the maximum tolerated dose as a single agent. In other embodiments, the suitable dosages of anti-MUC16^{c114}×CD3 multispecific (e.g., bispecific) immunoglobulin-related composition are from about 25% to about 90% of the maximum tolerated dose as a single agent. In some embodiments, the suitable dosages of anti-MUC16^{c114}×CD3 multispecific (e.g., bispecific) immunoglobulin-related composition are from about 30% to about 80% of the maximum tolerated dose as a single agent. In other embodiments, the suitable dosages of anti-MUC16^{c114}×CD3 multispecific (e.g., bispecific) immunoglobulin-related composition are from about 40% to about 75% of the maximum tolerated dose as a single agent. In some embodiments, the suitable dosages of anti-MUC16^{c114}×CD3 multispecific (e.g., bispecific) immunoglobulin-related composition are from about 45% to about 60% of the maximum tolerated dose as a single agent. In other embodiments, suitable dosages of anti-MUC16^{c114}×CD3 multispecific (e.g., bispecific) immunoglobulin-related composition are about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, or about 120% of the maximum tolerated dose as a single agent.

Suitable daily dosages of VEGF inhibitor can generally range, in single or divided or multiple doses, from about 10% to about 120% of the maximum tolerated dose as a single agent. In certain embodiments, the suitable dosages of VEGF inhibitor are from about 20% to about 100% of the maximum tolerated dose as a single agent. In some other embodiments, the suitable dosages of VEGF inhibitor are from about 25% to about 90% of the maximum tolerated dose as a single agent. In some other embodiments, the suitable dosages of VEGF inhibitor are from about 30% to about 80% of the maximum tolerated dose as a single agent. In some other embodiments, the suitable dosages of VEGF inhibitor are from about 40% to about 75% of the maximum tolerated dose as a single agent. In some other embodiments, the suitable dosages of VEGF inhibitor are from about 45% to about 60% of the maximum tolerated dose as a single agent. In other embodiments, suitable dosages of VEGF inhibitor are about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, or about 120% of the maximum tolerated dose as a single agent.

For example, when administered to the appropriate subject as determined by the methods of the present technology, a therapeutically effective amount of the anti-MUC16^{c114}×CD3 multispecific (e.g., bispecific) immunoglobulin-related composition and VEGF inhibitor may partially or completely alleviate one or more symptoms of gynecologic cancer and/or lead to increased survival, reduced tumor burden, reduced tumor relapse, reduction of the number of cancer cells, reduction of the tumor size, eradication of tumor, inhibition of cancer cell infiltration into peripheral organs, inhibition or stabilization of tumor growth, and stabilization or improvement of quality of life in the subject.

### Kits of the Present Technology

The present disclosure provides kits for treating gynecologic cancer comprising an anti-MUC16^{c114}×CD3 multispecific (e.g., bispecific) immunoglobulin-related composition disclosed herein, a VEGF inhibitor disclosed herein, and instructions for treating gynecologic cancers. When simultaneous administration is contemplated, the kit may comprise an anti-MUC16^{c114}×CD3 multispecific (e.g., bispecific) immunoglobulin-related composition and a VEGF inhibitor that has been formulated into a single pharmaceutical composition such as a tablet, or as separate pharmaceutical compositions. When the anti-MUC16^{c114}×CD3 multispecific (e.g., bispecific) immunoglobulin-related composition and the VEGF inhibitor are not administered simultaneously, the kit may comprise an anti-MUC16^{c114}×CD3 multispecific (e.g., bispecific) immunoglobulin-related composition and a VEGF inhibitor that has been formulated as separate pharmaceutical compositions either in a single package, or in separate packages.

Additionally or alternatively, in some embodiments, the kits further comprise at least one chemotherapeutic agent and/or at least one immune checkpoint inhibitors that are useful for treating gynecologic cancer. Examples of such chemotherapeutic agents include but are not limited to taxanes, alkylating agents, antitumor antibiotics, topoisomerase inhibitors (e.g., topoisomerase II inhibitors), endoplasmic reticulum stress inducing agents, antimetabolites, and mitotic inhibitors. In some embodiments, the chemotherapeutic agent is selected from the group consisting of chlorambucil, cyclophosphamide, ifosfamide, melphalan, streptozocin, carmustine, lomustine, bendamustine, uramustine, estramustine, carmustine, nimustine, ranimustine, mannosulfan busulfan, dacarbazine, temozolomide, thiotepa, altretamine, 5-fluorouracil (5-FU), 6-mercaptopurine (6-MP), capecitabine, cytarabine, floxuridine, fludarabine, gemcitabine, hydroxyurea, methotrexate, pemetrexed, daunorubicin, doxorubicin, epirubicin, idarubicin, SN-38, ARC, NPC, campothecin, topotecan, 9-nitrocamptothecin, 9-aminocamptothecin, rubifen, gimatecan, diflomotecan, BN80927, DX-8951f, MAG-CPT, amsacrine, etoposide, etoposide phosphate, teniposide, doxorubicin, paclitaxel, docetaxel, gemcitabine, accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephaolmannine, and mixtures thereof.

Examples of immune checkpoint inhibitors include immuno-modulating/stimulating antibodies such as an anti-PD-1 antibody, an anti-PD-L1 antibody, an anti-PD-L2 antibody, an anti-CTLA-4 antibody, an anti-TIM3 antibody, an anti-4-1BB antibody, an anti-CD73 antibody, an anti-GITR antibody, an anti-B7-H3 antibody, an anti-B7-H4 antibody, an anti-TIGIT antibody, an anti-CD80 antibody, an anti-CD86 antibody, an anti-ICOS antibody, an anti-BTLA antibody, and an anti-LAG-3 antibody. Specific immune checkpoint inhibitors include ipilimumab, nivolumab, pidilizumab, lambrolizumab, pembrolizumab, atezolizumab, avelumab, durvalumab, MPDL3280A, BMS-936559, MEDI-4736, MSB 00107180, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, DLBCL inhibitors, and any combination thereof.

The kits may further comprise pharmaceutically acceptable excipients, diluents, or carriers that are compatible with one or more kit components described herein. Optionally, the above described components of the kits of the present technology are packed in suitable containers and labeled for the treatment of gynecologic cancer (e.g., a MUC16 expressing gynecologic cancer). Examples of gynecologic cancers include, but are not limited to as ovarian cancer, fallopian tube cancer, uterine cancer and endometrial cancer.

The kits may optionally include instructions customarily included in commercial packages of therapeutic products, that contain information about, for example, the indications, usage, dosage, manufacture, administration, contraindications and/or warnings concerning the use of such therapeutic products.

### EXAMPLES

The following examples are provided to further illustrate the methods of the present disclosure. These examples are illustrative only and are not intended to limit the scope of the disclosure in any way. For each of the examples below, any anti-MUC16^{c114}×CD3 multispecific (e.g., bispecific) immunoglobulin-related composition or any VEGF inhibitors described herein could be used.

### Example 1: Materials and Methods

Human phage display panning. E-ALPHA® human phage display library was used to screen for clones that specifically bind to MUC16^{ecto}. Independent panning was carried out using 15 different phage sub-libraries. Individual scFv phage clones positive for MUC16^{ecto} were determined by FACS and the clones that possessed unique DNA coding sequences were subjected to further characterization. The positive phage clones were further validated for binding to MUC16^{ecto} overexpressing HEK293 cells. For FACS screening, phage clones were incubated with MUC16^{ecto} overexpressing HEK293 cells, then with anti-M13 mouse antibody. APC-labeled anti-mouse IgG 2nd antibody was added to the reaction after washing. Binding was measured by FACS and expressed as mean fluorescence intensity (MFI). Cells incubated with 2nd antibody alone, M13 K07 helper phage, and cells only were used as negative controls.

Cell lines and cytotoxicity. For phage display screening, unmodified HEK293, HEK293 expressing MUC16 (HEK-MUC16WT) or HEK293 cells expressing mutant MUC16 (HEK293-MUC16mut) were used. For cytotoxicity assays, SKOV3 (MUC16e) SKOV3-MUC16^{ecto} (MUC16^{Pos}), OVCAR3 (MUC16^{Pos}), OVCAR432 (MUC16^{Pos}), and SKOV8 (MUC16^{Pos}) cell lines were used. For luciferase-based cytotoxicity assays, imaging and survival assays; SKOV3 modified to express MUC16^{ecto} and the luciferase gene (SKOV3-MUC16^{ecto}-Luc), wild type isogenic SKOV3-Luc, and OVCAR3-Luc cells were used. All human ovarian cancer cell lines were maintained in RPMI (Invitrogen, Grand Island, NY, USA) supplemented with 10% heat-inactivated fetal calf serum (FBS), 100 U/ml penicillin and streptomycin (P/S), and 2 mM L-glutamine. Cells were validated using karyotype analysis and routinely checked for mycoplasma contamination. Flow cytometric analyses were performed using Gallios Flow Cytometer with Kaluza software (Beckman Coulter, Brea, CA, USA). MUC16^{ecto} expression was detected using APC-conjugated anti-Muc16 antibody. Human T cells were derived from fresh blood-derived leukocyte concentrate (Leukopack) obtained from the New York Blood Center, mononuclear cells were separated using density gradient centrifugation with Accu-prep (axis-Shield PoC AS). T cells were isolated, activated and expanded with PHA (Sigma Aldrich, St Louis, MO) at a concentration of 2×10⁶/ml. T cells were cultured in RPMI supplemented with 10% fetal calf serum, 100 U/ml penicillin and streptomycin, and 2 mM L-glutamine, in the presence of 100 IU/ml recombinant human IL-2 (Proleukin). Viable cells were enumerated using flow cytometry and counting beads (Ebioscience). For LDH-based cytotoxicity, tumor cells and activated T-cells were cocultured 1:1 in the presence of 0.2 μg/ml of the relevant BiTEDs for 16 hrs. LDH release assay was used to quantify dead cells according to the manufacturers protocol. For luciferase-based cytotoxicity assays, activated donor T-cells were cocultured with SKOV3-Luc, SKOV3-MUC16^{ecto}-Luc or OVCAR3-Luc at the indicated effector: target ratios in the presence of 0.5 μg/ml of BiTEDs for 48 hrs, and subsequently mixed with luciferase assay reagent (Promega). Luminescence of the lysates was analyzed using a plate spectrophotometer. All cytotoxicity experiments were performed with at least four separate donors and repeated a minimum of three times.

SDS-PAGE validation. Candidate Anti-CD3ε bispecific engagers were evaluated using SDS-PAGE. Samples were run using the NuPAGE® Novex® Bis-Tris gel (4-12%) and NuPAGE® MEX×1 running buffer under reducing conditions at 70° C. for 10 mins. The expected band size was 50 KDa.

Immunoprecipitation. For immunoprecipitation experiments, 1.5 μM of affinity-purified BTM protein (a synthetic fusion protein consisting of the highly conserved extracellular portion of MUC16^{c57-114} fused to a human Fc) was added to equal amounts of MUC16 BiTEDs in PBS buffer, and the mixture was incubated with rotation in the presence of Protein G agarose. The immunocomplex was adsorbed onto 25 μl suspension of protein G-agarose beads (millipore) (pre-washed 3 times with PBS buffer) by incubating the mixture for 90 minutes at 4° C. The beads were washed 3 times with 600 μl of PBS buffer. Finally, the beads were resuspended in 30 μl of 0.1 M Glycine pH, 2.7 for elution of the complex. The eluate was mixed with SDS sample buffer, heated for 8 min, and the proteins were separated by NuPAGE™ 4-12% Bis-Tris Protein Gels, (Life Technology, NP0335BOX). The protein bands were detected in the gel by Coomassie Blue staining.

Kinetic Analysis of BiTEDs binding to MUC16^{ecto}. The kinetic analysis of EXT170-8 BiTEDs and Muc16 BTM protein (55mer highly conserved ectodomain region of MUC16) was performed on a BiaCore X100 instrument loaded with an NTA sensor chip. EXT170-8 BiTED comprises the amino acid of SEQ ID NO: 89 and the leader sequence of SEQ ID NO: 93.

The full length nucleic acid sequence of the EXT170-8 BiTED is provided below:

(SEQ ID NO: 96)
ATGGAAACCGACACCCTGCTGCTGTGGGTGCTGCTGCTGTGGGTGCCAG
GATCTACCGGTGACATCCAGTTGACCCAGTCTCCATCTGCCGTGTCTGC
ATCTGTAGGAGACAGAGTCACTATCACTTGTCGGGCGAGTCAGGATGTT
AGCAAGTGGTTAGCCTGGTATCAGCAGAAGCCAGGGAAAGCCCCCAGGC
TCCTGATCTCTGCTGCATCCGGTCTGCAAAGTTGGGTCCCATCAAGGTT
CAGCGGCAGTGGATCTGGGACAGAATTCACTCTCTCCATCAGCAGCCTG
CAGCCTGAAGATTTTGCAACTTACTATTGTCAACAGGCTAATAGTTTCC
CGTGGACGTTCGGCCAAGGGACCAAGGTGGAAATCAAACGTTCTAGAGG
TGGTGGTGGTAGCGGCGGCGGCGGCTCTGGTGGTGGTGGATCCCTCGAG
ATGGCCCAGGTGCAGTTACAGCAGTGGGGCGCAGGACTGTTGAAGCCTT
CGGAGACCCTGTCCCTCACCTGCGCTGTCTATGGTGGGTCCTTCAGTGG
TTACTACTGGAGCTGGATCCGCCAGCCCCCAGGGAAGGGGCTGGAGTGG
ATTGGGGAAATCAATCATAGTGGAAGCACCAACTACAACCCGTCCCTCA
AGAGTCGAGTCACCATATCAGTAGACACGTCCAAGAACCAGTTCTCCCT
GAAGCTGAGCTCTGTGACCGCCGCGGACACGGCCGTGTATTACTGTGCG
CGCCAGTCTTACATCACTGATTCTTGGGGTCAAGGTACTCTGGTGACCG
TCTCCTCAACTAGTGGCGGGGGAGGATCCGACGTGCAGCTGGTGCAGAG
CGGAGCTGAAGTGAAGAAACCTGGCGCCTCCGTGAAGGTGTCCTGCAAA
GCTAGCGGCTATACCTTCACCCGGTACACCATGCACTGGGTGCGCCAGG
CACCTGGACAGGGACTGGAATGGATCGGCTACATCAACCCCTCCCGGGG
CTACACCAACTACGCCGACTCTGTGAAGGGCCGGTTCACCATCACCACC
GATAAGTCCACCAGCACCGCTTACATGGAACTGTCCTCCCTGAGATCCG
AGGACACCGCTACCTACTATTGCGCCCGGTACTACGACGACCACTACTG
CCTGGACTACTGGGGACAGGGAACCACAGTGACCGTGTCCTCTGGCGAG
GGCACCTCTACTGGATCTGGGGGAAGTGGTGGTTCTGGCGGCGCTGACG
ACATCGTGCTGACCCAGTCTCCAGCCACCCTGTCTCTGAGCCCAGGCGA
GAGAGCTACCCTGTCCTGCAGAGCCTCCCAGTCCGTGTCCTACATGAAT
TGGTATCAGCAGAAGCCTGGCAAGGCCCCTAAGCGGTGGATCTACGACA
CCTCCAAGGTGGCCTCTGGCGTGCCAGCCCGGTTTTCCGGATCTGGCTC
TGGCACCGACTACTCCCTGACCATCAACAGCCTGGAAGCCGAGGACGCT
GCCACCTATTACTGCCAGCAGTGGTCCTCCAACCCCCTGACCTTTGGAG
GCGGCACCAAGGTGGAAATCAAGCACCACCATCATCACCACTAA

The full length amino acid sequence of the EXT170-8 BiTED is provided below:

(SEQ ID NO: 97)
METDTLLLWVLLLWVPGSTGDIQLTQSPSAVSASVGDRVTITCRASQDVSKWLA
WYQQKPGKAPRLLISAASGLQSWVPSRFSGSGSGTEFTLSISSLQPEDFATYYCQ
QANSFPWTFGQGTKVEIKRSRGGGGSGGGGSGGGGSLEMAQVQLQQWGAGLLK
PSETLSLTCAVYGGSFSGYYWSWIRQPPGKGLEWIGEINHSGSTNYNPSLKSRVT
ISVDTSKNQFSLKLSSVTAADTAVYYCARQSYITDSWGQGTLVTVSSTSGGGGSD
VQLVQSGAEVKKPGASVKVSCKASGYTFTRYTMHWVRQAPGQGLEWIGYINPS
RGYTNYADSVKGRFTITTDKSTSTAYMELSSLRSEDTATYYCARYYDDHYCLDY
WGQGTTVTVSSGEGTSTGSGGSGGSGGADDIVLTQSPATLSLSPGERATLSCRAS
QSVSYMNWYQQKPGKAPKRWIYDTSKVASGVPARFSGSGSGTDYSLTINSLEAE

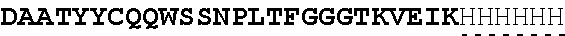

Leader sequence is underlined, V_{H} and V_{L} domains are indicated in boldface, His Tag is indicated by dash line.

Briefly, the His-tagged EXT170-8 BiTEDs was immobilized onto NTA sensor chip at a concentration of 20 g/ml, and MUC16 BTM protein (35 kDa) was injected at concentrations of 570, 285, 142.5, 71.25, and 35.625 nM (20, 10, 5, 2.5 and 1.25 ul/ml). The raw data was analyzed using the kinetic model 1:1 binding by Biacore X100 evaluation software.

ELISA. 96-well clear, flat-bottom plates (Thermo Scientific, 14-245-153) were coated with 1 μg/ml BTM (55mer highly conserved ectodomain region of MUC16) diluted in 0.1M sodium bicarbonate coating buffer (pH 8.0) overnight at 4° C. Each well was washed with PBS-T (0.05% Tween-20) and subsequently blocked with PBS+2% BSA at room temperature for 1 hour. Wells were then washed with PBS-T before the addition of either biotinylated BiTEDs (Biotin labeling kit; Roche, 11418165001) or biotinylated Muc16 BiTEDs with free rhCA-125 (R&D systems, 5609-MU). Ratios of 1:1, 5:1, and 10:1 rhCA-125 to coated BTM were used. Following a one-hour incubation at room temperature on a plate rocker, wells were washed with PBS-T. Streptavidin-HRP A (R&D systems, 890803) at 1:200 in PBS was then added to each well and incubated for 1 hour at room temperature under foil. Each well was washed with PBS-T before the addition of TMB ELISA Peroxidase Substrate (Rockland) and allowed to develop for 20 minutes under foil at room temperature. Reactions were then quenched with 0.6N sulfuric acid. Wavelengths of 450 nm and 540 nm (for plate refraction correction) were measured via SpectraMax iD3 Microplate Reader. Analyses were then performed averaging wavelengths of quadruplicate samples, accounting for both plate refraction (540 nm) as well as remaining HRP-TMB absorbance (residual detection from negative control conditions of BTM coat followed by Strep-HR, TMB substrate, and then stop solution; excludes biotinylated Muc16 BiTEDs). VEGF detection was performed on OVCAR 3 cells cultured in 6-well plates cultured for 48 hours in complete RPMI according to manufacturer's instructions (R&D systems, DVE00).

FACS analyses. Flow cytometric analyses were performed using Gallios Flow Cytometer with Kaluza software (Beckman Coulter, Brea, CA, USA). Muc16^{ecto} expression was detected using APC-conjugated anti-Muc16 antibody. Human cells were stained with mouse anti-human CD3 (PE/APC, Thermofisher UCHT1/OKT3), PD-1 (APC, Thermofisher MIH4), TIM3 (APC, Thermofisher F38-2E2), LAG3 (APC, Thermofisher 3DS223H), Granzyme B (APC, Thermofisher GB 11), CD4 (PE, Thermofisher RPA-T4), CD8 (PE, Thermofisher RPA-T8), VEGF (PE, R&D systems IC2931P) and CD45 (APC, Thermofisher HI30). Tumor, splenocytes or peritoneal cells were pelleted and washed 3 times with FACS buffer (PBS+2.5% FBS). Cells were resuspended with the appropriate antibody, diluted in FACS buffer and incubated at 4° C. for 30 min in the dark. The cells were subsequently washed 3 times with cold FACS buffer and resuspended in 1×DAPI prior to FACS analysis.

Cytokine measurement. Serum cytokines were measured from blood collected via retro-orbital bleeds from indicated animals and centrifuged to separate the serum fraction. Cytokine detection was performed using the MILLIPLEX MAP Human Cytokine/Chemokine, Premixed 13 Plex kit, and the Luminex IS100 system. Dedicated cytokine assays for IL-2 (abcam, ab174444) and IFN-γ (abcam, ab174443) were performed with commercial ELISA kits according the manufacturers protocol.

Animal imaging and in vivo experiments. Female NSG mice age 6-8 weeks were purchased from Jackson Laboratory, Bar Harbor, ME, USA. 3×10⁶ SKOV3-MUC16^{ecto}/-Luc, or OVCAR3 tumor cells were injected intraperitoneally (i.p.) on day 0, and animals were untreated, treated with T-cells intravenously (i.v) alone or treated with a combination of T-cells and 5 μg MUC16^{ecto}-BiTEDs on day 7. Animals in the BiTEDs treatment group received additional 5 g MUC16^{ecto}-BiTEDs treatment on days 9, 11, 14, 16, and 18 for a total of 6 treatments over 2 weeks. Tumor-bearing mice were injected intraperitoneally with D-Luciferin (Goldbio Technology) (150 mg/kg) and after 10 min were imaged under isofluorane anesthesia. Bioluminescent imaging was achieved using the Caliper IVIS imaging system and analyzed with Living Image 4.0 software (PerkinElmer). Image acquisition was achieved using a 25 cm field of view, medium binning level and 60s exposure time. Animals treated with αPD-1 blocking antibody (clone EH12.2H7, BioLegend) received 250 μg injected i.p on days 7, 14, 21 and 28 (weekly injections×4 weeks) after tumor inoculation (day 0). Animals treated with αVEGF blocking antibody (Invivo Gen, hvegf-mab1) received 5 mg/kg i.p injections on days 7, 11, 14, 18, and 21 after tumor inoculation. All mice were monitored for survival and were euthanized when showing signs of distress.

Statistical analysis. Survival curves were analyzed using Mantel-Cox (log-rank) test and other analysis were performed using unpaired two-tailed T test (p value <0.05 considered as significant). All calculations were performed using Prism 7 (GraphPad) software.

### Example 2: Human Phage Panning and Identification of MUC16^{ecto} Specific Clones

E-ALPHA® human phage display library was used to screen for clones that specifically bind to the retained portion of MUC16 (FIG. 1(a)). The positive clones were screened by flow cytometry using HEK293 cells modified to overexpress MUC16^{ecto} (MUC16^{c114}). Clones bound to MUC16^{ecto} but not the mutant form were selected for further development into bispecific diabodies (BiTEDs) (FIG. 1(b)). Bispecific T-cell Engaging Diabodies (BiTEDs) involve two covalently linked single chain variable fragments (scFv). One scFv is specific for a tumor-associated antigen (TAA), and the other binds to the CD3 subunit of the T-cell receptor (see Baeuerle PA, Reinhardt C. Cancer Res. 2009; 69(12):4941-4). A total of 15 libraries were screened, which yielded 540 clones that were subjected to verification by flow cytometry. Of these, 53 unique clones were identified, and, ultimately 16 clones showed preferential binding to MUC16^{ecto}.

### Example 3: Validation of MUC16^{ecto} Specific Bispecific T-Cell Engaging Diabodies

Single chain variable fragments (scFvs) derived from candidate phage libraries were cloned into a bispecific antibody construct with one arm expressing anti-human CD3ε scFv antibody. The amino acid sequence of anti-human CD3ε scFv antibody is represented by SEQ ID NO: 72.

MUC16^{ecto}-BiTEDs were purified and confirmed by SDS-page (FIG. 1(c)). To determine the binding affinity of BiTEDs to MUC16^{ecto}, a previously validated BTM protein (a synthetic fusion protein consisting of the highly conserved extracellular portion of MUC16^{c57-114} fused to a human Fc backbone pFUSE) was used, and kinetic analysis was performed using surface plasmon resonance. The dissociation constant (K_{D}) for the lead MUC16^{ecto}-BiTEDs was 69.9 nM (FIG. 1(d)).

### Example 4: MUC16^{ecto}-BITEDs Bind to the Extracellular Domain of MUC16 and not shed CA-125

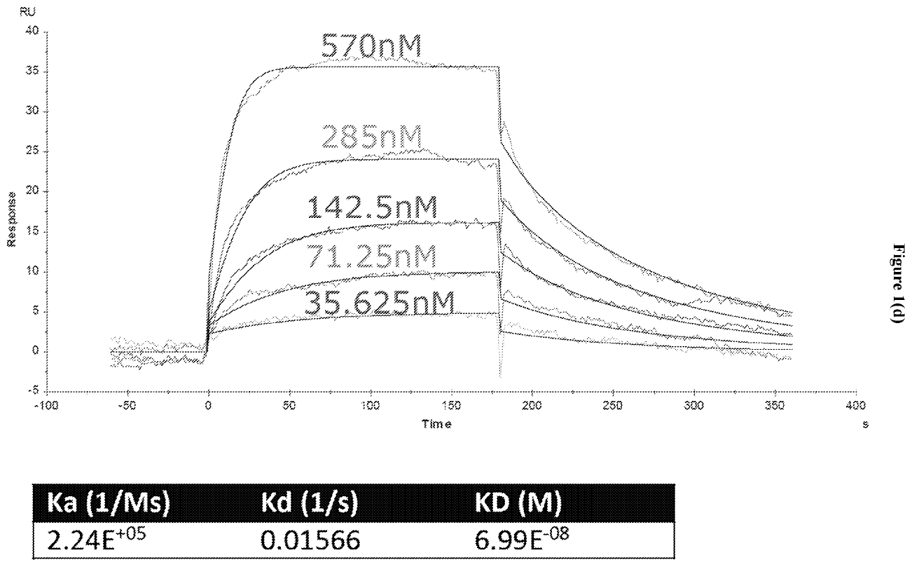

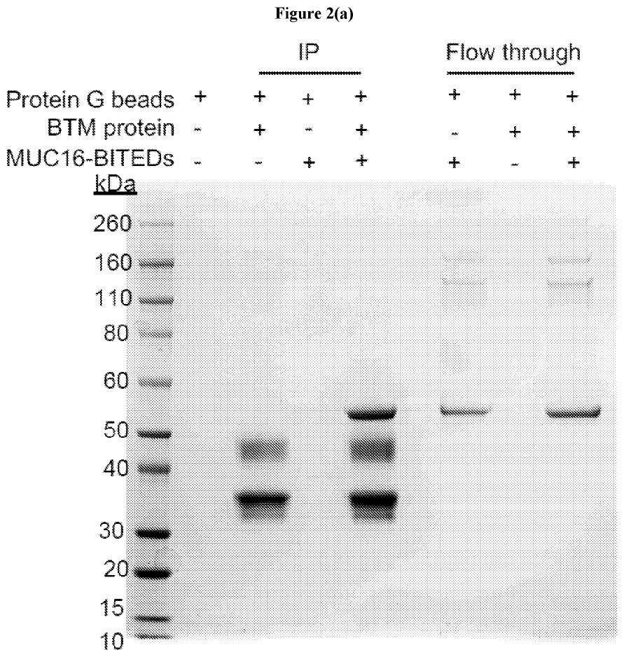

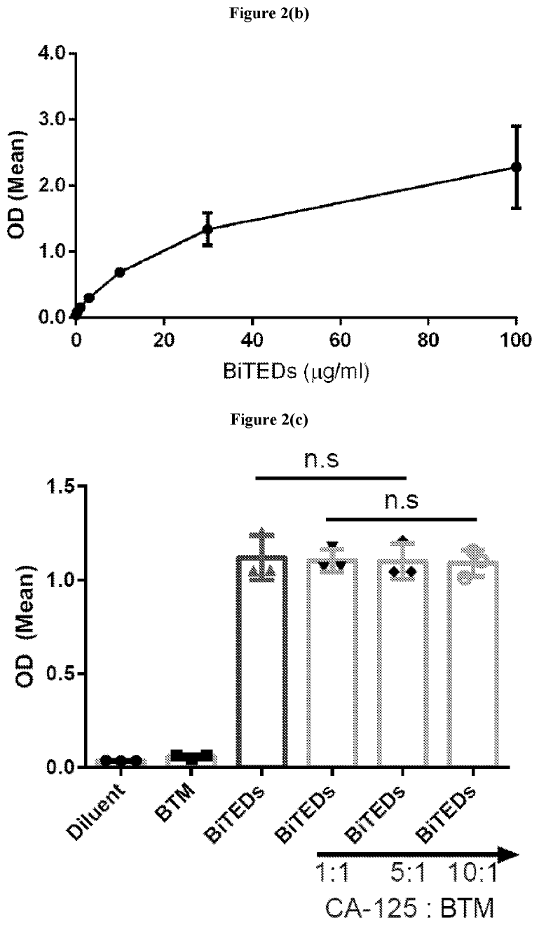

Prior studies have shown that the MUC16^{ecto} antigen binding fragments disclosed herein are specific for the retained domain and not the cleaved portion of MUC16 (CA-125) (Rao T D et al., ACS Chem Biol. 2017; 12(8):2085-96). The lead-candidate selection strategy was specifically designed to identify hybridoma clones that recognized the N-glycosylation site (N31) of MUC16^{ecto} (MUC16-C114) and not a mutant region (MUC16-N123) (FIGS. 1(a)-1(b)). Direct protein-protein binding of MUC16^{ecto}-BiTEDs (SEQ ID NO: 97) with MUC16^{ecto} was evaluated via co-immunoprecipitation (FIG. 2(a)). As shown in FIG. 2(a), a mixture of MUC16^{ecto}-BiTEDs with BTM produced the expected protein band after (lane 4, ˜55 kDa), but not BTM without MUC16^{ecto}-BiTEDs (lane 2) or vice versa (lane 3).

To further validate that the BiTEDs bound to MUC16^{ecto}, an ELISA assay was performed using plate-bound BTM. As shown in FIG. 2(b), MUC16^{ecto}-BiTEDs bound to BTM in a concentration-dependent manner. Next, to evaluate if this binding could be disrupted by shed CA-125, the ELISA assay was repeated in the presence of increasing concentrations of CA-125 to BTM. At 1:1, 5:1, and up to a 10-fold increase in CA-125 to BTM concentrations, no decrease in MUC16^{ecto}-BiTEDs binding was observed (FIG. 2(c)).

### Example 5: MUC16^{ecto}-BiTEDs Display Specific Cytotoxicity Against a Panel of Ovarian Cancer Cell Lines In Vitro

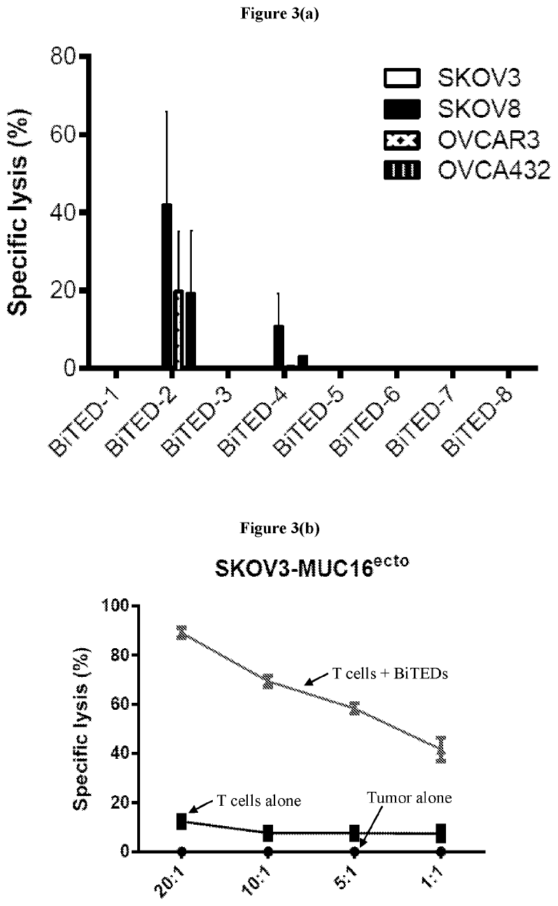

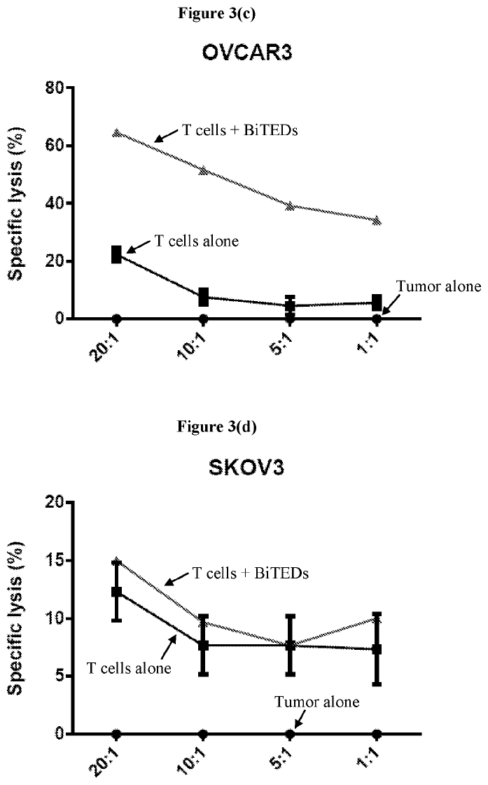

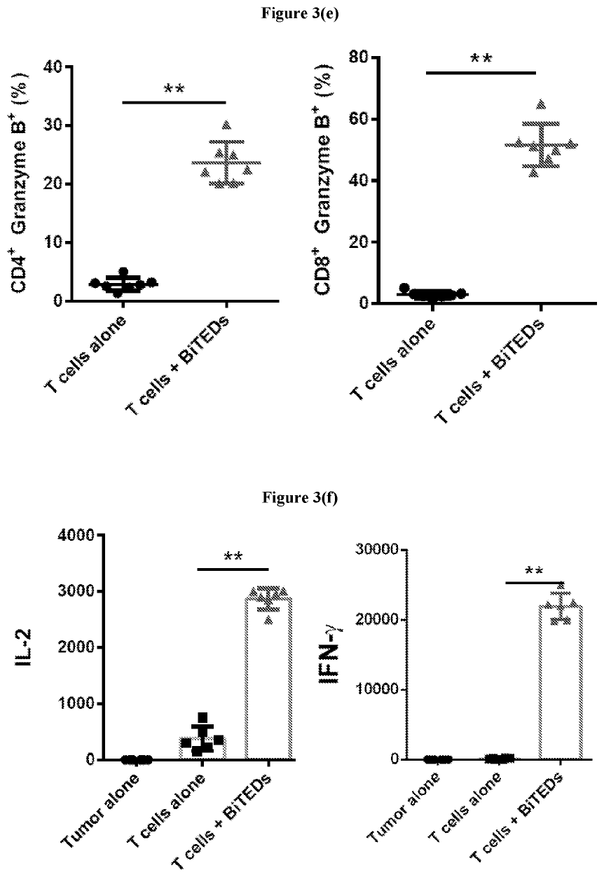

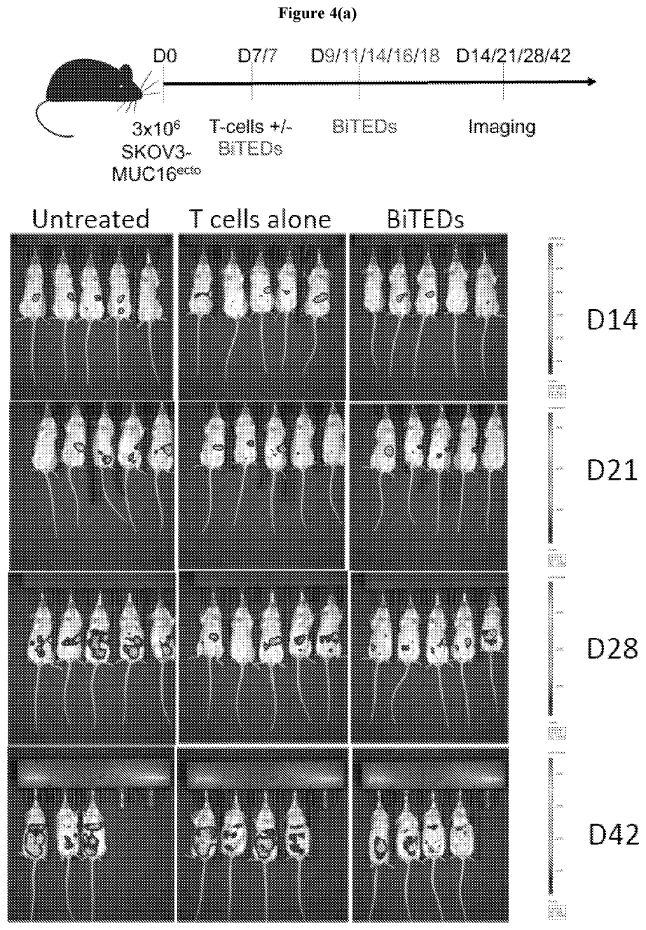

The in vitro potency of MUC16^{ecto}-BiTEDs against various ovarian cancer cell lines with varying degrees of MUC16 expression was evaluated. Using a panel including SKOV3 (MUC16^{neg}), SKOV8 (MUC16^{pos}), OVCAR3 (MUC16^{pos}), and OVCAR432 (MUC16^{pos}), a suitable BiTEDs candidate with cytotoxicity against MUC16^{pos} but not MUC16^{neg} ovarian cancer cell lines was identified (FIG. 3(a)). To minimize the potential that some of the cytotoxicity observed was due to cell line-specific differences, isogenic SKOV3 cell lines modified to express MUC16^{ecto} (Rafiq S et al., Nat Biotechnol. 2018; 36(9):847-56, Chekmasova A A et al., Clin Cancer Res. 2010; 16(14):3594-606) were used. As shown in FIG. 3(b), dose-dependent cytotoxicity at different effector to target ratios was observed.

OVCAR3 cells endogenously express MUC16 and shed high levels of CA-125 (Yin B W et al., Int J Cancer. 2002; 98(5):737-40), making this cell line an ideal candidate to test the hypothesis that MUC16^{ecto}-BiTEDs could mediate cytotoxicity in the presence of shed CA-125. OVCAR3 cells were effectively lysed at various E:T ratios in the presence of MUC16^{ecto}-BiTEDs (FIG. 3(c)). Treatment of wild type SKOV3 (MUC16^{neg}) cells with MUC16^{ecto}-BiTEDs did not result in any significant cytotoxicity compared to incubation with T-cells alone at different E:T ratios (FIG. 3(d)). Taken together, MUC16^{ecto}-BiTEDs exhibit specific cytotoxicity against MUC16^{pos} ovarian cancer cell lines, and this cytotoxicity is preserved in the presence of CA-125. To evaluate the mechanisms of BiTEDs-mediated cytotoxicity, intracellular granzyme-B and cytokine levels in T-cells cultured for 24 hours with MUC16^{pos} ovarian cancer cells with or without BiTEDs were evaluated. Both CD4⁺ and CD8⁺ T-cells showed increased levels of intracellular granzyme-B (FIG. 3(e)). Similarly, intracellular T-cell IL-2 and IFN-γ were significantly elevated in coculture conditions containing MUC16^{ecto}-BiTEDs (FIG. 3(f)).

### Example 6: MUC16^{ecto}-BiTEDs Delay Progression of Metastatic Ovarian Cancer In Vivo

To evaluate the in vivo efficacy of MUC16^{ecto} directed bispecific T-cell engagers, female NSG mice were intraperitoneally (i.p) injected with 3×10⁶ SKOV3 tumor cells modified to express MUC16^{ecto} and luciferase. Seven days after tumor inoculation, mice were either untreated, treated with intravenous (i.v) activated human T-cells alone, or i.v T-cells and MUC16^{ecto}-BiTEDs.

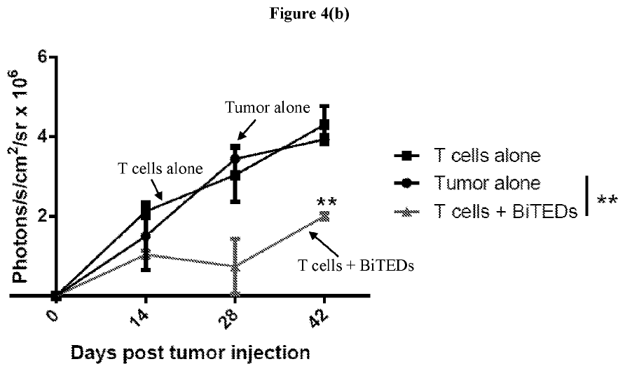

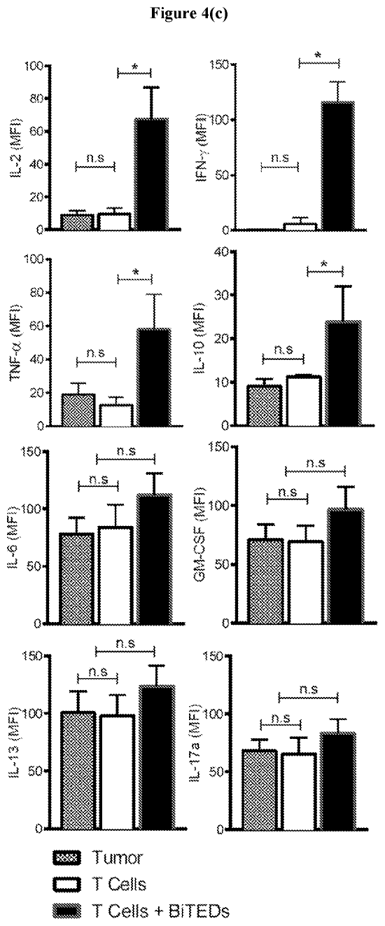

Tumor-bearing mice treated with MUC16^{ecto}-BiTEDs received additional BiTEDs injections on days 9, 11, 14, 16, and 18 for a total of 6 doses over two weeks. Animals were imaged on days 14, 21, 28, and 42 after tumor injection. As shown in FIGS. 4(a)-4(b), MUC16^{ecto}-BiTEDs delayed radiographic progression of disease compared to no treatment or treatment with T-cells alone. Using the same experimental model, serum cytokines in mice treated with BiTEDs were evaluated. Tumor-bearing mice treated with T-cells and BiTEDs showed significantly elevated levels of systemic IL-2, IFN-γ, TNF-α, and IL-10, seven days after treatment (FIG. 4(c)). We did not find any significant elevations in IL-6, GM-CSF, IL-13, or IL-17a levels.

### Example 7: MUC16^{ecto}-BiTEDs Increase Survival as Monotherapy and in Combination with Immune Checkpoint Blockade

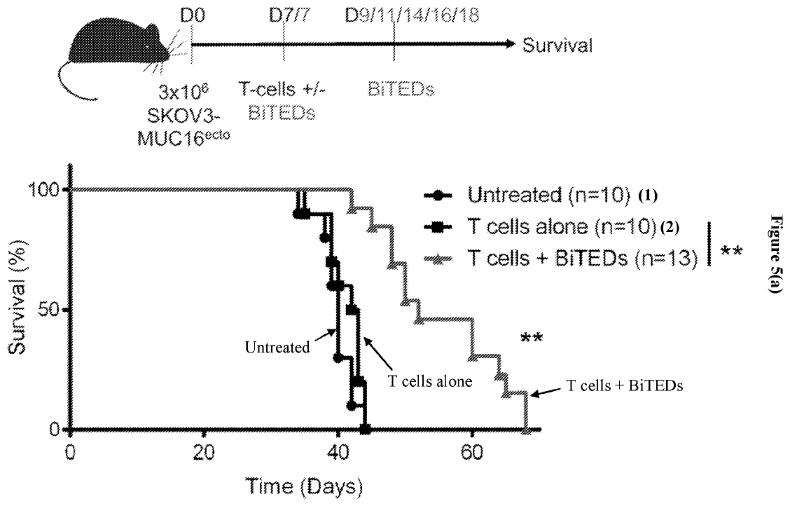

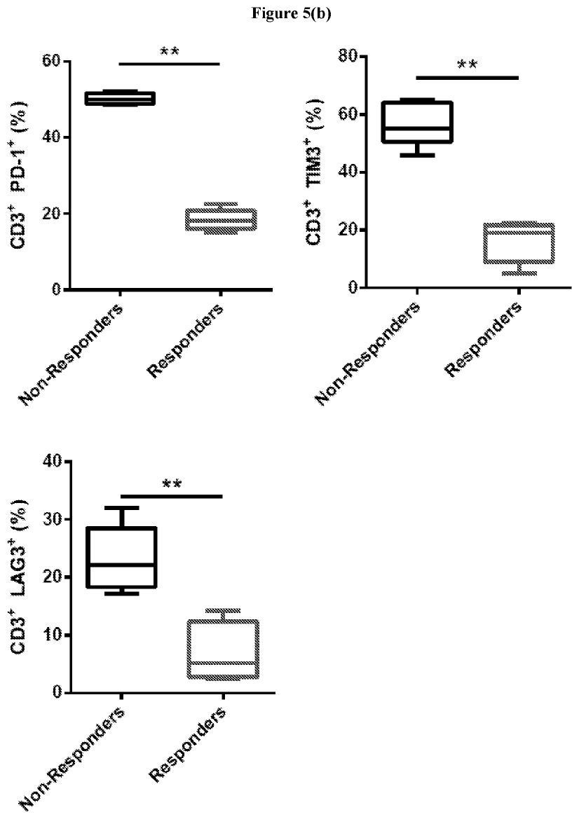

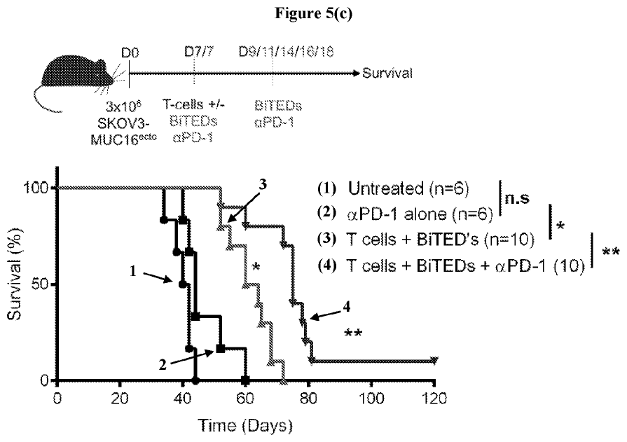

Next, the effects of treatment with MUC16^{ecto}-BiTEDs on the survival of tumor-bearing mice was assessed. As shown in FIG. 5(a), compared to treatment with T-cells alone, the administration of MUC16^{ecto}-BiTEDs led to significantly increased survival in SKOV3-MUC16^{ecto} tumor-bearing mice (median OS; 42.5 days vs. 52 days, **p<0.005).

Since the tumor model used for this experiment had sustained levels of MUC16 expression, immune escape by antigen loss or downregulation is an unlikely mechanism of disease progression. To better understand the potential mechanisms for BiTEDs treatment failure, spleens from BITED-treated animals that succumbed early to disease were harvested and human T-cell phenotypes were compared with responders. Responders were defined as treated animals living beyond 55 days. An increased proportion of CD3+human T-cells expressing PD-1, TIM-3, and LAG3 was found in non-responders compared to animals that responded better to therapy (FIG. 5(b)). These results suggested that some degree of T-cell dysfunction could underlie treatment failure. To test this hypothesis, anti-PD-1 (PD-1) immune checkpoint inhibition by EH12.2H7 (BioLegend, San Diego, CA) was combined with MUC16^{ecto}-BiTEDs. The combination significantly improved survival (median OS; 62 vs. 75 days, **p<0.05), including the proportion of animals surviving beyond 100 days (FIG. 5(c)). Treatment with BiTEDs was superior to αPD-1 therapy alone (median OS; 62 vs. 44 days, **p<0.05).

### Example 8: Inhibition of Tumor-Angiogenesis Synergistically Improves BiTEDs Immunotherapy

The critical role of angiogenesis in ovarian cancer has been extensively described (Mesiano S et al., Am J Pathol. 1998; 153(4):1249-56). Increased VEGF expression is a poor prognostic indicator in ovarian cancer (Paley P J et al., Cancer 1997; 80(1):98-106), and monoclonal antibodies against VEGF play an essential role in the clinical management of ovarian cancer (Colombo N et al., Crit Rev Oncol Hematol. 2016; 97:335-48). Further, VEGF inhibition has also been shown to reduce ascites, a well-described immunosuppressive tumor microenvironment.

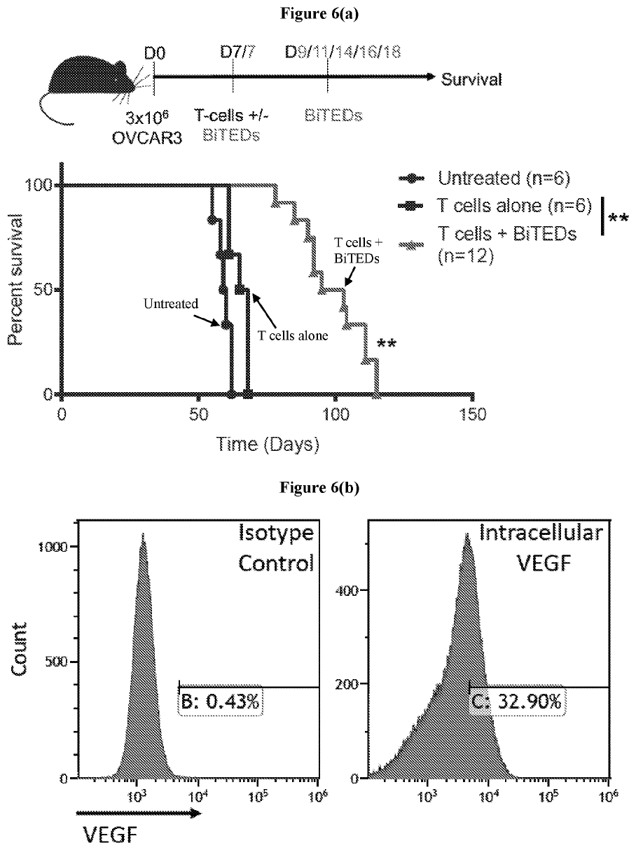

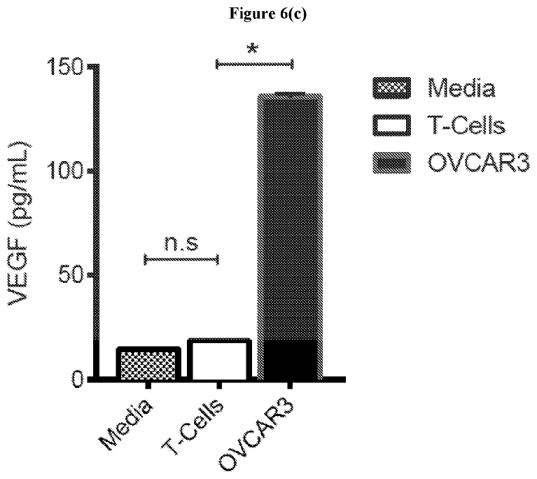

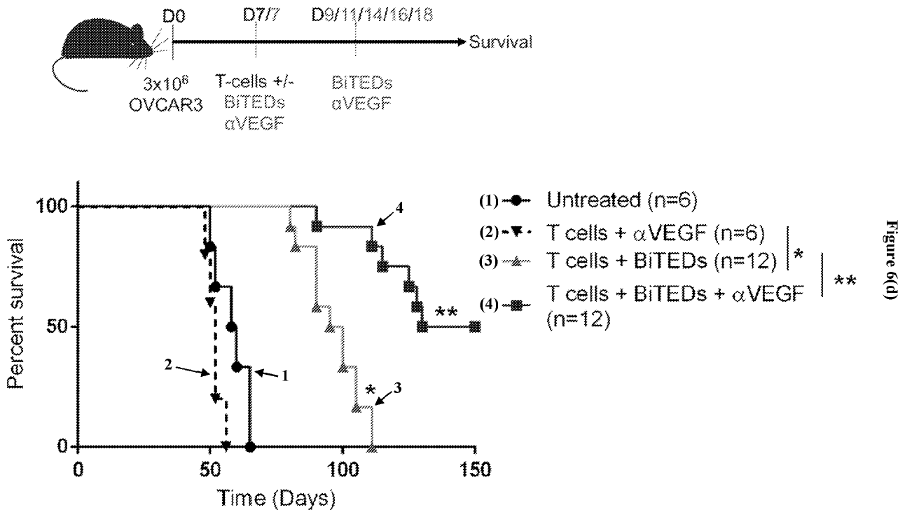

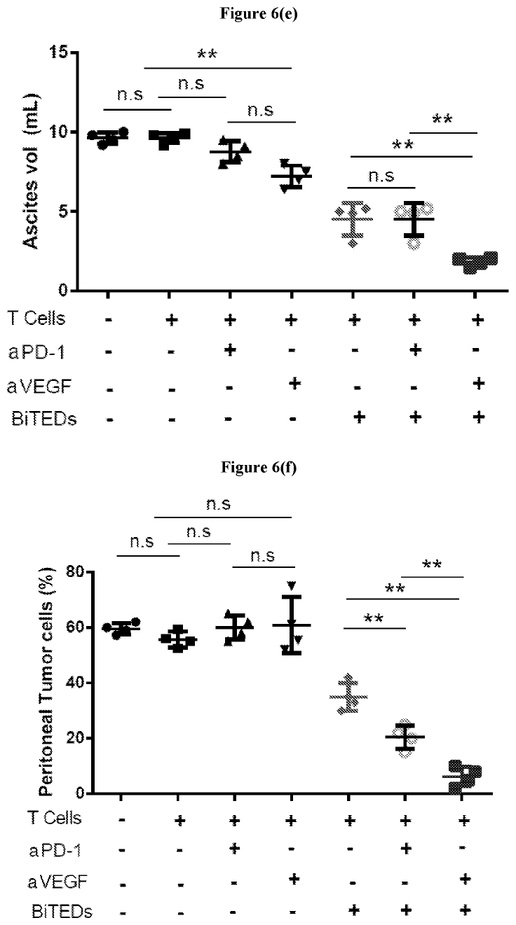

OVCAR3 cells have been shown to secrete VEGF (Bourgeois D L et al., Cancer Cell Int. 2015; 15:112). It was hypothesized that combining MUC16^{ecto}-BiTEDs with anti-VEGF (αVEGF) antibodies may substantially improve efficacy over BiTEDs monotherapy. First, female NSG mice were implanted with OVCAR3 cells. Treatment with MUC16^{ecto}-BiTEDs improved survival over the infusion of T-cells alone (FIG. 6(a)) (median OS; 66.5 vs. 99 days, **p<0.005). Expression of VEGF in the OVCAR3 cells used in these experiments was verified using intracellular flow cytometry (FIG. 6(b)) and ELISA (FIG. 6(c)). Next, OVCAR3 tumor-bearing mice were treated with MUC16^{ecto}-BiTEDs and αVEGF therapy. In combination with VEGF inhibition by hvegf-mab1 (InvivoGen, San Diego, CA), MUC16^{ecto}-BiTEDs led to significant improvements in overall survival over MUC16^{ecto}-BiTEDs treatment alone (median OS; 140 vs. 97.5 days, **p<0.005) (FIG. 6(d)). Treatment with T-cells plus αVEGF was inferior to therapy with MUC16^{ecto}-BiTEDs (median OS; 97.5 vs. 59 days, *p<0.005), suggesting that inhibition of angiogenesis alone is insufficient without concomitant cytotoxic T-cell engagement. Concordantly, significantly decreased ascites was found in mice treated with BiTEDs/αVEGF combination compared to BiTEDs/αPD-1 and BiTEDs monotherapy (FIG. 6(e)). Evaluation of peritoneal tumor cells showed significantly decreased tumor burden with BiTEDs/αVEGF combination compared to BiTEDs/αPD-1 and BiTEDs monotherapy (FIG. 6(f)). Although mice treated with αVEGF plus T cells had decreased ascites (FIG. 6(e)), there was no decrease in peritoneal tumor cells (FIG. 6(f)), demonstrating the lack of survival benefit with this combination (FIG. 6(d)).

These results demonstrate that combination therapy methods with an anti-MUC16×CD3 multispecific (e.g., bispecific) immunoglobulin-related composition that specifically binds to the C-terminal 114 amino acid residues of mature MUC16 (e.g., MUC16^{c114}) and T cells, and a VEGF inhibitor are useful for treating gynecologic cancer in a subject in need thereof.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A method for treating gynecologic cancer in a subject in need thereof, comprising:
administering to the subject an effective amount of an anti-MUC16×CD3 bispecific antibody or antigen binding fragment thereof, and an effective amount of a VEGF inhibitor, wherein the anti-MUC16×CD3 bispecific antibody or antigen binding fragment comprises a first antigen-binding site that specifically binds to a MUC16 polypeptide comprising an MUC16 ectodomain sequence, wherein the MUC16 ectodomain sequence consists of SEQ ID NO: 95.

2. The method of claim 1, wherein the MUC16 polypeptide has the amino acid sequence of SEQ ID NO: 3.

3. The method of claim 1, wherein the first antigen-binding site comprises a heavy chain immunoglobulin variable domain (V_{H}) and a light chain immunoglobulin variable domain (V_{L}), wherein
(a) the V_{H} comprises a V_{H}-CDR1 sequence of SEQ ID NO: 4, a V_{H}-CDR2 sequence of SEQ ID NO: 5, and a V_{H}-CDR3 sequence of SEQ ID NO: 6; and the V_{L} comprises a V_{L}-CDR1 sequence of SEQ ID NO: 7, a V_{L}-CDR2 sequence of SEQ ID NO: 8, and a V_{L}-CDR3 sequence of SEQ ID NO: 9; or
(b) the V_{H} comprises a V_{H}-CDR1 sequence of SEQ ID NO: 10, a V_{H}-CDR2 sequence of SEQ ID NO: 11, and a V_{H}-CDR3 sequence of SEQ ID NO: 12; and the V_{L} comprises a V_{L}-CDR1 sequence of SEQ ID NO: 13, a V_{L}-CDR2 sequence of SEQ ID NO: 14, and a V_{L}-CDR3 sequence of SEQ ID NO: 15; or
(c) the V_{H} comprises a V_{H}-CDR1 sequence of SEQ ID NO: 16, a V_{H}-CDR2 sequence of SEQ ID NO: 17, and a V_{H}-CDR3 sequence of SEQ ID NO: 18; and the V_{L} comprises a V_{L}-CDR1 sequence of SEQ ID NO: 19, a V_{L}-CDR2 sequence of SEQ ID NO: 20, and a V_{L}-CDR3 sequence of SEQ ID NO: 21; or
(d) the V_{H} comprises a V_{H}-CDR1 sequence of SEQ ID NO: 22, a V_{H}-CDR2 sequence of SEQ ID NO: 23, and a V_{H}-CDR3 sequence of SEQ ID NO: 24; and the V_{L} comprises a V_{L}-CDR1 sequence of SEQ ID NO: 25, a V_{L}-CDR2 sequence of SEQ ID NO: 26, and a V_{L}-CDR3 sequence of SEQ ID NO: 27.

4. The method of claim 1, wherein the first antigen-binding site comprises
(a) a heavy chain immunoglobulin variable domain (V_{H}) comprising a V_{H}-CDR1 sequence, a V_{H}-CDR2 sequence, and a V_{H}-CDR3 sequence of SEQ ID NO: 28 or SEQ ID NO: 29 and a light chain immunoglobulin variable domain (V_{L}) comprising a V_{L}-CDR1 sequence, a V_{L}-CDR2 sequence, and a V_{L}-CDR3 sequence of SEQ ID NO: 30 or SEQ ID NO: 31; or
(b) a heavy chain immunoglobulin variable domain (V_{H}) comprising a V_{H}-CDR1 sequence, a V_{H}-CDR2 sequence, and a V_{H}-CDR3 sequence of SEQ ID NO: 32 or SEQ ID NO: 33 and a light chain immunoglobulin variable domain (V_{L}) comprising a V_{L}-CDR1 sequence, a V_{L}-CDR2 sequence, and a V_{L}-CDR3 sequence of SEQ ID NO: 34 or SEQ ID NO: 35; or
(c) a heavy chain immunoglobulin variable domain (V_{H}) comprising a V_{H}-CDR1 sequence, a V_{H}-CDR2 sequence, and a V_{H}-CDR3 sequence of SEQ ID NO: 36 and a light chain immunoglobulin variable domain (V_{L}) comprising a V_{L}-CDR1 sequence, a V_{L}-CDR2 sequence, and a V_{L}-CDR3 sequence of SEQ ID NO: 37; or
(d) a heavy chain immunoglobulin variable domain (V_{H}) comprising a V_{H}-CDR1 sequence, a V_{H}-CDR2 sequence, and a V_{H}-CDR3 sequence of SEQ ID NO: 38 and a light chain immunoglobulin variable domain (V_{L}) comprising a V_{L}-CDR1 sequence, a V_{L}-CDR2 sequence, and a V_{L}-CDR3 sequence of SEQ ID NO: 39.

5. The method of claim 1, wherein the first antigen-binding site comprises
(a) a heavy chain immunoglobulin variable domain (V_{H}) comprising the amino acid sequence of SEQ ID NO: 28 or SEQ ID NO: 29 and a light chain immunoglobulin variable domain (V_{L}) comprising the amino acid sequence of SEQ ID NO: 30 or SEQ ID NO: 31; or
(b) a heavy chain immunoglobulin variable domain (V_{H}) comprising the amino acid sequence of SEQ ID NO: 32 or SEQ ID NO: 33 and a light chain immunoglobulin variable domain (V_{L}) comprising the amino acid sequence of SEQ ID NO: 34 or SEQ ID NO: 35; or
(c) a heavy chain immunoglobulin variable domain (V_{H}) comprising the amino acid sequence of SEQ ID NO: 36 and a light chain immunoglobulin variable domain (V_{L}) comprising the amino acid sequence of SEQ ID NO: 37; or
(d) a heavy chain immunoglobulin variable domain (V_{H}) comprising the amino acid sequence of SEQ ID NO: 38 and a light chain immunoglobulin variable domain (V_{L}) comprising the amino acid sequence of SEQ ID NO: 39.

6. The method of claim 1, wherein the anti-MUC16×CD3 bispecific antibody or antigen binding fragment further comprises a Fc domain of an isotype selected from the group consisting of IgG1, IgG2, IgG3, IgG4, IgA1, IgA2, IgM, IgD, and IgE.

7. The method of claim 1, wherein the anti-MUC16×CD3 bispecific antigen binding fragment is a Fab, a Fab′, a F(ab′)₂, an Fv, or a single chain Fv (scFv).

8. The method of claim 1, wherein the anti-MUC16×CD3 bispecific antibody or antigen binding fragment is human, or humanized.

9. The method of claim 1, wherein the anti-MUC16×CD3 bispecific antibody or antigen binding fragment is a tandem scFv, a diabody (Db), a single chain diabody (scDb), a dual-affinity retargeting (DART) antibody, a F(ab′)₂, a dual variable domain (DVD) antibody, a knob-into-hole (KiH) antibody, a dock and lock (DNL) antibody, a chemically cross-linked antibody, a heteromultimeric antibody, a monoclonal antibody, a full-length antibody, or a heteroconjugate antibody.

10. The method of claim 1, wherein the anti-MUC16×CD3 bispecific antibody or antigen binding fragment comprises a second antigen-binding site that specifically binds to T cells.

11. The method of claim 10, wherein the second antigen-binding site comprises a heavy chain immunoglobulin variable domain (V_{H}) comprising the amino acid sequence of SEQ ID NO: 70 and a light chain immunoglobulin variable domain (V_{L}) comprising the amino acid sequence of SEQ ID NO: 71.

12. The method of claim 10, wherein the second antigen-binding site comprises the amino acid sequence of SEQ ID NO: 72.

13. The method of claim 1, wherein the anti-MUC16×CD3 bispecific antibody or antigen binding fragment comprises the amino acid sequence of any one of SEQ ID NOs: 73-92.

14. The method of claim 1, wherein the VEGF inhibitor is a small molecule inhibitor, a siRNA, an antisense oligonucleotide, a shRNA, a sgRNA, a ribozyme, or an antibody or antigen binding fragment thereof.

15. The method of claim 14, wherein the VEGF inhibitor is bevacizumab, ranibizumab, vanucizumab, brolucizumab, hPV19, IBI305, or VEGF Trap.

16. The method of claim 14, wherein the VEGF inhibitor is selected from the group consisting of linifanib, AEE-788, axitinib (AG-13736), AG-028262, Angiostatin, combretastatin A4, cediranib, sorafenib, thalidomide, vatalanib, DC-101, SNS-032, sunitinib malate, semaxanib, CEP-7055, dovitinib, CP-547632, CP-564959, lenvatinib, pazopanib, GW-654652, tivozanib, benzoylstaurosporine, orantinib, tesevatinib, XL-999, foretinib, vandetanib, and ZK-304709.

17. The method of claim 1, wherein the VEGF inhibitor and the anti-MUC16×CD3 bispecific antibody or antigen binding fragment are administered separately, sequentially, or simultaneously.

18. The method of claim 1, wherein the gynecologic cancer is selected from the group consisting of ovarian cancer, fallopian tube cancer, uterine cancer and endometrial cancer.

19. The method of claim 1, wherein the VEGF inhibitor is administered orally, intranasally, parenterally, intravenously, intramuscularly, intraperitoneally, intramuscularly, intraarterially, subcutaneously, intrathecally, intracapsularly, intraorbitally, intratumorally, intradermally, transtracheally, intracerebroventricularly, or topically.

20. The method of claim 1, wherein the anti-MUC16×CD3 bispecific antibody or antigen binding fragment is administered orally, intranasally, parenterally, intravenously, intramuscularly, intraperitoneally, intramuscularly, intraarterially, subcutaneously, intrathecally, intracapsularly, intraorbitally, intratumorally, intradermally, transtracheally, intracerebroventricularly, or topically.


SEQUENCE LISTING (98 sequences)

SEQ ID NO: 1 - protein, 14507 aa, Homo sapiens
MLKPSGLPGSSSPTRSLMTGSRSTKATPEMDSGLTGATLSPKTSTGAIVVTEHTLPFTSPDKTLASPTSSVVGRTTQSLGVMSSALPESTSRGMTHSEQRTSPSLSPQVNGTPSRNYPATSMVSGLSSPRTRTSSTEGNFTKEASTYTLTVETTSGPVTEKYTVPTETSTTEGDSTETPWDTRYIPVKITSPMKTFADSTASKENAPVSMTPAETTVTDSHTPGRTNPSFGTLYSSFLDLSPKGTPNSRGETSLELILSTTGYPFSSPEPGSAGHSRISTSAPLSSSASVLDNKISETSIFSGQSLTSPLSPGVPEARASTMPNSAIPFSMTLSNAETSAERVRSTISSLGTPSISTKQTAETILTFHAFAETMDIPSTHIAKTLASEWLGSPGTLGGTSTSALTTTSPSTTLVSEETNTHHSTSGKETEGTLNTSMTPLETSAPGEESEMTATLVPTLGFTTLDSKIRSPSQVSSSHPTRELRTTGSTSGRQSSSTAAHGSSDILRATTSSTSKASSWTSESTAQQFSEPQHTQWVETSPSMKTERPPASTSVAAPITTSVPSVVSGFTTLKTSSTKGIWLEETSADTLIGESTAGPTTHQFAVPTGISMTGGSSTRGSQGTTHLLTRATASSETSADLTLATNGVPVSVSPAVSKTAAGSSPPGGTKPSYTMVSSVIPETSSLQSSAFREGTSLGLTPLNTRHPFSSPEPDSAGHTKISTSIPLLSSASVLEDKVSATSTFSHHKATSSITTGTPEISTKTKPSSAVLSSMTLSNAATSPERVRNATSPLTHPSPSGEETAGSVLTLSTSAETTDSPNIHPTGTLTSESSESPSTLSLPSVSGVKTTFSSSTPSTHLFTSGEETEETSNPSVSQPETSVSRVRTTLASTSVPTPVFPTMDTWPTRSAQFSSSHLVSELRATSSTSVTNSTGSALPKISHLTGTATMSQTNRDTFNDSAAPQSTTWPETSPRFKTGLPSATTTVSTSATSLSATVMVSKFTSPATSSMEATSIREPSTTILTTETTNGPGSMAVASTNIPIGKGYITEGRLDTSHLPIGTTASSETSMDFTMAKESVSMSVSPSQSMDAAGSSTPGRTSQFVDTFSDDVYHLTSREITIPRDGTSSALTPQMTATHPPSPDPGSARSTWLGILSSSPSSPTPKVTMSSTFSTQRVTTSMIMDTVETSRWNMPNLPSTTSLTPSNIPTSGAIGKSTLVPLDTPSPATSLEASEGGLPTLSTYPESTNTPSIHLGAHASSESPSTIKLTMASVVKPGSYTPLTFPSIETHIHVSTARMAYSSGSSPEMTAPGETNTGSTWDPTTYITTTDPKDTSSAQVSTPHSVRTLRTTENHPKTESATPAAYSGSPKISSSPNLTSPATKAWTITDTTEHSTQLHYTKLAEKSSGFETQSAPGPVSVVIPTSPTIGSSTLELTSDVPGEPLVLAPSEQTTITLPMATWLSTSLTEEMASTDLDISSPSSPMSTFAIFPPMSTPSHELSKSEADTSAIRNTDSTTLDQHLGIRSLGRTGDLTTVPITPLTTTWTSVIEHSTQAQDTLSATMSPTHVTQSLKDQTSIPASASPSHLTEVYPELGTQGRSSSEATTFWKPSTDTLSREIETGPTNIQSTPPMDNTTTGSSSSGVTLGIAHLPIGTSSPAETSTNMALERRSSTATVSMAGTMGLLVTSAPGRSISQSLGRVSSVLSESTTEGVTDSSKGSSPRLNTQGNTALSSSLEPSYAEGSQMSTSIPLTSSPTTPDVEFIGGSTFWTKEVTTVMTSDISKSSARTESSSATLMSTALGSTENTGKEKLRTASMDLPSPTPSMEVTPWISLTLSNAPNTTDSLDLSHGVHTSSAGTLATDRSLNTGVTRASRLENGSDTSSKSLSMGNSTHTSMTYTEKSEVSSSIHPRPETSAPGAETTLTSTPGNRAISLTLPFSSIPVEEVISTGITSGPDINSAPMTHSPITPPTIVWTSTGTIEQSTQPLHAVSSEKVSVQTQSTPYVNSVAVSASPTHENSVSSGSSTSSPYSSASLESLDSTISRRNAITSWLWDLTTSLPTTTWPSTSLSEALSSGHSGVSNPSSTTTEFPLFSAASTSAAKQRNPETETHGPQNTAASTLNTDASSVTGLSETPVGASISSEVPLPMAITSRSDVSGLTSESTANPSLGTASSAGTKLTRTISLPTSESLVSFRMNKDPWTVSIPLGSHPTTNTETSIPVNSAGPPGLSTVASDVIDTPSDGAESIPTVSFSPSPDTEVTTISHFPEKTTHSFRTISSLTHELTSRVTPIPGDWMSSAMSTKPTGASPSITLGERRTITSAAPTTSPIVLTASFTETSTVSLDNETTVKTSDILDARKTNELPSDSSSSSDLINTSIASSTMDVTKTASISPTSISGMTASSSPSLFSSDRPQVPTSTTETNTATSPSVSSNTYSLDGGSNVGGTPSTLPPFTITHPVETSSALLAWSRPVRTFSTMVSTDTASGENPTSSNSVVTSVPAPGTWTSVGSTTDLPAMGFLKTSPAGEAHSLLASTIEPATAFTPHLSAAVVTGSSATSEASLLTTSESKAIHSSPQTPTTPTSGANWETSATPESLLVVTETSDTTLTSKILVTDTILFSTVSTPPSKFPSTGTLSGASFPTLLPDTPAIPLTATEPTSSLATSFDSTPLVTIASDSLGTVPETTLTMSETSNGDALVLKTVSNPDRSIPGITIQGVTESPLHPSSTSPSKIVAPRNTTYEGSITVALSTLPAGTTGSLVFSQSSENSETTALVDSSAGLERASVMPLTTGSQGMASSGGIRSGSTHSTGTKTFSSLPLTMNPGEVTAMSEITTNRLTATQSTAPKGIPVKPTSAESGLLTPVSASSSPSKAFASLTTAPPTWGIPQSTLTFEFSEVPSLDTKSASLPTPGQSLNTIPDSDASTASSSLSKSPEKNPRARMMTSTKAISASSFQSTGFTETPEGSASPSMAGHEPRVPTSGTGDPRYASESMSYPDPSKASSAMTSTSLASKLTTLFSTGQAARSGSSSSPISLSTEKETSFLSPTASTSRKTSLFLGPSMARQPNILVHLQTSALTLSPTSTLNMSQEEPPELTSSQTIAEEEGTTAETQTLTFTPSETPTSLLPVSSPTEPTARRKSSPETWASSISVPAKTSLVETTDGTLVTTIKMSSQAAQGNSTWPAPAEETGSSPAGTSPGSPEMSTTLKIMSSKEPSISPEIRSTVRNSPWKTPETTVPMETTVEPVTLQSTALGSGSTSISHLPTGTTSPTKSPTENMLATERVSLSPSPPEAWTNLYSGTPGGTRQSLATMSSVSLESPTARSITGTGQQSSPELVSKTTGMEFSMWHGSTGGTTGDTHVSLSTSSNILEDPVTSPNSVSSLTDKSKHKTETWVSTTAIPSTVLNNKIMAAEQQTSRSVDEAYSSTSSWSDQTSGSDITLGASPDVTNTLYITSTAQTTSLVSLPSGDQGITSLTNPSGGKTSSASSVTSPSIGLETLRANVSAVKSDIAPTAGHLSQTSSPAEVSILDVTTAPTPGISTTITTMGTNSISTTTPNPEVGMSTMDSTPATERRTTSTEHPSTWSSTAASDSWTVTDMTSNLKVARSPGTISTMHTTSFLASSTELDSMSTPHGRITVIGTSLVTPSSDASAVKTETSTSERTLSPSDTTASTPISTFSRVQRMSISVPDILSTSWTPSSTEAEDVPVSMVSTDHASTKTDPNTPLSTFLFDSLSTLDWDTGRSLSSATATTSAPQGATTPQELTLETMISPATSQLPFSIGHITSAVTPAAMARSSGVTFSRPDPTSKKAEQTSTQLPTTTSAHPGQVPRSAATTLDVIPHTAKTPDATFQRQGQTALTTEARATSDSWNEKEKSTPSAPWITEMMNSVSEDTIKEVTSSSSVLRTLNTLDINLESGTTSSPSWKSSPYERIAPSESTTDKEAIHPSTNTVETTGWVTSSEHASHSTIPAHSASSKLTSPVVTTSTREQAIVSMSTTTWPESTRARTEPNSFLTIELRDVSPYMDTSSTTQTSIISSPGSTAITKGPRTEITSSKRISSSFLAQSMRSSDSPSEAITRLSNFPAMTESGGMILAMQTSPPGATSLSAPTLDTSATASWTGTPLATTQRFTYSEKTTLFSKGPEDTSQPSPPSVEETSSSSSLVPIHATTSPSNILLTSQGHSPSSTPPVTSVFLSETSGLGKTTDMSRISLEPGTSLPPNLSSTAGEALSTYEASRDTKAIHHSADTAVTNMEATSSEYSPIPGHTKPSKATSPLVTSHIMGDITSSTSVFGSSETTEIETVSSVNQGLQERSTSQVASSATETSTVITHVSSGDATTHVTKTQATFSSGTSISSPHQFITSTNTFTDVSTNPSTSLIMTESSGVTITTQTGPTGAATQGPYLLDTSTMPYLTETPLAVTPDFMQSEKTTLISKGPKDVSWTSPPSVAETSYPSSLTPFLVTTIPPATSTLQGQHTSSPVSATSVLTSGLVKTTDMLNTSMEPVTNSPQNLNNPSNEILATLAATTDIETIHPSINKAVTNMGTASSAHVLHSTLPVSSEPSTATSPMVPASSMGDALASISIPGSETTDIEGEPTSSLTAGRKENSTLQEMNSTTESNIILSNVSVGAITEATKMEVPSFDATFIPTPAQSTKFPDIFSVASSRLSNSPPMTISTHMTTTQTGSSGATSKIPLALDTSTLETSAGTPSVVTEGFAHSKITTAMNNDVKDVSQTNPPFQDEASSPSSQAPVLVTTLPSSVAFTPQWHSTSSPVSMSSVLTSSLVKTAGKVDTSLETVTSSPQSMSNTLDDISVTSAATTDIETTHPSINTVVTNVGTTGSAFESHSTVSAYPEPSKVTSPNVTTSTMEDTTISRSIPKSSKTTRTETETTSSLTPKLRETSISQEITSSTETSTVPYKELTGATTEVSRTDVTSSSSTSFPGPDQSTVSLDISTETNTRLSTSPIMTESAEITITTQTGPHGATSQDTFTMDPSNTTPQAGIHSAMTHGFSQLDVTTLMSRIPQDVSWTSPPSVDKTSSPSSFLSSPAMTTPSLISSTLPEDKLSSPMTSLLTSGLVKITDILRTRLEPVTSSLPNFSSTSDKILATSKDSKDTKEIFPSINTEETNVKANNSGHESHSPALADSETPKATTQMVITTTVGDPAPSTSMPVHGSSETTNIKREPTYFLTPRLRETSTSQESSFPTDTSFLLSKVPTGTITEVSSTGVNSSSKISTPDHDKSTVPPDTFTGEIPRVFTSSIKTKSAEMTITTQASPPESASHSTLPLDTSTTLSQGGTHSTVTQGFPYSEVTTLMGMGPGNVSWMTTPPVEETSSVSSLMSSPAMTSPSPVSSTSPQSIPSSPLPVTALPTSVLVTTTDVLGTTSPESVTSSPPNLSSITHERPATYKDTAHTEAAMHHSTNTAVTNVGTSGSGHKSQSSVLADSETSKATPLMSTTSTLGDTSVSTSTPNISQTNQIQTEPTASLSPRLRESSTSEKTSSTTETNTAFSYVPTGAITQASRTEISSSRTSISDLDRPTIAPDISTGMITRLFTSPIMTKSAEMTVTTQTTTPGATSQGILPWDTSTTLFQGGTHSTVSQGFPHSEITTLRSRTPGDVSWMTTPPVEETSSGFSLMSPSMTSPSPVSSTSPESIPSSPLPVTALLTSVLVTTTNVLGTTSPEPVTSSPPNLSSPTQERLTTYKDTAHTEAMHASMHTNTAVANVGTSISGHESQSSVPADSHTSKATSPMGITFAMGDTSVSTSTPAFFETRIQTESTSSLIPGLRDTRTSEEINTVTETSTVLSEVPTTTTTEVSRTEVITSSRTTISGPDHSKMSPYISTETITRLSTFPFVTGSTEMAITNQTGPIGTISQATLTLDTSSTASWEGTHSPVTQRFPHSEETTTMSRSTKGVSWQSPPSVEETSSPSSPVPLPAITSHSSLYSAVSGSSPTSALPVTSLLTSGRRKTIDMLDTHSELVTSSLPSASSFSGEILTSEASTNTETIHFSENTAETNMGTTNSMHKLHSSVSIHSQPSGHTPPKVTGSMMEDAIVSTSTPGSPETKNVDRDSTSPLTPELKEDSTALVMNSTTESNTVFSSVSLDAATEVSRAEVTYYDPTFMPASAQSTKSPDISPEASSSHSNSPPLTISTHKTIATQTGPSGVTSLGQLTLDTSTIATSAGTPSARTQDFVDSETTSVMNNDLNDVLKTSPFSAEEANSLSSQAPLLVTTSPSPVTSTLQEHSTSSLVSVTSVPTPTLAKITDMDTNLEPVTRSPQNLRNTLATSEATTDTHTMHPSINTAVANVGTTSSPNEFYFTVSPDSDPYKATSAVVITSTSGDSIVSTSMPRSSAMKKIESETTFSLIFRLRETSTSQKIGSSSDTSTVFDKAFTAATTEVSRTELTSSSRTSIQGTEKPTMSPDTSTRSVTMLSTFAGLTKSEERTIATQTGPHRATSQGTLTWDTSITTSQAGTHSAMTHGFSQLDLSTLTSRVPEYISGTSPPSVEKTSSSSSLLSLPAITSPSPVPTTLPESRPSSPVHLTSLPTSGLVKTTDMLASVASLPPNLGSTSHKIPTTSEDIKDTEKMYPSTNIAVTNVGTTTSEKESYSSVPAYSEPPKVTSPMVTSFNIRDTIVSTSMPGSSEITRIEMESTFSLAHGLKGTSTSQDPIVSTEKSAVLHKLTTGATETSRTEVASSRRTSIPGPDHSTESPDISTEVIPSLPISLGITESSNMTIITRTGPPLGSTSQGTFTLDTPTTSSRAGTHSMATQEFPHSEMTTVMNKDPEILSWTIPPSIEKTSFSSSLMPSPAMTSPPVSSTLPKTIHTTPSPMTSLLTPSLVMTTDTLGTSPEPTTSSPPNLSSTSHEILTTDEDTTAIEAMHPSTSTAATNVETTSSGHGSQSSVLADSEKTKATAPMDTTSTMGHTTVSTSMSVSSETTKIKRESTYSLTPGLRETSISQNASFSTDTSIVLSEVPTGTTAEVSRTEVTSSGRTSIPGPSQSTVLPEISTRTMTRLFASPTMTESAEMTIPTQTGPSGSTSQDTLTLDTSTTKSQAKTHSTLTQRFPHSEMTTLMSRGPGDMSWQSSPSLENPSSLPSLLSLPATTSPPPISSTLPVTISSSPLPVTSLLTSSPVTTTDMLHTSPELVTSSPPKLSHTSDERLTTGKDTTNTEAVHPSTNTAASNVEIPSSGHESPSSALADSETSKATSPMFITSTQEDTTVAISTPHFLETSRIQKESISSLSPKLRETGSSVETSSAIETSAVLSEVSIGATTEISRTEVTSSSRTSISGSAESTMLPEISTTRKIIKFPTSPILAESSEMTIKTQTSPPGSTSESTFTLDTSTTPSLVITHSTMTQRLPHSEITTLVSRGAGDVPRPSSLPVEETSPPSSQLSLSAMISPSPVSSTLPASSHSSSASVTSLLTPGQVKTTEVLDASAEPETSSPPSLSSTSVEILATSEVTTDTEKIHPFSNTAVTKVGTSSSGHESPSSVLPDSETTKATSAMGTISIMGDTSVSTLTPALSNTRKIQSEPASSLTTRLRETSTSEETSLATEANTVLSKVSTGATTEVSRTEAISFSRTSMSGPEQSTMSQDISIGTIPRISASSVLTESAKMTITTQTGPSESTLESTLNLNTATTPSWVETHSIVIQGFPHPEMTTSMGRGPGGVSWPSPPFVKETSPPSSPLSLPAVTSPHPVSTTFLAHIPPSPLPVTSLLTSGPATTTDILGTSTEPGTSSSSSLSTTSHERLTTYKDTAHTEAVHPSTNTGGTNVATTSSGYKSQSSVLADSSPMCTTSTMGDTSVLTSTPAFLETRRIQTELASSLTPGLRESSGSEGTSSGTKMSTVLSKVPTGATTEISKEDVTSIPGPAQSTISPDISTRTVSWFSTSPVMTESAEITMNTHTSPLGATTQGTSTLDTSSTTSLTMTHSTISQGFSHSQMSTLMRRGPEDVSWMSPPLLEKTRPSFSLMSSPATTSPSPVSSTLPESISSSPLPVTSLLTSGLAKTTDMLHKSSEPVTNSPANLSSTSVEILATSEVTTDTEKTHPSSNRTVTDVGTSSSGHESTSFVLADSQTSKVTSPMVITSTMEDTSVSTSTPGFFETSRIQTEPTSSLTLGLRKTSSSEGTSLATEMSTVLSGVPTGATAEVSRTEVTSSSRTSISGFAQLTVSPETSTETITRLPTSSIMTESAEMMIKTQTDPPGSTPESTHTVDISTTPNWVETHSTVTQRFSHSEMTTLVSRSPGDMLWPSQSSVEETSSASSLLSLPATTSPSPVSSTLVEDFPSASLPVTSLLNPGLVITTDRMGISREPGTSSTSNLSSTSHERLTTLEDTVDTEDMQPSTHTAVTNVRTSISGHESQSSVLSDSETPKATSPMGTTYTMGETSVSISTSDFFETSRIQIEPTSSLTSGLRETSSSERISSATEGSTVLSEVPSGATTEVSRTEVISSRGTSMSGPDQFTISPDISTEAITRLSTSPIMTESAESAITIETGSPGATSEGTLTLDTSTTTFWSGTHSTASPGFSHSEMTTLMSRTPGDVPWPSLPSVEEASSVSSSLSSPAMTSTSFFSTLPESISSSPHPVTALLTLGPVKTTDMLRTSSEPETSSPPNLSSTSAEILATSEVTKDREKIHPSSNTPVVNVGTVIYKHLSPSSVLADLVTTKPTSPMATTSTLGNTSVSTSTPAFPETMMTQPTSSLTSGLREISTSQETSSATERSASLSGMPTGATTKVSRTEALSLGRTSTPGPAQSTISPEISTETITRISTPLTTTGSAEMTITPKTGHSGASSQGTFTLDTSSRASWPGTHSAATHRSPHSGMTTPMSRGPEDVSWPSRPSVEKTSPPSSLVSLSAVTSPSPLYSTPSESSHSSPLRVTSLFTPVMMKTTDMLDTSLEPVTTSPPSMNITSDESLATSKATMETEAIQLSENTAVTQMGTISARQEFYSSYPGLPEPSKVTSPVVTSSTIKDIVSTTIPASSEITRIEMESTSTLTPTPRETSTSQEIHSATKPSTVPYKALTSATIEDSMTQVMSSSRGPSPDQSTMSQDISTEVITRLSTSPIKTESTEMTITTQTGSPGATSRGTLTLDTSTTFMSGTHSTASQGFSHSQMTALMSRTPGDVPWLSHPSVEEASSASFSLSSPVMTSSSPVSSTLPDSIHSSSLPVTSLLTSGLVKTTELLGTSSEPETSSPPNLSSTSAEILAITEVTTDTEKLEMTNVVTSGYTHESPSSVLADSVTTKATSSMGITYPTGDTNVLTSTPAFSDTSRIQTKSKLSLTPGLMETSISEETSSATEKSTVLSSVPTGATTEVSRTEAISSSRTSIPGPAQSTMSSDTSMETITRISTPLTRKESTDMAITPKTGPSGATSQGTFTLDSSSTASWPGTHSATTQRFPQSVVTTPMSRGPEDVSWPSPLSVEKNSPPSSLVSSSSVTSPSPLYSTPSGSSHSSPVPVTSLFTSIMMKATDMLDASLEPETTSAPNMNITSDESLAASKATTETEAIHVFENTAASHVETTSATEELYSSSPGFSEPTKVISPVVTSSSIRDNMVSTTMPGSSGITRIEIESMSSLTPGLRETRTSQDITSSTETSTVLYKMPSGATPEVSRTEVMPSSRTSIPGPAQSTMSLDISDEVVTRLSTSPIMTESAEITITTQTGYSLATSQVTLPLGTSMTFLSGTHSTMSQGLSHSEMTNLMSRGPESLSWTSPRFVETTRSSSSLTSLPLTTSLSPVSSTLLDSSPSSPLPVTSLILPGLVKTTEVLDTSSEPKTSSSPNLSSTSVEIPATSEIMTDTEKIHPSSNTAVAKVRTSSSVHESHSSVLADSETTITIPSMGITSAVDDTTVFTSNPAFSETRRIPTEPTFSLTPGFRETSTSEETTSITETSAVLYGVPTSATTEVSMTEIMSSNRIHIPDSDQSTMSPDIITEVITRLSSSSMMSESTQMTITTQKSSPGATAQSTLTLATTTAPLARTHSTVPPRFLHSEMTTLMSRSPENPSWKSSLFVEKTSSSSSLLSLPVTTSPSVSSTLPQSIPSSSFSVTSLLTPGMVKTTDTSTEPGTSLSPNLSGTSVEILAASEVTTDTEKIHPSSSMAVTNVGTTSSGHELYSSVSIHSEPSKATYPVGTPSSMAETSISTSMPANFETTGFEAEPFSHLTSGFRKTNMSLDTSSVTPTNTPSSPGSTHLLQSSKTDFTSSAKTSSPDWPPASQYTEIPVDIITPFNASPSITESTGITSFPESRFTMSVTESTHHLSTDLLPSAETISTGTVMPSLSEAMTSFATTGVPRAISGSGSPFSRTESGPGDATLSTIAESLPSSTPVPFSSSTFTTTDSSTIPALHEITSSSATPYRVDTSLGTESSTTEGRLVMVSTLDTSSQPGRTSSSPILDTRMTESVELGTVTSAYQVPSLSTRLTRTDGIMEHITKIPNEAAHRGTIRPVKGPQTSTSPASPKGLHTGGTKRMETTTTALKTTTTALKTTSRATLTTSVYTPTLGTLTPLNASMQMASTIPTEMMITTPYVFPDVPETTSSLATSLGAETSTALPRTTPSVFNRESETTASLVSRSGAERSPVIQTLDVSSSEPDTTASWVIHPAETIPTVSKTTPNFFHSELDTVSSTATSHGADVSSAIPTNISPSELDALTPLVTISGTDTSTTFPTLTKSPHETETRTTWLTHPAETSSTIPRTIPNFSHHESDATPSIATSPGAETSSAIPIMTVSPGAEDLVTSQVTSSGTDRNMTIPTLTLSPGEPKTIASLVTHPEAQTSSAIPTSTISPAVSRLVTSMVTSLAAKTSTTNRALTNSPGEPATTVSLVTHPAQTSPTVPWTTSIFFHSKSDTTPSMTTSHGAESSSAVPTPTVSTEVPGVVTPLVTSSRAVISTTIPILTLSPGEPETTPSMATSHGEEASSAIPTPTVSPGVPGVVTSLVTSSRAVTSTTIPILTFSLGEPETTPSMATSHGTEAGSAVPTVLPEVPGMVTSLVASSRAVTSTTLPTLTLSPGEPETTPSMATSHGAEASSTVPTVSPEVPGVVTSLVTSSSGVNSTSIPTLILSPGELETTPSMATSHGAEASSAVPTPTVSPGVSGVVTPLVTSSRAVTSTTIPILTLSSSEPETTPSMATSHGVEASSAVLTVSPEVPGMVTSLVTSSRAVTSTTIPTLTISSDEPETTTSLVTHSEAKMISAIPTLAVSPTVQGLVTSLVTSSGSETSAFSNLTVASSQPETIDSWVAHPGTEASSVVPTLTVSTGEPFTNISLVTHPAESSSTLPRTTSRFSHSELDTMPSTVTSPEAESSSAISTTISPGIPGVLTSLVTSSGRDISATFPTVPESPHESEATASWVTHPAVTSTTVPRTTPNYSHSEPDTTPSIATSPGAEATSDFPTITVSPDVPDMVTSQVTSSGTDTSITIPTLTLSSGEPETTTSFITYSETHTSSAIPTLPVSPGASKMLTSLVISSGTDSTTTFPTLTETPYEPETTAIQLIHPAETNTMVPRTTPKFSHSKSDTTLPVAITSPGPEASSAVSTTTISPDMSDLVTSLVPSSGTDTSTTFPTLSETPYEPETTATWLTHPAETSTTVSGTIPNFSHRGSDTAPSMVTSPGVDTRSGVPTTTIPPSIPGVVTSQVTSSATDTSTAIPTLTPSPGEPETTASSATHPGTQTGFTVPIRTVPSSEPDTMASWVTHPPQTSTPVSRTTSSFSHSSPDATPVMATSPRTEASSAVLTTISPGAPEMVTSQITSSGAATSTTVPTLTHSPGMPETTALLSTHPRTETSKTFPASTVFPQVSETTASLTIRPGAETSTALPTQTTSSLFTLLVTGTSRVDLSPTASPGVSAKTAPLSTHPGTETSTMIPTSTLSLGLLETTGLLATSSSAETSTSTLTLTVSPAVSGLSSASITTDKPQTVTSWNTETSPSVTSVGPPEFSRTVTGTTMTLIPSEMPTPPKTSHGEGVSPTTILRTTMVEATNLATTGSSPTVAKTTTTFNTLAGSLFTPLTTPGMSTLASESVTSRTSYNHRSWISTTSSYNRRYWTPATSTPVTSTFSPGISTSSIPSSTAATVPFMVPFTLNFTITNLQYEEDMRHPGSRKFNATERELQGLLKPLFRNSSLEYLYSGCRLASLRPEKDSSATAVDAICTHRPDPEDLGLDRERLYWELSNLTNGIQELGPYTLDRNSLYVNGFTHRSSMPTTSTPGTSTVDVGTSGTPSSSPSPTTAGPLLMPFTLNFTITNLQYEEDMRRTGSRKFNTMESVLQGLLKPLFKNTSVGPLYSGCRLTLLRPEKDGAATGVDAICTHRLDPKSPGLNREQLYWELSKLTNDIEELGPYTLDRNSLYVNGFTHQSSVSTTSTPGTSTVDLRTSGTPSSLSSPTIMAAGPLLVPFTLNFTITNLQYGEDMGHPGSRKFNTTERVLQGLLGPIFKNTSVGPLYSGCRLTSLRSEKDGAATGVDAICIHHLDPKSPGLNRERLYWELSQLTNGIKELGPYTLDRNSLYVNGFTHRTSVPTSSTPGTSTVDLGTSGTPFSLPSPATAGPLLVLFTLNFTITNLKYEEDMHRPGSRKFNTTERVLQTLLGPMFKNTSVGLLYSGCRLTLLRSEKDGAATGVDAICTHRLDPKSPGVDREQLYWELSQLTNGIKELGPYTLDRNSLYVNGFTHWIPVPTSSTPGTSTVDLGSGTPSSLPSPTTAGPLLVPFTLNFTITNLKYEEDMHCPGSRKFNTTERVLQSLLGPMFKNTSVGPLYSGCRLTLLRSEKDGAATGVDAICTHRLDPKSPGVDREQLYWELSQLTNGIKELGPYTLDRNSLYVNGFTHQTSAPNTSTPGTSTVDLGTSGTPSSLPSPTSAGPLLVPFTLNFTITNLQYEEDMHHPGSRKFNTTERVLQGLLGPMFKNTSVGLLYSGCRLTLLRPEKNGAATGMDAICSHRLDPKSPGLNREQLYWELSQLTHGIKELGPYTLDRNSLYVNGFTHRSSVAPTSTPGTSTVDLGTSGTPSSLPSPTTAVPLLVPFTLNFTITNLQYGEDMRHPGSRKFNTTERVLQGLLGPLFKNSSVGPLYSGCRLISLRSEKDGAATGVDAICTHHLNPQSPGLDREQLYWQLSQMTNGIKELGPYTLDRNSLYVNGFTHRSSGLTTSTPWTSTVDLGTSGTPSPVPSPTTTGPLLVPFTLNFTITNLQYEENMGHPGSRKFNITESVLQGLLKPLFKSTSVGPLYSGCRLTLLRPEKDGVATRVDAICTHRPDPKIPGLDRQQLYWELSQLTHSITELGPYTLDRDSLYVNGFTQRSSVPTTSTPGTFTVQPETSETPSSLPGPTATGPVLLPFTLNFTITNLQYEEDMRRPGSRKFNTTERVLQGLLMPLFKNTSVSSLYSGCRLTLLRPEKDGAATRVDAVCTHRPDPKSPGLDRERLYWKLSQLTHGITELGPYTLDRHSLYVNGFTHQSSMTTTRTPDTSTMHLATSRTPASLSGPMTASPLLVLFTINFTITNLRYEENMHHPGSRKFNTTERVLQGLLRPVFKNTSVGPLYSGCRLTLLRPKKDGAATKVDAICTYRPDPKSPGLDREQLYWELSQLTHSITELGPYTLDRDSLYVNGFTQRSSVPTTSIPGTPTVDLGTSGTPVSKPGPSAASPLLVLFTLNFTITNLRYEENMQHPGSRKFNTTERVLQGLLRSLFKSTSVGPLYSGCRLTLLRPEKDGTATGVDAICTHHPDPKSPRLDREQLYWELSQLTHNITELGPYALDNDSLFVNGFTHRSSVSTTSTPGTPTVYLGASKTPASIFGPSAASHLLILFTLNFTITNLRYEENMWPGSRKFNTTERVLQGLLRPLFKNTSVGPLYSGCRLTLLRPEKDGEATGVDAICTHRPDPTGPGLDREQLYLELSQLTHSITELGPYTLDRDSLYVNGFTHRSSVPTTSTGVVSEEPFTLNFTINNLRYMADMGQPGSLKFNITDNVMQHLLSPLFQRSSLGARYTGCRVIALRSVKNGAETRVDLLCTYLQPLSGPGLPIKQVFHELSQQTHGITRLGPYSLDKDSLYLNGYNEPGPDEPPTTPKPATTFLPPLSEATTAMGYHLKTLTLNFTISNLQYSPDMGKGSATFNSTEGVLQHLLRPLFQKSSMGPFYLGCQLISLRPEKDGAATGVDTTCTYHPDPVGPGLDIQQLYWELSQLTHGVTQLGFYVLDRDSLFINGYAPQNLSIRGEYQINFHIVNWNLSNPDPTSSEYITLLRDIQDKVTTLYKGSQLHDTFRFCLVTNLTMDSVLVTVKALFSSNLDPSLVEQVFLDKTLNASFHWLGSTYQLVDIHVTEMESSVYQPTSSSSTQHFYLNFTITNLPYSQDKAQPGTTNYQRNKRNIEDALNQLFRNSSIKSYFSDCQVSTFRSVPNRHHTGVDSLCNFSPLARRVDRVAIYEEFLRMTRNGTQLQNFTLDRSSVLVDGYSPNRNEPLTGNSDLPFWAVILIGLAGLLGVITCLICGVLVTTRRRKKEGEYNVQQQCPGYYQSHLDLEDLQ

SEQ ID NO: 2 - protein, 14447 aa, Artificial Sequence
  Description of Artificial Sequence Synthetic polypeptide
DKTLASPTSSVVGRTTQSLGVMSSALPESTSRGMTHSEQRTSPSLSPQVNGTPSRNYPATSMVSGLSSPRTRTSSTEGNFTKEASTYTLTVETTSGPVTEKYTVPTETSTTEGDSTETPWDTRYIPVKITSPMKTFADSTASKENAPVSMTPAETTVTDSHTPGRTNPSFGTLYSSFLDLSPKGTPNSRGETSLELILSTTGYPFSSPEPGSAGHSRISTSAPLSSSASVLDNKISETSIFSGQSLTSPLSPGVPEARASTMPNSAIPFSMTLSNAETSAERVRSTISSLGTPSISTKQTAETILTFHAFAETMDIPSTHIAKTLASEWLGSPGTLGGTSTSALTTTSPSTTLVSEETNTHHSTSGKETEGTLNTSMTPLETSAPGEESEMTATLVPTLGFTTLDSKIRSPSQVSSSHPTRELRTTGSTSGRQSSSTAAHGSSDILRATTSSTSKASSWTSESTAQQFSEPQHTQWVETSPSMKTERPPASTSVAAPITTSVPSVVSGFTTLKTSSTKGIWLEETSADTLIGESTAGPTTHQFAVPTGISMTGGSSTRGSQGTTHLLTRATASSETSADLTLATNGVPVSVSPAVSKTAAGSSPPGGTKPSYTMVSSVIPETSSLQSSAFREGTSLGLTPLNTRHPFSSPEPDSAGHTKISTSIPLLSSASVLEDKVSATSTFSHHKATSSITTGTPEISTKTKPSSAVLSSMTLSNAATSPERVRNATSPLTHPSPSGEETAGSVLTLSTSAETTDSPNIHPTGTLTSESSESPSTLSLPSVSGVKTTFSSSTPSTHLFTSGEETEETSNPSVSQPETSVSRVRTTLASTSVPTPVFPTMDTWPTRSAQFSSSHLVSELRATSSTSVTNSTGSALPKISHLTGTATMSQTNRDTFNDSAAPQSTTWPETSPRFKTGLPSATTTVSTSATSLSATVMVSKFTSPATSSMEATSIREPSTTILTTETTNGPGSMAVASTNIPIGKGYITEGRLDTSHLPIGTTASSETSMDFTMAKESVSMSVSPSQSMDAAGSSTPGRTSQFVDTFSDDVYHLTSREITIPRDGTSSALTPQMTATHPPSPDPGSARSTWLGILSSSPSSPTPKVTMSSTFSTQRVTTSMIMDTVETSRWNMPNLPSTTSLTPSNIPTSGAIGKSTLVPLDTPSPATSLEASEGGLPTLSTYPESTNTPSIHLGAHASSESPSTIKLTMASVVKPGSYTPLTFPSIETHIHVSTARMAYSSGSSPEMTAPGETNTGSTWDPTTYITTTDPKDTSSAQVSTPHSVRTLRTTENHPKTESATPAAYSGSPKISSSPNLTSPATKAWTITDTTEHSTQLHYTKLAEKSSGFETQSAPGPVSVVIPTSPTIGSSTLELTSDVPGEPLVLAPSEQTTITLPMATWLSTSLTEEMASTDLDISSPSSPMSTFAIFPPMSTPSHELSKSEADTSAIRNTDSTTLDQHLGIRSLGRTGDLTTVPITPLTTTWTSVIEHSTQAQDTLSATMSPTHVTQSLKDQTSIPASASPSHLTEVYPELGTQGRSSSEATTFWKPSTDTLSREIETGPTNIQSTPPMDNTTTGSSSSGVTLGIAHLPIGTSSPAETSTNMALERRSSTATVSMAGTMGLLVTSAPGRSISQSLGRVSSVLSESTTEGVTDSSKGSSPRLNTQGNTALSSSLEPSYAEGSQMSTSIPLTSSPTTPDVEFIGGSTFWTKEVTTVMTSDISKSSARTESSSATLMSTALGSTENTGKEKLRTASMDLPSPTPSMEVTPWISLTLSNAPNTTDSLDLSHGVHTSSAGTLATDRSLNTGVTRASRLENGSDTSSKSLSMGNSTHTSMTYTEKSEVSSSIHPRPETSAPGAETTLTSTPGNRAISLTLPFSSIPVEEVISTGITSGPDINSAPMTHSPITPPTIVWTSTGTIEQSTQPLHAVSSEKVSVQTQSTPYVNSVAVSASPTHENSVSSGSSTSSPYSSASLESLDSTISRRNAITSWLWDLTTSLPTTTWPSTSLSEALSSGHSGVSNPSSTTTEFPLFSAASTSAAKQRNPETETHGPQNTAASTLNTDASSVTGLSETPVGASISSEVPLPMAITSRSDVSGLTSESTANPSLGTASSAGTKLTRTISLPTSESLVSFRMNKDPWTVSIPLGSHPTTNTETSIPVNSAGPPGLSTVASDVIDTPSDGAESIPTVSFSPSPDTEVTTISHFPEKTTHSFRTISSLTHELTSRVTPIPGDWMSSAMSTKPTGASPSITLGERRTITSAAPTTSPIVLTASFTETSTVSLDNETTVKTSDILDARKTNELPSDSSSSSDLINTSIASSTMDVTKTASISPTSISGMTASSSPSLFSSDRPQVPTSTTETNTATSPSVSSNTYSLDGGSNVGGTPSTLPPFTITHPVETSSALLAWSRPVRTFSTMVSTDTASGENPTSSNSVVTSVPAPGTWTSVGSTTDLPAMGFLKTSPAGEAHSLLASTIEPATAFTPHLSAAVVTGSSATSEASLLTTSESKAIHSSPQTPTTPTSGANWETSATPESLLVVTETSDTTLTSKILVTDTILFSTVSTPPSKFPSTGTLSGASFPTLLPDTPAIPLTATEPTSSLATSFDSTPLVTIASDSLGTVPETTLTMSETSNGDALVLKTVSNPDRSIPGITIQGVTESPLHPSSTSPSKIVAPRNTTYEGSITVALSTLPAGTTGSLVFSQSSENSETTALVDSSAGLERASVMPLTTGSQGMASSGGIRSGSTHSTGTKTFSSLPLTMNPGEVTAMSEITTNRLTATQSTAPKGIPVKPTSAESGLLTPVSASSSPSKAFASLTTAPPTWGIPQSTLTFEFSEVPSLDTKSASLPTPGQSLNTIPDSDASTASSSLSKSPEKNPRARMMTSTKAISASSFQSTGFTETPEGSASPSMAGHEPRVPTSGTGDPRYASESMSYPDPSKASSAMTSTSLASKLTTLFSTGQAARSGSSSSPISLSTEKETSFLSPTASTSRKTSLFLGPSMARQPNILVHLQTSALTLSPTSTLNMSQEEPPELTSSQTIAEEEGTTAETQTLTFTPSETPTSLLPVSSPTEPTARRKSSPETWASSISVPAKTSLVETTDGTLVTTIKMSSQAAQGNSTWPAPAEETGSSPAGTSPGSPEMSTTLKIMSSKEPSISPEIRSTVRNSPWKTPETTVPMETTVEPVTLQSTALGSGSTSISHLPTGTTSPTKSPTENMLATERVSLSPSPPEAWTNLYSGTPGGTRQSLATMSSVSLESPTARSITGTGQQSSPELVSKTTGMEFSMWHGSTGGTTGDTHVSLSTSSNILEDPVTSPNSVSSLTDKSKHKTETWVSTTAIPSTVLNNKIMAAEQQTSRSVDEAYSSTSSWSDQTSGSDITLGASPDVTNTLYITSTAQTTSLVSLPSGDQGITSLTNPSGGKTSSASSVTSPSIGLETLRANVSAVKSDIAPTAGHLSQTSSPAEVSILDVTTAPTPGISTTITTMGTNSISTTTPNPEVGMSTMDSTPATERRTTSTEHPSTWSSTAASDSWTVTDMTSNLKVARSPGTISTMHTTSFLASSTELDSMSTPHGRITVIGTSLVTPSSDASAVKTETSTSERTLSPSDTTASTPISTFSRVQRMSISVPDILSTSWTPSSTEAEDVPVSMVSTDHASTKTDPNTPLSTFLFDSLSTLDWDTGRSLSSATATTSAPQGATTPQELTLETMISPATSQLPFSIGHITSAVTPAAMARSSGVTFSRPDPTSKKAEQTSTQLPTTTSAHPGQVPRSAATTLDVIPHTAKTPDATFQRQGQTALTTEARATSDSWNEKEKSTPSAPWITEMMNSVSEDTIKEVTSSSSVLRTLNTLDINLESGTTSSPSWKSSPYERIAPSESTTDKEAIHPSTNTVETTGWVTSSEHASHSTIPAHSASSKLTSPVVTTSTREQAIVSMSTTTWPESTRARTEPNSFLTIELRDVSPYMDTSSTTQTSIISSPGSTAITKGPRTEITSSKRISSSFLAQSMRSSDSPSEAITRLSNFPAMTESGGMILAMQTSPPGATSLSAPTLDTSATASWTGTPLATTQRFTYSEKTTLFSKGPEDTSQPSPPSVEETSSSSSLVPIHATTSPSNILLTSQGHSPSSTPPVTSVFLSETSGLGKTTDMSRISLEPGTSLPPNLSSTAGEALSTYEASRDTKAIHHSADTAVTNMEATSSEYSPIPGHTKPSKATSPLVTSHIMGDITSSTSVFGSSETTEIETVSSVNQGLQERSTSQVASSATETSTVITHVSSGDATTHVTKTQATFSSGTSISSPHQFITSTNTFTDVSTNPSTSLIMTESSGVTITTQTGPTGAATQGPYLLDTSTMPYLTETPLAVTPDFMQSEKTTLISKGPKDVSWTSPPSVAETSYPSSLTPFLVTTIPPATSTLQGQHTSSPVSATSVLTSGLVKTTDMLNTSMEPVTNSPQNLNNPSNEILATLAATTDIETIHPSINKAVTNMGTASSAHVLHSTLPVSSEPSTATSPMVPASSMGDALASISIPGSETTDIEGEPTSSLTAGRKENSTLQEMNSTTESNIILSNVSVGAITEATKMEVPSFDATFIPTPAQSTKFPDIFSVASSRLSNSPPMTISTHMTTTQTGSSGATSKIPLALDTSTLETSAGTPSVVTEGFAHSKITTAMNNDVKDVSQTNPPFQDEASSPSSQAPVLVTTLPSSVAFTPQWHSTSSPVSMSSVLTSSLVKTAGKVDTSLETVTSSPQSMSNTLDDISVTSAATTDIETTHPSINTVVTNVGTTGSAFESHSTVSAYPEPSKVTSPNVTTSTMEDTTISRSIPKSSKTTRTETETTSSLTPKLRETSISQEITSSTETSTVPYKELTGATTEVSRTDVTSSSSTSFPGPDQSTVSLDISTETNTRLSTSPIMTESAEITITTQTGPHGATSQDTFTMDPSNTTPQAGIHSAMTHGFSQLDVTTLMSRIPQDVSWTSPPSVDKTSSPSSFLSSPAMTTPSLISSTLPEDKLSSPMTSLLTSGLVKITDILRTRLEPVTSSLPNFSSTSDKILATSKDSKDTKEIFPSINTEETNVKANNSGHESHSPALADSETPKATTQMVITTTVGDPAPSTSMPVHGSSETTNIKREPTYFLTPRLRETSTSQESSFPTDTSFLLSKVPTGTITEVSSTGVNSSSKISTPDHDKSTVPPDTFTGEIPRVFTSSIKTKSAEMTITTQASPPESASHSTLPLDTSTTLSQGGTHSTVTQGFPYSEVTTLMGMGPGNVSWMTTPPVEETSSVSSLMSSPAMTSPSPVSSTSPQSIPSSPLPVTALPTSVLVTTTDVLGTTSPESVTSSPPNLSSITHERPATYKDTAHTEAAMHHSTNTAVTNVGTSGSGHKSQSSVLADSETSKATPLMSTTSTLGDTSVSTSTPNISQTNQIQTEPTASLSPRLRESSTSEKTSSTTETNTAFSYVPTGAITQASRTEISSSRTSISDLDRPTIAPDISTGMITRLFTSPIMTKSAEMTVTTQTTTPGATSQGILPWDTSTTLFQGGTHSTVSQGFPHSEITTLRSRTPGDVSWMTTPPVEETSSGFSLMSPSMTSPSPVSSTSPESIPSSPLPVTALLTSVLVTTTNVLGTTSPEPVTSSPPNLSSPTQERLTTYKDTAHTEAMHASMHTNTAVANVGTSISGHESQSSVPADSHTSKATSPMGITFAMGDTSVSTSTPAFFETRIQTESTSSLIPGLRDTRTSEEINTVTETSTVLSEVPTTTTTEVSRTEVITSSRTTISGPDHSKMSPYISTETITRLSTFPFVTGSTEMAITNQTGPIGTISQATLTLDTSSTASWEGTHSPVTQRFPHSEETTTMSRSTKGVSWQSPPSVEETSSPSSPVPLPAITSHSSLYSAVSGSSPTSALPVTSLLTSGRRKTIDMLDTHSELVTSSLPSASSFSGEILTSEASTNTETIHFSENTAETNMGTTNSMHKLHSSVSIHSQPSGHTPPKVTGSMMEDAIVSTSTPGSPETKNVDRDSTSPLTPELKEDSTALVMNSTTESNTVFSSVSLDAATEVSRAEVTYYDPTFMPASAQSTKSPDISPEASSSHSNSPPLTISTHKTIATQTGPSGVTSLGQLTLDTSTIATSAGTPSARTQDFVDSETTSVMNNDLNDVLKTSPFSAEEANSLSSQAPLLVTTSPSPVTSTLQEHSTSSLVSVTSVPTPTLAKITDMDTNLEPVTRSPQNLRNTLATSEATTDTHTMHPSINTAVANVGTTSSPNEFYFTVSPDSDPYKATSAVVITSTSGDSIVSTSMPRSSAMKKIESETTFSLIFRLRETSTSQKIGSSSDTSTVFDKAFTAATTEVSRTELTSSSRTSIQGTEKPTMSPDTSTRSVTMLSTFAGLTKSEERTIATQTGPHRATSQGTLTWDTSITTSQAGTHSAMTHGFSQLDLSTLTSRVPEYISGTSPPSVEKTSSSSSLLSLPAITSPSPVPTTLPESRPSSPVHLTSLPTSGLVKTTDMLASVASLPPNLGSTSHKIPTTSEDIKDTEKMYPSTNIAVTNVGTTTSEKESYSSVPAYSEPPKVTSPMVTSFNIRDTIVSTSMPGSSEITRIEMESTFSLAHGLKGTSTSQDPIVSTEKSAVLHKLTTGATETSRTEVASSRRTSIPGPDHSTESPDISTEVIPSLPISLGITESSNMTIITRTGPPLGSTSQGTFTLDTPTTSSRAGTHSMATQEFPHSEMTTVMNKDPEILSWTIPPSIEKTSFSSSLMPSPAMTSPPVSSTLPKTIHTTPSPMTSLLTPSLVMTTDTLGTSPEPTTSSPPNLSSTSHEILTTDEDTTAIEAMHPSTSTAATNVETTSSGHGSQSSVLADSEKTKATAPMDTTSTMGHTTVSTSMSVSSETTKIKRESTYSLTPGLRETSISQNASFSTDTSIVLSEVPTGTTAEVSRTEVTSSGRTSIPGPSQSTVLPEISTRTMTRLFASPTMTESAEMTIPTQTGPSGSTSQDTLTLDTSTTKSQAKTHSTLTQRFPHSEMTTLMSRGPGDMSWQSSPSLENPSSLPSLLSLPATTSPPPISSTLPVTISSSPLPVTSLLTSSPVTTTDMLHTSPELVTSSPPKLSHTSDERLTTGKDTTNTEAVHPSTNTAASNVEIPSSGHESPSSALADSETSKATSPMFITSTQEDTTVAISTPHFLETSRIQKESISSLSPKLRETGSSVETSSAIETSAVLSEVSIGATTEISRTEVTSSSRTSISGSAESTMLPEISTTRKIIKFPTSPILAESSEMTIKTQTSPPGSTSESTFTLDTSTTPSLVITHSTMTQRLPHSEITTLVSRGAGDVPRPSSLPVEETSPPSSQLSLSAMISPSPVSSTLPASSHSSSASVTSLLTPGQVKTTEVLDASAEPETSSPPSLSSTSVEILATSEVTTDTEKIHPFSNTAVTKVGTSSSGHESPSSVLPDSETTKATSAMGTISIMGDTSVSTLTPALSNTRKIQSEPASSLTTRLRETSTSEETSLATEANTVLSKVSTGATTEVSRTEAISFSRTSMSGPEQSTMSQDISIGTIPRISASSVLTESAKMTITTQTGPSESTLESTLNLNTATTPSWVETHSIVIQGFPHPEMTTSMGRGPGGVSWPSPPFVKETSPPSSPLSLPAVTSPHPVSTTFLAHIPPSPLPVTSLLTSGPATTTDILGTSTEPGTSSSSSLSTTSHERLTTYKDTAHTEAVHPSTNTGGTNVATTSSGYKSQSSVLADSSPMCTTSTMGDTSVLTSTPAFLETRRIQTELASSLTPGLRESSGSEGTSSGTKMSTVLSKVPTGATTEISKEDVTSIPGPAQSTISPDISTRTVSWFSTSPVMTESAEITMNTHTSPLGATTQGTSTLDTSSTTSLTMTHSTISQGFSHSQMSTLMRRGPEDVSWMSPPLLEKTRPSFSLMSSPATTSPSPVSSTLPESISSSPLPVTSLLTSGLAKTTDMLHKSSEPVTNSPANLSSTSVEILATSEVTTDTEKTHPSSNRTVTDVGTSSSGHESTSFVLADSQTSKVTSPMVITSTMEDTSVSTSTPGFFETSRIQTEPTSSLTLGLRKTSSSEGTSLATEMSTVLSGVPTGATAEVSRTEVTSSSRTSISGFAQLTVSPETSTETITRLPTSSIMTESAEMMIKTQTDPPGSTPESTHTVDISTTPNWVETHSTVTQRFSHSEMTTLVSRSPGDMLWPSQSSVEETSSASSLLSLPATTSPSPVSSTLVEDFPSASLPVTSLLNPGLVITTDRMGISREPGTSSTSNLSSTSHERLTTLEDTVDTEDMQPSTHTAVTNVRTSISGHESQSSVLSDSETPKATSPMGTTYTMGETSVSISTSDFFETSRIQIEPTSSLTSGLRETSSSERISSATEGSTVLSEVPSGATTEVSRTEVISSRGTSMSGPDQFTISPDISTEAITRLSTSPIMTESAESAITIETGSPGATSEGTLTLDTSTTTFWSGTHSTASPGFSHSEMTTLMSRTPGDVPWPSLPSVEEASSVSSSLSSPAMTSTSFFSTLPESISSSPHPVTALLTLGPVKTTDMLRTSSEPETSSPPNLSSTSAEILATSEVTKDREKIHPSSNTPVVNVGTVIYKHLSPSSVLADLVTTKPTSPMATTSTLGNTSVSTSTPAFPETMMTQPTSSLTSGLREISTSQETSSATERSASLSGMPTGATTKVSRTEALSLGRTSTPGPAQSTISPEISTETITRISTPLTTTGSAEMTITPKTGHSGASSQGTFTLDTSSRASWPGTHSAATHRSPHSGMTTPMSRGPEDVSWPSRPSVEKTSPPSSLVSLSAVTSPSPLYSTPSESSHSSPLRVTSLFTPVMMKTTDMLDTSLEPVTTSPPSMNITSDESLATSKATMETEAIQLSENTAVTQMGTISARQEFYSSYPGLPEPSKVTSPVVTSSTIKDIVSTTIPASSEITRIEMESTSTLTPTPRETSTSQEIHSATKPSTVPYKALTSATIEDSMTQVMSSSRGPSPDQSTMSQDISTEVITRLSTSPIKTESTEMTITTQTGSPGATSRGTLTLDTSTTFMSGTHSTASQGFSHSQMTALMSRTPGDVPWLSHPSVEEASSASFSLSSPVMTSSSPVSSTLPDSIHSSSLPVTSLLTSGLVKTTELLGTSSEPETSSPPNLSSTSAEILAITEVTTDTEKLEMTNVVTSGYTHESPSSVLADSVTTKATSSMGITYPTGDTNVLTSTPAFSDTSRIQTKSKLSLTPGLMETSISEETSSATEKSTVLSSVPTGATTEVSRTEAISSSRTSIPGPAQSTMSSDTSMETITRISTPLTRKESTDMAITPKTGPSGATSQGTFTLDSSSTASWPGTHSATTQRFPQSVVTTPMSRGPEDVSWPSPLSVEKNSPPSSLVSSSSVTSPSPLYSTPSGSSHSSPVPVTSLFTSIMMKATDMLDASLEPETTSAPNMNITSDESLAASKATTETEAIHVFENTAASHVETTSATEELYSSSPGFSEPTKVISPVVTSSSIRDNMVSTTMPGSSGITRIEIESMSSLTPGLRETRTSQDITSSTETSTVLYKMPSGATPEVSRTEVMPSSRTSIPGPAQSTMSLDISDEVVTRLSTSPIMTESAEITITTQTGYSLATSQVTLPLGTSMTFLSGTHSTMSQGLSHSEMTNLMSRGPESLSWTSPRFVETTRSSSSLTSLPLTTSLSPVSSTLLDSSPSSPLPVTSLILPGLVKTTEVLDTSSEPKTSSSPNLSSTSVEIPATSEIMTDTEKIHPSSNTAVAKVRTSSSVHESHSSVLADSETTITIPSMGITSAVDDTTVFTSNPAFSETRRIPTEPTFSLTPGFRETSTSEETTSITETSAVLYGVPTSATTEVSMTEIMSSNRIHIPDSDQSTMSPDIITEVITRLSSSSMMSESTQMTITTQKSSPGATAQSTLTLATTTAPLARTHSTVPPRFLHSEMTTLMSRSPENPSWKSSLFVEKTSSSSSLLSLPVTTSPSVSSTLPQSIPSSSFSVTSLLTPGMVKTTDTSTEPGTSLSPNLSGTSVEILAASEVTTDTEKIHPSSSMAVTNVGTTSSGHELYSSVSIHSEPSKATYPVGTPSSMAETSISTSMPANFETTGFEAEPFSHLTSGFRKTNMSLDTSSVTPTNTPSSPGSTHLLQSSKTDFTSSAKTSSPDWPPASQYTEIPVDIITPFNASPSITESTGITSFPESRFTMSVTESTHHLSTDLLPSAETISTGTVMPSLSEAMTSFATTGVPRAISGSGSPFSRTESGPGDATLSTIAESLPSSTPVPFSSSTFTTTDSSTIPALHEITSSSATPYRVDTSLGTESSTTEGRLVMVSTLDTSSQPGRTSSSPILDTRMTESVELGTVTSAYQVPSLSTRLTRTDGIMEHITKIPNEAAHRGTIRPVKGPQTSTSPASPKGLHTGGTKRMETTTTALKTTTTALKTTSRATLTTSVYTPTLGTLTPLNASMQMASTIPTEMMITTPYVFPDVPETTSSLATSLGAETSTALPRTTPSVFNRESETTASLVSRSGAERSPVIQTLDVSSSEPDTTASWVIHPAETIPTVSKTTPNFFHSELDTVSSTATSHGADVSSAIPTNISPSELDALTPLVTISGTDTSTTFPTLTKSPHETETRTTWLTHPAETSSTIPRTIPNFSHHESDATPSIATSPGAETSSAIPIMTVSPGAEDLVTSQVTSSGTDRNMTIPTLTLSPGEPKTIASLVTHPEAQTSSAIPTSTISPAVSRLVTSMVTSLAAKTSTTNRALTNSPGEPATTVSLVTHPAQTSPTVPWTTSIFFHSKSDTTPSMTTSHGAESSSAVPTPTVSTEVPGVVTPLVTSSRAVISTTIPILTLSPGEPETTPSMATSHGEEASSAIPTPTVSPGVPGVVTSLVTSSRAVTSTTIPILTFSLGEPETTPSMATSHGTEAGSAVPTVLPEVPGMVTSLVASSRAVTSTTLPTLTLSPGEPETTPSMATSHGAEASSTVPTVSPEVPGVVTSLVTSSSGVNSTSIPTLILSPGELETTPSMATSHGAEASSAVPTPTVSPGVSGVVTPLVTSSRAVTSTTIPILTLSSSEPETTPSMATSHGVEASSAVLTVSPEVPGMVTSLVTSSRAVTSTTIPTLTISSDEPETTTSLVTHSEAKMISAIPTLAVSPTVQGLVTSLVTSSGSETSAFSNLTVASSQPETIDSWVAHPGTEASSVVPTLTVSTGEPFTNISLVTHPAESSSTLPRTTSRFSHSELDTMPSTVTSPEAESSSAISTTISPGIPGVLTSLVTSSGRDISATFPTVPESPHESEATASWVTHPAVTSTTVPRTTPNYSHSEPDTTPSIATSPGAEATSDFPTITVSPDVPDMVTSQVTSSGTDTSITIPTLTLSSGEPETTTSFITYSETHTSSAIPTLPVSPGASKMLTSLVISSGTDSTTTFPTLTETPYEPETTAIQLIHPAETNTMVPRTTPKFSHSKSDTTLPVAITSPGPEASSAVSTTTISPDMSDLVTSLVPSSGTDTSTTFPTLSETPYEPETTATWLTHPAETSTTVSGTIPNFSHRGSDTAPSMVTSPGVDTRSGVPTTTIPPSIPGVVTSQVTSSATDTSTAIPTLTPSPGEPETTASSATHPGTQTGFTVPIRTVPSSEPDTMASWVTHPPQTSTPVSRTTSSFSHSSPDATPVMATSPRTEASSAVLTTISPGAPEMVTSQITSSGAATSTTVPTLTHSPGMPETTALLSTHPRTETSKTFPASTVFPQVSETTASLTIRPGAETSTALPTQTTSSLFTLLVTGTSRVDLSPTASPGVSAKTAPLSTHPGTETSTMIPTSTLSLGLLETTGLLATSSSAETSTSTLTLTVSPAVSGLSSASITTDKPQTVTSWNTETSPSVTSVGPPEFSRTVTGTTMTLIPSEMPTPPKTSHGEGVSPTTILRTTMVEATNLATTGSSPTVAKTTTTFNTLAGSLFTPLTTPGMSTLASESVTSRTSYNHRSWISTTSSYNRRYWTPATSTPVTSTFSPGISTSSIPSSTAATVPFMVPFTLNFTITNLQYEEDMRHPGSRKFNATERELQGLLKPLFRNSSLEYLYSGCRLASLRPEKDSSATAVDAICTHRPDPEDLGLDRERLYWELSNLTNGIQELGPYTLDRNSLYVNGFTHRSSMPTTSTPGTSTVDVGTSGTPSSSPSPTTAGPLLMPFTLNFTITNLQYEEDMRRTGSRKFNTMESVLQGLLKPLFKNTSVGPLYSGCRLTLLRPEKDGAATGVDAICTHRLDPKSPGLNREQLYWELSKLTNDIEELGPYTLDRNSLYVNGFTHQSSVSTTSTPGTSTVDLRTSGTPSSLSSPTIMAAGPLLVPFTLNFTITNLQYGEDMGHPGSRKFNTTERVLQGLLGPIFKNTSVGPLYSGCRLTSLRSEKDGAATGVDAICIHHLDPKSPGLNRERLYWELSQLTNGIKELGPYTLDRNSLYVNGFTHRTSVPTSSTPGTSTVDLGTSGTPFSLPSPATAGPLLVLFTLNFTITNLKYEEDMHRPGSRKFNTTERVLQTLLGPMFKNTSVGLLYSGCRLTLLRSEKDGAATGVDAICTHRLDPKSPGVDREQLYWELSQLTNGIKELGPYTLDRNSLYVNGFTHWIPVPTSSTPGTSTVDLGSGTPSSLPSPTTAGPLLVPFTLNFTITNLKYEEDMHCPGSRKFNTTERVLQSLLGPMFKNTSVGPLYSGCRLTLLRSEKDGAATGVDAICTHRLDPKSPGVDREQLYWELSQLTNGIKELGPYTLDRNSLYVNGFTHQTSAPNTSTPGTSTVDLGTSGTPSSLPSPTSAGPLLVPFTLNFTITNLQYEEDMHHPGSRKFNTTERVLQGLLGPMFKNTSVGLLYSGCRLTLLRPEKNGAATGMDAICSHRLDPKSPGLNREQLYWELSQLTHGIKELGPYTLDRNSLYVNGFTHRSSVAPTSTPGTSTVDLGTSGTPSSLPSPTTAVPLLVPFTLNFTITNLQYGEDMRHPGSRKFNTTERVLQGLLGPLFKNSSVGPLYSGCRLISLRSEKDGAATGVDAICTHHLNPQSPGLDREQLYWQLSQMTNGIKELGPYTLDRNSLYVNGFTHRSSGLTTSTPWTSTVDLGTSGTPSPVPSPTTTGPLLVPFTLNFTITNLQYEENMGHPGSRKFNITESVLQGLLKPLFKSTSVGPLYSGCRLTLLRPEKDGVATRVDAICTHRPDPKIPGLDRQQLYWELSQLTHSITELGPYTLDRDSLYVNGFTQRSSVPTTSTPGTFTVQPETSETPSSLPGPTATGPVLLPFTLNFTITNLQYEEDMRRPGSRKFNTTERVLQGLLMPLFKNTSVSSLYSGCRLTLLRPEKDGAATRVDAVCTHRPDPKSPGLDRERLYWKLSQLTHGITELGPYTLDRHSLYVNGFTHQSSMTTTRTPDTSTMHLATSRTPASLSGPMTASPLLVLFTINFTITNLRYEENMHHPGSRKFNTTERVLQGLLRPVFKNTSVGPLYSGCRLTLLRPKKDGAATKVDAICTYRPDPKSPGLDREQLYWELSQLTHSITELGPYTLDRDSLYVNGFTQRSSVPTTSIPGTPTVDLGTSGTPVSKPGPSAASPLLVLFTLNFTITNLRYEENMQHPGSRKFNTTERVLQGLLRSLFKSTSVGPLYSGCRLTLLRPEKDGTATGVDAICTHHPDPKSPRLDREQLYWELSQLTHNITELGPYALDNDSLFVNGFTHRSSVSTTSTPGTPTVYLGASKTPASIFGPSAASHLLILFTLNFTITNLRYEENMWPGSRKFNTTERVLQGLLRPLFKNTSVGPLYSGCRLTLLRPEKDGEATGVDAICTHRPDPTGPGLDREQLYLELSQLTHSITELGPYTLDRDSLYVNGFTHRSSVPTTSTGVVSEEPFTLNFTINNLRYMADMGQPGSLKFNITDNVMQHLLSPLFQRSSLGARYTGCRVIALRSVKNGAETRVDLLCTYLQPLSGPGLPIKQVFHELSQQTHGITRLGPYSLDKDSLYLNGYNEPGPDEPPTTPKPATTFLPPLSEATTAMGYHLKTLTLNFTISNLQYSPDMGKGSATFNSTEGVLQHLLRPLFQKSSMGPFYLGCQLISLRPEKDGAATGVDTTCTYHPDPVGPGLDIQQLYWELSQLTHGVTQLGFYVLDRDSLFINGYAPQNLSIRGEYQINFHIVNWNLSNPDPTSSEYITLLRDIQDKVTTLYKGSQLHDTFRFCLVTNLTMDSVLVTVKALFSSNLDPSLVEQVFLDKTLNASFHWLGSTYQLVDIHVTEMESSVYQPTSSSSTQHFYLNFTITNLPYSQDKAQPGTTNYQRNKRNIEDALNQLFRNSSIKSYFSDCQVSTFRSVPNRHHTGVDSLCNFSPLARRVDRVAIYEEFLRMTRNGTQLQNFTLDRSSVLVDGYSPNRNEPLTGNSDLPFWAVILIGLAGLLGVITCLICGVLVTTRRRKKEGEYNVQQQCPGYYQSHLDLEDLQ

SEQ ID NO: 3 - protein, 114 aa, Artificial Sequence
  Description of Artificial Sequence Synthetic polypeptide
NFSPLARRVDRVAIYEEFLRMTRNGTQLQNFTLDRSSVLVDGYSPNRNEPLTGNSDLPFWAVILIGLAGLLGLITCLICGVLVTTRRRKKEGEYNVQQQCPGYYQSHLDLEDLQ

SEQ ID NO: 4 - protein, 8 aa, Artificial Sequence
  Description of Artificial Sequence Synthetic peptide
GFTFSSYA

SEQ ID NO: 5 - protein, 8 aa, Artificial Sequence
  Description of Artificial Sequence Synthetic peptide
ISSAGGYI

SEQ ID NO: 6 - protein, 16 aa, Artificial Sequence
  Description of Artificial Sequence Synthetic peptide
ARQGFGNYGDYYAMDY

SEQ ID NO: 7 - protein, 12 aa, Artificial Sequence
  Description of Artificial Sequence Synthetic peptide
QSLLNSRTRKNQ

SEQ ID NO: 8 - protein, 3 aa, Artificial Sequence
  Description of Artificial Sequence Synthetic peptide
WAS

SEQ ID NO: 9 - protein, 8 aa, Artificial Sequence
  Description of Artificial Sequence Synthetic peptide
QQSYNLLT

SEQ ID NO: 10 - protein, 10 aa, Artificial Sequence
  Description of Artificial Sequence Synthetic peptide
GFSLSTVGMG

SEQ ID NO: 11 - protein, 8 aa, Artificial Sequence
  Description of Artificial Sequence Synthetic peptide
IWWDDEDK

SEQ ID NO: 12 - protein, 14 aa, Artificial Sequence
  Description of Artificial Sequence Synthetic peptide
TRIGTAQATDALDY

SEQ ID NO: 13 - protein, 11 aa, Artificial Sequence
  Description of Artificial Sequence Synthetic peptide
KSLLHSNGNTY

SEQ ID NO: 14 - protein, 3 aa, Artificial Sequence
  Description of Artificial Sequence Synthetic peptide
YMS

SEQ ID NO: 15 - protein, 9 aa, Artificial Sequence
  Description of Artificial Sequence Synthetic peptide
MQSLEYPLT

SEQ ID NO: 16 - protein, 8 aa, Artificial Sequence
  Description of Artificial Sequence Synthetic peptide
GGSFSGYY

SEQ ID NO: 17 - protein, 7 aa, Artificial Sequence
  Description of Artificial Sequence Synthetic peptide
INHSGST

SEQ ID NO: 18 - protein, 9 aa, Artificial Sequence
  Description of Artificial Sequence Synthetic peptide
ARQSYITDS

SEQ ID NO: 19 - protein, 6 aa, Artificial Sequence
  Description of Artificial Sequence Synthetic peptide
QDVSKW

SEQ ID NO: 20 - protein, 3 aa, Artificial Sequence
  Description of Artificial Sequence Synthetic peptide
AAS

SEQ ID NO: 21 - protein, 9 aa, Artificial Sequence
  Description of Artificial Sequence Synthetic peptide
QQANSFPWT

SEQ ID NO: 22 - protein, 8 aa, Artificial Sequence
  Description of Artificial Sequence Synthetic peptide
GGSFSGYY

SEQ ID NO: 23 - protein, 7 aa, Artificial Sequence
  Description of Artificial Sequence Synthetic peptide
INHSGST

SEQ ID NO: 24 - protein, 14 aa, Artificial Sequence
  Description of Artificial Sequence Synthetic peptide
ARWSPFSYKQMYDY

SEQ ID NO: 25 - protein, 8 aa, Artificial Sequence
  Description of Artificial Sequence Synthetic peptide
RGSIASAY

SEQ ID NO: 26 - protein, 3 aa, Artificial Sequence
  Description of Artificial Sequence Synthetic peptide
EDY

SEQ ID NO: 27 - protein, 10 aa, Artificial Sequence
  Description of Artificial Sequence Synthetic peptide
QSYDDNDHVI

SEQ ID NO: 28 - protein, 123 aa, Artificial Sequence
  Description of Artificial Sequence Synthetic polypeptide
EVKLQESGGGFVKPGGSLRVSCAASGFTFSSYAMSWVRLAPEMRLEWVATISSAGGYIFYSDSVQGRFTISRDNAKNSLHLQMGSLRSGDTAMYYCARQGFGNYGDYYAMDYWGQGTTVTVSS

SEQ ID NO: 29 - protein, 123 aa, Artificial Sequence
  Description of Artificial Sequence Synthetic polypeptide
EVQLVESGGGLVKPGGSLRVSCAASGFTFSSYAMSWVRLAPGKGLEWVATISSAGGYIFYSDSVQGRFTISRDNAKNSLYLQMNSLRAEDTAMYYCARQGFGNYGDYYAMDYWGQGTLVTVSS

SEQ ID NO: 30 - protein, 113 aa, Artificial Sequence
  Description of Artificial Sequence Synthetic polypeptide
DIELTQSPSSLAVSAGERVTMNCKSSQSLLNSRTRKNQLAWYQQKPGQSPELLIYWASTRQSGVPDRFSGSGSGTDFTLTISSVQAEDVAVYYCQQSYNLLTFGPGTKLEIKR

SEQ ID NO: 31 - protein, 113 aa, Artificial Sequence
  Description of Artificial Sequence Synthetic polypeptide
DIVLTQSPDSLAVSLGERVTMNCKSSQSLLNSRTRKNQLAWYQQKPGQSPELLIYWASTRQSGVPDRFSGSGSGTDFTLTISSVQAEDVAVYYCQQSYNLLTFGQGTKLEIKR

SEQ ID NO: 32 - protein, 123 aa, Artificial Sequence
  Description of Artificial Sequence Synthetic polypeptide
QVTLKESGPGILQPTQTLTLTCTFSGFSLSTVGMGVGWSRQPSGKGLEWLAHIWWDDEDKYYNPALKSRLTITKDTSKNQVFLKITNVDTADTATYYCTRIGTAQATDALDYWGQGTLVTVSS

SEQ ID NO: 33 - protein, 123 aa, Artificial Sequence
  Description of Artificial Sequence Synthetic polypeptide
QVTLKESGPTLVKPTQTLTLTCTFSGFSLSTVGMGVGWSRQPSGKGLEWLAHIWWDDEDKYYNPALKSRLTITKDTSKNQVVLTITNVDPVDTATYYCTRIGTAQATDALDYWGQGTLVTVSS

SEQ ID NO: 34 - protein, 113 aa, Artificial Sequence
  Description of Artificial Sequence Synthetic polypeptide
DIVMTQSAPSVPVTPGESVSISCRSSKSLLHSNGNTYLYWFLQKPGQSPQRLIYYMSNLASGVPDRFSGRGSGTDFTLKISRVEAEDVGVYYCMQSLEYPLTFGGGTKLEIKR

SEQ ID NO: 35 - protein, 113 aa, Artificial Sequence
  Description of Artificial Sequence Synthetic polypeptide
DIVMTQSALSLPVTPGEPVSISCRSSKSLLHSNGNTYLYWFLQKPGQSPQRLIYYMSNLASGVPDRFSGSGSGTDFTLKISRVEAEDVGVYYCMQSLEYPLTFGGGTKLEIKR

SEQ ID NO: 36 - protein, 115 aa, Artificial Sequence
  Description of Artificial Sequence Synthetic polypeptide
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARQSYITDSWGQGTLVTVSS

SEQ ID NO: 37 - protein, 108 aa, Artificial Sequence
  Description of Artificial Sequence Synthetic polypeptide
DIQLTQSPSAVSASVGDRVTITCRASQDVSKWLAWYQQKPGKAPRLLISAASGLQSWVPSRFSGSGSGTEFTLSISSLQPEDFATYYCQQANSFPWTFGQGTKVEIKR

SEQ ID NO: 38 - protein, 120 aa, Artificial Sequence
  Description of Artificial Sequence Synthetic polypeptide
QVQLQQWGAGLLKPSETLSLTCAVYGGSFSGYYWSWIRQPPGKGLEWIGEINHSGSTNYNPSLKSRIIMSVDTSKRQFSLKLRSATAADTAVYYCARWSPFSYKQMYDYWGQGTLVTVSS

SEQ ID NO: 39 - protein, 112 aa, Artificial Sequence
  Description of Artificial Sequence Synthetic polypeptide
NFMLTQPHSVSESPGKTVTISCTRSRGSIASAYVQWYQQRPGSAPITVIYEDYERPSEIPDRFSGSIDSSSNSASLTISGLKTEDEADYYCQSYDDNDHVIFGGGTKVTVLG

SEQ ID NO: 40 - protein, 384 aa, Homo sapiens
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 41 - protein, 330 aa, Homo sapiens [sequence omitted]

SEQ ID NO: 42 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 43 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 44 - protein, 452 aa, Homo sapiens
GSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 45 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 46 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 47 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRMAGKPTHVNVSVVMAEVDGTCY

SEQ ID NO: 48 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 105 aa, Homo sapiens
QPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 50 - protein, 244 aa, Artificial Sequence
  Description of Artificial Sequence Synthetic polypeptide
DIQLTQSPSAVSASVGDRVTITCRASQDVSKWLAWYQQKPGKAPRLLISAASGLQSWVPSRFSGSGSGTEFTLSISSLQPEDFATYYCQQANSFPWTFGQGTKVEIKRSRGGGGSGGGGSGGGGSLEMAQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARQSYITDSWGQGTLVTVSS

SEQ ID NO: 51 - protein, 244 aa, Artificial Sequence
  Description of Artificial Sequence Synthetic polypeptide
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARQSYITDSWGQGTLVTVSSSRGGGGSGGGGSGGGGSLEMADIQLTQSPSAVSASVGDRVTITCRASQDVSKWLAWYQQKPGKAPRLLISAASGLQSWVPSRFSGSGSGTEFTLSISSLQPEDFATYYCQQANSFPWTFGQGTKVEIKR

SEQ ID NO: 52 - protein, 253 aa, Artificial Sequence
  Description of Artificial Sequence Synthetic polypeptide
NFMLTQPHSVSESPGKTVTISCTRSRGSIASAYVQWYQQRPGSAPITVIYEDYERPSEIPDRFSGSIDSSSNSASLTISGLKTEDEADYYCQSYDDNDHVIFGGGTKVTVLGSRGGGGSGGGGSGGGGSLEMAQVQLQQWGAGLLKPSETLSLTCAVYGGSFSGYYWSWIRQPPGKGLEWIGEINHSGSTNYNPSLKSRIIMSVDTSKRQFSLKLRSATAADTAVYYCARWSPFSYKQMYDYWGQGTLVTVSS

SEQ ID NO: 53 - protein, 253 aa, Artificial Sequence
  Description of Artificial Sequence Synthetic polypeptide
QVQLQQWGAGLLKPSETLSLTCAVYGGSFSGYYWSWIRQPPGKGLEWIGEINHSGSTNYNPSLKSRIIMSVDTSKRQFSLKLRSATAADTAVYYCARWSPFSYKQMYDYWGQGTLVTVSSSRGGGGSGGGGSGGGGSLEMANFMLTQPHSVSESPGKTVTISCTRSRGSIASAYVQWYQQRPGSAPITVIYEDYERPSEIPDRFSGSIDSSSNSASLTISGLKTEDEADYYCQSYDDNDHVIFGGGTKVTVLG

SEQ ID NO: 54 - protein, 251 aa, Artificial Sequence
  Description of Artificial Sequence Synthetic polypeptide
DIELTQSPSSLAVSAGERVTMNCKSSQSLLNSRTRKNQLAWYQQKPGQSPELLIYWASTRQSGVPDRFSGSGSGTDFTLTISSVQAEDVAVYYCQQSYNLLTFGPGTKLEIKRGGGGSGGGGSGGGGSEVKLQESGGGFVKPGGSLRVSCAASGFTFSSYAMSWVRLAPEMRLEWVATISSAGGYIFYSDSVQGRFTISRDNAKNSLHLQMGSLRSGDTAMYYCARQGFGNYGDYYAMDYWGQGTTVTVSS

SEQ ID NO: 55 - protein, 251 aa, Artificial Sequence
  Description of Artificial Sequence Synthetic polypeptide
DIVLTQSPDSLAVSLGERVTMNCKSSQSLLNSRTRKNQLAWYQQKPGQSPELLIYWASTRQSGVPDRFSGSGSGTDFTLTISSVQAEDVAVYYCQQSYNLLTFGQGTKLEIKRGGGGSGGGGSGGGGSEVKLQESGGGFVKPGGSLRVSCAASGFTFSSYAMSWVRLAPEMRLEWVATISSAGGYIFYSDSVQGRFTISRDNAKNSLHLQMGSLRSGDTAMYYCARQGFGNYGDYYAMDYWGQGTTVTVSS

SEQ ID NO: 56 - protein, 251 aa, Artificial Sequence
  Description of Artificial Sequence Synthetic polypeptide
DIELTQSPSSLAVSAGERVTMNCKSSQSLLNSRTRKNQLAWYQQKPGQSPELLIYWASTRQSGVPDRFSGSGSGTDFTLTISSVQAEDVAVYYCQQSYNLLTFGPGTKLEIKRGGGGSGGGGSGGGGSEVQLVESGGGLVKPGGSLRVSCAASGFTFSSYAMSWVRLAPGKGLEWVATISSAGGYIFYSDSVQGRFTISRDNAKNSLYLQMNSLRAEDTAMYYCARQGFGNYGDYYAMDYWGQGTLVTVSS

SEQ ID NO: 57 - protein, 251 aa, Artificial Sequence
  Description of Artificial Sequence Synthetic polypeptide
DIVLTQSPDSLAVSLGERVTMNCKSSQSLLNSRTRKNQLAWYQQKPGQSPELLIYWASTRQSGVPDRFSGSGSGTDFTLTISSVQAEDVAVYYCQQSYNLLTFGQGTKLEIKRGGGGSGGGGSGGGGSEVQLVESGGGLVKPGGSLRVSCAASGFTFSSYAMSWVRLAPGKGLEWVATISSAGGYIFYSDSVQGRFTISRDNAKNSLYLQMNSLRAEDTAMYYCARQGFGNYGDYYAMDYWGQGTLVTVSS

SEQ ID NO: 58 - protein, 251 aa, Artificial Sequence
  Description of Artificial Sequence Synthetic polypeptide
DIVMTQSAPSVPVTPGESVSISCRSSKSLLHSNGNTYLYWFLQKPGQSPQRLIYYMSNLASGVPDRFSGRGSGTDFTLKISRVEAEDVGVYYCMQSLEYPLTFGGGTKLEIKRGGGGSGGGGSGGGGSQVTLKESGPGILQPTQTLTLTCTFSGFSLSTVGMGVGWSRQPSGKGLEWLAHIWWDDEDKYYNPALKSRLTITKDTSKNQVFLKITNVDTADTATYYCTRIGTAQATDALDYWGQGTLVTVSS

SEQ ID NO: 59 - protein, 251 aa, Artificial Sequence
  Description of Artificial Sequence Synthetic polypeptide
DIVMTQSALSLPVTPGEPVSISCRSSKSLLHSNGNTYLYWFLQKPGQSPQRLIYYMSNLASGVPDRFSGSGSGTDFTLKISRVEAEDVGVYYCMQSLEYPLTFGGGTKLEIKRGGGGSGGGGSGGGGSQVTLKESGPGILQPTQTLTLTCTFSGFSLSTVGMGVGWSRQPSGKGLEWLAHIWWDDEDKYYNPALKSRLTITKDTSKNQVFLKITNVDTADTATYYCTRIGTAQATDALDYWGQGTLVTVSS

SEQ ID NO: 60 - protein, 251 aa, Artificial Sequence
  Description of Artificial Sequence Synthetic polypeptide
DIVMTQSAPSVPVTPGESVSISCRSSKSLLHSNGNTYLYWFLQKPGQSPQRLIYYMSNLASGVPDRFSGRGSGTDFTLKISRVEAEDVGVYYCMQSLEYPLTFGGGTKLEIKRGGGGSGGGGSGGGGSQVTLKESGPTLVKPTQTLTLTCTFSGFSLSTVGMGVGWSRQPSGKGLEWLAHIWWDDEDKYYNPALKSRLTITKDTSKNQVVLTITNVDPVDTATYYCTRIGTAQATDALDYWGQGTLVTVSS

SEQ ID NO: 61 - protein, 251 aa, Artificial Sequence
  Description of Artificial Sequence Synthetic polypeptide
DIVMTQSALSLPVTPGEPVSISCRSSKSLLHSNGNTYLYWFLQKPGQSPQRLIYYMSNLASGVPDRFSGSGSGTDFTLKISRVEAEDVGVYYCMQSLEYPLTFGGGTKLEIKRGGGGSGGGGSGGGGSQVTLKESGPTLVKPTQTLTLTCTFSGFSLSTVGMGVGWSRQPSGKGLEWLAHIWWDDEDKYYNPALKSRLTITKDTSKNQVVLTITNVDPVDTATYYCTRIGTAQATDALDYWGQGTLVTVSS

SEQ ID NO: 62 - protein, 251 aa, Artificial Sequence
  Description of Artificial Sequence Synthetic polypeptide
EVKLQESGGGFVKPGGSLRVSCAASGFTFSSYAMSWVRLAPEMRLEWVATISSAGGYIFYSDSVQGRFTISRDNAKNSLHLQMGSLRSGDTAMYYCARQGFGNYGDYYAMDYWGQGTTVTVSSGGGGSGGGGSGGGGSDIELTQSPSSLAVSAGERVTMNCKSSQSLLNSRTRKNQLAWYQQKPGQSPELLIYWASTRQSGVPDRFSGSGSGTDFTLTISSVQAEDVAVYYCQQSYNLLTFGPGTKLEIKR

SEQ ID NO: 63 - protein, 251 aa, Artificial Sequence
  Description of Artificial Sequence Synthetic polypeptide
EVKLQESGGGFVKPGGSLRVSCAASGFTFSSYAMSWVRLAPEMRLEWVATISSAGGYIFYSDSVQGRFTISRDNAKNSLHLQMGSLRSGDTAMYYCARQGFGNYGDYYAMDYWGQGTTVTVSSGGGGSGGGGSGGGGSDIVLTQSPDSLAVSLGERVTMNCKSSQSLLNSRTRKNQLAWYQQKPGQSPELLIYWASTRQSGVPDRFSGSGSGTDFTLTISSVQAEDVAVYYCQQSYNLLTFGQGTKLEIKR

SEQ ID NO: 64 - protein, 251 aa, Artificial Sequence
  Description of Artificial Sequence Synthetic polypeptide
EVQLVESGGGLVKPGGSLRVSCAASGFTFSSYAMSWVRLAPGKGLEWVATISSAGGYIFYSDSVQGRFTISRDNAKNSLYLQMNSLRAEDTAMYYCARQGFGNYGDYYAMDYWGQGTLVTVSSGGGGSGGGGSGGGGSDIELTQSPSSLAVSAGERVTMNCKSSQSLLNSRTRKNQLAWYQQKPGQSPELLIYWASTRQSGVPDRFSGSGSGTDFTLTISSVQAEDVAVYYCQQSYNLLTFGPGTKLEIKR

SEQ ID NO: 65 - protein, 251 aa, Artificial Sequence
  Description of Artificial Sequence Synthetic polypeptide
EVQLVESGGGLVKPGGSLRVSCAASGFTFSSYAMSWVRLAPGKGLEWVATISSAGGYIFYSDSVQGRFTISRDNAKNSLYLQMNSLRAEDTAMYYCARQGFGNYGDYYAMDYWGQGTLVTVSSGGGGSGGGGSGGGGSDIVLTQSPDSLAVSLGERVTMNCKSSQSLLNSRTRKNQLAWYQQKPGQSPELLIYWASTRQSGVPDRFSGSGSGTDFTLTISSVQAEDVAVYYCQQSYNLLTFGQGTKLEIKR

SEQ ID NO: 66 - protein, 251 aa, Artificial Sequence
  Description of Artificial Sequence Synthetic polypeptide
QVTLKESGPGILQPTQTLTLTCTFSGFSLSTVGMGVGWSRQPSGKGLEWLAHIWWDDEDKYYNPALKSRLTITKDTSKNQVFLKITNVDTADTATYYCTRIGTAQATDALDYWGQGTLVTVSSGGGGSGGGGSGGGGSDIVMTQSAPSVPVTPGESVSISCRSSKSLLHSNGNTYLYWFLQKPGQSPQRLIYYMSNLASGVPDRFSGRGSGTDFTLKISRVEAEDVGVYYCMQSLEYPLTFGGGTKLEIKR

SEQ ID NO: 67 - protein, 251 aa, Artificial Sequence
  Description of Artificial Sequence Synthetic polypeptide
QVTLKESGPGILQPTQTLTLTCTFSGFSLSTVGMGVGWSRQPSGKGLEWLAHIWWDDEDKYYNPALKSRLTITKDTSKNQVFLKITNVDTADTATYYCTRIGTAQATDALDYWGQGTLVTVSSGGGGSGGGGSGGGGSDIVMTQSALSLPVTPGEPVSISCRSSKSLLHSNGNTYLYWFLQKPGQSPQRLIYYMSNLASGVPDRFSGSGSGTDFTLKISRVEAEDVGVYYCMQSLEYPLTFGGGTKLEIKR

SEQ ID NO: 68 - protein, 251 aa, Artificial Sequence
  Description of Artificial Sequence Synthetic polypeptide
QVTLKESGPTLVKPTQTLTLTCTFSGFSLSTVGMGVGWSRQPSGKGLEWLAHIWWDDEDKYYNPALKSRLTITKDTSKNQVVLTITNVDPVDTATYYCTRIGTAQATDALDYWGQGTLVTVSSGGGGSGGGGSGGGGSDIVMTQSAPSVPVTPGESVSISCRSSKSLLHSNGNTYLYWFLQKPGQSPQRLIYYMSNLASGVPDRFSGRGSGTDFTLKISRVEAEDVGVYYCMQSLEYPLTFGGGTKLEIKR

SEQ ID NO: 69 - protein, 251 aa, Artificial Sequence
  Description of Artificial Sequence Synthetic polypeptide
QVTLKESGPTLVKPTQTLTLTCTFSGFSLSTVGMGVGWSRQPSGKGLEWLAHIWWDDEDKYYNPALKSRLTITKDTSKNQVVLTITNVDPVDTATYYCTRIGTAQATDALDYWGQGTLVTVSSGGGGSGGGGSGGGGSDIVMTQSALSLPVTPGEPVSISCRSSKSLLHSNGNTYLYWFLQKPGQSPQRLIYYMSNLASGVPDRFSGSGSGTDFTLKISRVEAEDVGVYYCMQSLEYPLTFGGGTKLEIKR

SEQ ID NO: 70 - protein, 119 aa, Artificial Sequence
  Description of Artificial Sequence Synthetic polypeptide
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSS

SEQ ID NO: 71 - protein, 106 aa, Artificial Sequence
  Description of Artificial Sequence Synthetic polypeptide
DIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 72 - protein, 243 aa, Artificial Sequence
  Description of Artificial Sequence Synthetic polypeptide
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 73 - protein, 507 aa, Artificial Sequence
  Description of Artificial Sequence Synthetic polypeptide
DIELTQSPSSLAVSAGERVTMNCKSSQSLLNSRTRKNQLAWYQQKPGQSPELLIYWASTRQSGVPDRFSGSGSGTDFTLTISSVQAEDVAVYYCQQSYNLLTFGPGTKLEIKRGGGGSGGGGSGGGGSEVKLQESGGGFVKPGGSLRVSCAASGFTFSSYAMSWVRLAPEMRLEWVATISSAGGYIFYSDSVQGRFTISRDNAKNSLHLQMGSLRSGDTAMYYCARQGFGNYGDYYAMDYWGQGTTVTVSSTSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKHHHHHH

SEQ ID NO: 74 - protein, 507 aa, Artificial Sequence
  Description of Artificial Sequence Synthetic polypeptide
DIVLTQSPDSLAVSLGERVTMNCKSSQSLLNSRTRKNQLAWYQQKPGQSPELLIYWASTRQSGVPDRFSGSGSGTDFTLTISSVQAEDVAVYYCQQSYNLLTFGQGTKLEIKRGGGGSGGGGSGGGGSEVKLQESGGGFVKPGGSLRVSCAASGFTFSSYAMSWVRLAPEMRLEWVATISSAGGYIFYSDSVQGRFTISRDNAKNSLHLQMGSLRSGDTAMYYCARQGFGNYGDYYAMDYWGQGTTVTVSSTSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKHHHHHH

SEQ ID NO: 75 - protein, 507 aa, Artificial Sequence
  Description of Artificial Sequence Synthetic polypeptide
DIELTQSPSSLAVSAGERVTMNCKSSQSLLNSRTRKNQLAWYQQKPGQSPELLIYWASTRQSGVPDRFSGSGSGTDFTLTISSVQAEDVAVYYCQQSYNLLTFGPGTKLEIKRGGGGSGGGGSGGGGSEVQLVESGGGLVKPGGSLRVSCAASGFTFSSYAMSWVRLAPGKGLEWVATISSAGGYIFYSDSVQGRFTISRDNAKNSLYLQMNSLRAEDTAMYYCARQGFGNYGDYYAMDYWGQGTLVTVSSTSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKHHHHHH

SEQ ID NO: 76 - protein, 507 aa, Artificial Sequence
  Description of Artificial Sequence Synthetic polypeptide
DIVLTQSPDSLAVSLGERVTMNCKSSQSLLNSRTRKNQLAWYQQKPGQSPELLIYWASTRQSGVPDRFSGSGSGTDFTLTISSVQAEDVAVYYCQQSYNLLTFGQGTKLEIKRGGGGSGGGGSGGGGSEVQLVESGGGLVKPGGSLRVSCAASGFTFSSYAMSWVRLAPGKGLEWVATISSAGGYIFYSDSVQGRFTISRDNAKNSLYLQMNSLRAEDTAMYYCARQGFGNYGDYYAMDYWGQGTLVTVSSTSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKHHHHHH

SEQ ID NO: 77 - protein, 507 aa, Artificial Sequence
  Description of Artificial Sequence Synthetic polypeptide
DIVMTQSAPSVPVTPGESVSISCRSSKSLLHSNGNTYLYWFLQKPGQSPQRLIYYMSNLASGVPDRFSGRGSGTDFTLKISRVEAEDVGVYYCMQSLEYPLTFGGGTKLEIKRGGGGSGGGGSGGGGSQVTLKESGPGILQPTQTLTLTCTFSGFSLSTVGMGVGWSRQPSGKGLEWLAHIWWDDEDKYYNPALKSRLTITKDTSKNQVFLKITNVDTADTATYYCTRIGTAQATDALDYWGQGTLVTVSSTSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKHHHHHH

SEQ ID NO: 78 - protein, 507 aa, Artificial Sequence
  Description of Artificial Sequence Synthetic polypeptide
DIVMTQSALSLPVTPGEPVSISCRSSKSLLHSNGNTYLYWFLQKPGQSPQRLIYYMSNLASGVPDRFSGSGSGTDFTLKISRVEAEDVGVYYCMQSLEYPLTFGGGTKLEIKRGGGGSGGGGSGGGGSQVTLKESGPGILQPTQTLTLTCTFSGFSLSTVGMGVGWSRQPSGKGLEWLAHIWWDDEDKYYNPALKSRLTITKDTSKNQVFLKITNVDTADTATYYCTRIGTAQATDALDYWGQGTLVTVSSTSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKHHHHHH

SEQ ID NO: 79 - protein, 507 aa, Artificial Sequence
  Description of Artificial Sequence Synthetic polypeptide
DIVMTQSAPSVPVTPGESVSISCRSSKSLLHSNGNTYLYWFLQKPGQSPQRLIYYMSNLASGVPDRFSGRGSGTDFTLKISRVEAEDVGVYYCMQSLEYPLTFGGGTKLEIKRGGGGSGGGGSGGGGSQVTLKESGPTLVKPTQTLTLTCTFSGFSLSTVGMGVGWSRQPSGKGLEWLAHIWWDDEDKYYNPALKSRLTITKDTSKNQVVLTITNVDPVDTATYYCTRIGTAQATDALDYWGQGTLVTVSSTSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKHHHHHH

SEQ ID NO: 80 - protein, 507 aa, Artificial Sequence
  Description of Artificial Sequence Synthetic polypeptide
DIVMTQSALSLPVTPGEPVSISCRSSKSLLHSNGNTYLYWFLQKPGQSPQRLIYYMSNLASGVPDRFSGSGSGTDFTLKISRVEAEDVGVYYCMQSLEYPLTFGGGTKLEIKRGGGGSGGGGSGGGGSQVTLKESGPTLVKPTQTLTLTCTFSGFSLSTVGMGVGWSRQPSGKGLEWLAHIWWDDEDKYYNPALKSRLTITKDTSKNQVVLTITNVDPVDTATYYCTRIGTAQATDALDYWGQGTLVTVSSTSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKHHHHHH

SEQ ID NO: 81 - protein, 507 aa, Artificial Sequence
  Description of Artificial Sequence Synthetic polypeptide
EVKLQESGGGFVKPGGSLRVSCAASGFTFSSYAMSWVRLAPEMRLEWVATISSAGGYIFYSDSVQGRFTISRDNAKNSLHLQMGSLRSGDTAMYYCARQGFGNYGDYYAMDYWGQGTTVTVSSGGGGSGGGGSGGGGSDIELTQSPSSLAVSAGERVTMNCKSSQSLLNSRTRKNQLAWYQQKPGQSPELLIYWASTRQSGVPDRFSGSGSGTDFTLTISSVQAEDVAVYYCQQSYNLLTFGPGTKLEIKRTSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKHHHHHH

SEQ ID NO: 82 - protein, 507 aa, Artificial Sequence
  Description of Artificial Sequence Synthetic polypeptide
EVKLQESGGGFVKPGGSLRVSCAASGFTFSSYAMSWVRLAPEMRLEWVATISSAGGYIFYSDSVQGRFTISRDNAKNSLHLQMGSLRSGDTAMYYCARQGFGNYGDYYAMDYWGQGTTVTVSSGGGGSGGGGSGGGGSDIVLTQSPDSLAVSLGERVTMNCKSSQSLLNSRTRKNQLAWYQQKPGQSPELLIYWASTRQSGVPDRFSGSGSGTDFTLTISSVQAEDVAVYYCQQSYNLLTFGQGTKLEIKRTSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKHHHHHH

SEQ ID NO: 83 - protein, 507 aa, Artificial Sequence
  Description of Artificial Sequence Synthetic polypeptide
EVQLVESGGGLVKPGGSLRVSCAASGFTFSSYAMSWVRLAPGKGLEWVATISSAGGYIFYSDSVQGRFTISRDNAKNSLYLQMNSLRAEDTAMYYCARQGFGNYGDYYAMDYWGQGTLVTVSSGGGGSGGGGSGGGGSDIELTQSPSSLAVSAGERVTMNCKSSQSLLNSRTRKNQLAWYQQKPGQSPELLIYWASTRQSGVPDRFSGSGSGTDFTLTISSVQAEDVAVYYCQQSYNLLTFGPGTKLEIKRTSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKHHHHHH

SEQ ID NO: 84 - protein, 507 aa, Artificial Sequence
  Description of Artificial Sequence Synthetic polypeptide
EVQLVESGGGLVKPGGSLRVSCAASGFTFSSYAMSWVRLAPGKGLEWVATISSAGGYIFYSDSVQGRFTISRDNAKNSLYLQMNSLRAEDTAMYYCARQGFGNYGDYYAMDYWGQGTLVTVSSGGGGSGGGGSGGGGSDIVLTQSPDSLAVSLGERVTMNCKSSQSLLNSRTRKNQLAWYQQKPGQSPELLIYWASTRQSGVPDRFSGSGSGTDFTLTISSVQAEDVAVYYCQQSYNLLTFGQGTKLEIKRTSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKHHHHHH

SEQ ID NO: 85 - protein, 507 aa, Artificial Sequence
  Description of Artificial Sequence Synthetic polypeptide
QVTLKESGPGILQPTQTLTLTCTFSGFSLSTVGMGVGWSRQPSGKGLEWLAHIWWDDEDKYYNPALKSRLTITKDTSKNQVFLKITNVDTADTATYYCTRIGTAQATDALDYWGQGTLVTVSSGGGGSGGGGSGGGGSDIVMTQSAPSVPVTPGESVSISCRSSKSLLHSNGNTYLYWFLQKPGQSPQRLIYYMSNLASGVPDRFSGRGSGTDFTLKISRVEAEDVGVYYCMQSLEYPLTFGGGTKLEIKRTSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKHHHHHH

SEQ ID NO: 86 - protein, 507 aa, Artificial Sequence
  Description of Artificial Sequence Synthetic polypeptide
QVTLKESGPGILQPTQTLTLTCTFSGFSLSTVGMGVGWSRQPSGKGLEWLAHIWWDDEDKYYNPALKSRLTITKDTSKNQVFLKITNVDTADTATYYCTRIGTAQATDALDYWGQGTLVTVSSGGGGSGGGGSGGGGSDIVMTQSALSLPVTPGEPVSISCRSSKSLLHSNGNTYLYWFLQKPGQSPQRLIYYMSNLASGVPDRFSGSGSGTDFTLKISRVEAEDVGVYYCMQSLEYPLTFGGGTKLEIKRTSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKHHHHHH

SEQ ID NO: 87 - protein, 507 aa, Artificial Sequence
  Description of Artificial Sequence Synthetic polypeptide
QVTLKESGPTLVKPTQTLTLTCTFSGFSLSTVGMGVGWSRQPSGKGLEWLAHIWWDDEDKYYNPALKSRLTITKDTSKNQVVLTITNVDPVDTATYYCTRIGTAQATDALDYWGQGTLVTVSSGGGGSGGGGSGGGGSDIVMTQSAPSVPVTPGESVSISCRSSKSLLHSNGNTYLYWFLQKPGQSPQRLIYYMSNLASGVPDRFSGRGSGTDFTLKISRVEAEDVGVYYCMQSLEYPLTFGGGTKLEIKRTSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKHHHHHH

SEQ ID NO: 88 - protein, 507 aa, Artificial Sequence
  Description of Artificial Sequence Synthetic polypeptide
QVTLKESGPTLVKPTQTLTLTCTFSGFSLSTVGMGVGWSRQPSGKGLEWLAHIWWDDEDKYYNPALKSRLTITKDTSKNQVVLTITNVDPVDTATYYCTRIGTAQATDALDYWGQGTLVTVSSGGGGSGGGGSGGGGSDIVMTQSALSLPVTPGEPVSISCRSSKSLLHSNGNTYLYWFLQKPGQSPQRLIYYMSNLASGVPDRFSGSGSGTDFTLKISRVEAEDVGVYYCMQSLEYPLTFGGGTKLEIKRTSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKHHHHHH

SEQ ID NO: 89 - protein, 500 aa, Artificial Sequence
  Description of Artificial Sequence Synthetic polypeptide
DIQLTQSPSAVSASVGDRVTITCRASQDVSKWLAWYQQKPGKAPRLLISAASGLQSWVPSRFSGSGSGTEFTLSISSLQPEDFATYYCQQANSFPWTFGQGTKVEIKRSRGGGGSGGGGSGGGGSLEMAQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARQSYITDSWGQGTLVTVSSTSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKHHHHHH

SEQ ID NO: 90 - protein, 500 aa, Artificial Sequence
  Description of Artificial Sequence Synthetic polypeptide
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARQSYITDSWGQGTLVTVSSSRGGGGSGGGGSGGGGSLEMADIQLTQSPSAVSASVGDRVTITCRASQDVSKWLAWYQQKPGKAPRLLISAASGLQSWVPSRFSGSGSGTEFTLSISSLQPEDFATYYCQQANSFPWTFGQGTKVEIKRTSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKHHHHHH

SEQ ID NO: 91 - protein, 509 aa, Artificial Sequence
  Description of Artificial Sequence Synthetic polypeptide
QVQLQQWGAGLLKPSETLSLTCAVYGGSFSGYYWSWIRQPPGKGLEWIGEINHSGSTNYNPSLKSRIIMSVDTSKRQFSLKLRSATAADTAVYYCARWSPFSYKQMYDYWGQGTLVTVSSSRGGGGSGGGGSGGGGSLEMANFMLTQPHSVSESPGKTVTISCTRSRGSIASAYVQWYQQRPGSAPITVIYEDYERPSEIPDRFSGSIDSSSNSASLTISGLKTEDEADYYCQSYDDNDHVIFGGGTKVTVLGTSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKHHHHHH

SEQ ID NO: 92 - protein, 509 aa, Artificial Sequence
  Description of Artificial Sequence Synthetic polypeptide
NFMLTQPHSVSESPGKTVTISCTRSRGSIASAYVQWYQQRPGSAPITVIYEDYERPSEIPDRFSGSIDSSSNSASLTISGLKTEDEADYYCQSYDDNDHVIFGGGTKVTVLGSRGGGGSGGGGSGGGGSLEMAQVQLQQWGAGLLKPSETLSLTCAVYGGSFSGYYWSWIRQPPGKGLEWIGEINHSGSTNYNPSLKSRIIMSVDTSKRQFSLKLRSATAADTAVYYCARWSPFSYKQMYDYWGQGTLVTVSSTSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKHHHHHH

SEQ ID NO: 93 - protein, 20 aa, Artificial Sequence
  Description of Artificial Sequence Synthetic peptide
METDTLLLWVLLLWVPGSTG

SEQ ID NO: 94 - protein, 18 aa, Artificial Sequence
  Description of Artificial Sequence Synthetic peptide
MGWSCIILFLVATATGKL

SEQ ID NO: 95 - protein, 58 aa, Artificial Sequence
  Description of Artificial Sequence Synthetic polypeptide
NFSPLARRVDRVAIYEEFLRMTRNGTQLQNFTLDRSSVLVDGYSPNRNEPLTGNSDLP

SEQ ID NO: 96 - DNA, 1563 bp, Artificial Sequence
  Description of Artificial Sequence Synthetic polynucleotide
atggaaaccgacaccctgctgctgtgggtgctgctgctgtgggtgccaggatctaccggtgacatccagttgacccagtctccatctgccgtgtctgcatctgtaggagacagagtcactatcacttgtcgggcgagtcaggatgttagcaagtggttagcctggtatcagcagaagccagggaaagcccccaggctcctgatctctgctgcatccggtctgcaaagttgggtcccatcaaggttcagcggcagtggatctgggacagaattcactctctccatcagcagcctgcagcctgaagattttgcaacttactattgtcaacaggctaatagtttcccgtggacgttcggccaagggaccaaggtggaaatcaaacgttctagaggtggtggtggtagcggcggcggcggctctggtggtggtggatccctcgagatggcccaggtgcagttacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggccgtgtattactgtgcgcgccagtcttacatcactgattcttggggtcaaggtactctggtgaccgtctcctcaactagtggcgggggaggatccgacgtgcagctggtgcagagcggagctgaagtgaagaaacctggcgcctccgtgaaggtgtcctgcaaagctagcggctataccttcacccggtacaccatgcactgggtgcgccaggcacctggacagggactggaatggatcggctacatcaacccctcccggggctacaccaactacgccgactctgtgaagggccggttcaccatcaccaccgataagtccaccagcaccgcttacatggaactgtcctccctgagatccgaggacaccgctacctactattgcgcccggtactacgacgaccactactgcctggactactggggacagggaaccacagtgaccgtgtcctctggcgagggcacctctactggatctgggggaagtggtggttctggcggcgctgacgacatcgtgctgacccagtctccagccaccctgtctctgagcccaggcgagagagctaccctgtcctgcagagcctcccagtccgtgtcctacatgaattggtatcagcagaagcctggcaaggcccctaagcggtggatctacgacacctccaaggtggcctctggcgtgccagcccggttttccggatctggctctggcaccgactactccctgaccatcaacagcctggaagccgaggacgctgccacctattactgccagcagtggtcctccaaccccctgacctttggaggcggcaccaaggtggaaatcaagcaccaccatcatcaccactaa

SEQ ID NO: 97 - protein, 520 aa, Artificial Sequence
  Description of Artificial Sequence Synthetic polypeptide
METDTLLLWVLLLWVPGSTGDIQLTQSPSAVSASVGDRVTITCRASQDVSKWLAWYQQKPGKAPRLLISAASGLQSWVPSRFSGSGSGTEFTLSISSLQPEDFATYYCQQANSFPWTFGQGTKVEIKRSRGGGGSGGGGSGGGGSLEMAQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARQSYITDSWGQGTLVTVSSTSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKHHHHHH

SEQ ID NO: 98 - protein, 75 aa, Artificial Sequence
  Description of Artificial Sequence Synthetic polypeptide
  SITE (1)..(75): This sequence may encompass 1-15 “Gly Gly Gly Gly Ser” repeating units
GGGGSGGGGSGGGGSGGGGSGGGGSGGGGSGGGGSGGGGSGGGGSGGGGSGGGGSGGGGSGGGGSGGGGSGGGGS